(19) United States
(12) Patent Application Publication
(10) Pub. No.: US 2002/0042375 A1
(43) Pub. Date: Apr. 11, 2002

(54) Method of treating cancer

(76) Inventors: David C. Heimbrook, Coopersburg, PA (US); Siu-Long Yao, West Windsor, NJ (US)

Correspondence Address: MERCK AND CO INC, P O BOX 2000, RAHWAY, NJ, 070650907

(21) Appl. No.: 09/896,245

(22) Filed: Jun. 29, 2001

Related U.S. Application Data
(60) Provisional application No. 60/216,217, filed on Jul. 5, 2000.

(51) Int. Cl. A61K 38/08; A61K 31/444; A61K 31/415; A61K 31/365; A61K 31/454

(52) U.S. Cl. 514/16; 514/473; 514/407; 514/380; 514/334; 514/336; 514/341; 514/326

(57) ABSTRACT

The present invention relates to methods of treating cancer using a combination of a compound which is a PSA conjugate and an NSAID compound, which methods comprise administering to said mammal, either sequentially in any order or simultaneously, amounts of at least two therapeutic agents selected from a group consisting of a compound which is a PSA conjugate and an NSAID compound. The invention also relates to methods of preparing such compositions.

### RELATED APPLICATION

The present patent application claims the benefit of provisional application Serial No. 60/216,217, filed Jul. 5, 2000, which was pending on the date of the filing of the present invention.

### BACKGROUND OF THE INVENTION

The present invention relates to methods of treating cancer, and more particularly cancer associated with cells that produce prostate specific antigen (PSA), which comprise administering to a patient in need thereof at least one non-steroidal anti-inflammatory agent (NSAID) and at least one conjugate, which comprises an oligopeptide that is selectively cleaved by PSA and a cytotoxic agent.

In 1999 new cases of cancer of the prostate gland were expected to be diagnosed in 179,300 men in the U.S. and 37,000 American males were expected to die from this disease (Landis, S. H. et al. CA Cancer J. Clin. 49:8-31 (1999)). Prostate cancer is the most frequently diagnosed malignancy (other than that of the skin) in U.S. men and the second leading cause of cancer-related deaths (behind lung cancer) in that group.

Prostate specific Antigen (PSA) is a single chain 33 kDa glycoprotein that is produced almost exclusively by the human prostate epithelium and occurs at levels of 0.5 to 2.0 mg/ml in human seminal fluid (Nadji, M., Taber, S. Z., Castro, A., et al. (1981) Cancer 48:1229; Papsidero, L., Kuriyama, M., Wang, M., et al. (1981). JNCI 66:37; Qui, S. D., Young, C. Y. F., Bihartz, D. L., et al. (1990), J. Urol. 144:1550; Wang, M. C., Valenzuela, L. A., Murphy, G. P., et al. (1979). Invest. Urol. 17:159). The single carbohydrate unit is attached at asparagine residue number 45 and accounts for 2 to 3 kDa of the total molecular mass. PSA is a protease with chymotrypsin-like specificity (Christensson, A., Laurell, C. B., Lilja, H. (1990). Eur. J. Biochem. 194:755-763). It has been shown that PSA is mainly responsible for dissolution of the gel structure formed at ejaculation by proteolysis of the major proteins in the sperm entrapping gel, Semenogelin I and Semenogelin II, and fibronectin (Lilja, H. (1985). J. Clin. Invest. 76:1899; Lilja, H., Oldbring, J., Rannevik, G., et al. (1987). J. Clin. Invest. 80:281; McGee, R. S., Herr, J. C. (1988). Biol. Reprod. 39:499). The PSA mediated proteolysis of the gel-forming proteins generates several soluble Semenogelin I and Semenogelin II fragments and soluble fibronectin fragments with liquefaction of the ejaculate and release of progressively motile spermatoza (Lilja, H., Laurell, C. B. (1984). Scand. J. Clin. Lab. Invest. 44:447; McGee, R. S., Herr, J. C. (1987). Biol. Reprod. 37:431). Furthermore, PSA may proteolytically degrade IGFBP-3 (insulin-like growth factor binding protein 3) allowing IGF to stimulate specifically the growth of PSA secreting cells (Cohen et al., (1992) J. Clin. Endo. & Meta. 75:1046-1053).

PSA complexed to alpha 1-antichymotrypsin is the predominant molecular form of serum PSA and may account for up to 95% of the detected serum PSA (Christensson, A., Björk, T., Nilsson, O., et al. (1993). J. Urol. 150:100-105; Lilja, H., Christensson, A., Dahlén, U. (1991). Clin. Chem. 37:1618-1625; Stenman, U. H., Leinoven, J., Alfthan, H., et al. (1991). Cancer Res. 51:222-226). The prostatic tissue (normal, benign hyperplastic, or malignant tissue) is implicated to predominantly release the mature, enzymatically active form of PSA, as this form is required for complex formation with alpha 1-antichymotrypsin (Mast, A. E., Enghild, J. J., Pizzo, S. V., et al. (1991). Biochemistry 30:1723-1730; Perlmutter, D. H., Glover, G. I., Rivetna, M., et al. (1990). Proc. Natl. Acad. Sci. USA 87:3753-3757). Therefore, in the microenvironment of prostatic PSA secreting cells the PSA is believed to be processed and secreted in its mature enzymatically active form not complexed to any inhibitory molecule. PSA also forms stable complexes with alpha 2-macroglobulin, but as this results in encapsulation of PSA and complete loss of the PSA epitopes, the in vivo significance of this complex formation is unclear. A free, noncomplexed form of PSA constitutes a minor fraction of the serum PSA (Christensson, A., Björk, T., Nilsson, O., et al. (1993). J. Urol. 150:100-105; Lilja, H., Christensson, A., Dahlén, U. (1991). Clin. Chem. 37:1618-1625). The size of this form of serum PSA is similar to that of PSA in seminal fluid (Lilja, H., Christensson, A., Dahlén, U. (1991). Clin. Chem. 37:1618-1625) but it is yet unknown as to whether the free form of serum PSA may be a zymogen; an internally cleaved, inactive form of mature PSA; or PSA manifesting enzyme activity. However, it seems unlikely that the free form of serum PSA manifests enzyme activity, since there is considerable (100 to 1000 fold) molar excess of both unreacted alpha 1-antichymotrypsin and alpha 2-macroglobulin in serum as compared with the detected serum levels of the free 33 kDa form of PSA (Christensson, A., Björk, T., Nilsson, O., et al. (1993). J. Urol. 150:100-105; Lilja, H., Christensson, A., Dahlén, U. (1991). Clin. Chem. 37:1618-1625).

Serum measurements of PSA are useful for monitoring the treatment of adenocarcinoma of the prostate (Duffy, M. S. (1989). Ann. Clin. Biochem. 26:379-387; Brawer, M. K. and Lange, P. H. (1989). Urol. Suppl. 5:11-16; Hara, M. and Kimura, H. (1989). J. Lab. Clin. Med. 113:541-548), although above normal serum concentrations of PSA have also been reported in benign prostatic hyperplasia and subsequent to surgical trauma of the prostate (Lilja, H., Christensson, A., Dahlén, U. (1991). Clin. Chem. 37:1618-1625). Prostate metastases are also known to secrete immunologically reactive PSA since serum PSA is detectable at high levels in prostatectomized patients showing widespread metatstatic prostate cancer (Ford, T. F., Butcher, D. N., Masters, R. W., et al. (1985). Brit. J. Urology 57:50-55). Therefore, a cytotoxic compound that could be activated by the proteolytic activity of PSA should be prostate cell specific as well as specific for PSA secreting prostate metastases.

Conjugates which comprise an oligopeptide which can be selectively cleaved by enzymatically active PSA attached, either directly or via a linker to a cytotoxic agent and which are useful in the treatment of prostate cancer and benign prostatic hyperplasia have been previously described (U.S. Pat. Nos. 5,599,686 and 5,866,679).

The enzyme cyclooxygenase (COX) is a key enzyme in the biosynthetic pathway leading to the formation of prostaglandins (Watkins, W. D., Peterson, M. B., and Fletcher, J. R. ed. Prostaglandins in Clinical Practice. New York: Raven, 1989 and Dewitt, D. L. Prostaglandin endoperoxide synthase: regulation and enzyme expression. Biochim. Biophys. Acta, 1083; 121-134, 1991). These prostanoids are potent biological mediators with diverse normal physiological effects and are also implicated in a variety of pathological conditions including inflammation and neoplastic transformation (Watkins, W. D., Peterson, M. B., and Fletcher, J. R. ed. Prostaglandins in Clinical Practice. New York: Raven, 1989 and Dewitt, D. L. Prostaglandin endoperoxide synthase: regulation and enzyme expression. Biochim. Biophys. Acta, 1083; 121-134, 1991 and Xie, W., Robertson, D. L., and Simmons, D. L. Mitogen-inducible prostaglandin G/H synthase: a new target for nonsteroidal anti-inflammatory drugs. Drug Dev. Res., 25; 249-265, 1992). Two isoforms of COX have been identified (Loll, P. J. and Garavito, R. M. The isoforms of cyclooxygenase: structure and function. Expert Opin. Invest. Drugs, 3; 1171-1180, 1994). COX-1 is constitutively expressed in most tissues and has been proposed to generate prostaglandins for normal physiological functions. The second isoform, COX-2 (also known as prostaglandin G/H synthase-2 or PGHS-2), is characterized by a rapid induction by a variety of stimuli, including mitogens, hormones, cytokines and growth factors (Loll, P. J. and Garavito, R. M. The isoforms of cyclooxygenase: structure and function. Expert Opin. Invest. Drugs, 3; 1171-1180, 1994 and Battistini, B., Botting, R., and Bakhle, Y. S. COX-1 and COX-2: Toward the development of more selective NSAIDS. Drug News Perspectives, 7; 501-512, 1994). In conditions such as inflammation, COX-2-derived prostaglandins may be the predominant effectors (Masferrer, J. L., Zweifel, B. S., Manning, P. T., Hauser, S. D., Leahy, K. M., Smith, W. G., Isakson, P. C., and Seibert, K. Selective inhibition of inducible cyclooxygenase 2 in vivo is anti-inflammatory and non-ulcerogenic. Proc. Natl. Acad. Sci. USA, 91; 3228-3232, 1994). Both COX-1 and COX-2 have been shown to be the target of nonsteroidal anti-inflammatory drugs (NSAIDs) (Battistini, B., Botting, R., and Bakhle, Y. S. COX-1 and COX-2: Toward the development of more selective NSAIDS. Drug News Perspectives, 7; 501-512, 1994 and O'Neill, G. P., Mancini, J. A., Kargman, S., Yergey, J., Kwan, M. Y., Falgueyret, J. -P., Abramovitz, M., Kennedy, B. P., Ouellet, M., Cromlish, W., Culp, S., Evans, J. F., Ford-Hutchinson, A. W. and Vickers, P. J. Overexpression of human prostaglandin G/H synthase-1 and -2 by recombinant vaccinia virus: inhibition by nonsteroidal anti-inflammatory drugs and biosynthesis of 15-hydroxyeicosatetraenoic acid. Mol. Pharmacol., 45; 245-254, 1994 and DeWitt, D. L., Meade, E. A., and Smith, W. L. PGH synthase isoenzyme selectivity: the potential for safer nonsteroidal anti-inflammatory drugs. Am. J. Med. (Suppl.), 95; 40S-44S, 1993). See also WO 94/14977, published Jul. 7, 1994, which discloses a method of evaluating the potency of COX-2 inhibiting agents as well as the selectivity for COX-2 over COX-1.

Elevated levels of prostaglandins have been demonstrated in various cancers including lung and colon carcinomas (McLemore, T .L., Hubbard, W. C., Litterst, C. L., Liu, M. C., Miller, S., McMahon, N. A., Eggleston, J. C., and Boyd, M. R. Profiles of prostaglandin biosynthesis in normal lung and tumor tissue from lung cancer patients. Cancer Res., 48; 3140-3147, 1988 and Rigas, B., Goldman, I. S., and Levine, L. Altered eicosanoid levels in human colon cancer. J. Lab. Clin. Med., 122; 518-523, 1993). In particular, prostaglandin levels have been shown to be elevated in benign adenomatous polyps and further increased in cancerous colon tissue, as compared to histologically normal mucosa. Since prostanoids have been shown to be immunosuppressive, they may play a role in tumor development (Earnest, D. L., Hixson, L. J., and Alberts, D. S. Piroxicam and other cyclooxygenase inhibitors: potential for cancer chemoprevention. J. Cell. Biochem., 161 (Suppl.); 156-166, 1992). Recent evidence indicates that COX-2 may play a role in the etiology of several cancers including that of the prostate. COX-2 levels, as assesssed by rt-PCR, immunoblotting and immunohistochemistry, are increased in tumor specimens obtained from patients with prostate cancer and selective COX-2 inhibitors such as NS398 and celecoxib induce apoptosis in LNCaP and PC-3 prostate cancer cells. In addition, exogenous administration of dimethylprostaglandin E2 to PC-3 and LNCaP cells caused increased proliferation in vitro. Administration of flurbiprofen in this model inhibited PC-3 growth.

Prostaglandin E₂ production has been documented in freshly excised prostate tumors and NSAIDs such as NS398, indomethacin, piroxicam, aspirin, celecoxib, ketoprofen and flurbiprofen have all been shown to induce prostate cancer cell apoptosis in vitro.

Several published and unpublished epidemiological studies have evaluated the relationship between prostate cancer risk and previous use of aspirin or other NSAIDs. Although most studies did not have sufficient power to document a statistically significant effect, effects across studies generally showed a trend towards a lower risk of prostate cancer in patients utilizing NSAIDs. Paganini-Hill evaluated the risks of an extensive array of geriatric diseases including prostate cancer in 5106 men and found a statistically non-significant reduction in risk for aspirin consumers. The Olmstead County study was a prospective cohort study which evaluated the relationship between NSAID/ASA use in 2,423 men 40-79 years of age; again, a non-statistically significant reduction was found (RR 0.79; 95% CI 0.53-1.16). Norrish et al. performed a population-based case control study (317 cases, 480 controls) and documented a non-statistically significant reduction in the risk of advanced prostate cancer (RR 0.73; 95% CI 0.50-1.07) and evaluation of prospectively acquired data from 887 men in the Baltimore Longitudinal Study on Aging (BLSA) cohort revealed similar, non-statistically significant effects (RR 0.76, 95% CI 0.48-1.22). In contrast to the aforementioned studies, however, a recent case-control study (417 cases, 420 controls) by Nelson et al. was able to demonstrate a statistically significant reduction in prostate cancer risk with use of NSAIDs/ASA (RR 0.34, 95% CI 0.23-0.58). The one study (Langman) which suggested an increase in prostate cancer risk with NSAID use evaluated the relationship between NSAID prescriptions and the risk of prostate cancer. The study, however, was unable to exclude existent cases of cancer and may have been confounded by patients utilizing NSAIDs for the treatment of pain from existing cancer.

The first epidemiological study suggesting that aspirin might reduce the risk of colorectal cancer came in 1988 in a retrospective, exploratory analysis from Melboum, Australia (Cancer, Res., 48: 4399-4404, 1988). The study found a 40 percent lower risk of incident colon cancer among persons who regularly used aspirin compared to those who used no aspirin. More recently the data of Heath, et al, suggests the possible benefit of NSAIDs for prevention of colorectal neoplasms (Heath, C. W., Jr., Thun, M. J., Greenberg, E. R., Levin, B., and Marnett, L. J. Nonsteroidal anti-inflammatory drugs and human cancer. Cancer, 74; 2885-2888, 1994).

It is the object of the instant invention to provide a method for treating cancer, and more particularly cancer associated with cells that produce prostate specific antigen (PSA), which offers advantages over previously disclosed methods of treatment.

### SUMMARY OF THE INVENTION

A method of treating cancer, and more particularly cancer associated with cells that produce prostate specific antigen (PSA), is disclosed which is comprised of administering to a patient in need of such treatment amounts of at least one non-steroidal anti-inflammatory agent (NSAID) and at least one conjugate, which comprises an oligopeptide that is selectively cleaved by PSA and a cytotoxic agent.

### DETAILED DESCRIPTION OF THE INVENTION

The present invention relates to a method of treating cancer, and more particularly cancer associated with cells that produce prostate specific antigen (PSA), which is comprised of administering to a patient in need of such treatment amounts of at least one non-steroidal anti-inflammatory agent (NSAID) and at least one conjugate (hereinafter referred to as a PSA conjugate), which comprises an oligopeptide that is selectively cleaved by PSA and a cytotoxic agent. Such a combination of an NSAID and a PSA conjugate may also be useful in treating prostatic diseases in general, including prostatic cancer, benign prostatic hyperplasia and prostatic intraepithelial neoplasia.

In practicing the instant method of treatment, it is understood that the NSAID(s) and the PSA conjugate(s) may be administered either simultaneously in a single pharmaceutical composition or individually in separate pharmaceutical compositions. If the NSAID(s) and the PSA conjugate(s) are administered in separate compositions, such compositions may be administered simultaneously or consecutively.

The term “consecutively” when used in the context of administration of two or more separate pharmaceutical compositions means that administrations of the separate pharmaceutical compositions are at separate times. The term “consecutively” also includes administration of two or more separate pharmaceutical compositions wherein administration of one or more pharmaceutical compositions is a continuous administration over a prolonged period of time and wherein administration of another of the compositions occur at a discrete time during the prolonged period.

For purposes of this specification, such NSAIDs includes, but are not limited to aspirin, ibuprofen, INDOMETHACIN, SULINDAC, DOLOBID, DICLOFENAC, NAPROXEN, PIROXICAN, ETODOLAC, KETOPROFEN, FLURBIPROFEN, MELOXICAM, FLOSULIDE, NABUMETONE and COX-2 selective inhibiting agents.

In one genus of this embodiment, this invention is directed to the use of NSAID's which are potent COX-2 inhibiting agents. For purposes of this specification an NSAID is potent if it possess an IC₅₀ for the inhibition of COX-2 of 1 μM or less as measured by the cell or microsomal assay disclosed herein.

In one subgenus of this genus the invention is directed to the use of NSAIDS which are selective inhibitors of COX-2. For purposes of this specification NSAIDS which are selective inhibitors of COX-2 is defined as those which possess a specificity for inhibiting COX-2 over COX-1 of at least 100 fold as measured by the ratio of IC₅₀ for COX-2 over IC₅₀ for COX-1 evaluated by the cell or micromsal assay disclosed hereinunder. Such compounds include, but are not limited to those disclosed in U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995 , U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, U.S. Pat. No. 5,710,140, issued Jan. 20, 1998, WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999, all of which are hereby incorporated by reference.

The instant method of treatment also comprises a PSA conjugate. The PSA conjugate comprises an oligopeptide, which is specifically recognized by the free prostate specific antigen (PSA) and are capable of being proteolytically cleaved by the enzymatic activity of the free prostate specific antigen, covalently bonded directly, or through a chemical linker, to a cytotoxic agent. Ideally, the cytotoxic activity of the cytotoxic agent is greatly reduced or absent when the oligopeptide containing the PSA proteolytic cleavage site is bonded directly, or through a chemical linker, to the cytotoxic agent and is intact. Also ideally, the cytotoxic activity of the cytotoxic agent increases significantly or returns to the activity of the unmodified cytotoxic agent upon proteolytic cleavage of the attached oligopeptide at the cleavage site. While it is not necessary for practicing this aspect of the invention, a preferred embodiment of this aspect of the invention is a conjugate wherein the oligopeptide, and the chemical linker if present, are detached from the cytotoxic agent by the proteolytic activity of the free PSA and any other native proteolytic enzymes present in the tissue proximity, thereby releasing unmodified cytotoxic agent into the physiological environment at the place of proteolytic cleavage. Pharmaceutically acceptable salts of the conjugates are also included.

Oligopeptides that are selectively cleaved by enzymatically active PSA can be identified by a number of assays, in particularly the assays described in the Biological Assays section in the Examples.

In one embodiment of the instant invention, the oligopeptide component of the PSA conjugate incorporates a cyclic amino acid having a hydrophilic substituent as part of the oligopeptides, said cyclic amino acid which contributes to the aqueous solubility of the conjugate. Examples of such hydrophilic cyclic amino acids include but are not limited to hydroxylated, polyhydroxylated and alkoxylated proline and pipecolic acid moieties.

In a prefered embodiment of the invention the oligopeptide component of the PSA conjugate is characterized by having a protecting group on the terminus amino acid moiety that is not attached to the cytotoxic agent. Such protection of the terminal amino acid reduces or eliminates the enzymatic degradation of such peptidyl therapeutic agents by the action of exogenous aminopeptidases and carboxypeptidases which are present in the blood plasma of warm blooded animals. Examples of protecting groups that may be attached to the amino moiety of an N-terminus oligopeptide include, but are not limited to acetyl, benzoyl, pivaloyl, succinyl, glutaryl, hydoxyalkanoyl, polyhydroxyalkanoyl, polyethylene glycol (PEG) containing alkanoyl and the like. Examples of protecting groups that may be attached to the carboxylic acid of a C-terminus oligopeptide include, but are not limited to, formation of an organic or inorganic ester of the carboxylic acid, such as an alkyl, aralkyl, aryl, polyether ester, phosphoryl and sulfuryl, or conversion of the carboxylic acid moiety to a substituted or unsubstituted amide moiety. The N-terminus or C-terminus of the oligopeptide may also be substituted with a unnatural amino acid, such as P-alanine, or a D-amino acid, such as a D-valyl or D-alanyl group.

It is understood that the oligopeptide which is conjugated to the cytotoxic agent, whether through a direct covalent bond or through a chemical linker, does not need to be the oligopeptide that has the greatest recognition by free PSA and is most readily proteolytically cleaved by free PSA. Thus, the oligopeptide that is selected for incorporation in such conjugate will be chosen both for its selective, proteolytic cleavage by free PSA and for the cytotoxic activity of the cytotoxic agent-proteolytic residue conjugate (or, in what is felt to be an ideal situation, the unmodified cytotoxic agent) which results from such a cleavage.

Because the PSA conjugates useful in the instant compositions can be used for modifying a given biological response, cytotoxic agent component of the PSA conjugate is not to be construed as limited to classical chemical therapeutic agents. For example, the cytotoxic agent may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, b-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

The preferred cytotoxic agents include, in general, alkylating agents, antiproliferative agents, tubulin binding agents and the like. Preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, doxorubicin, carminomycin, daunorubicin, aminopterin, methotrexate, methopterin, dichloro-methotrexate, mitomycin C, porfiromycin, 5-fluorouracil, 6-mercaptopurine, cytosine arabinoside, podophyllotoxin, or podo-phyllotoxin derivatives such as etoposide or etoposide phosphate, melphalan, vinblastine, vincristine, leurosidine, vindesine, leurosine and the like. Other useful cytotoxic agents include estramustine, cisplatin and cyclophosphamide. One skilled in the art may make chemical modifications to the desired cytotoxic agent in order to make reactions of that compound more convenient for purposes of preparing PSA conjugates of the invention.

Preferably the cytotoxic agent component of the PSA conjugate is selected from a member of a class of cytotoxic agents selected from the vinca alkaloid drugs and the anthracyclines.

A pharmaceutical composition which is useful for the treatments of the instant invention may comprise one or more NSAIDs, one or more PSA conjugates, or a combination thereof, preferably, in combination with pharmaceutically acceptable carriers, excipients or diluents, according to standard pharmaceutical practice. Preferably the pharmaceutical composition comprises one NSAID and one PSA conjugate. The composition may be administered to mammals, preferably humans. The composition can be administered orally or parenterally, including the intravenous, intramuscular, intraperitoneal, subcutaneous, rectal and topical routes of administration.

The pharmaceutical compositions containing the active ingredients may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions useful in the instant methods of treatment may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavouring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The instant compositions may also be administered in the form of a suppositories for rectal administration of the drug. These compositions can be prepared by mixing the instant composition with a suitable non-irritating excipient which is solid at ordinary temperatures but liquid at the rectal temperature and will therefore melt in the rectum to release the composition. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the combination of NSAID(s) and PSA conjugate(s) are employed. (For purposes of this application, topical application shall include mouth washes and gargles.)

The compositions useful in the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

As used herein, the term “composition” is intended to encompass a product comprising the specified ingredients in the specific amounts, as well as any product which results, directly or indirectly, from combination of the specific ingredients in the specified amounts.

The composition of an NSAID(s), a PSA conjugate(s), or a combination thereof useful in the instant methods of treatment may also be co-administered with other well known therapeutic agents that are selected for their particular usefulness against the condition that is being treated.

The instant method of treatment may also be combined with surgical treatment (such as surgical removal of tumor and/or prostatic tissue) where appropriate.

If formulated as a fixed dose, the compositions useful in the instant invention employ the NSAID(s) and the PSA conjugate(s) within within the dosage ranges described below.

When compositions according to this invention are administered into a human subject, the daily dosage will normally be determined by the prescribing physician with the dosage generally varying according to the age, weight, and response of the individual patient, as well as the severity of the patient's symptoms.

In one exemplary application, a suitable amount of an NSAID and a suitable amount of a PSA conjugate are administered to a mammal undergoing treatment for prostate cancer. Administration occurs in an amount of NSAID(s) of between about from about 0.01 mg to about 140 mg/kg of body weight per day are useful in the treatment of the above-indicated conditions, or alternatively about 0.5 mg to about 7 g per patient per day. Administration of the PSA conjugate occurs in an amount between about 10 mg/m² of body surface area to about 5 g/m² of body surface area per day, preferably between about 50 mg/m² of body surface area to about 3 g/m² of body surface area per day.

Compounds that are selective inhibitors of COX-2 and are therefore useful in the instant methods of treatment include:

a) a compound of the formula I

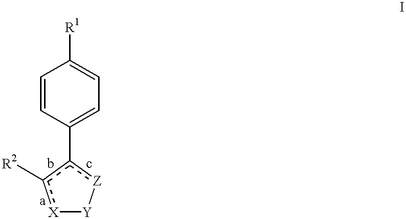

or a pharmaceutically acceptable salt thereof,

wherein: X-Y-Z-is selected from the group consisting of:

(a) —CH₂CH₂CH₂—,

(b) —C(O)CH₂CH₂—,

(c) —CH₂CH₂C(O)—,

(d) —CR⁵(R^{5′})-O—C(O)—,

(e) —C(O)—O—CR⁵(R^{5′})-,

(f) —CH₂—NR³-CH₂—,

(g) —CR⁵(R^{5′})-NR³-C(O)—,

(h) —CR⁴=CR^{4′}-S—,

(i) —S—CR⁴=CR^{4′}-,

(j) —S—N═CH—,

(k) —CH═N—S—,

(l) —N═CR⁴-O—,

(m) —O—CR⁴=N—

(n) —N═CR⁴-NH—,

(o) —N═CR⁴-S—, and

(p) —S—CR⁴=N—,

(q) —C(O)—NR³-CR⁵(R^{5′})-,

(r) —NR³-CH═CH— provided R¹ is other than —S(O)₂Me,

(s) —CH═CH—NR³- provided R¹ is other than —S(O)₂Me,when side b is a double bond, and sides a an c are single bonds; and X-Y-Z-is selected from the group consisting of:

(a) ═CH—O—CH═, and

(b) ═CH—NR³-CH═,

(c) ═N—S—CH═,

(d) ═CH—S—N═,

(e) ═N—O—CH═,

(f) ═CH—O—N═,

(g) ═N—S—N═,

(h) ═N—O—N═, when sides a and c are double bonds and side b is a single bond;R¹ is selected from the group consisting of

(a) S(O)₂CH₃,

(b) S(O)₂NH₂,

(c) S(O)₂NHC(O)CF₃,

(d) S(O)(NH)CH₃,

(e) S(O)(NH)NH₂,

(f) S(O)(NH)NHC(O)CF₃,

R² is selected from the group consisting of

(a) C₁₋₆alkyl,

(b) C₃, C₄, C₅, C₆, and C₇, cycloalkyl,

(c) mono-, di- or tri-substituted phenyl wherein the substituent is selected from the group consisting of

(1) hydrogen,

(2) halo,

(3) C₁₋₆alkoxy,

(4) C₁₋₆alkylthio,

(5) CN,

(6) CF₃,

(7) C₁₋₆alkyl,

(8) N₃,

(9) —CO₂H,

(10) —CO₂—C₁₋₄alkyl,

(11) —C(R⁵)(R⁶)-OH,

(12) —C(R⁵)(R⁶)-O—C₁₋₄alkyl, and

(13) —C₁₋₆alkyl-CO₂-R⁵;

(d) mono-, di- or tri-substituted heteroaryl wherein the heteroaryl is a monocyclic aromatic ring of 5 atoms, said ring having one hetero atom which is S, O, or N, and optionally 1, 2, or 3 additional N atoms; or the heteroaryl is a monocyclic ring of 6 atoms, said ring having one hetero atom which is N, and optionally 1, 2 or 3 additional N atoms; said substituents are selected from the group consisting of

(1) hydrogen,

(2) halo, including fluoro, chloro, bromo and iodo,

(3) C₁₋₆alkyl,

(4) C₁₋₆alkoxy,

(5) C₁₋₆alkylthio,

(6) CN,

(7) CF₃,

(8) N₃,

(9) —C(R⁵)(R⁶)-OH,

(10) —C(R⁵)(R⁶)-O—C₁₋₄alkyl;

R³ is selected from the group consisting of

(a) hydrogen,

(b) CF₃,

(c) CN,

(d) C₁₋₆alkyl,

(e) hydroxyC₁₋₆alkyl, and

(f) —C(O)—C₁₋₆alkyl,

(g) optionally substituted

(1) —C₁₋₅ alkyl-Q,

(2) —C₁₋₃alkyl-O—C₁₋₃alkyl-Q,

(3) —C₁₋₃alkyl-S—C₁₋₃alkyl-Q,

(4) —C₁₋₅ alkyl-O-Q, or

(5) —C₁₋₅ alkyl-S-Q,

wherein the substituent resides on the alkyl and the substituent is C₁₋₃alkyl;

(h) -Q

R⁴ and R^{4′} are each independently selected from the group consisting of

(a) hydrogen,

(b) CF₃,

(c) CN,

(d) C₁₋₆alkyl,

(e) -Q,

(f) —O-Q;

(g) —S-Q, and

(h) optionally substituted

(1) —C₁₋₅ alkyl-Q,

(2) —O—C₁₋₅ alkyl-Q,

(3) —S—C₁₋₅ alkyl-Q,

(4) —C₁₋₃alkyl-O—C₁₋₃alkyl-Q,

(5) —C₁₋₃alkyl-S—C₁₋₃alkyl-Q,

(6) —C₁₋₅ alkyl-O-Q,

(7) —C₁₋₅ alkyl-S-Q,

wherein the substituent resides on the alkyl and the substituent is C₁₋₃alkyl, and R⁵, R^{5′} and R⁶, R⁷ and R⁸ are each independently selected from the group consisting of

(a) hydrogen,

(b) C₁₋₆alkyl,

or R⁵ and R⁶ or R⁷ and R⁸ together with the carbon to which they are attached form a monocyclic saturated carbon ring of 3, 4, 5, 6 or 7 atoms;

Q is CO₂H, CO₂-C₁₋₄alkyl, tetrazolyl-5-yl, C(R⁷)(R⁸)(OH), or C(R⁷)(R⁸)(O—C₁₋₄alkyl);

provided that when X-Y-Z is —S—CR⁴=CR^{4′}, then R⁴ and R^{4′} are other than CF₃;

b) a compound of the formula II

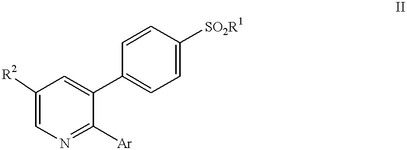

or a pharmaceutically acceptable salt thereof,

wherein:

R¹ is selected from the group consisting of

(a) CH₃,

(b) NH₂,

(c) NHC(O)CF₃,

(d) NHCH₃;

Ar is a mono-, di-, or trisubstituted phenyl or pyridinyl (or the N-oxide thereof), wherein the substituents are chosen from the group consisting of

(a) hydrogen,

(b) halo,

(c) C₁₋₆alkoxy,

(d) C₁₋₆alkylthio,

(e) CN,

(f) C₁₋₆alkyl,

(g) C₁₋₆fluoroalkyl,

(h) N₃,

(i) —CO₂R³,

(j) hydroxy,

(k) —C(R⁴)(R⁵)-OH,

(l) —C₁₋₆alkyl-CO₂-R⁶,

(m) C₁₋₆fluoroalkoxy;

R² is chosen from the group consisting of

(a) halo,

(b) C₁₋₆alkoxy,

(c) C₁₋₆alkylthio,

(d) CN,

(e) C₁₋₆alkyl,

(f) C₁₋₆fluoroalkyl,

(g) N₃,

(h) —CO₂R⁷,

(i) hydroxy,

(j) —C(R⁸)(R⁹)-OH,

(k) —C₁₋₆alkyl-CO₂-R¹⁰,

(l) C₁₋₆fluoroalkoxy,

(m) NO₂,

(n) NR¹¹R¹², and

(o) NHCOR¹³,

R³, R⁴, R⁵, R⁶, R⁷, R⁸, R⁹, R¹⁰, R¹¹, R¹², R¹³, are each independantly chosen from the group consisting of

(a) hydrogen, and

(b) C₁₋₆alkyl,

or R⁴ and R⁵, R⁸ and R⁹ or R¹¹ and R¹² together with the atom to which they are attached form a saturated monocyclic ring of 3, 4, 5, 6 or 7 atoms;

(c) a compound of the formula III:

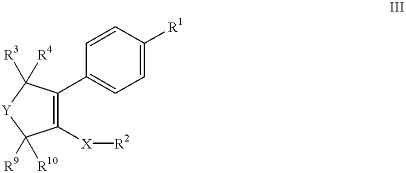

or a pharmaceutically acceptable salt thereof,

wherein:

X is selected from the group consisting of

(a) CH₂,

(b) CHOH,

(c) CO,

(d) O,

(e) S, and

(f) N(R¹⁵), with the proviso that when R³ and R⁴ are other than

(1) both hydrogen,

(2) both C₁₋₁₀ alkyl, or

(3) joined together with the carbon to which they are attached form a saturated monocyclic carbon ring of 3, 4, 5, 6 or 7 atoms, then

X is selected from CO, O, S or N(R¹⁵);

Y is selected from the group consisting of

(a) C(R¹¹)(R¹²),

(b) CO,

(c) O, and

(d) S;

R¹ is selected from the group consisting of

(a) SO₂CH₃,

(b) SO₂NR¹⁶R¹⁷,

(c) SO₂NHC(O)CF₃,

(d) S(O)(NH)NH₂,

(e) S(O)(NH)NHC(O)CF₃,

(f) P(O)(CH₃)NH₂, and

(g) P(O)(CH₃)₂,

R² is selected from the group consisting of

(a) C₁₋₁₀alkyl,

(b) mono-, di- or tri-substituted phenyl or naphthyl wherein the substituents are selected from the group consisting of

(1) hydrogen,

(2) halo,

(3) C₁₋₁₀alkoxy,

(4) C₁₋₁₀alkylthio,

(5) CN,

(6) C₁₋₆ fluoroalkyl

(7) C₁₋₁₀ alkyl,

(8) N₃,

(9) —CO₂H,

(10) —CO₂—C₁₋₁₀alkyl,

(11) —C(R⁵)(R⁶)-OH,

(12) —C(R⁵)(R⁶)-O—C₁₋₄alkyl, and

(13) —C₁₋₆alkyl-CO₂-R⁵,

(14) benzyloxy,

(15) —O—(C₁₋₆alkyl)-CO₂R⁵, and

(16) —O—(C₁₋₆alkyl)-NR⁵R⁶,

(c) mono- , di- or tri-substituted heteroaryl wherein the heteroaryl is a monocyclic aromatic ring of 5 atoms, said ring having one hetero atom which is S, O, or N, and optionally 1, 2, or 3 additional N atoms; or the heteroaryl is a monocyclic ring of 6 atoms, said ring having one hetero atom which is N, and optionally 1, 2, or 3 additional N atoms, wherein the substituents are selected from the group consisting of

(1) hydrogen,

(2) halo,

(3) C₁₋₁₀alkyl,

(4) C₁₋₁₀alkoxy,

(5) C₁₋₁₀alkylthio,

(6) CN,

(7) CF₃,

(8) N₃,

(9) —C(R⁵)(R⁶)-OH,

(10) —C(R⁵)(R⁶)-O—C₁₋₁₀alkyl, and

(11) C₁₋₆fluoroalkyl;

(d) a mono- or di- substituted benzoheterocycle in which the heterocycle is a 5, 6, or 7-membered ring which may contain 1 or 2 heteroatoms chosen independently from O, S, or N and which may contain a carbonyl group or a sulfonyl group; wherein the substituents are selected from the group consisting of

(1) hydrogen,

(2) halo,

(3) C₁₋₁₀alkyl,

(4) C₁₋₁₀alkoxy,

(5) C₁₋₁₀alkylthio,

(6) CN,

(7) CF₃,

(8) N₃,

(9) —C(R⁵)(R⁶)-OH,

(10) —C(R⁵)(R⁶)-O—C₁₋₁₀alkyl, and

(11) C₁₋₆fluoroalkyl;

(e) a heterocycloalkyl group of 5, 6 or 7 members which contains 1 or 2 heteroatoms chosen from O, S, or N and optionally contains a carbonyl group or a sulfonyl group.

(f) a mono- or di- substituted benzocarbocycle in which the carbocycle is a 5, 6, or 7-membered ring which optionally contains a carbonyl group, wherein the substituents are selected from the group consisting of

(1) hydrogen,

(2) halo,

(3) C₁₋₁₀alkyl,

(4) C₁₋₁₀alkoxy,

(5) C₁₋₁₀alkylthio,

(6) CN,

(7) CF₃,

(8) N₃,

(9) —C(R⁵)(R⁶)-OH,

(10) —C(R⁵)(R⁶)-O—C₁₋₁₀alkyl, and

(11) C₁₋₆fluoroalkyl;

(g) a mono- or di-substituted bicyclic heteroaryl of 8, 9, or 10 members, containing 2 to 5 heteroatoms chosen independently from O, S or N, and in which each ring contains at least one heteroatom, wherein the substituents are selected from the group consisting of

(1) hydrogen,

(2) halo,

(3) C₁₋₁₀alkyl,

(4) C₁₋₁₀alkoxy,

(5) C₁₋₁₀alkylthio,

(6) CN,

(7) CF₃,

(8) N₃,

(9) —C(R⁵)(R⁶)-OH,

(10) —C(R⁵)(R⁶)-O—C₁₋₁₀alkyl, and

(11) C₁₋₆fluoroalkyl;

R³ is hydrogen, C₁₋₁₀ alkyl, CH₂OR⁷, CN, CH₂CN, C₁₋₆fluoroalkyl, F, CON(R⁷)₂, mono- or di-substituted phenyl, mono or di-substituted benzyl, mono- or di-substituted heteroaryl, mono or di-substituted heteroarylmethyl, wherein the substituents are selected from the group consisting of

(1) hydrogen,

(2) halo,

(3) C₁₋₆alkyl,

(4) C₁₋₆alkoxy,

(5) C₁₋₆alkylthio,

(6) CN,

(7) CF₃,

(8) N₃,

(9) —C(R⁵)(R⁶)-OH,

(10) —C(R⁵)(R⁶)-O—C₁₋₄alkyl, and

(11) C₁₋₆fluoroalkyl;

R⁴ is

(a) hydrogen

(b) C₁₋₁₀alkyl,

(c) C₁₋₁₀alkoxy,

(d) C₁₋₁₀alkylthio,

(e) —OH,

(f) —OCOR⁷,

(g) —SH,

(h) —SCOR⁷,

(i) —OCO₂R⁸,

(j) —SCO₂R⁸,

(k) OCON(R⁷)₂,

(l) SCON(R⁷)₂, and

(m) C₁₋₆fluoroalkyl;

or R³ andR⁴ together with the carbon to which they are attached form a saturated monocyclic carbon ring of 3, 4, 5, 6 or 7 atoms; R⁵ and R⁶ are each independently selected from the group consisting of

(a) hydrogen, and

(b) C₁₋₁₀alkyl, or R⁵ and R⁶ together with the atom to which they are attached form a saturated monocyclic ring of 3, 4, 5, 6 or 7 atoms; each R⁷ is independently selected from the group consisting of

(a) hydrogen,

(b) C₁₋₆alkyl,

(c) phenyl or monosubstituted phenyl wherein the substituents may be halo, C₁₋₆alkyl, C₁₋₆alkoxy, C₁₋₆alkylthio, CN, or CF₃, and

(d) benzyl or monosubstituted benzyl wherein the substituents may be halo, C₁₋₆alkyl, C₁₋₆alkoxy, C₁₋₆alkylthio, CN, or CF₃, or two R₇ groups taken together with the nitrogen to which they are attached form a saturated monocyclic ring of 5, 6 or 7 atoms, optionally containing an additional O, S or NR⁵;each R⁸ is independently selected from the group consisting of

(a) C₁₋₆alkyl,

(b) phenyl or monosubstituted phenyl wherein the substituents may be halo, C₁₋₆alkyl, C₁₋₆alkoxy, C₁₋₆alkylthio, CN, or CF₃, and

(c) benzyl or monosubstituted benzyl wherein the substituents may be halo, C₁₋₆alkyl, C₁₋₆alkoxy, C₁₋₆alkylthio, CN, or CF₃;

R⁹ and R¹⁰ are independently selected from the group consisting of:

(a) hydrogen, and

(b) C₁₋₇alkyl, or

R⁹ and R¹⁰ together with the carbon atom to which they are attached form a carbonyl or thiocarbonyl group; R¹¹ and R¹² are independently

(a) hydrogen,

(b) mono- or di-substituted phenyl or mono- or di-substituted benzyl or mono- or di-substituted heteroaryl or mono- or di-substituted heteroarylmethyl, wherein the substituents are selected from the group consisting of

(1) hydrogen,

(2) fluoro, chloro, bromo and iodo,

(3) C₁₋₆alkyl,

(4) C₁₋₆alkoxy,

(5) C₁₋₆alkylthio,

(6) CN,

(7) CF₃,

(8) N₃,

(9) —C(R¹³)(R¹⁴)-OH,

(10) —C(R¹³)(R¹⁴)-O—C₁₋₄alkyl, and

(11) C₁₋₆fluoroalkyl, or

(c) C₁₋₇alkyl, CH₂OR⁷, CN, CH₂CN, C₁₋₆fluoroalkyl, CON(R⁷)₂, F, or OR⁷; or

R¹¹ and R¹² together with the carbon to which they are attached form a saturated monocyclic carbon ring of 3, 4, 5, 6 or 7 atoms; R¹³ and R¹⁴ are independently selected from the group consisting of:

(a) hydrogen,

(b) C₁₋₇alkyl, or

R¹³ and R¹⁴ together with the carbon to which they are attached form a carbonyl, —C(═S)—, or a saturated monocyclic carbon ring of 3, 4, 5, 6, or 7 atoms. R¹⁵ is selected from the group consisting of:

(a) hydrogen,

(b) C₁₋₁₀alkyl,

(c) mono-, di- or tri-substituted phenyl or naphthyl wherein the substituents are selected from the group consisting of

(1) hydrogen,

(2) halo,

(3) C₁₋₁₀alkoxy,

(4) C₁₋₁₀alkylthio,

(5) CN,

(6) C₁₋₆ fluoroalkyl

(7) C₁₋₁₀alkyl,

(8) N₃,

(9) —CO₂H,

(10) —CO₂—C₁₋₁₀alkyl,

(11) —C(R⁵)(R⁶)-OH,

(12) —C(R⁵)(R⁶)-O—C₁₋₄alkyl, and

(13) —C₁₋₆alkyl-CO₂-R⁵;

(14) benzyloxy,

(15) —O—(C₁₋₆alkyl)-CO₂R⁵, and

(16) —O—(C₁₋₆alkyl)-NR⁵R⁶,

(d) mono- , di- or tri-substituted heteroaryl wherein the heteroaryl is a monocyclic aromatic ring of 5 atoms, said ring having one hetero atom which is S, O, or N, and optionally 1, 2, or 3 additional N atoms; or the heteroaryl is a monocyclic ring of 6 atoms, said ring having one hetero atom which is N, and optionally 1, 2, or 3 additional N atoms, wherein the substituents are selected from the group consisting of

(1) hydrogen,

(2) halo,

(3) C₁₋₁₀alkyl,

(4) C₁₋₁₀alkoxy,

(5) C₁₋₁₀alkylthio,

(6) CN,

(7) CF₃,

(8) N₃,

(9) —C(R⁵)(R⁶)-OH,

(10) —C(R⁵)(R⁶)-O—C₁₋₁₀alkyl, and

(11) C₁₋₆fluoroalkyl;

(e) a mono- or di- substituted benzoheterocycle in which the heterocycle is a 5, 6, or 7-membered ring which may contain 1 or 2 heteroatoms chosen independently from O, S, or N and which may contain a carbonyl group or a sulfonyl group; wherein the substituents are selected from the group consisting of

(1) hydrogen,

(2) halo,

(3) C₁₋₁₀alkyl,

(4) C₁₋₁₀alkoxy,

(5) C₁₋₁₀alkylthio,

(6) CN,

(7) CF₃,

(8) N₃,

(9) —C(R⁵)(R⁶)-OH,

(10) —C(R⁵)(R⁶)-O—C₁₋₁₀alkyl, and

(11) C₁₋₆fluoroalkyl;

(f) a heterocycloalkyl group of 5, 6 or 7 members which contains 1 or 2 heteroatoms chosen from O, S, or N and optionally contains a carbonyl group or a sulfonyl group.

(g) a mono- or di- substituted benzocarbocycle in which the carbocycle is a 5, 6, or 7-membered ring which optionally contains a carbonyl group, wherein the substituents are selected from the group consisting of

(1) halo,

(3) C₁₋₁₀alkyl,

(4) C₁₋₁₀alkoxy,

(5) C₁₋₁₀alkylthio,

(6) CN,

(7) CF₃,

(8) N₃,

(9) —C(R⁵)(R⁶)-OH,

(10) —C(R⁵)(R⁶)-O—C₁₋₄alkyl, and

(11) C₁₋₆fluoroalkyl;

R¹⁶ and R¹⁷ are independently selected from the group consisting of

(a) hydrogen

(b) C₁₋₁₀alkyl,

(c) C₁₋₁₀alkanoic acid,

(d) C₁₋₁₀alkyl amine,

(e) phenyl or monosubstituted phenyl wherein the substituents are halo, C₁₋₁₀alkyl, C₁₋₁₀alkoxy, C₁₋₁₀alkylthio, C₁₋₁₀alkanoic acid, C₁₋₁₀alkylamine, CN, CO₂H or CF₃, and

(f) benzyl or monosubstituted benzyl wherein the substituents are halo, C₁₋₁₀alkyl, C₁₋₁₀alkoxy, C₁₋₁₀alkylthio, C₁₋₁₀alkanoic acid, C₁₋₁₀alkylamine, CN, COOH or CF₃, orR¹⁶ and R¹⁷ together with the nitrogen to which they are attached form a saturated monocyclic ring of 5, 6 or 7 atoms, optionally containing an additional O, S or NR⁵.

Examples of specific inhibitors of COX-2 include the following:

3-(3-Fluorophenyl)-4-(4-(methylsulfonyl)phenyl)-2-(5H)-furanone,

3-(3,4-Difluorophenyl)-4-(4-(methylsulfonyl)phenyl)-2-(5H)-furanone,

3-(3,4-Dichlorophenyl)-4-(4-(methylsulfonyl)phenyl)-2-(5H)-furanone,

3-phenyl-4-(4-(methylsulfonyl)phenyl)-2-(5H)-furanone;

5,5-Dimethyl-3-(3-fluorophenyl)-4-(methylsulfonyl)phenyl)-2-(5H)-furanone;

3-(4-Methylsulfonyl)phenyl-2-phenyl-5-trifluoromethylpyridine;

2-(3-Chlorophenyl)-3-(4-methylsulfonyl)phenyl-5-trifluoromethyl-pyridine;

2-(4-Chlorophenyl)-3-(4-methylsulfonyl)phenyl-5-trifluoromethyl-pyridine;

2-(4-Fluorophenyl)-3-(4-methylsulfonyl)phenyl-5-trifluoromethyl-pyridine;

3-(4-methylsulfonyl)phenyl-2-(3-pyridinyl)-5-trifluoromethylpyridine;

5-Methyl-3-(4-methylsulfonyl)phenyl-2-phenylpyridine;

2-(4-Chlorophenyl)-5-Methyl-3-(4-methylsulfonyl)phenylpyridine;

5-Methyl-3-(4-methylsulfonyl)phenyl-2-(3-pyridinyl) pyridine;

5-Chloro-2-(4-chlorophenyl)-3-(4-methylsulfonyl) phenylpyridine;

5-Chloro-3-(4-methylsulfonyl)phenyl-2-(2-pyridinyl) pyridine;

5-Chloro-3-(4-methylsulfonyl)phenyl-2-(3-pyridinyl) pyridine;

5-Chloro-3-(4-methylsulfonyl)phenyl-2-(4-pyridinyl) pyridine;

5-Chloro-3-(4-methylsulfonyl)phenyl-2-(2-methyl-5-pyridinyl)pyridine;

2-(4-Chlorophenyl)-3-(4-methylsulfonyl)phenylpyridinyl-5-carboxylic acid methyl ester;

2-(4-Chlorophenyl)-3-(4-methylsulfonyl)phenylpyridinyl-5-carboxylic acid;

5-Cyano-2-(4-chlorophenyl)-3-(4-methylsulfonyl) phenylpyridine;

5-Chloro-3-(4-methylsulfonyl)phenyl-2-(3-pyridyl)pyridine hydromethanesulfonate;

5-Chloro-3-(4-methylsulfonyl)phenyl-2-(3-pyridyl)pyridine hydrochloride;

5-Chloro-3-(4-methylsulfonyl)phenyl-2-(2-methyl-5-pyridinyl)pyridine Hydrochloride;

5-Chloro-3-(4-methylsulfonyl)phenyl-2-(2-ethyl-5-pyridinyl)pyridine;

5-Chloro-3-(4-methylsulfonyl)phenyl-2-(2-ethyl-5-pyridinyl)pyridine hydromethanesulfonate;

3-(3,4-Difluorophenoxy)-5,5-dimethyl-4-(4-(methylsulfonyl)phenyl)-5H-furan-2-one,

3-(3-Fluorophenoxy)-5,5-dimethyl-4-(4-(methylsulfonyl)phenyl)- 5H-furan-2-one,

3-(3,5-Difluorophenoxy)-5,5-dimethyl-4-(methylsulfonyl) phenyl)- 5H-furan-2-one,

3-Phenoxy-5,5-dimethyl-4-(4-(methylsulfonyl)phenyl)-5H-furan-2-one,(5) 3-(2,4-Difluorophenoxy)-5, 5-dimethyl-4-(4-(methylsulfonyl)phenyl)-5H-furan-2-one,

3-(4-Chlorophenoxy)-5,5-dimethyl-4-(4-(methylsulfonyl) phenyl)- 5H-furan-2-one,

3-(3,4-Dichlorophenoxy)-5,5-dimethyl-4-(methylsulfonyl) phenyl)-5H-furan-2-one,

3-(4-Fluorophenoxy)-5,5-dimethyl-4-(4-(methylsulfonyl) phenyl)-5H-furan-2-one,

3-(4-Fluorophenylthio)-5,5-dimethyl-4-(4-(methylsulfonyl)phenyl)-5H-furan-2-one,

3-(3,5-Difluorophenylthio)-5,5-dimethyl-4-(4-(methylsulfonyl)phenyl)-5H-furan-2-one,

3-Phenylthio-5,5-dimethyl-4-(4-(methylsulfonyl)phenyl)-5H-furan-2-one,

3-(N-Phenylamino)-5,5-dimethyl-4-(4-(methylsulfonyl) phenyl)-5H-furan-2-one,

3-(N-Methyl-N-phenylamino)-5,5-dimethyl-4-(4-(methylsulfonyl)phenyl)-5H-furan-2-one,

3-Cyclohexyloxy-5,5-dimethyl-4-(4-(methylsulfonyl)phenyl)-5H-furan-2-one,

3-Phenylthio-4-(4-(methylsulfonyl)phenyl)-5H-furan-2-one,

3-Benzyl-5,5-dimethyl-4-(4-(methylsulfonyl)phenyl)-5H-furan-2-one,

3-(3,4-Difluorophenylhydroxymethyl)-5,5-dimethyl-4-(4-(methylsulfonyl)phenyl)-5H-furan-2-one,

3-(3,4-Difluorobenzoyl)-5,5-dimethyl-4-(4-(methylsulfonyl)phenyl)-5H-furan-2-one,

3-Benzoyl-5,5-dimethyl-4-(4-(methylsulfonyl)phenyl)-5H-furan-2-one,

4-(4-(Methylsulfonyl)phenyl)-3-phenoxy-1-oxaspiro[4.4]non-3-en- 2-one,

4-(4-(Methylsulfonyl)phenyl)-3-phenylthio-1-oxaspiro[4.4]non-3-en-2-one,

4-(2-Oxo-3-phenylthio-1-oxa-spiro[4,4]non-3-en-4-yl) benzenesulfonamide,

3-(4-Fluorobenzyl)-5,5-dimethyl-4-(4-(methylsulfonyl) phenyl)-5H-furan-2-one,

3-(3,4-Difluorophenoxy)-5-methoxy-5-methyl-4-(4- (methylsulfonyl)phenyl)-5H-furan-2-one,

3-(5-Chloro-2-pyridyloxy)-5,5-dimethyl-4-(4-(methylsulfonyl)phenyl)-5H-furan-2-one,

3-(2-pyridyloxy)-5,5-dimethyl-4-(4-(methylsulfonyl)phenyl)-5H-furan-2-one,

3-(6-Methyl-2-pyridyloxy)-5,5-dimethyl-4-(4-(methylsulfonyl) phenyl)-5H-furan-2-one,

3-(3-Isoquinolinoxy)-5,5-dimethyl-4-(4-(methylsulfonyl)phenyl)-5H-furan-2-one,

3-(4-(Methylsulfonyl)phenyl)-2-phenoxycyclopent-2-enone,

3-(4-(Methylsulfonyl)phenyl)-2-(3,4-difluorophenoxy) cyclopent-2-enone,

(a) 5,5-Dimethyl-4-(4-methylsulfonylphenyl)-3-(5-bromopyridin-2-yloxy)-5H-furan-2-one,

(b) 5,5-Dimethyl-4-(4-methylsulfonylphenyl)-3-(2-propoxy)-5H-furan-2-one,

2-(3,4-difluorophenoxy)-3-(4-methylsulfonylphenyl)-cyclopent-2-enone,

3-(5-Benzothiophenyloxy)-5,5-dimethyl-4-(4-(methylsulfonyl) phenyl)-5H-furan-2-one,

5,5-dimethyl-4-(4-methylsulfonyl-phenyl)-3-(pyridyl-4-oxy)-5H-furan-2-one,

5,5-dimethyl-4-(4-methylsulfonyl-phenyl)-3-(pyridyl-3-oxy)-5H-furan-2-one,

3-(2-Methyl-5-pyridyloxy)-5,5-dimethyl-4-(4-(methylsulfonyl) phenyl)-5H-furan-2-one,

3(2-Fluoro-4-trifluoromethyl)phenoxy-4-(4-methylsulfonyl)phenyl)-5,5-dimethyl-5H-furan-2-one,

3-(5-Chloro-2-pyridylthio)-5,5-dimethyl-4-(4-methylsulfonyl)phenyl-5H-furan-2-one,

2-(3,5-Difluorophenoxy)-3-(4-methylsulfonylphenyl)-cyclopent-2-enone,

3-(2-Pyrimidinoxy)-5,5-dimethyl-4-(4-methylsulfonyl)phenyl-5H-furan-2-one,

3-(3-Methyl-2-pyridyloxy)-5,5-dimethyl-4-(4-methylsulfonyl)phenyl-5H-furan-2-one,

3-(3-Chloro-5-pyridyloxy)-5,5-dimethyl-4-(4-(methylsulfonyl) phenyl)-5H-furan-2-one,

3-(3-(1,2,5-Thiadiazolyl)oxy)-4-(4-(methylsulfonyl)phenyl)-5,5-dimethyl-5H-furan-2-one,

3-(5-Isoquinolinoxy)-5,5-dimethyl-4-(4-methylsulfonyl)phenyl-5H-furan-2-one,

3-(6-Amino-2-pyridyloxy)-5,5-dimethyl-4-(4-(methylsulfonyl) phenyl)-5H-furan-2-one,

3-(3-Chloro-4-fluoro)phenoxy-4-(methylsulfonyl)phenyl)-5,5-dimethyl-5H-furan-2-one,

3-(6-Quinolinoxy)-5,5-dimethyl-4-(4-(methylsulfonyl)phenyl)-5H-furan-2-one,

3-(5-Nitro-2-pyridyloxy)-5,5-dimethyl-4-(4-methylsulfonyl)phenyl-5H-furan-2-one,

3-(2-Thiazolylthio)-5,5-dimethyl-4-(4-(methylsulfonyl)phenyl)-5H-furan-2-one,

3-(3-Chloro-5-pyridyloxy)-5,5-dimethyl-4-(4-(methylsulfonyl) phenyl)-5H-furan-2-one,

5,5-Dimethyl-4-(4-methylsulfonylphenyl)-3-(2-propoxy)-5H-furan-2-one,

3-(3-Trifluoromethyl)phenoxy-4-(4-methylsulfonyl)phenyl)-5,5-dimethyl-5H-furan-2-one,

5,5-Dimethyl-(4-(4-methylsulfonyl)phenyl)-3-(piperidine- 1 -carbonyl)-5-H-furan-2-one,

5,5-Dimethyl-3-(2-Butoxy)-4-(4-methylsulfonylphenyl)-5H-furan-2-one,

5,5-Dimethyl-4-(4-methylsulfonylphenyl)-3-(3-pentoxy)-5H-furan-2-one,

2-(5-Chloro-2-pyridyloxy)-3-(4-methylsulfonyl)phenylcyclopent-2-enone,

3-(4-Methyl-2-pyridyloxy)-5,5-dimethyl-4-(4-methylsulfonyl)phenyl-5H-furan-2-one,

(5R)-3-(3,4-Difluorophenoxy)-5-ethyl-5-methyl-4-(4-methylsulfonyl)phenyl-5H-furan-2-one,

(5R)-3-(4-Chlorophenoxy)-5-ethyl-5-methyl-4-(4-methylsulfonyl)phenyl-5H-furan-2-one,

3-(2-Methyl-3-pyridyloxy)-5,5-dimethyl-4-(4-methylsulfonyl)phenyl-5H-furan-2-one,

(30) 3-(4-Methyl-5-nitro-2-pyridyloxy)-5,5-dimethyl-4-(4-methylsulfonyl)phenyl-5H-furan-2-one,

3-(5-Chloro-4-methyl-2-pyridyloxy)-5,5-dimethyl-4-(4-methylsulfonyl)phenyl-5H-furan-2-one,

3-(5-Fluoro-4-methyl-2-pyridyloxy)-5,5-dimethyl-4-(4-methylsulfonyl)phenyl-5H-furan-2-one,

3-(3-Chloro-2-pyridyloxy)-5,5-dimethyl-4-(4-methylsulfonyl)phenyl-5H-furan-2-one,

3-(4-Fluorophenoxy)-5-methyl-4-(4-methylsulfonyl)phenyl-5-propyl-5H-furan-2-one,

3-(N,N-Diethylamino)-5,5-dimethyl-4-(4-(methylsulfonyl) phenyl)-5H-furan-2-one,

5,5-dimethyl-4-(4-methylsulfonyl-phenyl)-3-(3,5-dichloro-2-pyridyloxy)-5H-furan-2-one,

(5R)-3-(4-Bromophenoxy)-5-ethyl-5-methyl-4-(4-methylsulfonyl)phenyl-5H-furan-2-one,

(5R)-3-(4-Methoxyphenoxy)-5-ethyl-5-methyl-4-(4-methylsulfonyl)phenyl-5H-furan-2-one,

(5R)-3-(5-Chloro-2-pyridyloxy)-5-methyl-4-(4-methylsulfonyl)phenyl-5-(2,2,2-trifluoroethyl)-5H-furan-2-one,

3-(5-Chloro-2-pyridyloxy)-5-methyl-4-(4-methylsulfonyl)phenyl-5-propyl-5H-furan-2-one,

3-(1-Cyclopropyl-ethoxy)-5,5-dimethyl-4-(4-methyl sulfonyl)phenyl)-5H-furan-2-one,

5-Methyl-4-(4-(methylsulfonyl)phenyl)-3-(2-(propoxy)-5-(2-trifluoroethyl)-5H-furan-2-one,

5(R)-5-ethyl-5-methyl-4-(4-(methylsulfonyl)phenyl)-3-(2-propoxy)-5H-furan-2-one,

5,5-dimethyl-3-(2,2-dimethylpropyloxy)-4-(4-(methylsulfonyl)phenyl)-5H-furan-2-one,

5(R) 3-(1-cyclopropyl-ethoxy)-5-ethyl-5-methyl-4-(4-(methyl sulfonyl)phenyl-5H-furan-2-one,

5(S) 5-Ethyl-5-methyl-4-(4-(methylsulfonyl)phenyl-3-(2-propoxy)-5H-furan-2-one,

3-(1-cyclopropylethoxy)-5,5-dimethyl-4-(4-(methylsulfonyl)phenyl)-5H-furan-2-one,

3-(1-cyclopropylethoxy)-5,5-dimethyl-4-(4-(methylsulfonyl)phenyl)-5H-furan-2-one,

5,5-dimethyl-3-(isobutoxy)-4-(4-(methylsulfonyl)phenyl)-5H-furan-2-one,

3-(4-Bromophenoxy)-5,5-dimethyl-4-(4-(methylsulfonyl)phenyl)-5H-furan-2-one,

3-(2-Quinolinoxy)-5,5-dimethyl-4-(4-(methylsulfonyl)phenyl)-5H-furan-2-one,

3-(2-Chloro-5-pyridyloxy)-5,5-dimethyl-4-(4-(methylsulfonyl)phenyl)-5H-furan-2-one,

3-(6-benzothiazolyloxy)-5,5-dimethyl-4-(4-(methylsulfonyl) phenyl)-5H-furan-2-one,

3-(6-Chloro-2-pyridyloxy)-5,5-dimethyl-4-(4-(methylsulfonyl) phenyl)-5H-furan-2-one,

3-(4-Quinazolyloxy)-5,5-dimethyl-4-(4-(methylsulfonyl)phenyl)-5H-furan-2-one,

(5R)-3-(5-Fluoro-2-pyridyloxy)-5-ethyl-5-methyl4-(4-methylsulfonyl)phenyl-5H-furan-2-one,

(5R)-3-(4-Fluorophenoxy)-5-ethyl-5-methyl-4-(4-methylsulfonyl)phenyl-5H-furan-2-one,

(5R)-3-(5-Fluoro-2-pyridyloxy)-5-methyl-4-(4-methylsulfonyl)phenyl-5-(2,2,2-trifluoroethyl)-5H-furan-2-one,

3-(1-Isoquinolinyloxy)-5,5-dimethyl-4-(methylsulfonyl)phenyl-5H-furan-2-one,

(5R)-3-(4-fluorophenoxy)-5-methyl-4-(4-methylsulfonyl)phenyl-5-(2,2,2-trifluoroethyl)-5H-furan-2-one,

3-(3-Fluoro-2-pyridyloxy)-5,5-dimethyl-4-(4-methylsulfonyl) phenyl-5H-furan-2-one,

(5R)-3-(3,4-difluorophenoxy)-5-methyl-4-(4-methylsulfonyl) phenyl-5-(2,2,2-trifluoroethyl)-5H-furan-2-one,

(5R)-3-(5-chloro-2-pyridyloxy)-5-ethyl-5-methyl-4-(4-methylsulfonyl)phenyl-5H-furan-2-one,

3-(3,4-difluorophenoxy)-5-methyl-5-trifluoromethyl-4-(4-methylsulfonyl)phenyl-5H-furan-2-one,

3-(3,4-Difluorophenoxy)-5-methyl-4-(4-(methylsulfonyl)phenyl)-5-propyl-5H-furan-2-one,

3-Cyclobutyloxy-5,5-dimethyl-4-(4-methylsulfonylphenyl-5H-furan-2-one,

3-(1-Indanyloxy)-5,5-dimethyl-4-(4-(methylsulfonyl)phenyl)-5H-furan-2-one,

3-(2-Indanyloxy)-5,5-dimethyl-4-(4-methylsulfonyl)phenyl)-5H-furan-2-one,

3-Cyclopentyloxy-5,5-dimethyl-4-(4-methylsulfonylphenyl)5H-furan-2-one,

3-(3,3-Dimethylcyclopentyloxy)-5,5-dimethyl-4-(4-methylsulfonyl-phenyl)-5H-furan-2-one,

3-Isopropoxy-5-methyl-4-(4-methylsulfonylphenyl)-5-propyl-5H-furan-2-one,

3-(2-Methoxy-5-pyridyloxy)-5,5-dimethyl-4-(4-methylsulfonyl)phenyl-5H-furan-2-one,

3-(5-Methyl-2-pyridyloxy)-5,5-dimethyl-4-(4-methylsulfonyl)phenyl-5H-furan-2-one,

(5RS)-3-(3,4-Difluorophenoxy)-5-methyl-4-(4-methylsulfonyl)phenyl-5-(2,2,2-trifluoroethyl)-5H-furan-2-one,

3-(3-Chloro-4-methoxyphenoxy)-5,5-dimethyl-4-(4-methylsulfonyl)phenyl-5H-furan-2-one,

(5R)-3-(3-Chloro-4-methoxyphenoxy)-5-ethyl-5-methyl-4-(4-methylsulfonyl)phenyl-5H-furan-2-one,

(5R)-3-(4-Chlorophenoxy)-5-trifluoroethyl-5-methyl-4-(4-methylsulfonyl)phenyl-5H-furan-2-one,

(5R)-3-(4-Bromophenoxy)-5-trifluoroethyl-5-methyl-4-(4-methylsulfonyl)phenyl-5H-furan-2-one,

5-Cyclopropylmethyl-3-(3,4-difluorophenoxy)-5-methyl-(4-methylsulfonyl)phenyl-5H-furan-2-one,

(5R)-3-(3-Fluorophenoxy)-5-ethyl-5-methyl-4-(4-methylsulfonyl)phenyl-5H-furan-2-one,

(5R)-3-(4-Chloro-3-fluorophenoxy)-5-ethyl-5-methyl-4-(4-methylsulfonyl)phenyl-5H-furan-2-one,

(5R)-3-Phenoxy-5-ethyl-5-methyl-4-(4-methylsulfonyl)phenyl-5H-furan-2-one,

(5R)-3-(4-Chloro-3-methylphenoxy)-5-ethyl-5-methyl-4-(4-methylsulfonyl)phenyl-5H-furan-2-one,

3-(4-Chloro-3-methylphenoxy)-5-5-dimethyl-4-(4-methylsulfonyl)phenyl-5H-furan-2-one,

(5R)-3-(5-bromo-2-pyridyloxy)-4-(4-methylsulfonylphenyl)-5-methyl-5-(2,2,2-trifluoroethyl)-5H-furan-2-one,

(5R)-3-(5-bromo-2-pyridyloxy)-4-(4-methylsulfonylphenyl)-5-ethyl-5-methyl-5H-furan-2-one,

3-(5-chloro-6-methyl-2-pyridyloxy)-5,5-dimethyl-4-(4-methylsulfonyl)phenyl-5H-furan-2-one,

3-(5-cyclopropyl-2-pyridyloxy)-5,5-dimethyl-4-(4-methylsulfonyl)phenyl-5H-furan-2-one,

3-(1-cyclopropylethoxy)-4-(4-methylsulfonyl)phenyl-5H-furan-2-one, and

3-(cyclopropylmethoxy)-4-(4-methylsulfonyl)phenyl-5H-furan-2-one.

or a pharmaceutically acceptable salt or optical isomer thereof.

Inhibitors of COX-2 that are particularly useful in the instant method of treatment are:

3-phenyl-4-(4-(methylsulfonyl)phenyl)-2-(5H)-furanone

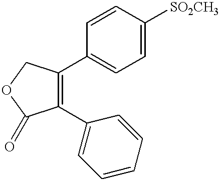

5-Chloro-3-(4-methylsulfonyl)phenyl-2-(2-methyl-5-pyridinyl)pyridine;

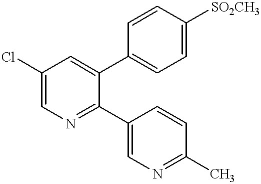

or a pharmaceutically acceptable salt thereof.

General and specific synthetic procedures for the preparation of the COX-2 inhibitor compounds described above are found in U.S. Pat. No. 5,474,995, issued Dec. 12, 1995, U.S. Pat. No. 5,861,419, issued Jan. 19, 1999, and U.S. Pat. No. 6,001,843, issued Dec. 14, 1999, all of which are herein incorporated by reference.

Compounds that have been described as specific inhibitors of COX-2 and may therefore be useful in the present invention include but are not limited to:

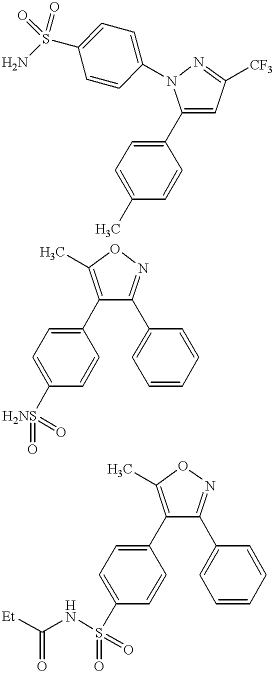

or a pharmaceutically acceptable salt thereof.

Compounds which are described as specific inhibitors of COX-2 and may therefore useful in the present invention, and methods of synthesis thereof, can be found in the following patents, pending applications and publications, which are herein incorporated by reference:

WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999.

Compounds which are specific inhibitors of COX-2 and are therefore useful in the present invention, and methods of synthesis thereof, can be found in the following patents, pending applications and publications, which are herein incorporated by reference:

U.S. Pat. No. 5,474,995, issued Dec. 12, 1995 , U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, and U.S. Pat. No. 5,710,140, issued Jan. 20,1998.

PSA conjugates that are useful in the methods of the instant invention and are identified by the properties described hereinabove include:

a) a compound represented by the formula IV:

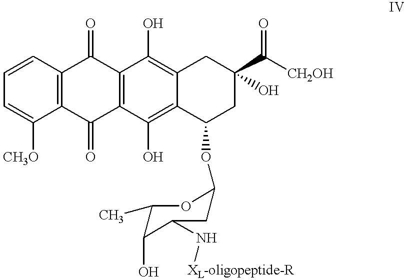

wherein:

oligopeptide is an oligopeptide which is selectively recognized by the free prostate specific antigen (PSA) and is capable of being proteolytically cleaved by the enzymatic activity of the free prostate specific antigen;

XL is absent or is an amino acid selected from:

a) phenylalanine,

b) leucine,

c) valine,

d) isoleucine,

e) (2-naphthyl)alanine,

f) cyclohexylalanine,

g) diphenylalanine,

h) norvaline, and

j) norleucine;

R is hydrogen or —(C═O)R¹; and

R¹ is C₁-C₆-alkyl or aryl,

or the pharmaceutically acceptable salt thereof;

b) a compound represented by the formula V:

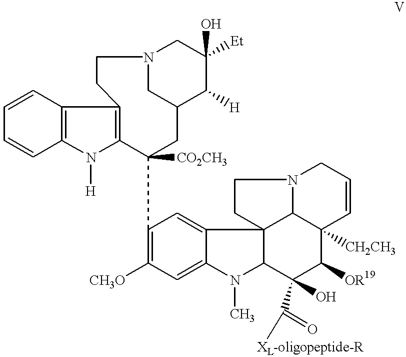

wherein:

oligopeptide is an oligopeptide which is selectively recognized by the free prostate specific antigen (PSA) and is capable of being proteolytically cleaved by the enzymatic activity of the free prostate specific antigen;

XL is absent or is an amino acid selected from:

a) phenylalanine,

b) leucine,

c) valine,

d) isoleucine,

e) (2-naphthyl)alanine,

f) cyclohexylalanine,

g) diphenylalanine,

h) norvaline, and

j) norleucine; or

X_{L} is —NH—(CH₂)ₙ—NH—

R is hydrogen or —(C═O)R¹;

R¹ is C₁-C₆-alkyl or aryl;

R¹⁹ is hydrogen or acetyl; and

n is 1, 2, 3, 4 or 5,

or the pharmaceutically acceptable salt thereof;

c) a compound represented by the formula VI:

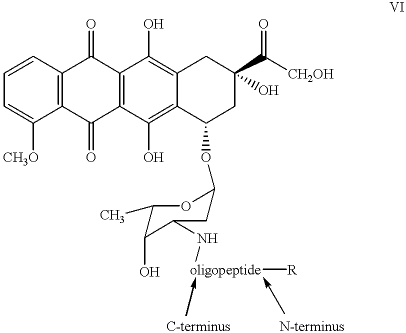

wherein:

oligopeptide is an oligopeptide which is selectively recognized by the free prostate specific antigen (PSA) and is capable of being proteolytically cleaved by the enzymatic activity of the free prostate specific antigen, wherein the oligopeptide comprises a cyclic amino acid of the formula:

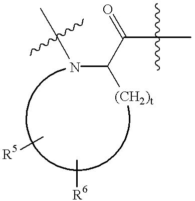

and wherein the C-terminus carbonyl is covalently bound to the amine of doxorubicin;

R is selected from

a) hydrogen,

b) —(C═O)R^{1a},

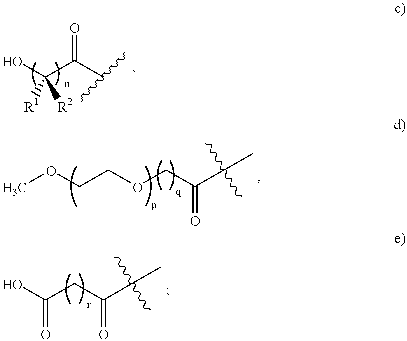

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated aryl, polyhydroxylated aryl or aryl;

R⁵ is selected from HO— and C₁-C₆ alkoxy;

R⁶ is selected from hydrogen, halogen, C₁-C₆ alkyl, HO— and C₁-C₆ alkoxy; and

n is 1, 2, 3 or 4;

p is zero or an integer between 1 and 100;

q is 0 or 1, provided that if p is zero, q is 1;

r is an integer between 1 and 10; and

t is 3 or 4;

or a pharmaceutically acceptable salt thereof;

d) a compound represented by the formula VII:

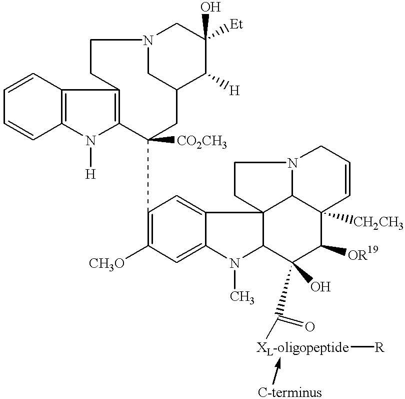

wherein:

oligopeptide is an oligopeptide which is selectively recognized by the free prostate specific antigen (PSA) and is capable of being proteolytically cleaved by the enzymatic activity of the free prostate specific antigen, and the oligopeptide comprises a cyclic amino acid of the formula:

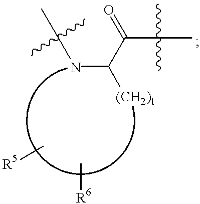

X_{L} is —NH—(CH₂)ᵤ—NH—

R is selected from

a) hydrogen,

b) —(C═O)R^{1a},

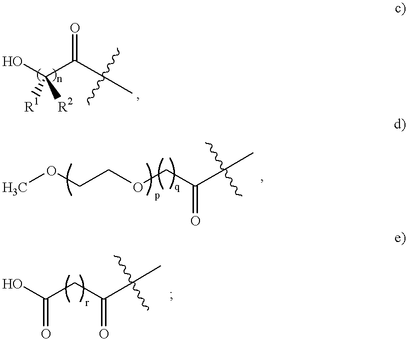

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated aryl, polyhydroxylated aryl or aryl,

R¹⁹ is hydrogen, (C₁-C₃ alkyl)-CO, or chlorosubstituted (C₁-C₃ alkyl)-CO;

n is 1, 2, 3 or 4;

p is zero or an integer between 1 and 100;

q is 0 or 1, provided that if p is zero, q is 1;

r is 1, 2 or 3;

t is 3 or 4;

u is 1, 2, 3, 4 or 5,

or the pharmaceutically acceptable salt thereof;

e) a compound represented by the formula VIII:

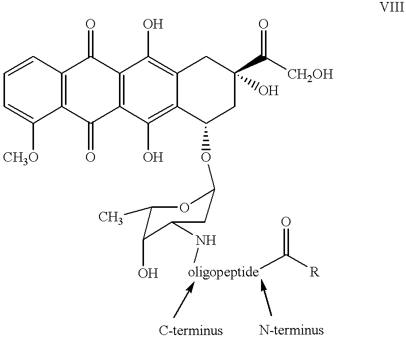

wherein:

oligopeptide is an oligopeptide which is selectively recognized by the free prostate specific antigen (PSA) and is capable of being proteolytically cleaved by the enzymatic activity of the free prostate specific antigen, and wherein the C-terminus carbonyl is covalently bound to the amine of doxorubicin and the N-terminus amine is covalently bound to the carbonyl of the blocking group;

R is selected from

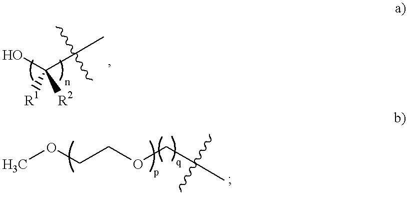

R¹ and R² are independently selected from: hydrogen, OH, C₁-C₆ alkyl, C₁-C₆ alkoxy, C₁-C₆ aralkyl and aryl;

n is 1, 2, 3 or 4;

p is zero or an integer between 1 and 100;

q is 0 or 1, provided that if p is zero, q is 1;

or the pharmaceutically acceptable salt thereof;

f) a compound represented by the formula IX:

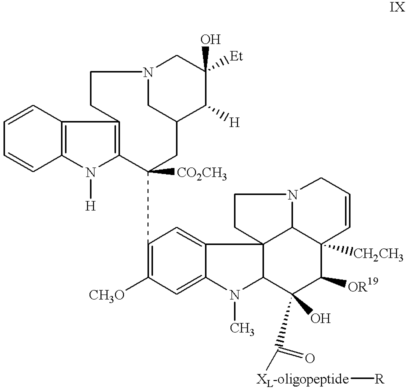

wherein:

oligopeptide is an oligopeptide which is selectively recognized by the free prostate specific antigen (PSA) and is capable of being proteolytically cleaved by the enzymatic activity of the free prostate specific antigen;

X_{L} is —NH—(CH₂)ᵣ—NH—

R is selected from

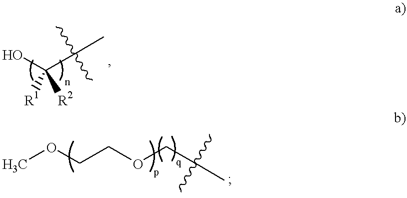

R¹ and R² are independently selected from: hydrogen, OH, C₁-C₆ alkyl, C₁-C₆ alkoxy, C₁-C₆ aralkyl and aryl;

R¹⁹ is hydrogen, (C₁-C₃ alkyl)-CO, or chlorosubstituted (C₁-C₃ alkyl)-CO;

n is 1, 2, 3 or 4;

p is zero or an integer between 1 and 100;

q is 0 or 1, provided that if p is zero, q is 1;

r is 1, 2, 3, 4 or 5,

or the pharmaceutically acceptable salt thereof;

g) a compound represented by the formula X:

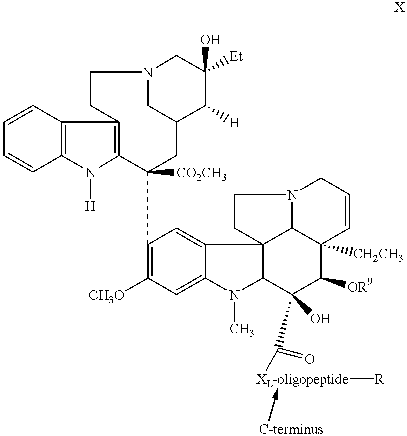

wherein:

oligopeptide is an oligopeptide which is selectively recognized by the free prostate specific antigen (PSA) and is capable of being proteolytically cleaved by the enzymatic activity of the free prostate specific antigen,

X_{L} is —NH—(CH₂)ᵤ—W—(CH₂)ᵤ—NH—

R is selected from

a) hydrogen,

b) —(C═O)R^{1a},

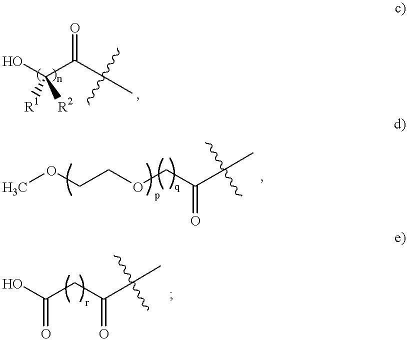

f) ethoxysquarate, and

g) cotininyl;

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;

R⁹ is hydrogen, (C₁-C₃ alkyl)-CO, or chlorosubstituted (C₁-C₃ alkyl)-CO;

W is selected from cyclopentyl, cyclohexyl, cycloheptyl or bicyclo[2.2.2]octanyl;

n is 1, 2, 3 or 4;

p is zero or an integer between 1 and 100;

q is 0 or 1, provided that if p is zero, q is 1;

r is 1, 2 or 3;

t is 3 or 4;

u is 0, 1, 2 or 3,

or the pharmaceutically acceptable salt thereof; and

h) a compound represented by the formula XI:

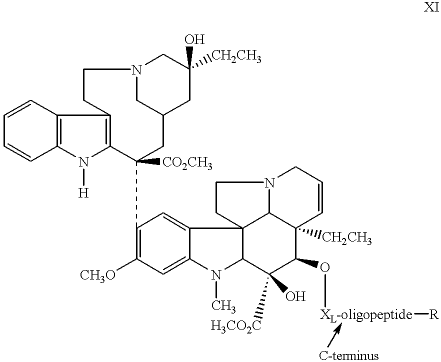

wherein:

oligopeptide is an oligopeptide which is selectively recognized by the free prostate specific antigen (PSA) and is capable of being proteolytically cleaved by the enzymatic activity of the free prostate specific antigen,

X_{L} is selected from: a bond, —C(O)—(CH₂)ᵤ—W—(CH₂)ᵤ—O— and —C(O)—(CH₂)ᵤ—W—(CH₂)ᵤ—NH—;

R is selected from

a) hydrogen,

b) —(C═O)R^{1a},

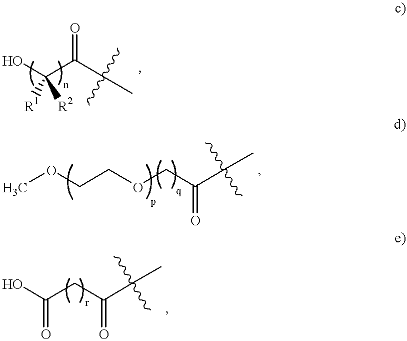

f) ethoxysquarate, and

g) cotininyl;

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;

R⁹ is hydrogen, (C₁-C₃ alkyl)-CO, or chlorosubstituted (C₁-C₃ alkyl)-CO;

W is selected from a branched or straight chain C₁-C₆-alkyl, cyclopentyl, cyclohexyl, cycloheptyl or bicyclo[2.2.2]octanyl;

n is 1, 2, 3 or 4;

p is zero or an integer between 1 and 100;

q is 0 or 1, provided that if p is zero, q is 1;

r is 1, 2 or 3;

t is 3 or 4;

u is 0, 1, 2 or 3;

or the pharmaceutically acceptable salt or optical isomer thereof.

Examples of compounds which are PSA conjugates include the following:

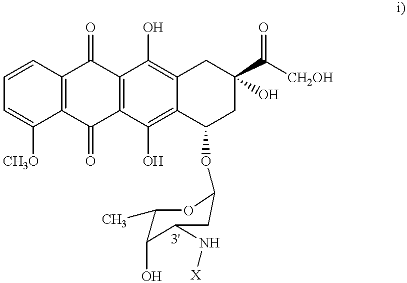

wherein X is:

AsnLysIleSerTyrGlnSer—(SEQ.ID.NO.: 1),

AsnLysIleSerTyrGlnSerSer—(SEQ.ID.NO.: 2),

AsnLysIleSerTyrGlnSerSerSer—(SEQ.ID.NO.:3 ),

AsnLysIleSerTyrGlnSerSerSerThr—(SEQ.ID.NO.:4),

AsnLysIleSerTyrGlnSerSerSerThrGlu—(SEQ.ID.NO.: 5),

AlaAsnLysIleSerTyrGlnSerSerSerThrGlu—(SEQ.ID.NO.: 6),

Ac-AlaAsnLysIleSerTyrGlnSerSerSerThr—(SEQ.ID.NO.: 7),

Ac-AlaAsnLysIleSerTyrGlnSerSerSerThrLeu—(SEQ.ID.NO.: 8),

Ac-AlaAsnLysAlaSerTyrGlnSerAlaSerThrLeu—(SEQ.ID.NO.: 9),

Ac-AlaAsnLysAlaSerTyrGlnSerAlaSerLeu—(SEQ.ID.NO.: 10),

Ac-AlaAsnLysAlaSerTyrGlnSerSerSerLeu—(SEQ.ID.NO.: 11),

Ac-AlaAsnLysAlaSerTyrGlnSerSerLeu—(SEQ.ID.NO.: 12),

Ac-SerTyrGlnSerSerSerLeu—(SEQ.ID.NO.: 13),

Ac-hArgTyrGlnSerSerSerLeu—(SEQ.ID.NO.: 14).

Ac-LysTyrGlnSerSerSerLeu—(SEQ.ID.NO.: 15),

Ac-LysTyrGlnSerSerNle—(SEQ.ID.NO.: 16),

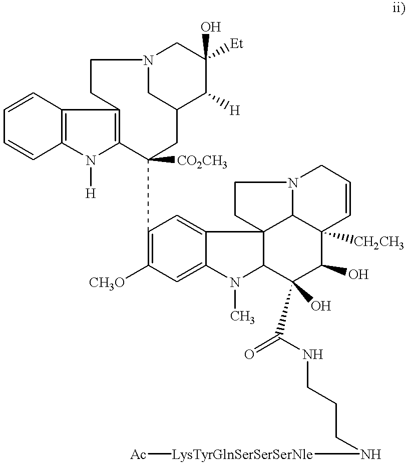

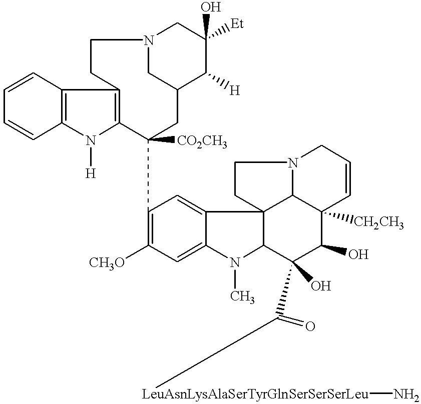

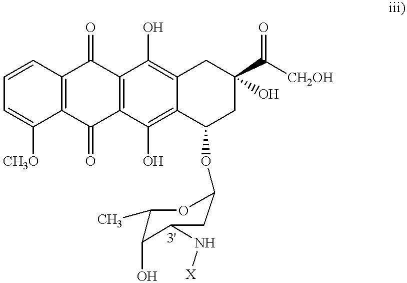

wherein X is:

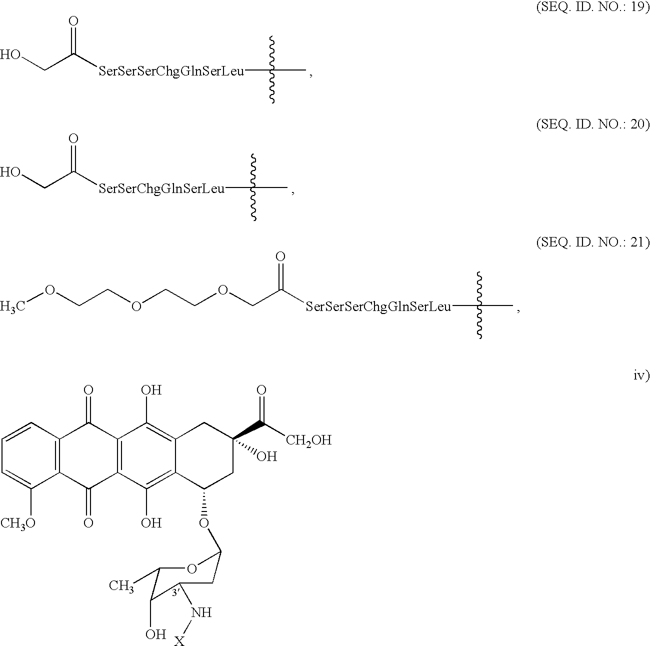

(SEQ.ID.NO.: 21),

wherein X is:

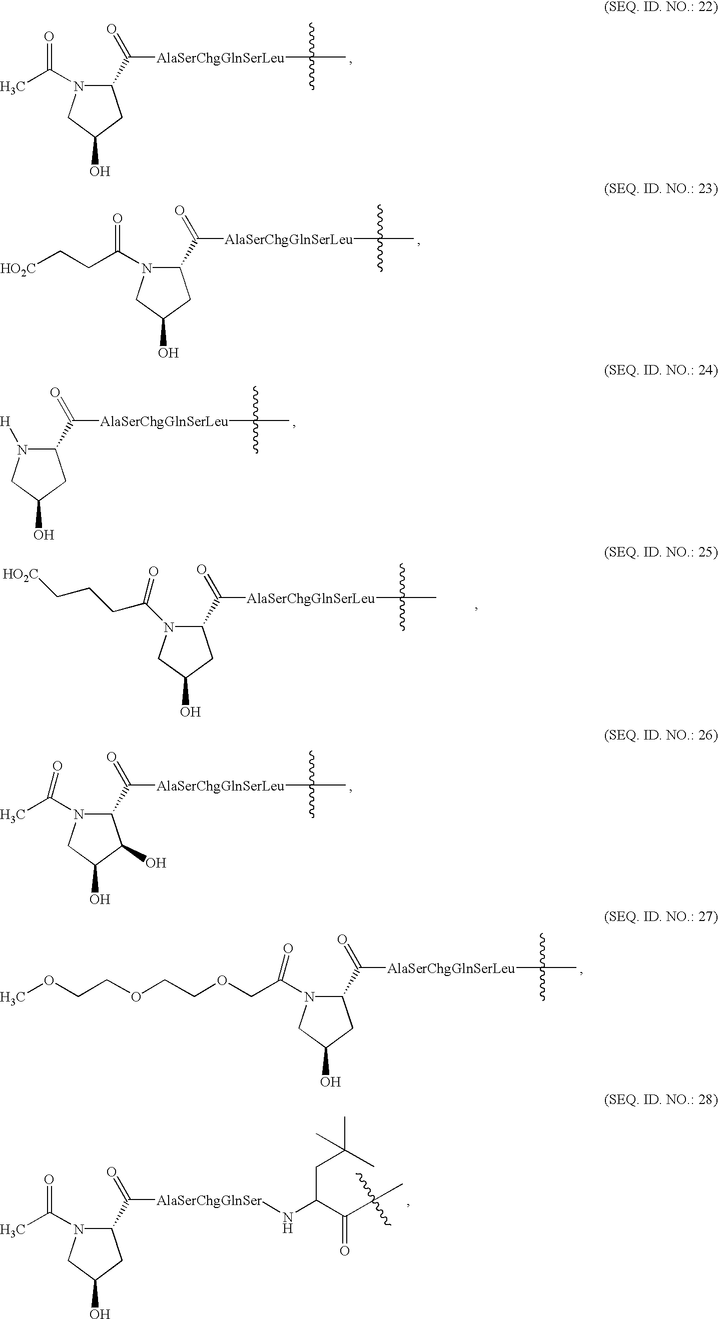

wherein X is

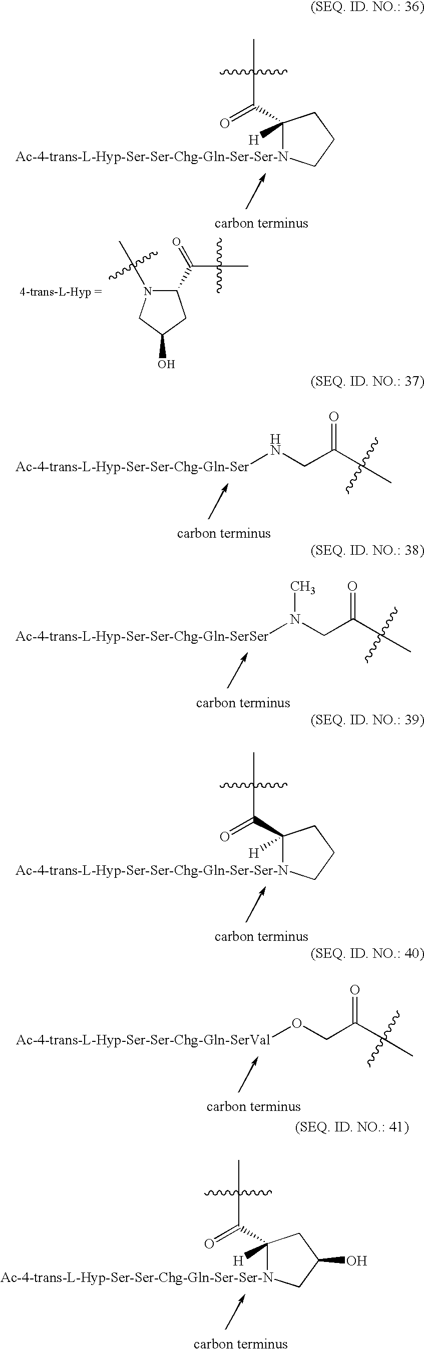

or the pharmaceutically acceptable salt or optical isomer thereof.

Preferably the method of the instant invention comprises the PSA conjugate

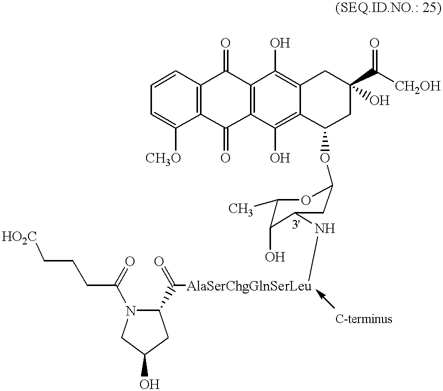

or the pharmaceutically acceptable salt thereof.

Compounds which are PSA conjugates and are therefore useful in the present invention, and methods of synthesis thereof, can be found in the following patents, pending applications and publications, which are herein incorporated by reference:

U.S. Pat. No. 5,599,686 granted on Feb. 4, 1997;

WO 96/00503 (Jan. 11, 1996); U.S. Pat. No. 6,143,864, granted on Nov. 7, 2000;

U.S. Pat. No. 5,866,679 granted on Feb. 2, 1999;

WO 98/10651 (Mar. 19, 1998); U.S. Pat. No. 5,998,362, granted on Dec. 7, 1999;

WO 98/18493 (May 7, 1998); U.S. Pat. No. 5,948,750, granted on Sep. 7, 1999

U.S. Pat. No. 6,127,333, granted on Oct. 3, 2000; and

U.S. Pat. No. 6,174,858, granted on Jan. 16, 2001.

Compounds which are described as prodrugs wherein the active therapeutic agent is release by the action of enzymatically active PSA and therefore may be useful in the present invention, and methods of synthesis thereof, can be found in the following patents, pending applications and publications, which are herein incorporated by reference:

WO 98/52966 (Nov. 26, 1998).

All patents, publications and pending patent applications identified are hereby incorporated by reference.

With respect to the compounds of formulas I to III the following definitions apply:

“Alkyl” means linear branched and cyclic structures, and combinations thereof, containing the indicated number of carbon atoms. Examples of alkyl groups include methyl, ethyl, propyl, isopropyl, butyl, s- and t-butyl, pentyl, hexyl, heptyl, octyl, nonyl, undecyl, dodecyl, tridecyl, tetradecyl, pentadecyl, eicosyl, 3,7-diethyl-2,2-dimethyl- 4-propylnonyl, cyclopropyl, cyclopentyl, cycloheptyl, adamantyl, cyclododecylmethyl, 2-ethyl-1- bicyclo[4.4.0]decyl and the like.

“Fluoro alkyl” means alkyl groups in which one or more hydrogen is replaced by fluorine. Examples are —CF₃, —CH₂CH₂F, —CH₂CF₃, c-Pr-F₅, c-Hex-F₁₁ and the like. Similarly, fluoroalkoxy means linear, branched and cyclic structures, with the indicated number of carbon atoms.

For purposes of this specification “Alkoxy” means alkoxy groups of the indicated number of carbon atoms of a straight, branched, or cyclic configuration. Examples of alkoxy groups include methoxy, ethoxy, propoxy, isopropoxy, cyclopropyloxy, cyclohexyloxy, and the like.

“Alkylthio” means alkylthio groups of the indicated number of carbon atoms of a straight, branched or cyclic configuration. Examples of alkylthio groups include methylthio, propylthio, isopropylthio, cycloheptylthio, etc. By way of illustration, the propylthio group signifies —SCH₂CH₂CH₃.

“Halo” means F, Cl, Br, or I.

Unless otherwise defined, heteroaryl includes aromatic and partially aromatic groups which contain one or more heteroatoms. Examples of this type are thiophene, purine, imidazopyridine, pyridine, oxazole, thiazole, oxazine, pyrazole, tetrazole, imidazole, pyridine, pyrimidine, pyrazine and triazine. Examples of partially aromatic groups are tetrahydro-imidazo[4,5-c]pyridine, phthalidyl and saccharinyl, as defined below.

In situations in which a term occurs two or more times, the definition of the term in each occurrence is independent of the definition in each additional occurrence.

The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like, and basic ion exchange resins.

With respect to the compounds of formulas IV through XI the following definitions apply:

As used herein, “oligopeptide” is preferably a peptide comprising from about 5 amino acids to about 100 amino acids. More preferably, “oligopeptide” is a peptide comprising from about 5 amino acids to about 15 amino acids.

The terms “selective” and “selectively” as used in connection with recognition by PSA and the proteolytic PSA cleavage mean a greater rate of cleavage of an oligopeptide component of the instant invention by free PSA relative to cleavage of an oligopeptide which comprises a random sequence of amino acids. Therefore, the oligopeptide component of the instant invention is a preferred substrate of free PSA. The terms “selective” and “selectively” also indicate that the oligopeptide is proteolytically cleaved by free PSA between two specific amino acids in the oligopeptide.

As used herein, “alkyl” and the alkyl portion of aralkyl and similar terms, is intended to include both branched and straight-chain saturated aliphatic hydrocarbon groups having the specified number of carbon atoms; “alkoxy” represents an alkyl group of indicated number of carbon atoms attached through an oxygen bridge.

As used herein, “cycloalkyl” is intended to include non-aromatic cyclic hydrocarbon groups having the specified number of carbon atoms. Examples of cycloalkyl groups include cyclopropyl, cyclobutyl, cyclopentyl, cyclohexyl and the like.

“Halogen” or “halo” as used herein means fluoro, chloro, bromo and iodo.

As used herein, “aryl,” and the aryl portion of aralkyl and aroyl, is intended to mean any stable monocyclic or bicyclic carbon ring of up to 7 members in each ring, wherein at least one ring is aromatic. Examples of such aryl elements include phenyl, naphthyl, tetrahydro-naphthyl, indanyl, biphenyl, phenanthryl, anthryl or acenaphthyl.

As used herein, the term “hydroxylated” represents substitution on a substitutable carbon of the ring system being so described by a hydroxyl moiety. As used herein, the term “poly-hydroxylated” represents substitution on two or more substitutable carbon of the ring system being so described by 2, 3 or 4 hydroxyl moieties.

As used herein, the term “chlorosubstituted C₁-C₃-alkyl-CO—” represents a acyl moiety having the designated number of carbon atoms attached to a carbonyl moiety wherein one of the carbon atoms is substituted with a chlorine. Example of such chlorosubstituted elements include but are not limited to chloroacetyl, 2-chloropropionyl, 3-chloropropionyl and 2-chlorobutyroyl.

As used herein, the term “PEG” represents certain polyethylene glycol containing substituents having the designated number of ethyleneoxy subunits. Thus the term PEG(2) represents

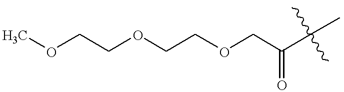

and the term PEG(6) represents

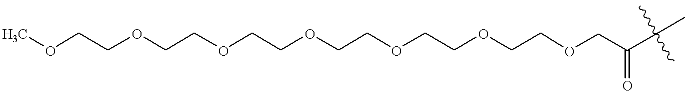

As used herein, the term “(d)(2,3-dihydroxypropionyl)” represents the following structure:

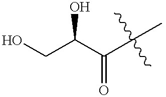

As used herein, the term “(2R,3S) 2,3,4-trihydroxybutanoyl” represents the following structure:

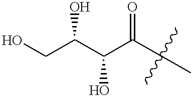

As used herein, the term “quinyl” represents the following structure:

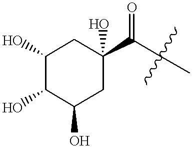

or the diastereomer thereof.

As used herein, the term “cotininyl” represents the following structure:

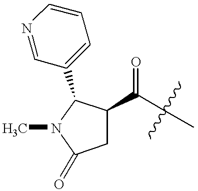

or the diastereomer thereof.

As used herein, the term “gallyl” represents the following structure:

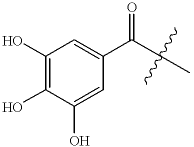

As used herein, the term “4-ethoxysquarate” represents the following structure:

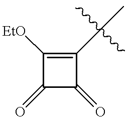

The structure

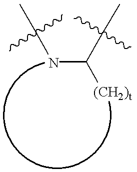

represents a cyclic amine moiety having 5 or 6 members in the ring, such a cyclic amine which may be optionally fused to a phenyl or cyclohexyl ring. Examples of such a cyclic amine moiety include, but are not limited to, the following specific structures:

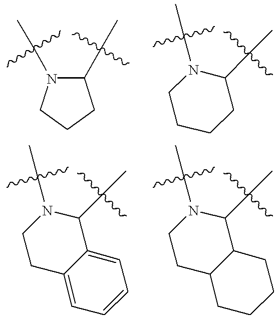

The pharmaceutically acceptable salts of the compounds of this invention include the conventional non-toxic salts of the compounds of this invention as formed, e.g., from non-toxic inorganic or organic acids. For example, such conventional non-toxic salts include those derived from inorganic acids such as hydrochloric, hydrobromic, sulfuric, sulfamic, phosphoric, nitric and the like: and the salts prepared from organic acids such as acetic, propionic, succinic, glycolic, stearic, lactic, malic, tartaric, citric, ascorbic, pamoic, maleic, hydroxymaleic, phenyl-acetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

The term “pharmaceutically acceptable salts” also refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like, and basic ion exchange resins.

It is intended that the definition of any substituent or variable (e.g., R¹⁰, Z, n, etc.) at a particular location in a molecule be independent of its definitions elsewhere in that molecule. Thus, —N(R¹⁰)₂ represents —NHH, —NHCH₃, —NHC₂H₅, etc. It is understood that substituents and substitution patterns on the compounds of the instant invention can be selected by one of ordinary skill in the art to provide compounds that are chemically stable and that can be readily synthesized by techniques known in the art as well as those methods set forth below.

The pharmaceutically acceptable salts of the compounds of this invention can be synthesized from the compounds of this invention which contain a basic moiety by conventional chemical methods. Generally, the salts are prepared by reacting the free base with stoichiometric amounts or with an excess of the desired salt-forming inorganic or organic acid in a suitable solvent or various combinations of solvents available Nₐ-Z-L-2,3-diaminopropionic acid (Fluka) as a starting material is preferred.

Abbreviations used in the description of the chemistry and in the Examples that follow are:

| | |
|---|---|
| Ac₂O | Acetic anhydride; |
| Boc | t-Butoxycarbonyl; |
| DBU | 1, 8-diazabicyclo[5.4.0]undec-7-ene; |
| DMAP | 4-Dimethylaminopyridine; |
| DME | 1, 2-Dimethoxyethane; |
| DMF | Dimethylformamide; |
| EDC | 1-(3-dimethylaminopropyl)-3-ethyl-carbodiimide-hydrochlo- |
| | ride; |
| HOBT | 1-Hydroxybenzotriazole hydrate; |
| Et₃N | Triethylamine; |
| EtOAc | Ethyl acetate; |
| FAB | Fast atom bombardment; |
| HOOBT | 3-Hydroxy-1, 2, 2-benzotriazin-4(3H)-one; |
| HPLC | High-performance liquid chromatography; |
| MCPBA | m-Chloroperoxybenzoic acid; |
| MsCl | Methanesulfonyl chloride; |
| NaHMDS | Sodium bis(trimethylsilyl)amide; |
| Py | Pyridine; |
| TFA | Trifluoroacetic acid; |
| THF | Tetrahydrofuran. |

The compounds are useful in various pharmaceutically acceptable salt forms. The term “pharmaceutically acceptable salt” refers to those salt forms which would be apparent to the pharma-ceutical chemist. i.e., those which are substantially non-toxic and which provide the desired pharmacokinetic properties, palatability, absorption, distribution, metabolism or excretion. Other factors, more practical in nature, which are also important in the selection, are cost of the raw materials, ease of crystallization, yield, stability, hygroscopicity and flowability of the resulting bulk drug. Conveniently, pharmaceutical compositions may be prepared from the active ingredients in combination with pharmaceutically acceptable carriers.

Pharmaceutically acceptable salts include conventional non-toxic salts or quarternary ammonium salts formed, e.g., from non-toxic inorganic or organic acids. Non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, sulfanilic, 2-acetoxybenzoic, fumaric, toluenesulfonic, methane-sulfonic, ethane disulfonic, oxalic, isethionic, trifluoroacetic and the like.

The pharmaceutically acceptable salts of the present invention can be synthesized by conventional chemical methods. Generally, the salts are prepared by reacting the free base or acid with stoichiometric amounts or with an excess of the desired salt-forming inorganic or organic acid or base, in a suitable solvent or solvent combination.

The inhibitors of COX-2 of formula I can be synthesized in accordance with Methods A-P, in addition to other standard manipulations such as ester hydrolysis, cleavage of protecting groups, etc., as may be known in the literature or exemplified in the experimental procedures.

Method A

The β-chlorovinylaldehyde III can be obtained from the ketone II and the Vilsmeier reagent (DMF-POCl₃) using the general method described by Weissenfels (Z. Chem. 1966, 6, 471). The thiophene compound IV is obtained from III using the general method described by Weissenfels (Z. Chem., 1973, 13, 57). The thiol compound V can be obtained after oxidation of compound IV (R^{a}=-SMe) with one equivalent of m-CPBA followed by treatment of the resulting sulfoxide with TFAA at reflux. The sulfonamide group (VI) can then be formed by the method of Kharash (J. Amer. Chem. Soc. 1951, 73, 3240). The hydrolysis of compound VI and decarboxylation with Cu bronze in quinoline provides compound VII. Compound VII (R⁴=H) can be treated with halogenating agent such as bromine in acetic acid to allow the preparation of the 5-bromothiophene (VII, R⁴=Br). When it is desired to have a nitrile group at C-5, this can be accomplished from VI via amide formation using the Weinreb methodology (Tetrahedron Letters, 1977, 4171) followed by dehydration with TFAA. The CF₃ group can be introduced at C-5 of VII via the method of Girard (J. Org. Chem. 1983, 48, 3220).

The introduction of an alkyl group at C-5 can be achieved via a Friedel-Crafts reaction on VII (R⁴=H) and an acyl chloride, Cl—CO-lower alkyl and a catalyst such as TiCl₄, followed by reduction. For R⁴=Me, this can be achieved from the ester (R⁴=CO₂Me) via a DIBAL-H reduction followed by deoxygenation using the method of Lau (J. Org. Chem. 1986, 51, 3038). Tertiary alcohols (R⁴=-C(CH₃)₂OH) can be obtained from VI and MeMgBr. These tertiary alcohols can also be deoxygenated using the method of Lau. Similarly, the thiophene IX can be prepared from ketone VIII.

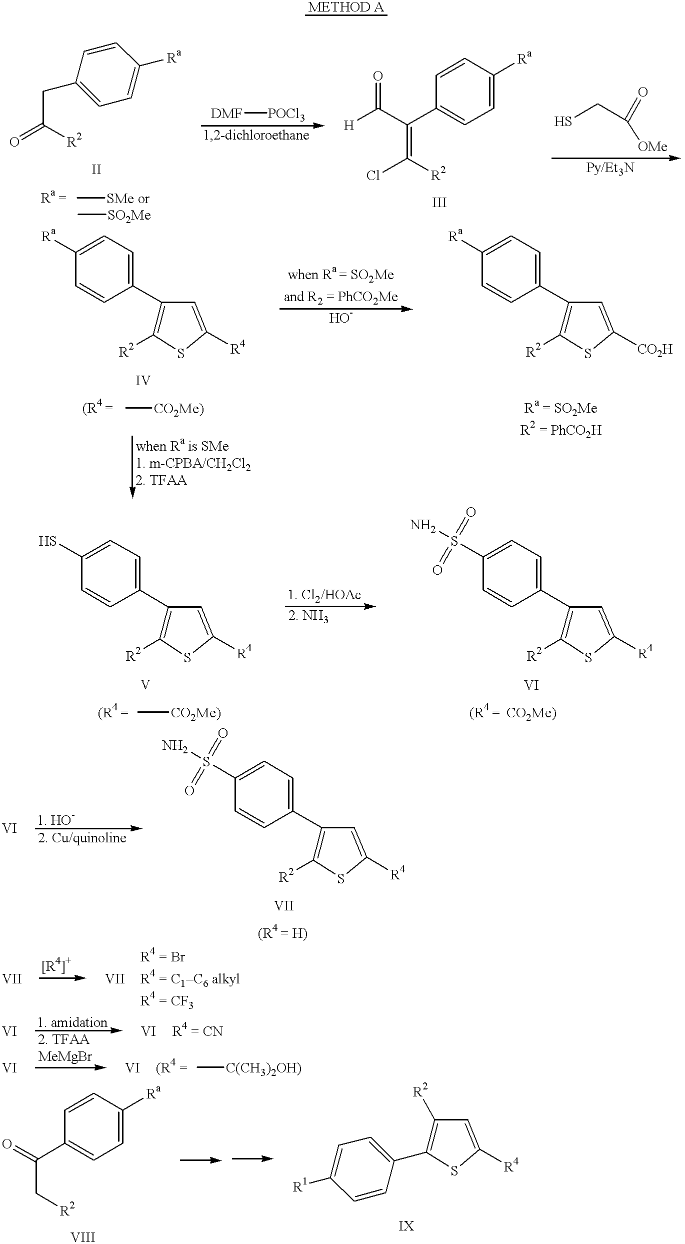

Method B

Ketone X can be converted to the thiophene compound XI using general methods already described in Method A. The thiophene XII can be prepared by metallation of XI with n-BuLi, quenching with methyl phosphonic dichloride and addition of water or ammonia (X′=OH or NH₂). Similarly, the other regioisomer XIV can be prepared from ketone XIII.

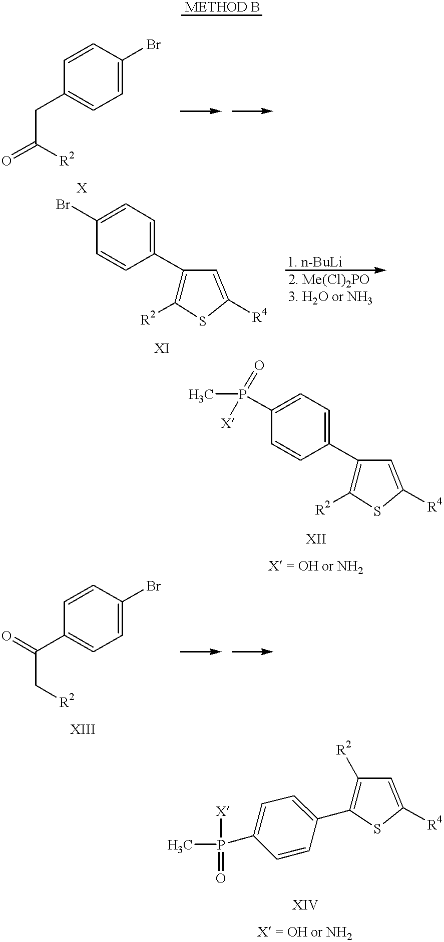

Method C

Bromination of ketone II gives the α-bromoketone XV which is then converted to the thiazole XVI after treatment with a thioamide. Similarly, ketone VIII can be converted to thiazole XVII.

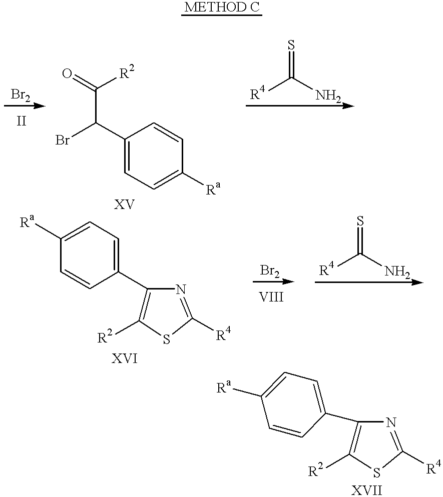

Method D:

Ketone XV can be converted to the imidazole compound XVIII after treatment witih formamide using the preparation of Brederick et al, Chem. Ber. 1953, p. 88.

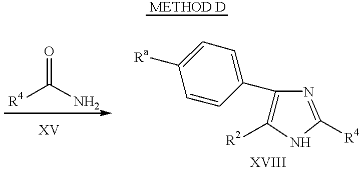

Method E

Pyrole compound XX can be obtained from diketone XIX using the general procedures of Friedman et al, J. Org. Chem. 1965, 30, p. 854, K. Dimroth et al, Ber. 1956, 56, 2602, K.Dimroth et al, Ann. 1961, 634, 102. The free NH of the pyrole can be acylated with Cl—CO-lower alkyl in the presence of a base such as Et₃N. Also alkylated products can be prepared using alkyl halides as reagents with a base such as NaH.

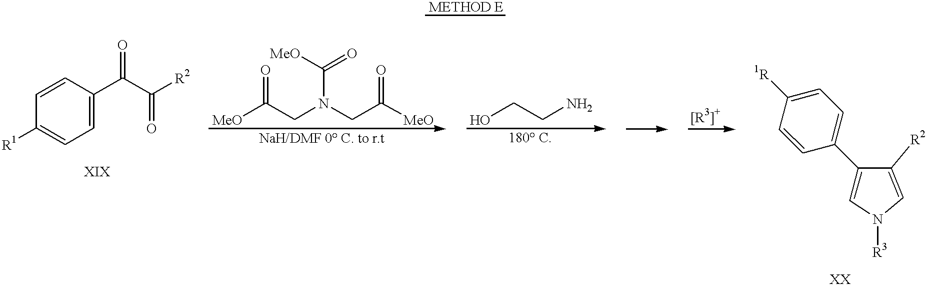

Method F

The compounds of type XXV can be prepared from readily available 4-substituted phenylacetyl chlorides XXIa. Reaction of di(3-butenyl)cadmium with a 4-substituted phenylacetyl chloride provides ketone XXI. Ozonolysis of XXI affords keto aldehyde XXIb which is cyclized by base to give cyclopentenone XXII. Addition of arylmagnesium bromide or aryllithium to XXII gives allylic alcohol XXIV. Oxidation of XXIV with pyridinium chlorochromate affords the desired 2,3-disubstituted cyclopentenone XXV. For preparation of compound XXV (R¹=SO₂Me), 4-methylthiophenyllithium is used followed by oxidation with the magesium salt of monoperoxyphthalic acid (MMPP) or m-chloroperoxybenzoic acid (mCPBA) to introduce the required methylsulfonyl group in XXV.

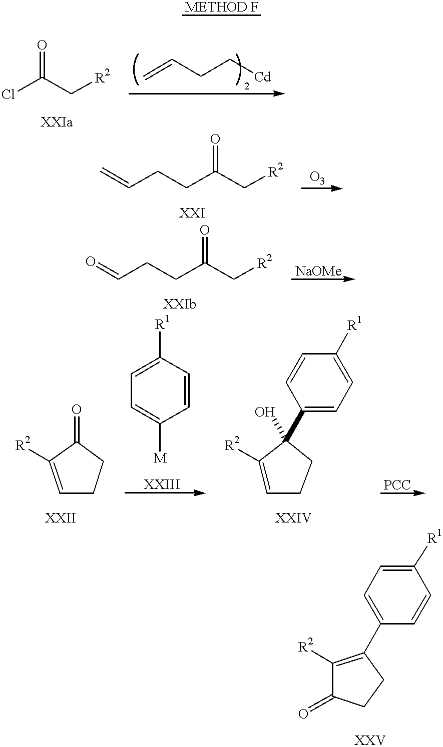

Method G

The sequence of Method G is the same as in Method F except R¹ containing acid chloride is used as starting material. R² is introduced at a later stage via a carbonyl addition reaction, followed by PCC oxidation.

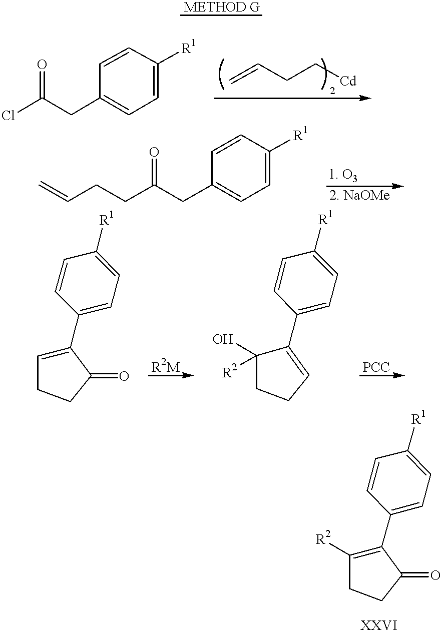

Method H

The 4,5-disubstituted isothiazoles and isothiazol-3(2H)-one-1,1-dioxides can be prepared by the general method described by B. Schulze et al, Helvetica Chimica Acta, 1991, 74, 1059. Thus, aldehyde III (R^{a}=SO₂Me) or XXVII is treated with excess NH₄SCN in refluxing acetone to provide the corresponding 4,5-disubstituted isothiazoles XXX and XXVIII, oxidation of which with hydrogen peroxide yields XXXI and XXIX.

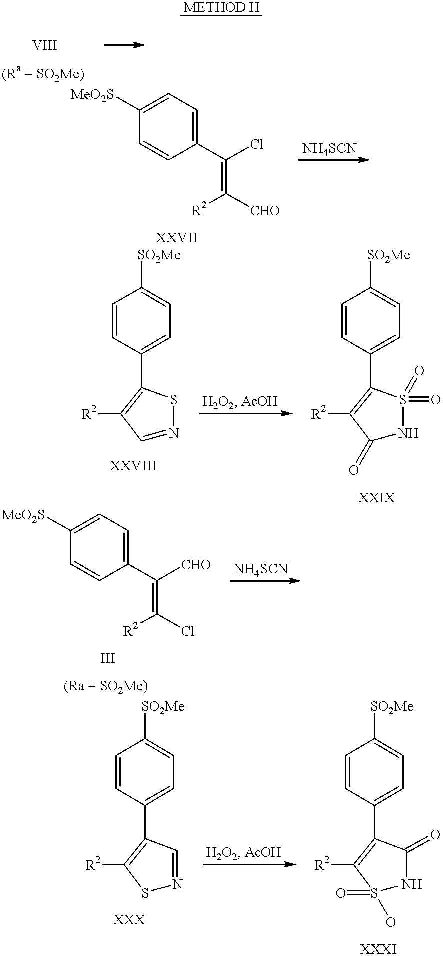

Method I

An appropriately substituted aryl bromomethyl ketone is reacted with an appropriately substituted aryl acetic acid in a solvent such as acetonitrile in the presence of a base such as triethylamine and then treated with 1,8-diazabicyclo [5.4.0]undec-7-ene (DBU) to afford either the lactone XXXIII or XXXV.

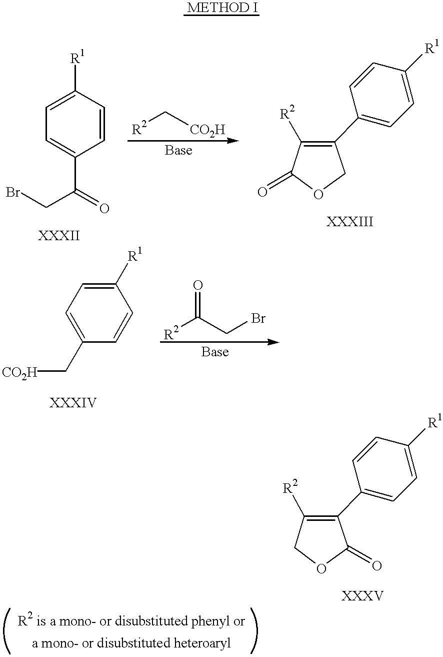

Method J

Either of the lactones XXXIII or XXXV in a solvent such as THF is reacted with a reducing agent such as diisobutyl aluminium hydride or lithium borohydride at −78° C., to yield the furan XXXVI.

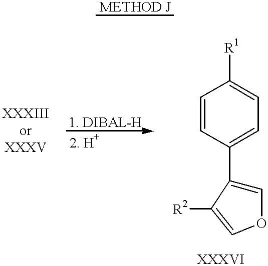

Method K

The preparation of lactams XXXVII and XXXIX can be achieved by the same reaction as described in Method I, except an appropriate amide is used.

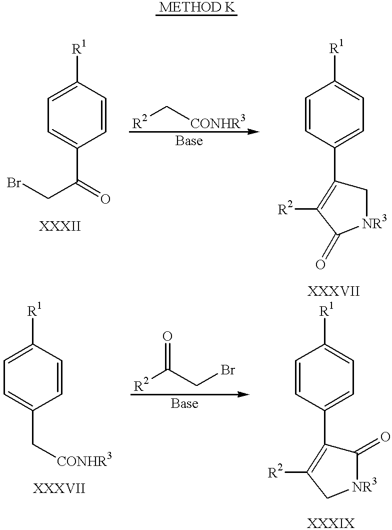

Method L

Methyl 2-hydroxy isobutyrate is silylated with TMSCI to give the TMS ether XXXXI, which is treated with 4-methylthiophenyllithium to provide ketone XXXXII. Desilylation followed by acylation yields keto-ester XXXXIV, which can be cyclized to lactone XXXXV by base catalysis. Oxidation of XXXXV with MMPP or mCPBA affords the desired product XXXXVI.

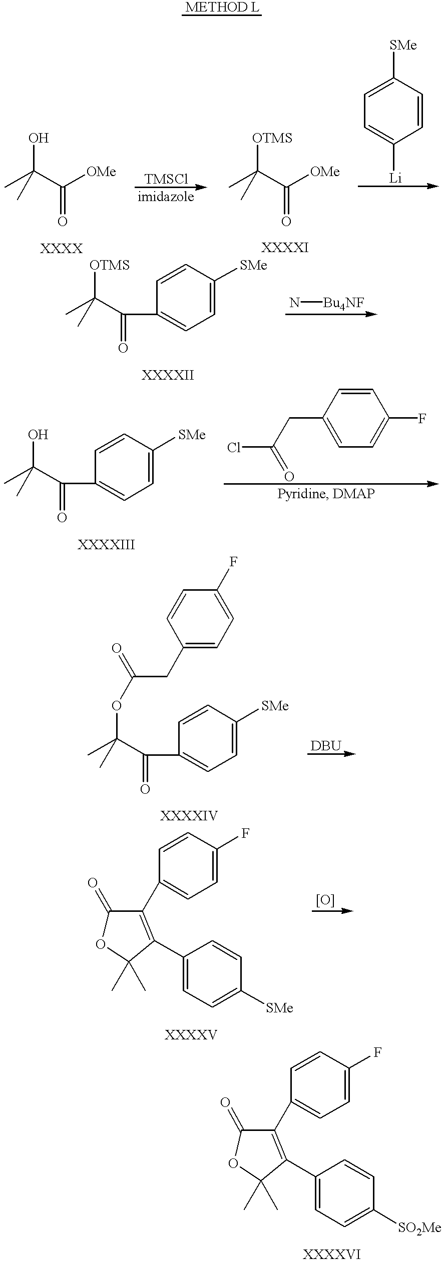

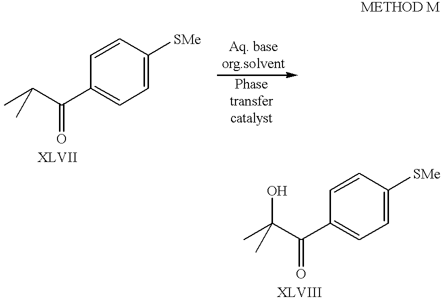

An alternative preparation of the hydroxy ketone XLIII is the oxidation of the known (J. Org. Chem. 1991 56, 5955-8; Sulfur Lett. 1991, 12, 123-32) ketone XLVII. A mixture of XLVII, aqueous base, such as NaOH, organic solvents such as carbon tetrachloride/toluene and a phase transfer catalyst such as ALIQUAT 336 is stirred in air at room temperature to provide XLIII. Compound XLIII is also described in U.S. Pat. No. 4,321,118 and Org. Coat. 1986, 6, 175-95.

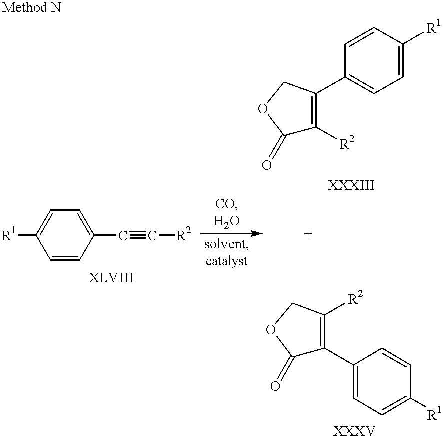

By reacting an acetylene XLVIII with carbon monoxide and water in the presence of suitable catalysts, a mixture of compound XXXIII and its isomer XXXV is obtained. The isomers are separable by standard procedures in the art such as chromatography or crystallization. Examples of useful catalysts and conditions are PdCl₂ in aqueous HCl and EtOH, heated at 50-150° C. and 50-150 atmospheres of pressure, or Rh₄ (CO)₁₂ (or Rh₆(CO)₁₆) in aqueous THF (or acetone, acetonitrile, benzene, toluene, EtOH, MeOH) containing a trialkylamine, at 50-150° C. and 20-300 atmospheres pressure. See Takahashi et al., Organomettallics 1991, 10, 2493-2498; and Tsuji et. al., J. Am. Chem. Soc. 1966, 88, 1289-1292.

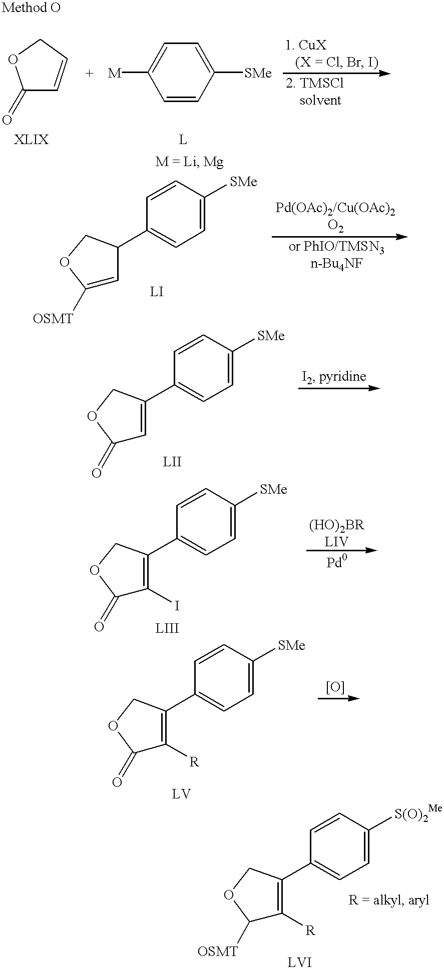

1, 4-Addition to XLIX of 4-methylthiophenyl organometallic reagents L in the presence of copper salts and the trapping of the resultant enolate with trialkyl silyl chloride such as TMSCl or TIPSCl provide the ketene acetal LI. The ketene acetal can then be oxidized to the substituted butenolide LII by the method of Ito using catalytic amounts of Pd₂(OAC)₂ and Cu(OAc)₂ and O₂ in MeOH or by the method of Magnus using PhIO/TMSN₃ and Bu₄NF. Introduction of the iodine can be accomplished by treating LII with I₂ in the presence of pyridine to afford LIII. Palladium catalyzed Susuki or Stille coupling of LIII with the appropriate aryl or alkyl partner such as the boronic acid LIV provides the butenolide LV. The sulfide can be oxidized to a sulfone by various oxidizing agents such as peracetic acid, MPPM, MMPP or H₂O₂ to give the desired compound LVI. See Y. Ito et. al., J. Am. Chem. Soc. 1979,101, 494; and P. Magnus et. al., Tet. Lett. 1992, 2933.

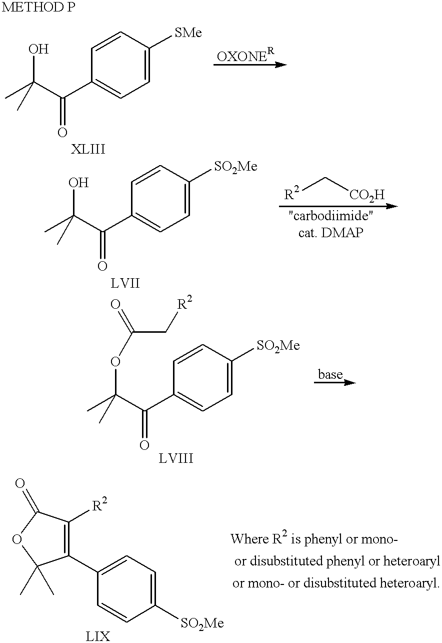

Hydroxy ketone XLIII can be oxidized to the sulfone LVII by a suitable oxidizing agent such as OXONE^{R}. By reacting the hydroxy sulfone LVII with an appropriately substituted aryl acetic acid in an inert solvent in the presence of a dehydrating agent such as a carbodiimide and a catalytic amount of DMAP, ester LVIII can be obtained. Treatment of ester LVIII with a base such as DBU in an inert solvent affords the lactone LIX.

See also WO 95/00501, published Jan. 5, 1995, which is hereby incorporated by reference.

The inhibitors of COX-2 of formula II can be synthesized in accordance with Schemes 1 and 2, in addition to other standard manipulations such as ester hydrolysis, cleavage of protecting groups, etc., as may be known in the literature or exemplified in the experimental procedures.

### Scheme 1

The pyridines of Formula Ia and Ib may be prepared in a multi-step sequence from the requisite 2-aminopyridine II. Initial bromination of II with bromine in acetic acid provides the bromide III. A palladium-catalyzed coupling of III with 4-(methylthio)phenyl-boronic acid in the presence of a suitable base, such as sodium carbonate, provides the sulfide IV which can be oxidized using one of several oxidants, such as MMPP, oxone®, or OsO₄/NMO to the corresponding sulfone V. The amino pyridine V can be converted to the halide VI via one of several methods. For example, treatment of V with sodium nitrite in the presence of HCl and bromine provides the bromide VI (X=Br). Alternatively, treatment of V with sodium nitrite and HCl followed by reaction with POCl₃ affords the corresponding chloride VI (X=Cl). A second palladium-catalyzed coupling of VI with an appropriately substituted metalated aromatic, such as an aryl boronic acid or an aryl stannane, provides the pyridine of Formula Ia. Suitable modification of the R² substituent in Ia provides additional examples of Ia. For example when R²=Me, oxidation with an oxidant such as KMnO₄ provides the corresponding acid (Ia R²=CO₂H) which can then be converted to the methyl ester (Ia R²=CO₂Me), using a reagent such as diazomethane. Alternatively, treatment of the acid with chlorosulfonylisocyanate and DMF provides the nitrile (Ia R²=CN). The pyridine methyl sulfones Ia can be converted to the corresponding pyridine sulfonamides Ib by using procedures described in the literature (Huang et. al. Tetrahedron Lett. 1994, 39, 7201).

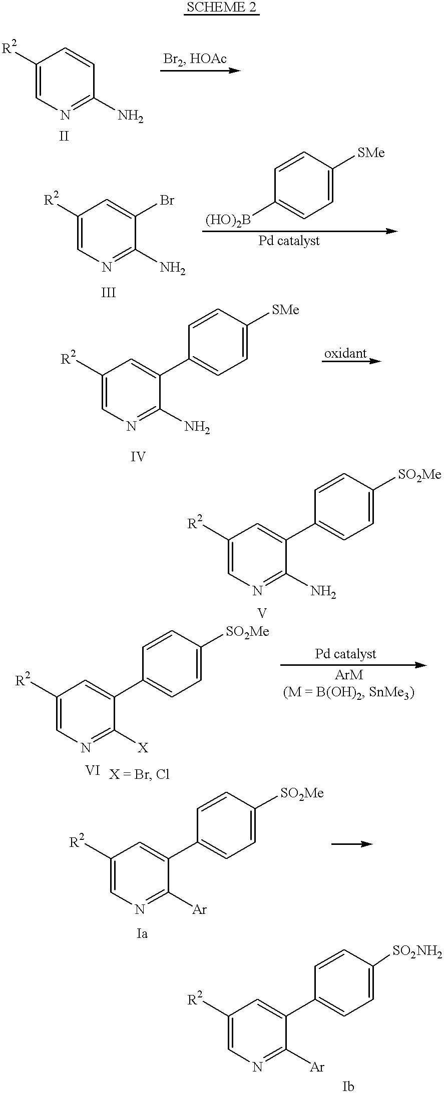

The 2-halopyridines VI of Scheme 1 can also be prepared in a multi-step process from the appropriate 2-hydroxypyridines VII. First, treatment of VII with bromine in acetic acid provides the bromide VIII. Subsequent reaction of VIII with benzyl bromide in the presence of a base such as silver carbonate yields the benzyl ether IX which can be converted to the sulfone X via a sequence of reactions similar to those described for the conversion of bromide III to V in Scheme 1. The benzyl protecting group can be removed by treatment of IX with an acid such as trifluoroacetic acid to afford the hydroxypyridine X. Heating X with POBr₃ or POCl₃ provides the corresponding 2-halopyridines VI (X=Br, Cl) of Scheme 1.

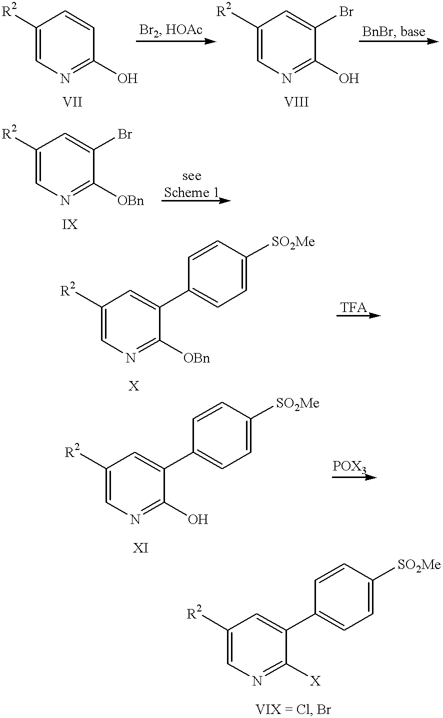

The inhibitors of COX-2 of formula III can be synthesized in accordance with Methods A-L-1 below, in addition to other standard manipulations such as ester hydrolysis, cleavage of protecting groups, etc., as may be known in the literature or exemplified in the experimental procedures.

Method A

An appropriately substituted acid halide is reacted with thioanisole in a solvent such as chloroform in the presence of a Lewis acid such as aluminum chloride to afford a ketone which is then hydroxylated with base such as aqueous sodium hydroxide in a solvent such as carbon tetrachloride with a phase transfer agent such as Aliquat 336. Then treatment with an oxidizing agent such as MMPP in solvents such as CH₂Cl₂/MeOH, affords an sulfone which is reacted with an appropriately substituted acetic acid in a solvent such as CH₂Cl₂ in the presence of an esterifying agent such as CMC and DMAP and then treated with DBU to afford lactone Ia.

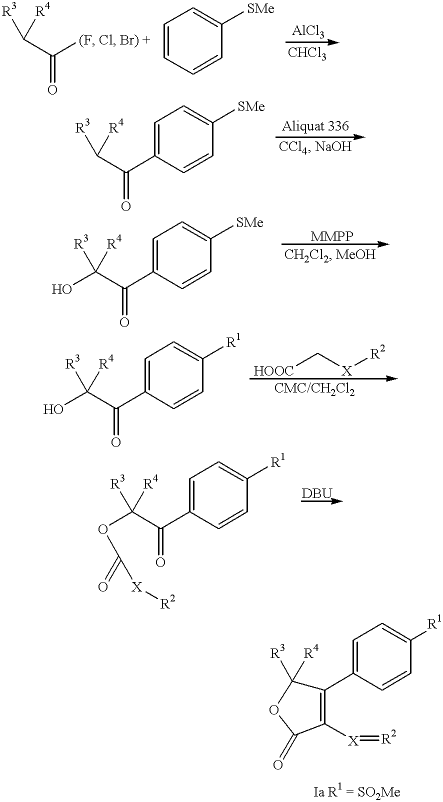

Method B

An appropriately substituted hydroxyketone is acylated withn appropriately substituted acid halide in a solvent such as dichloromethane in the presence of a base such as pyridine. The ester obtained is then reacted with an appropriately substituted nucleophile R²XH in a solvent such as DMF and with a base such as sodium hydride, then treatment with DBU in a solvent such as acetonitrile affords lactone Ia.

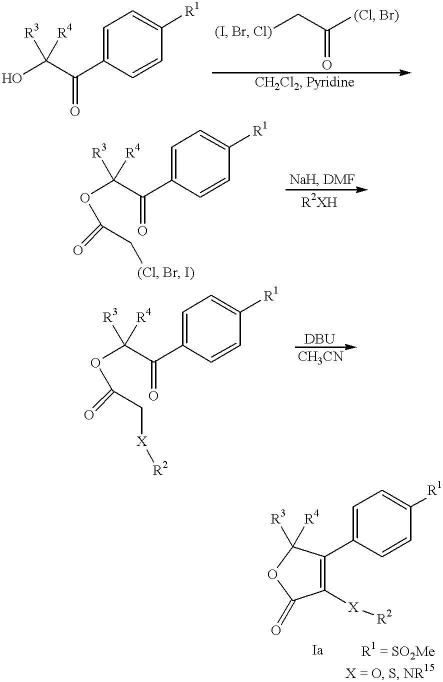

Method C

A halo ester of acetic acid is coupled with an appropriately substituted nucleophile in water with sodium hydroxide to give an appropriately substituted acetic acid which is then reacted as in method A to afford lactone Ia.

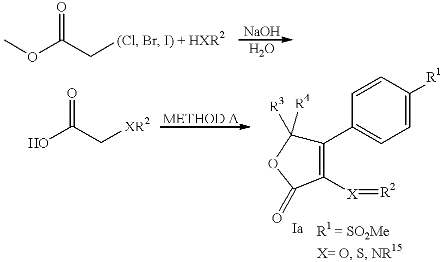

Method D

A halo ester is reacted with an appropriately substituted amine R²R¹⁵NH in a solvent such as toluene to give an intermediate which is then reacted with DBU in a solvent such as acetonitrile to afford lactone Ia.

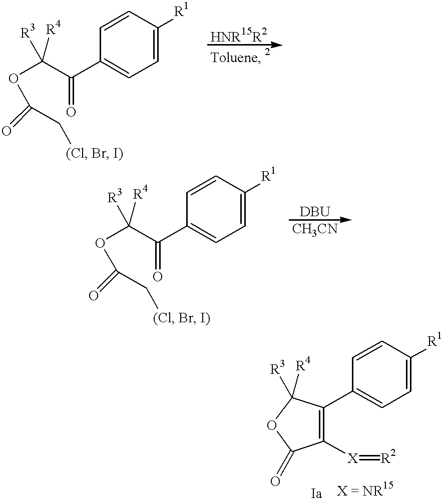

Method E

An appropriately substituted bromoketone is reacted with an appropriately substituted acid in a solvent such as ethanol or acetonitrile in the presence of a base such as diisopropylethylamine or triethylamine to afford an ester which is then treated with DBU in a solvent such as acetonitrile to afford lactone Ia.

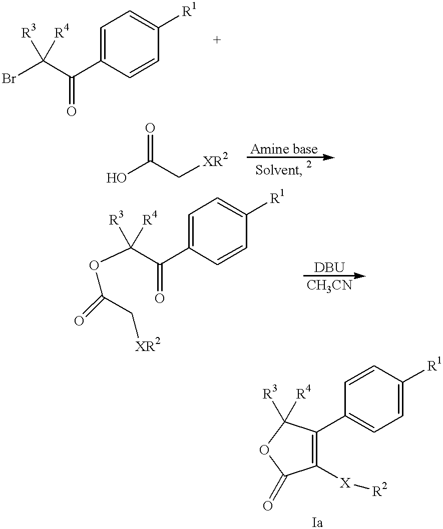

Method F

An appropriately substituted hydroxyketone is reacted with an appropriately substituted acid halide in a solvent such as dichloromethane and with a base such as pyridine to afford an ester which is then cyclized using sodium hydride in a mixture of THF and DMF to afford a lactone. The lactone is then oxidized with an oxidizing agent such as MMPP, mCPBA or OXONE® in solvents such as dichloromethane and/or methanol to afford lactone Ia.

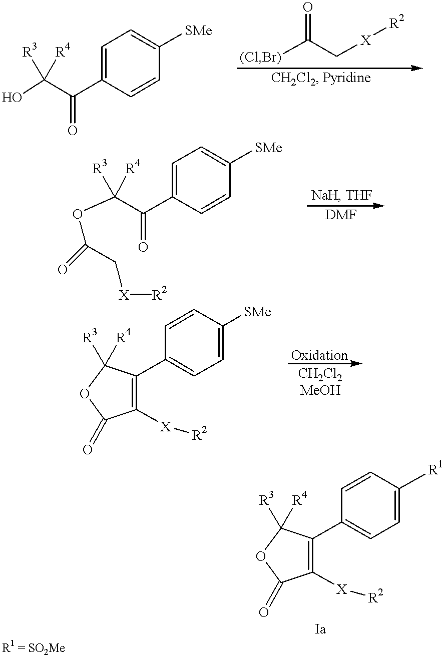

Method G

An appropriately substituted hydroxyketone is acylated with acetyl bromide or chloride in a solvent such as dichloromethane with a base such as DBU and DMAP. Further treatment with a base such as sodium hydride in a solvent such as DMF effects cyclization to afford the 5-membered lactone. Treatment of this lactone with a base such as LDA and an appropriately substituted acid halide in a solvent such as THF, followed by oxidation with a reagent such as MMPP in solvent such as CH₂Cl₂/MeOH and hydrolysis by a base such as NaOH in a solvent such as MeOH/THF gives an alcohol Ib which is then oxidized to lactone Ic by a reagent such as Jone's reagent in a solvent such as acetone(the initially formed ketone is reduced in the reaction and acylated, thus requiring hydrolysis and re-oxidation to obtain ketone Ic). Alternatively, alcohol Ib can be obtained by using an aldehyde R²CHO as the electrophile instead of an acid halide.

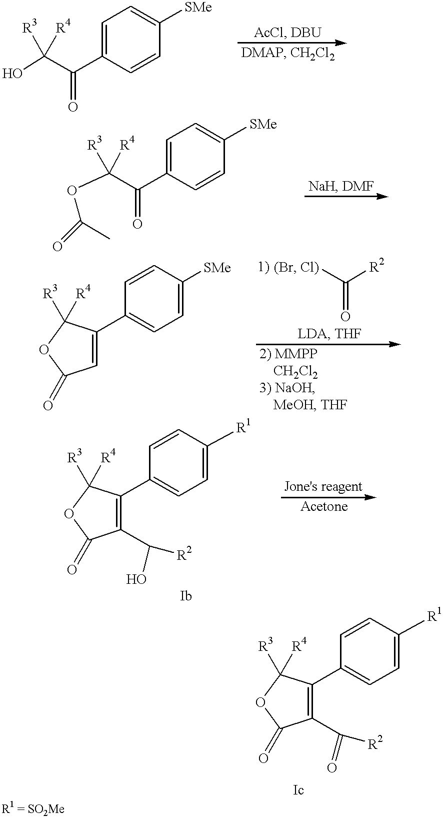

Method H

An appropriately substituted methyl sulfide is oxidized to the sulfoxide with a reagent such as MMPP in solvents such as dichloromethane and methanol followed by treatment with trifluoroacetic anhydride, then aqueous sodium hydroxide. Further treatment by Cl₂ in aqueous acetic acid followed by treatment by an amine affords an intermediate sulfonamide. This sulfonamide is then esterified with an appropriately substituted acid in the presence of a reagent such as CMC and further treatment with a base such as DBU affords the lactone. In the case where the amine group is protected by an acid labile group treatment with an acid such as trifluoroacetic acid in a solvent such as dichloromethane affords compound Ia.

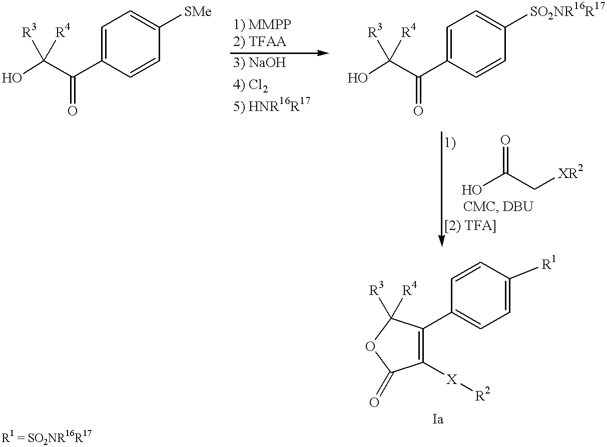

Method I

An appropriately substituted bromoketone is reacted with an appropriately substituted acid in a solvent such as acetonitrile and with a base such as Et₃N. Treatment with DBUand then O₂ gives a hydroxy compound Id. Etherification of this hydroxy with an alcohol in a solvent such as THF and with an acid such HCl gives Ie. By oxidation of the sulfide into a sulfone by a reagent such as m-CPBA and then displacement of this sulfone by an appropriately substituted nucleophile compound If is obtained.

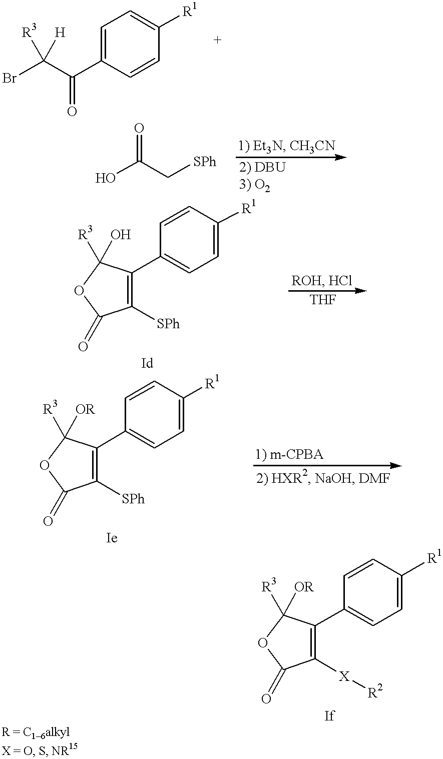

Method J

An appropriately substituted nucleophile is reacted with an appropriately substituted haloacetate in a solvent such as acetonitrile with a base such as DBU to afford compound Ia.

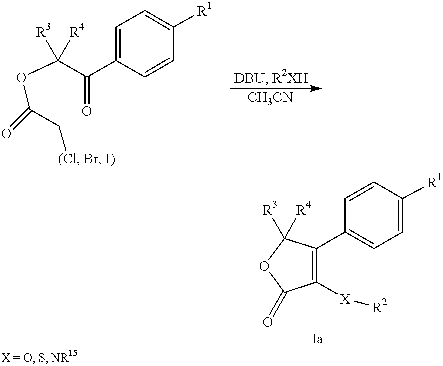

Method K

An appropriately substituted vinyl ketone is coupled with an appropriately substituted benzaldehyde with a catalyst such as 3-benzyl-5-(2-hydroxyethyl)-4-methylthiazolium chloride in the presence of a base such as triethylamine in a solvent such as 1,4-dioxane to form a diketone. The diketone is cyclized in a solvent such as methanol with a base such as DBU to the final product Ig. When R¹=SO₂Me, the starting material can also be a p-methylthiobenzaldehyde, with the methylthio group being oxidized to SO₂Me using MMPP, mCPBA or OXONE® in the last step.

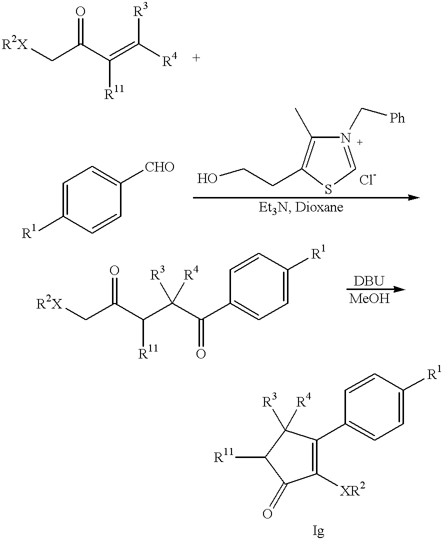

Method L

An appropriately substituted halide is reacted with a base such as DBU in a solvent such as acetonitrile to afford an epoxide which is then reacted with an appropriately substituted nucleophile in solvents such as DMF and a base to afford lactone Ia.

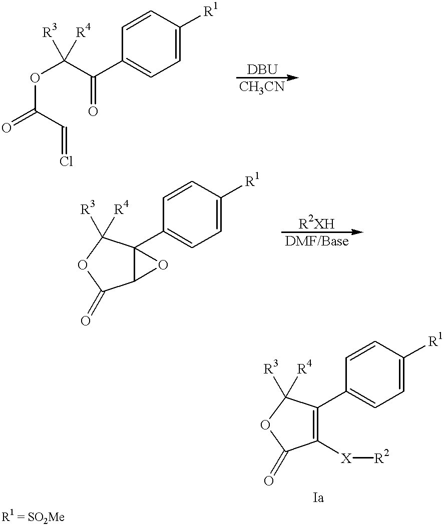

Method M

An appropriately substituted acid halide is reacted with an appropriately substituted hydroxyketone in the presence of a base such as pyridine in a solvent such as acetonitrile, further treatment with a base such as DBU gives an hydroxylactone. The hydroxylactone is reacted with an appropriately substituted halide in a solvent such as benzene with a reagent such as Ag₂CO₃ to afford the lactone Ih.

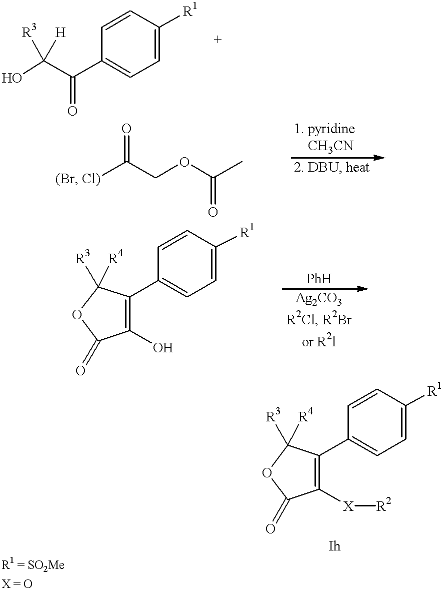

Method N

An appropriately substituted hydroxyketone is reacted with an appropriately substituted carboxylic acid with an esterifying agent such as CMC in the presence of DMAP in a solvent such CH₂Cl₂, followed by treatment with a base such as DBU to afford a lactone ester. This lactone ester is then reacted with a reagent such as the one formed with piperidine and trimethylaluminium to afford the lactone Ic.

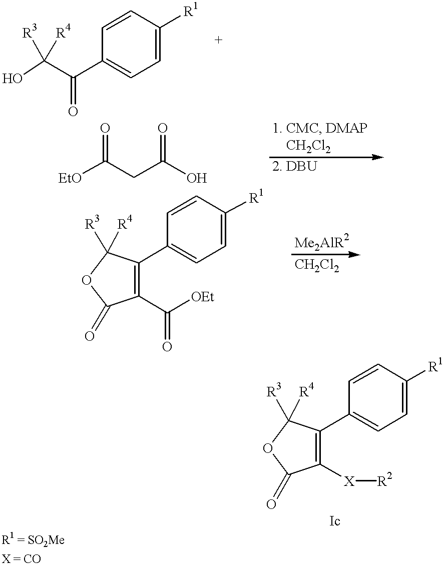

Method O

An appropriately substituted nucleophile such as pentan-3-ol is treated with a base such as sodium hydride in a solvent such as benzene and then reacted with an electrophile such as sodium chloroacetate to afford an acid. This acid is then reacted with an appropriately substituted hydroxyketone with an esterifying reagent such as CMC in a solvent such as dichloromethane to give an ester which is cyclized upon treatment with a base such as sodium hydride in a solvent such as DMF to afford lactone Ia.

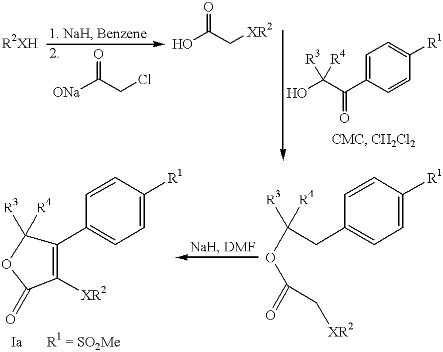

Method P

An appropriately substituted nucleophile is reacted with an appropriately substituted haloacetate alkaline salt (such as sodium) in a solvent such as benzene and with a reagent such as Ag₂CO₃ to give an ester which is then hydrolyzed with a reagent such as NaOH in solvents such as water and methanol to give an acid. The acid is then esterified with an appropriately substituted hydroxyketone with reagents such as CMC and DMAP in a solvent such as dichloromethane to give an ester which is then cyclized with a base such as DBU in a solvent such as CH₃CN to afford a lactone. The sulfide is then oxidized with a reagent such as MMPP in solvents such as CH₂Cl₂, MeOH and water to afford lactone Ia.

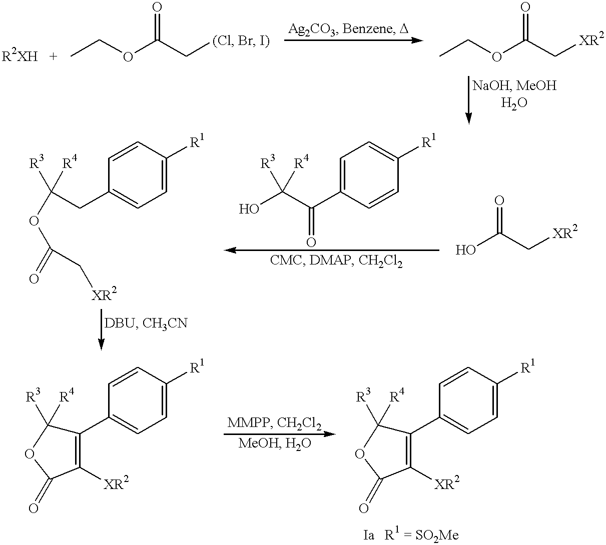

Method Q

An appropriately substituted acetic acid salt is reacted with a nucleophile such as vinyl magnesium bromide in a solvent such as DME to afford a ketone, which is then reacted as in method K to afford cyclopentone Ig.

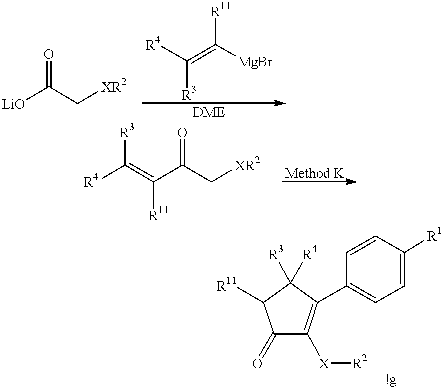

Method R

4-Bromothioanisole is reacted with a base such as n-BuLi in a solvent such as THF to form the corresponding lithium reagent which is then reacted with an appropriately substituted lactone (Tetrahedron, 1984, 40, 1313) to give a hemiketal. The acetal is then cleaved with an acid such as p-TsOH in a solvent such as water to give a hydroxyketone. The sulfide is then oxidized with a reagent such as Oxone®, in the presence of a phase transfer reagent such as Aliquat 336® ( in solvents such as t-BuOH and water to give a sulfone. The hydroxyketone is then esterified with an appropriately substituted acetic acid with reagents such as CMC and DMAP in a solvent such as CH₂Cl₂ to give an intermediate ester which is cyclized with a base such as DBU to give lactone Im.

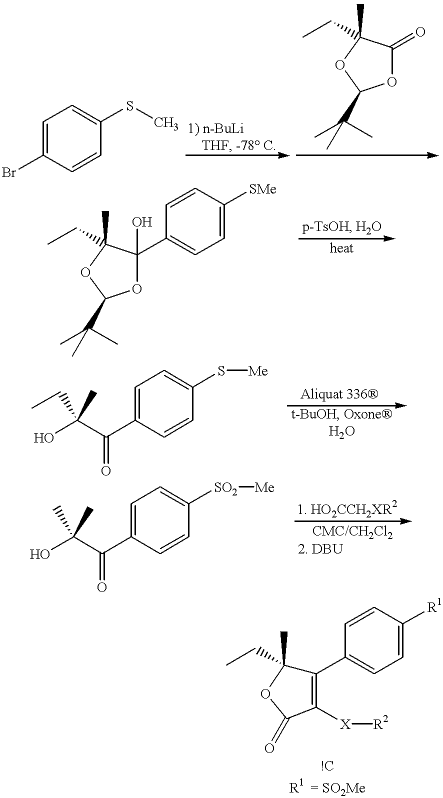

Method S

An appropriately substituted aminopyrydine is diazotized with NaNO₂ in an acid such as H₂SO₄ in water, followed by neutralization with NaOH affords an hydroxypyridine which is reacted following the method J.

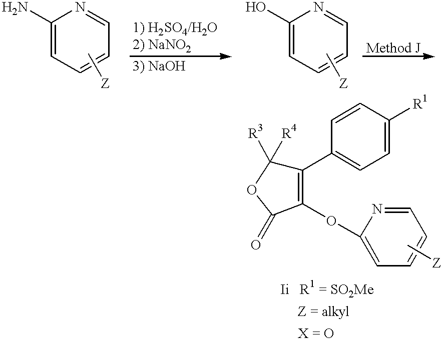

Method T

An appropriately substituted hydroxylactone is treated with a base such as KOH in a solvent such as DMF, followed by treatment with an appropriately substituted halopyridine afford lactone Ii.

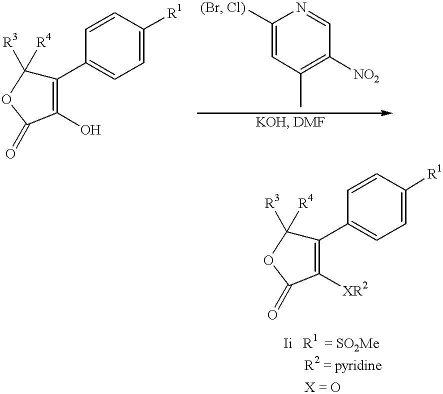

Method U

An appropriately substituted nitropyridine is reduced with a reagent such as Fe (powder) and NH₄Cl in solvents such as ethanol and water to give an aminopyridine which is diazotized with NaNO₂ in aqueous HCl, the diazonium salt is decomposed with copper salts such as CuCl in HCl to give lactone Ii.

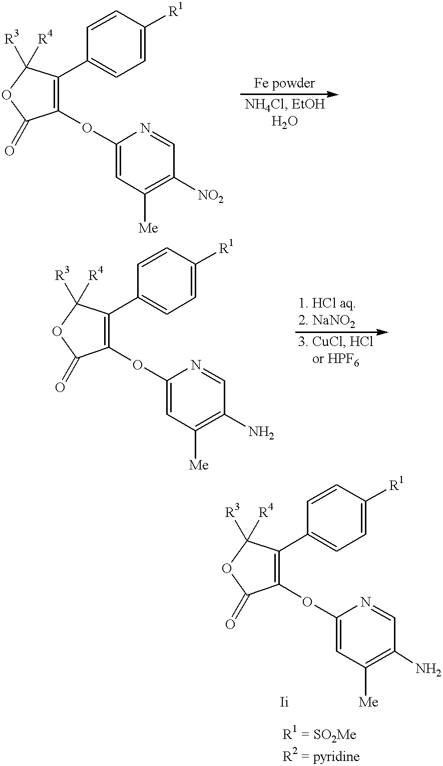

Method W

An appropriately substituted halo acetate is reacted with an appropriate secondary amine (R²(R¹⁵)NH) in a solvent such as CH₃CN; further treatment with a base such as NaH in a solvent such as DMF affords lactone Ia.

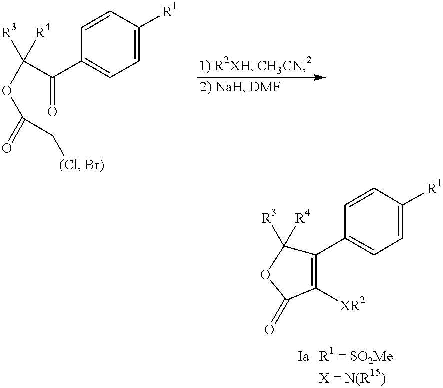

Method X

An appropriately substituted lactone (Tetrahedron, 1984, 40, 1313) is treated with a base such as LDA and reacted with 2,2,2-trifluoroiodoethane. Further treatment with the lithium salt of 4-bromothioanisole gives the desired hemiketal, which is then reacted as in method R to give the desired lactone Im.

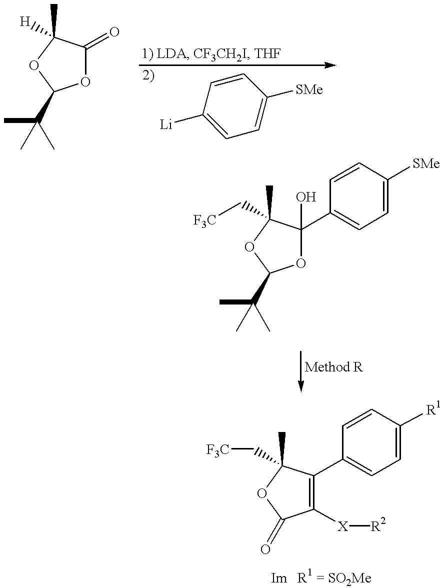

Method Y

An appropriately substituted alcohol is reacted with an appropriate haloacid such as bromoacetic acid with a base such as NaH in a solvent such as THF to afford an acid ether which is then esterified with an appropriately substituted hydroxyketone with reagents such as CMC and DMAP in a solvent such as CH₂Cl₂ to give a ketoester. The ketoester is then cyclized in the presence of a base such as DBU and a dehydrating reagent such as iso-propyl trifluoroacetate in a solvent such as CH₃CN to afford lactone Ia.

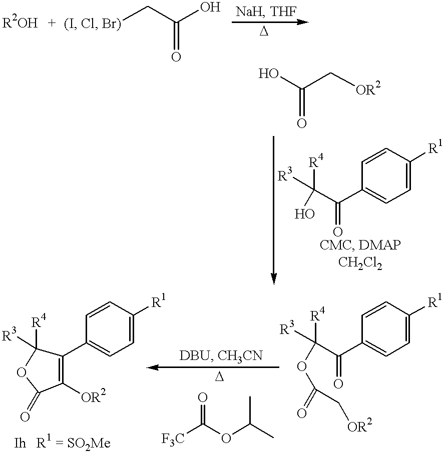

Method Z

An appropriately substituted hydroxylactone is reacted with an appropriate halide in the presence of a base such as NaH, with a reagent such as Bu₄NI in a solvent such as DMF to afford lactone Ih.

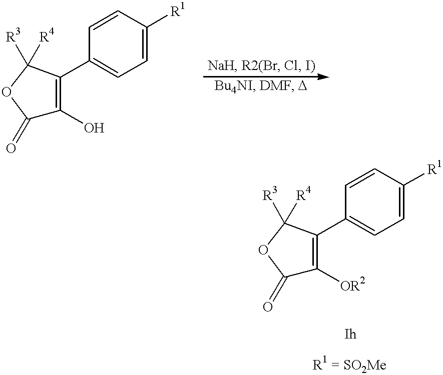

Method A-1

An appropriately substituted carboxylic acid is esterified with an appropriately substituted haloketone in the presence of a base such as (iPr)₂NEt in a solvent such as EtOH; further treatment with a base such as DBU and a reagent such as iso-propyl 2,2,2-trifluoroacetate in a solvent such as CH₃CN affords lactone Ia.

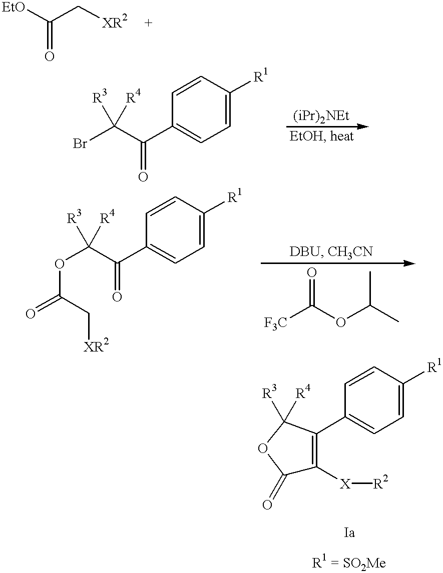

Method B-1

An appropriately substituted ketone is reacted with a reagent such as TMSCN in the presence of a Lewis acid such as ZnI₂, further treatment with a metal salt of thioanisole followed by hydrolysis affords an hydroxyketone. Oxidation of the sulfide with an oxidizing reagent such as Oxone® in solvents such as t-BuOH, EtOAc and water gives the sulfone. Esterification of the alcohol and an appropriately substituted acetic acid with a reagent such as CMC and DMAP in a solvent such as CH₂Cl₂ followed by treatment with a base such as DBU gives the lactone In.

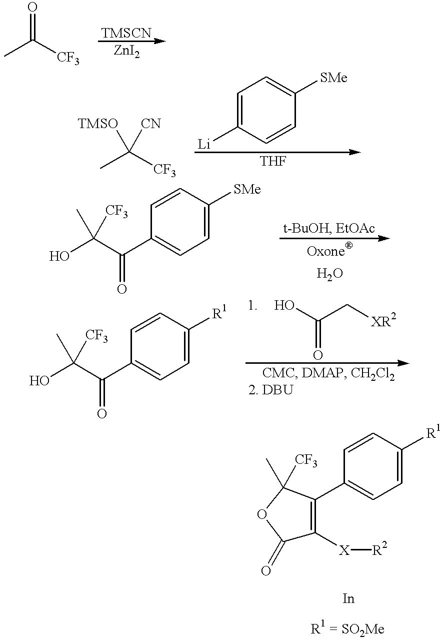

Method C-1

An appropriately substituted enone is reduced with hydrogen in a solvent such as ethyl acetate with a catalyst such as palladium on activated carbon to give an alcohol. This alcohol was transformed into a leaving group by treatment with reagents such as methanesulfonyl chloride and triethylamine in a solvent such as methylene chloride, followed by treatment in a solvent such as acetone with a reagent such as lithium iodide to afford a compound which was then reacted as in method M to afford lactone Ij.

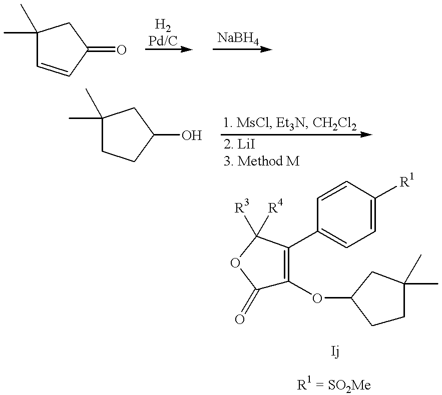

Method D-1

5-Amino-2-alkoxypyridine is converted to the corresponding diazonium salt and heated with acetic anhydride at 100-110° C. The corresponding 5-acetoxy-2-alkoxypyridine is then hydrolysed with sodium hydroxide to give the 5-hydroxy-2-alkoxypyridine which is reacted according to method J.

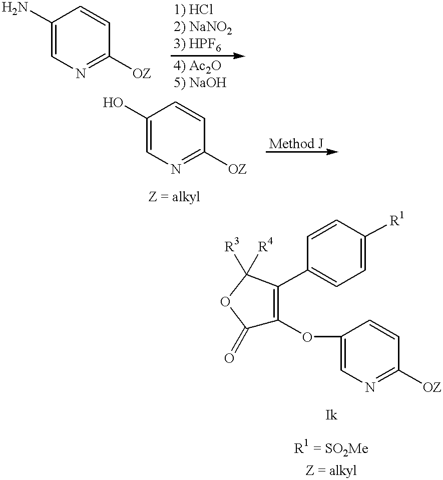

Method E-1

2(RS)-2-Methyl-4,4,4-trifluorobutyryl chlolide(GB 2238790-A) is reacted with thioanisole in the presence of a Lewis acid such as AlCl₃. The ketone is then hydroxylated by air in the presence of potassium t-butoxide and triethyl phosphite, and the sulfide is then oxidized with m-CPBA to the sulfone. The hydroxyketone is then esterified with an appropriately substituted acid in the presence of CMC and DMAP in a solvent such as CH₂Cl₂ to give an intermediate ester which is cyclized with a base such as DBU to give lactone Io.

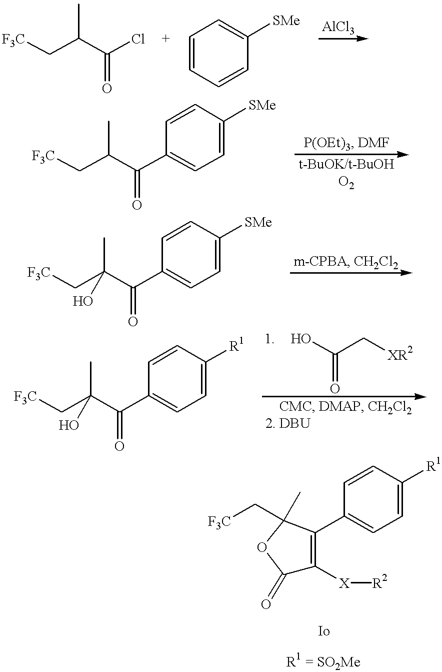

Method F-1

Hydroquinone is reacted with a halosubstituted acetate, chlorinated with sulfuryl chloride, methylated with iodomethane in the presence of a base and followed by hydrolysis with sodium hydroxide to give the substituted phenoxy acetic acid, which is reacted according to method A to afford lactone Ia.

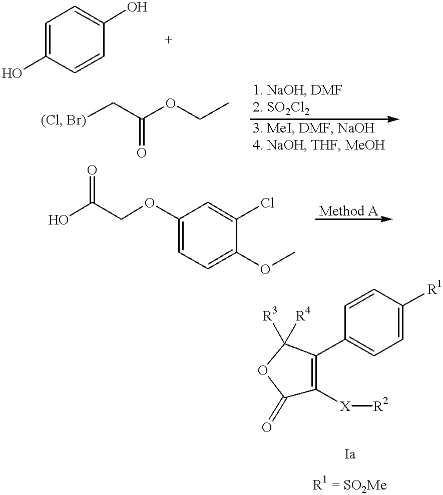

Method G-1

An appropriately substituted 3-(4-(1-hydroxy-1-methyl)ethylphenoxy)-5H-furan-2-one is reduced with NaBH₃CN in the presence of ZnI₂ to give lactone Il.

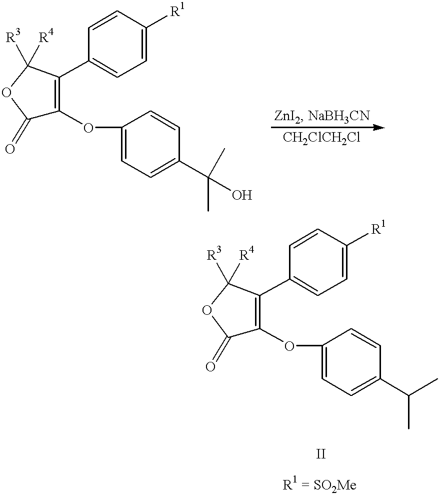

Method H-1

An appropriately substituted alkyl (4-thiomethyl)phenyl ketone is alkylated with bromomethylcyclopropane using a base such as KHMDS. The methyl sulfide is oxidized with MMPP to the corresponding sulfone and hydroxylated by NaOH and CCl₄ in toluene in the presence of a phase transfer catalyst such as Aliquat 336®. The hydroxyketone is then esterified with an appropriately substituted acid in the presence of CMC and DMAP in a solvent such as CH₂Cl₂ to give an intermediate ester which is cyclized with a base such as DBU to give lactone Ip.

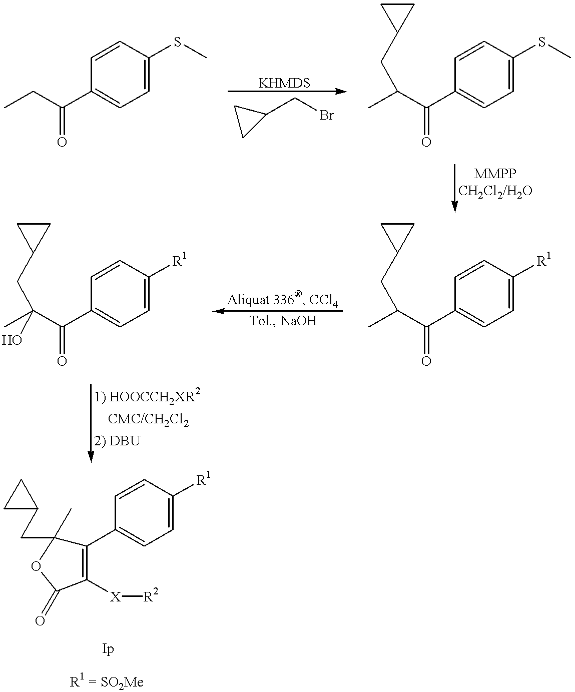

Method I-1

An appropriately substituted hydroxylactone is reacted with an appropriately substituted nitropyridine in the presence of a base such as NaOH in DMF at 100-110° C. The nitro group of the coupling product is then reduced with Fe (powder) and NH₄Cl in solvents such as ethanol and water. The amino group is diazotized and the resulting diazonium salt is decomposed in the presence of appropriate copper salt such as CuCl or CuBr to give lactone 1q. Alternatively, the diazonium salt is treated HBF₄ or HPF₆ to give after heating the fluoro-substituted lactone pyridine Iq.

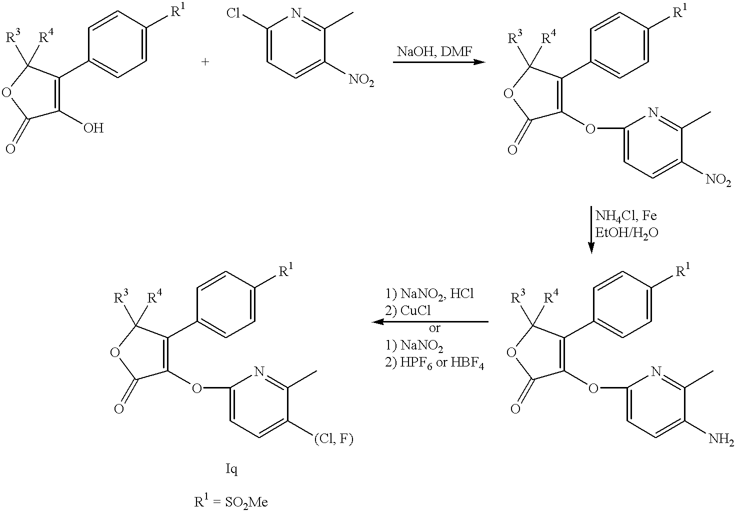

Method J-1

The lithium reagent prepared from 4-bromothioanisole and n-BuLi at −72° C. is reacted with methacrolein and the resulting product is oxidized with an oxidizing reagent such as Oxone® to the methyl sulfone. A kinetic resolution by Sharpless epoxidation reaction using (+)-diisopropyl tartrate and t-butyl hydroperoxide provides the (S)-allylic alcohol, which is epoxidized by (−)-diisopropyl tartrate and t-butyl hydroperoxide. The alcohol of the epoxy alcohol is protected as an ethoxyethyl ether and the epoxide is reacted with dimethyl cuprate(from methyllithium and copper(I) iodide. The ethoxyethyl ether is then cleaved and the resulting diol is treated with (Bu₃Sn)₂O and oxidized with Br₂ to give the (S)-alcohol. The hydroxyketone is then esterified with an appropriately substituted acid in the presence of CMC and DMAP in a solvent such as CH₂Cl₂ to give an intermediate ester which is cyclized with a base such as DBU to give lactone Ir.

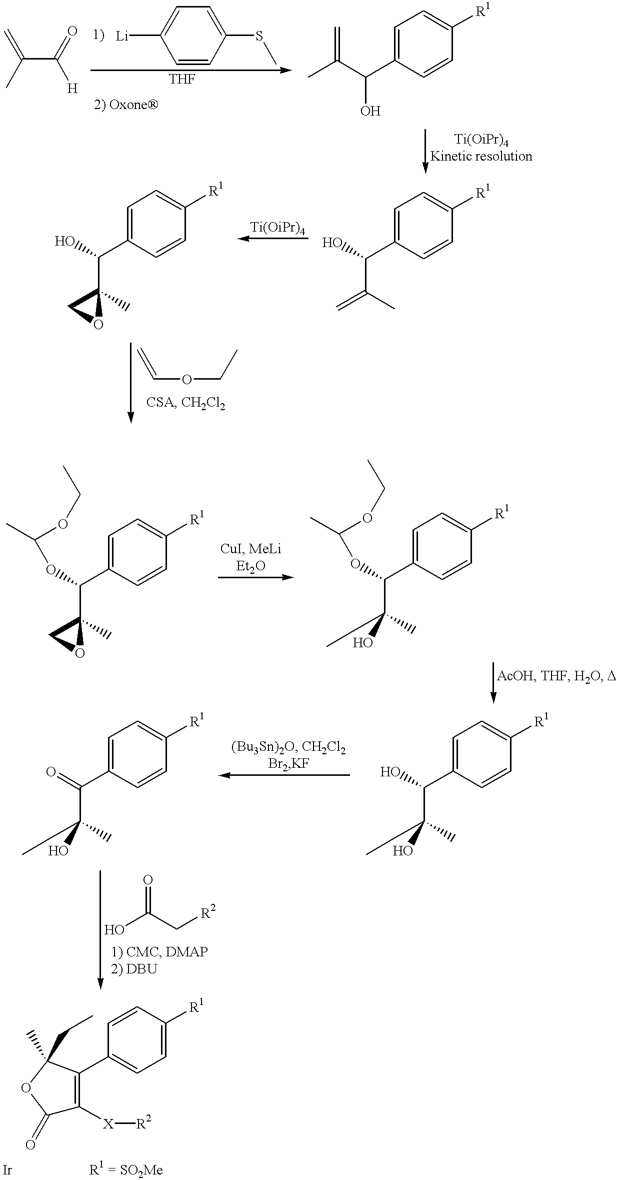

Method K-1

4-Bromothioanisole is reacted with isobutyryl chloride in the presence of aluminum chloride in o-dichlorobenzene(ODCB). The resulting ketone is brominated and oxidized with Na₂WO₄ and H₂O₂ in the presence of Aliquat 336 to the bromoketone methyl sulfone. The bromoketone is then reacted with an appropriate alkoxy or aryloxy acetic acid in the presence DIEA and the ester intermediate is cyclized and dehydrated with DBU in the presence of isopropyl trifluoroacetate to give lactone 1a.

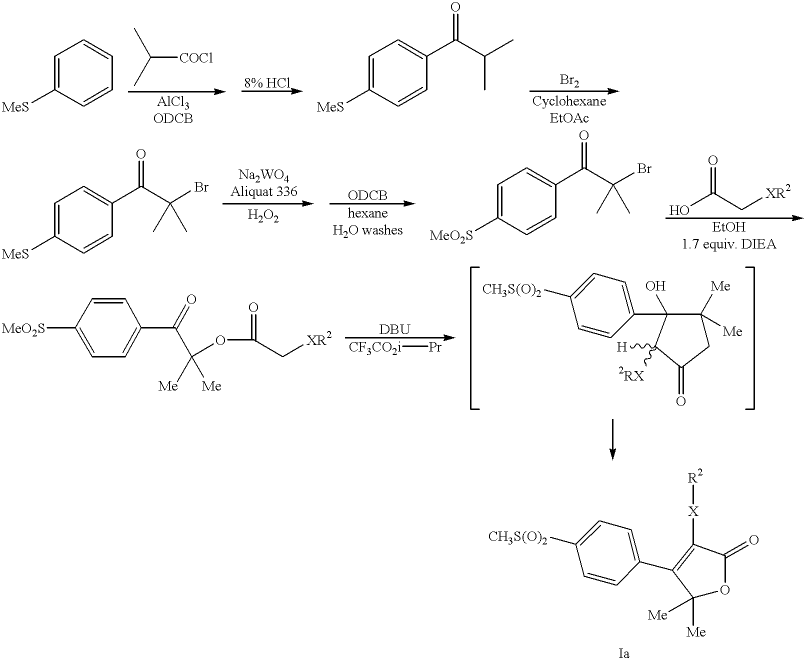

Method L-1

Tetronic acid is converted to the α-diazoketone derivative with tosyl azide (see Stachel et al., Liebigs Ann. Chem. 1994, P.129 for a similar preparation). The diazo compound is reacted with an appropriately substituted alcohol in the presence of rhodium acetate (see Stachel et al., Liebigs Ann. Chem. 1994 P. 129) to give an ether. This compound is treated with triflic anhydride followed by a Suzuki type coupling reaction with 4-methylthiophenyl boronic acid (Wong et al., Tetrahedron Lett. 1993, p. 8237.) The sulfide is then oxidized with OXONE® to provide Is.

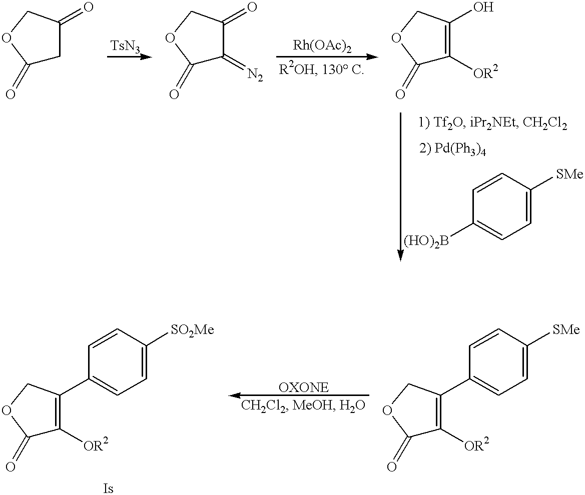

The PSA conjugates of formulae IV, VI and VIII can be synthesized in accordance with Schemes 3-7, in addition to other standard manipulations such as ester hydrolysis, cleavage of protecting groups, etc., as may be known in the literature or exemplified in the experimental procedures.

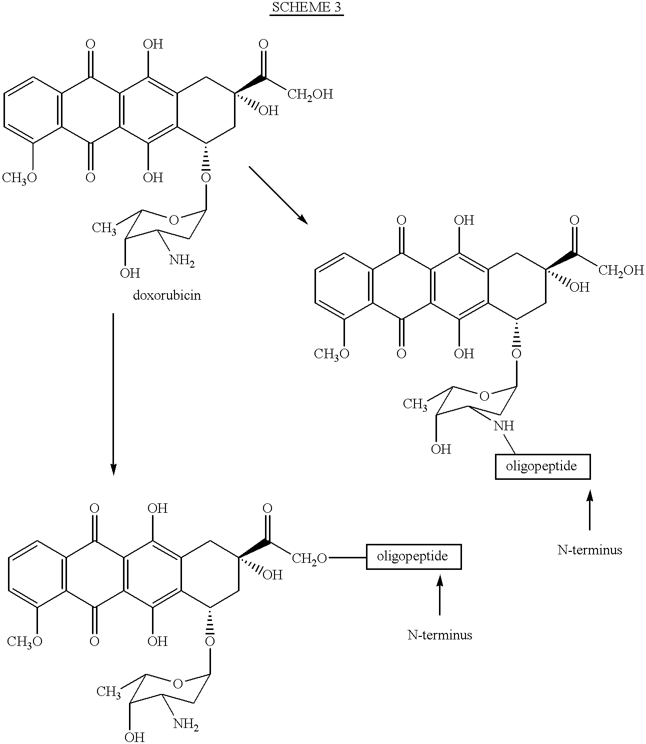

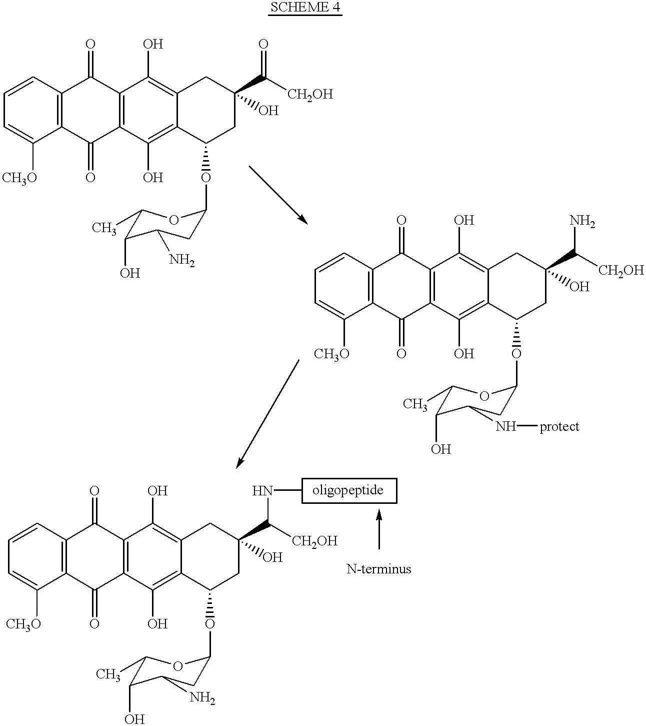

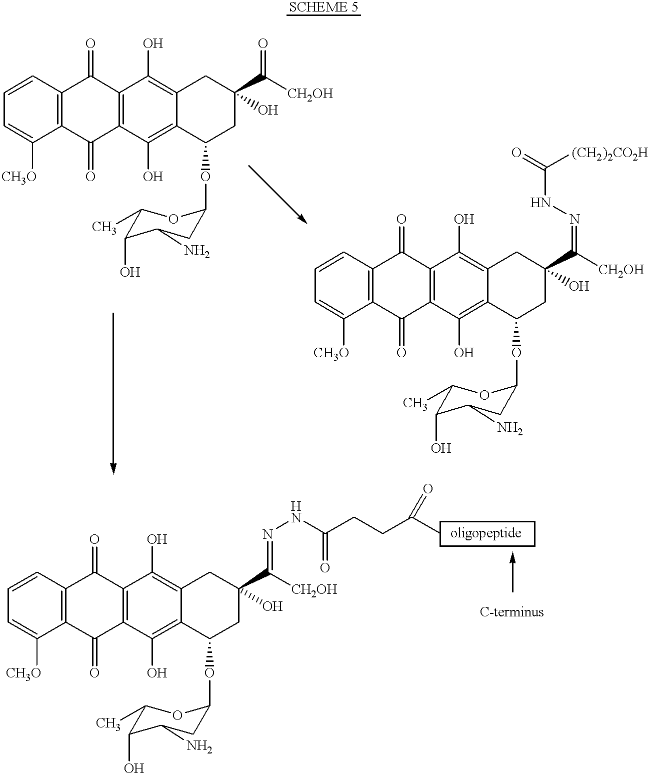

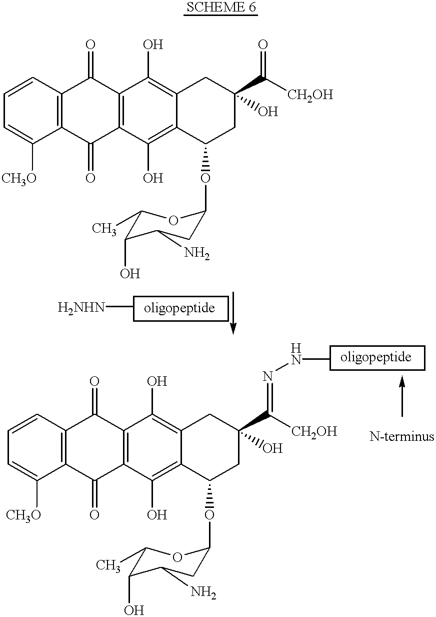

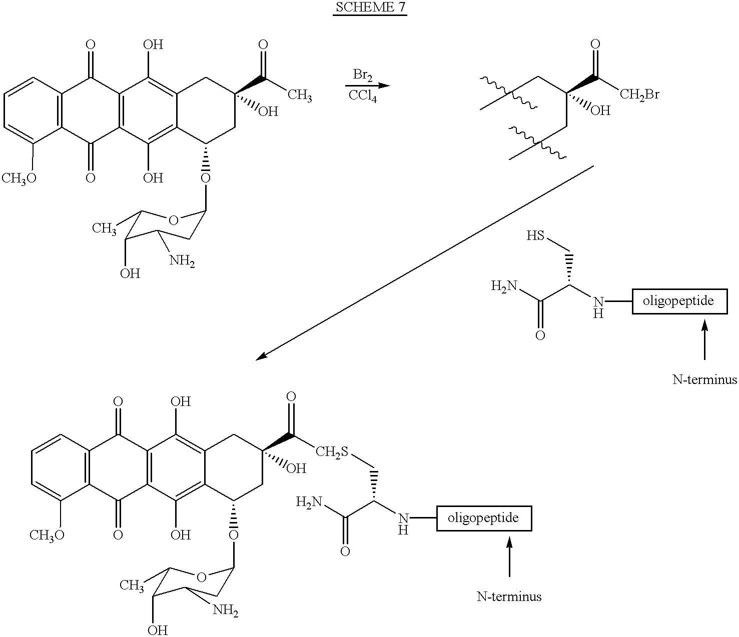

Scheme 8 illustrates preparation of conjugates utilized in the instant method of treatment wherein the oligopeptides are combined with the vinca alkaloid cytotoxic agent vinblastine. Attachment of the N-terminus of the oligopeptide to vinblastine is illustrated (S. P. Kandukuri et al. J. Med. Chem. 28:1079-1088 (1985)).

Scheme 9 illustrates preparation of conjugates of the oligopeptides of the instant invention and the vinca alkaloid cytotoxic agent vinblastine wherein the attachment of vinblastine is at the C-terminus of the oligopeptide. The use of the 1,3-diaminopropane linker is illustrative only; other spacer units between the carbonyl of vinblastine and the C-terminus of the oligopeptide are also envisioned. Furthermore, Scheme 9 illustrates a synthesis of conjugates wherein the C-4-position hydroxy moiety is reacetylated following the addition of the linker unit. Applicants have discovered that the desacetyl vinblastine conjugate is also efficacious and may be prepared by eliminating the steps shown in Scheme 9 of protecting the primary amine of the linker and reacting the intermediate with acetic anhydride, followed by deprotection of the amine. Conjugation of the oligopeptide at other positions and functional groups of vinblastine may be readily accomplished by one of ordinary skill in the art and is also expected to provide compounds useful in the treatment of prostate cancer.

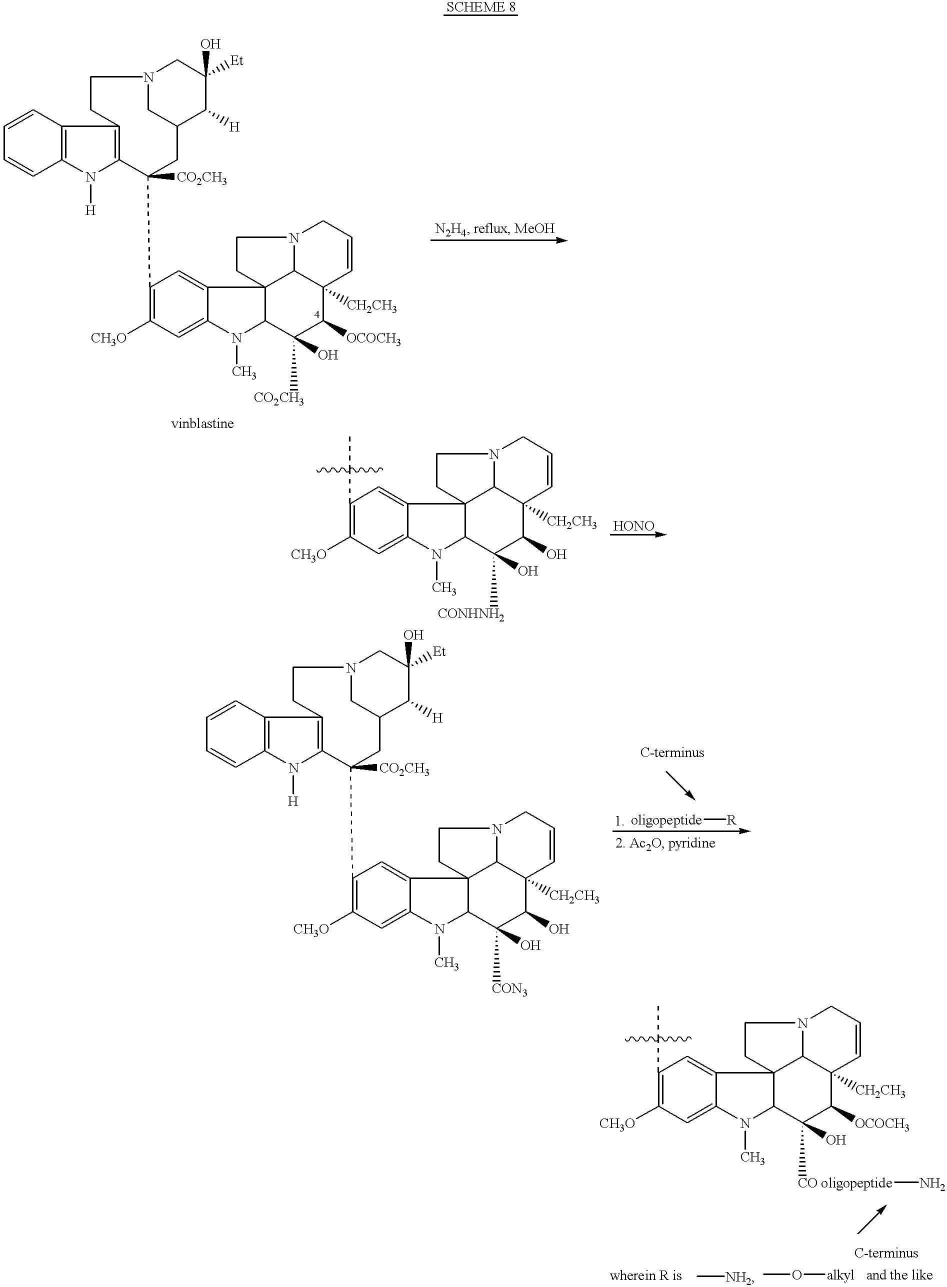

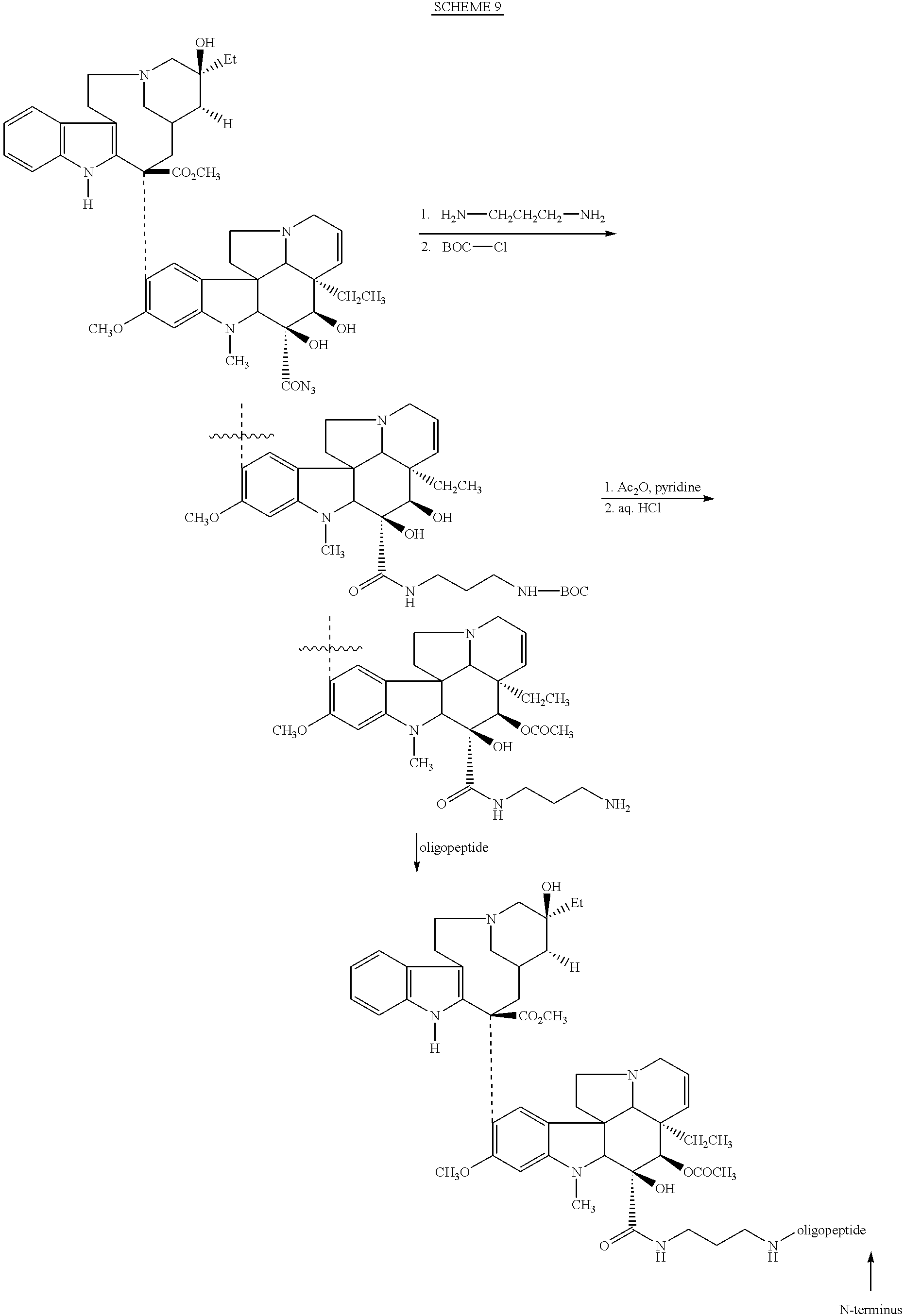

The PSA conjugates of formula VI and VII can be synthesized in accordance with Scheme 10, in addition to other standard manipulations such as ester hydrolysis, cleavage of protecting groups, etc., as may be known in the literature or exemplified in the experimental procedures.

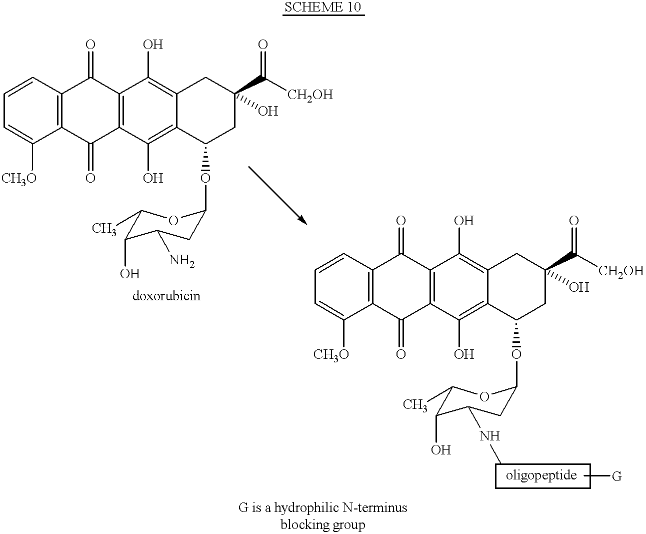

Scheme 11 illustrates preparation of PSA conjugates of the formula IX wherein the attachment of vinblastine is at the C-terminus of the oligopeptide. Furthermore, Scheme 11 illustrates a synthesis of conjugates wherein the C-4-position hydroxy moiety is reacetylated following the addition of the linker unit. Applicants have discovered that the desacetyl vinblastine conjugate is also efficacious and may be prepared by eliminating the steps shown in Scheme 11 of protecting the primary amine of the linker and reacting the intermediate with acetic anhydride, followed by deprotection of the amine. Conjugation of the oligopeptide at other positions and functional groups of vinblastine may be readily accomplished by one of ordinary skill in the art and is also expected to provide compounds useful in the treatment of prostate cancer.

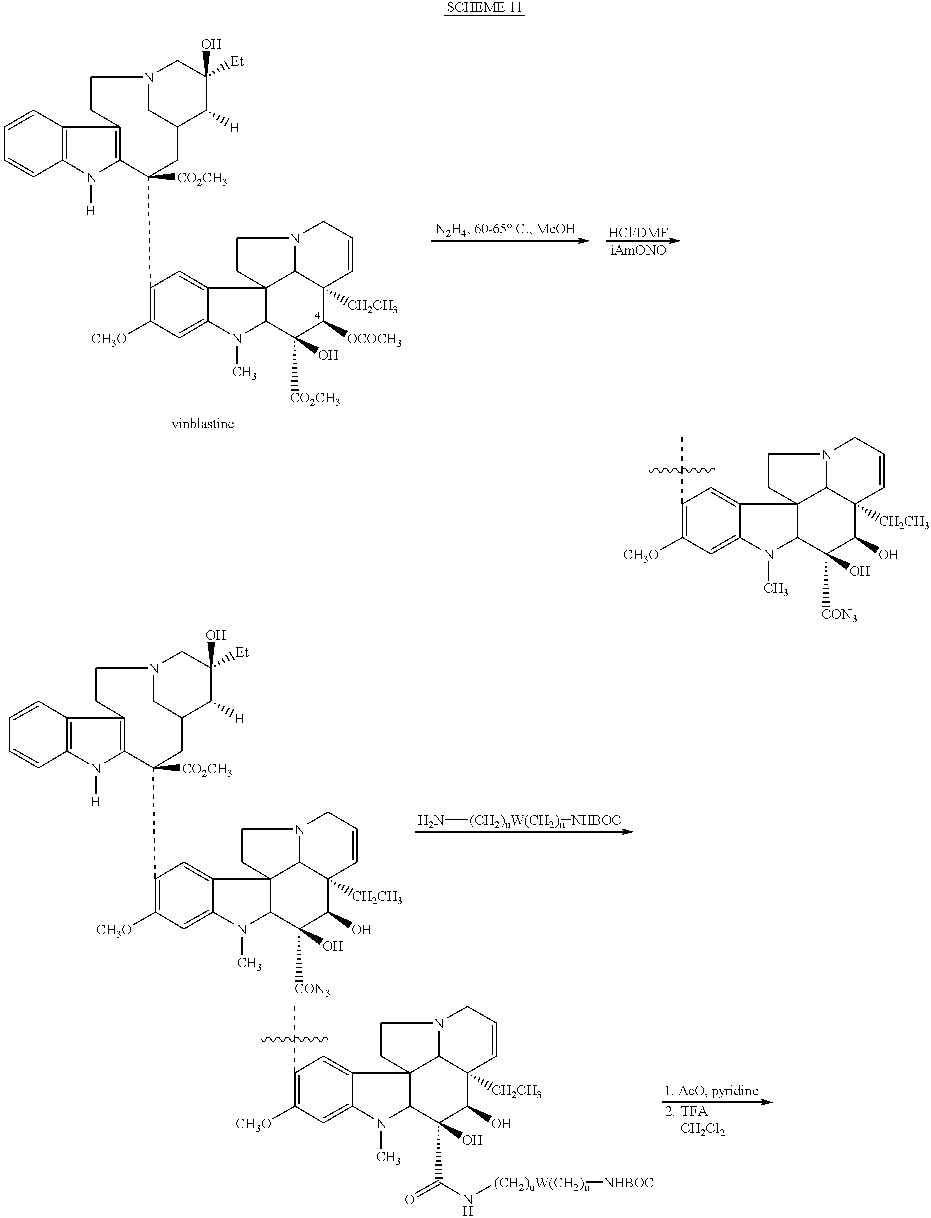

The PSA conjugates of formula X can be synthesized in accordance with Schemes 12-13, in addition to other standard manipulations such as ester hydrolysis, cleavage of protecting groups, etc., as may be known in the literature or exemplified in the experimental procedures.

Reaction Scheme 12 illustrates preparation of conjugates of the oligopeptides of the instant invention and the vinca alkaloid cytotoxic agent vinblastine wherein the attachment of the oxygen of the 4-desacetylvinblastine is at the C-terminus of the oligopeptide. While other sequences of reactions may be useful in forming such conjugates, it has been found that initial attachment of a single amino acid to the 4-oxygen and subsequent attachment of the remaining oligopeptide sequence to that amino acid is a preferred method. It has also been found that 3,4-dihydro-3-hydroxy-4-oxo-1,2,3-benzotriazine (ODHBT) may be utilized in place of HOAt in the final coupling step.

Reaction Scheme 13 illustrates preparation of conjugates of the oligopeptides of the instant invention wherein a hydroxy alkanolyl acid is used as a linker between the vinca drug and the oligopeptide.

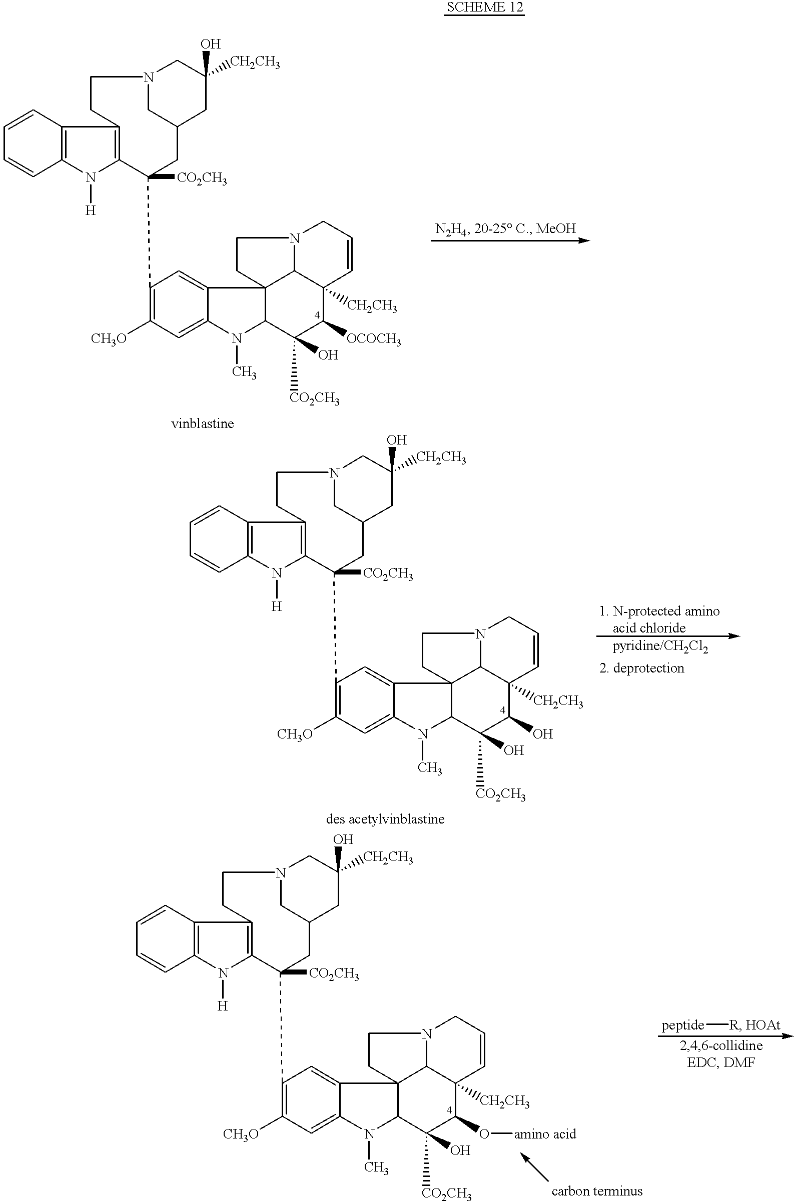

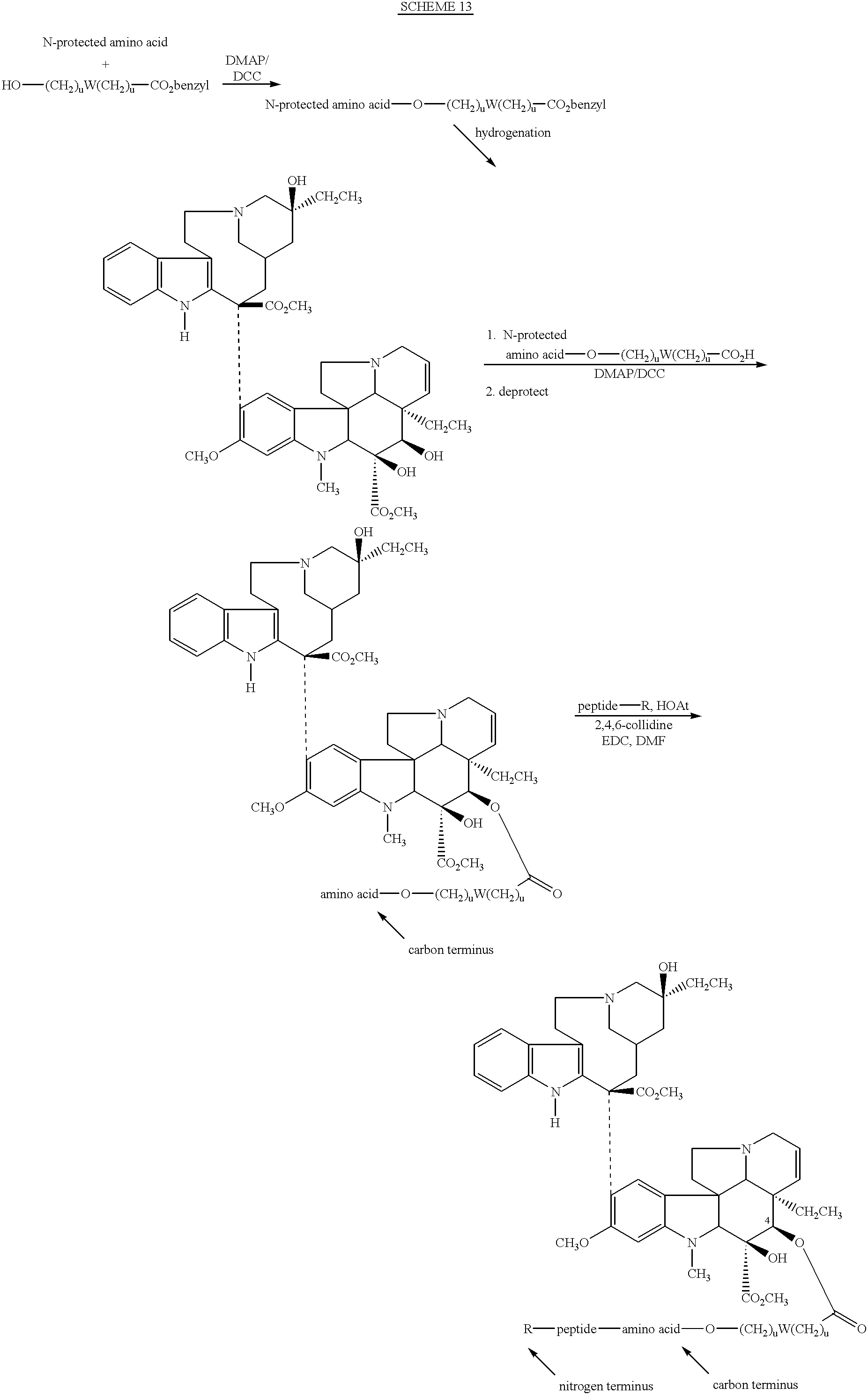

### EXAMPLES

Examples provided are intended to assist in a further understanding of the invention. Particular materials employed, species and conditions are intended to be further illustrative of the invention and not limitative of the reasonable scope thereof.

The standard workup referred to in the examples refers to solvent extraction and washing the organic solution with 10% citric acid, 10% sodium bicarbonate and brine as appropriate. Solutions were dried over sodium sulfate and evaporated in vacuo on a rotary evaporator.

### Example 1

3-phenyl-4-(4-methylsulfonyl)phenyl-2(5H)-furanone.

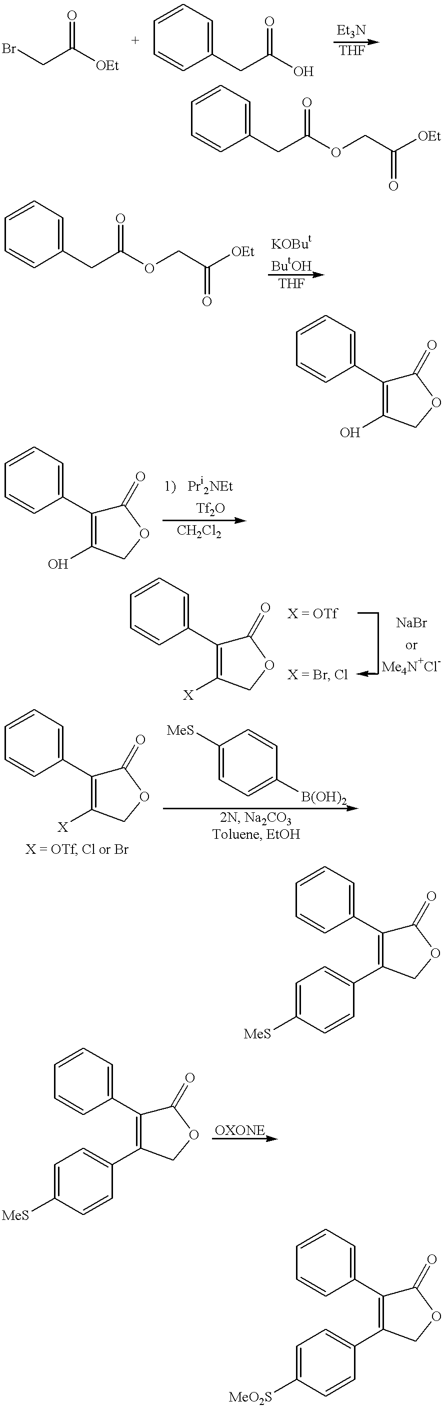

To a stirred solution of phenylacetic acid (100 g, 1 equiv) and ethyl bromoacetate (77.4 mL, 0.95 equiv.) in THF (1000 mL) was added triethylamine (107.5 mL, 1.05 equiv.), and the resulting mixture was heated to 60° C. for 1 h (nitrogen atmosphere). The mixtue was cooled to 25° C. and was then poured into a stirred mixture of ethyl acetate (1000 mL) and water (1000 mL). The layers were separated, the aq. layer was extracted with ethyl acetate (600 mL) and the combined organic extracts were washed with sat. sodium bicarbonate solution (500 mL) and with brine (300 mL). The solvent was removed under reduced pressure to give the ester (145 g, 94%). 1H nmr, 300 MHz, CDCl3), 1.25 (3H, t, J=7 Hz), 3.75 (2H, s), 4.20 (2H, q, J=7 Hz), 4.60 (2H, s), 7.2-7.4 (5H, m).

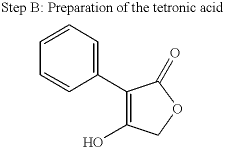

To a stirred solution of potassium tert-butoxide (10 g, 1 equiv.) in tert-butanol (80 mL) at 25° C. (nitrogen atmosphere) was added the ester (10 g, 1 equiv.). The mixture was heated to 70° C. for 60 min and then water (100 mL) and methyl tert-butyl ether (100 mL) were added. The layers were separated and the organic layer extracted with H₂O (50 mL). The combined aqueous extracts were washed with methyl tert-butyl ether (50 mL) and were then acidified to pH 4 with 2N aqueous HCl. The white precipitate thus obtained was filtered and dried by suction to give the tetronic acid (5.65 g, 72%). ¹H nmr (300 MHz, DMSO), 4.75 (s, 2H), 7.22 (1H, t, J=8 Hz), 7.45 (2h, t, J=8 Hz), 7.9 (2H, d, J=8 Hz).

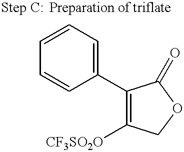

To a cold (0° C.), stirred solution of the tetronic acid (680 mg, 1 equiv.) in CH₂Cl₂ (10 mL) was added N,N-diisopropylethylamine (0.74 mL, 1.1 equiv.) followed by trifluoromethanesulfonyl anhydride (0.65 mL, 1 equiv) (nitrogen atmosphere). The solution was stirred for 10 min at 0° C. and was then poured into a stirred mixture of H₂O (10 mL) and ethyl acetate (20 mL). The layers wetre separated and the organic layer was washed with 1N HCl (10 mL) and then with water (10 mL). The solvent was removed to give the triflate as a yellow oil (which solidified upon storage at −10° C.). ¹H nmr (300 MHz, CDCl₃) 5.08 (2H, s), 7.4-7.5 (3H, m), 7.7-7.8 (2H, m).

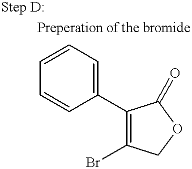

A mixture of the triflate (15.3 g, 1 equiv) and lithium bromide (25 g, 5 equiv) in acetone (100 mL) was heated at 50° C. for 30 min (nitrogen atmosphere). The mixture was cooled to 25° C. and was then partitioned between ethyl acetate (200 mL) and H₂O (100 mL). The aqueous layer was extracted with ethyl acetate (50 mL) and the combined organic extracts were washed with brine (50 mL) and were then concentrated to give the bromide (11.28 g, 90%). 1H nmr (300 MHz, CDCl3), 4.85 (2H, s), 7.4-7.5 (3H, m), 7.7-7.8 (2H, m).

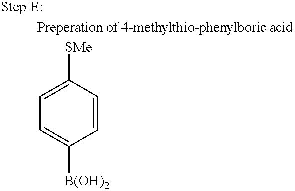

To a cold (−75 to −78° C.), stirred solution of 4-bromothioanisole (20 g, 1 equiv.) and triisopropyl borate (33 mL, 1.45 equiv.) in dry THF (350 mL) was slowly added a solution of n-butyllithium (1.5 M in hexanes) over 3 h (nitrogen atmosphere). The resulting mixture was then stirred at −78° C. for 30 min and then was allowed to warm to 25° C. over 1 h. Aqueous sulfuric acid (2 M, 200 mL) was added slowly (internal temperature rose to 30° C. during addition) and the mixture was stirred at 25° C. for 2 h. The layers were separated and the aqueous layer was extracted with ethyl acetate (2×100 mL). The combined organic extracts were then concentrated to approx. 30 mL and to the resulting slurry was added water (100 mL). The mixture was stirred for 10 h, and the solid was filtered and was the washed with toluene and dried by suction, to give the boronic acid (15.9 g, 95%). 1H nmr (300 MHz, DMSO), 2.5 (3H, s), 7.18 (2H, d, J=8 Hz), 7.70 (2H, d, J=8 Hz), 7.95 (2H, br s).

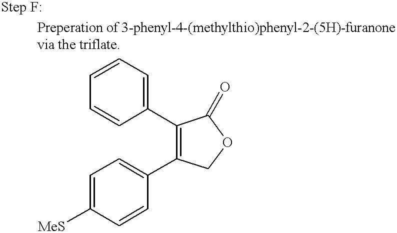

To a solution of the triflate (465 mg, 1 equiv.), Pd(tri-phenyl-P)₄ (87 mg, 0.05 equiv.) in degassed toluene (3 mL) (nitrogen atmosphere) was added aq sodium carbonate (2.1 equiv, 1.6 mL of a 2M solution) and the stirred mixture was heated to 60° C. A solution of the boronic acid (304 mg, 1.2 equiv.) in ethanol (1.6 mL) was added in one batch and the resulting mixture was heated at 60° C. for 2 h. The mixture was cooled to 25° C. and was then partitioned between ethyl acetate (10 mL) and water (5 mL). The organic layer was then concentrated to give a crude oil which was chromatographed on silica gel (30% EtOAc-70% hexanes) to give the furanone (317 mg, 70%). 1H nmr (300 MHz, CDCl3), 2.5 (3H, s), 5.17 (2H, s), 7.16 (2H, d, J=8 Hz), 7.23 (2H, d, J=8 Hz), 7.35-7.45 (5H, m).

Preparation of 3-phenyl-4-(methylthio)phenyl-2-(5H)-furanone via the bromide.

To a solution of the bromide (234 mg, 1 equiv.), Pd(PPh3)4 (55 mg, 0.05 equiv.) in degassed toluene (2 mL) (nitrogen atmosphere) was added aq sodium carbonate (2.1 equiv, 1.0 mL of a 2M solution) and water (1 mL) and the stirred mixture was heated to 60° C. A solution of the boronic acid (198 mg, 1.2 equiv.) in ethanol (0.75 mL) was added in one batch and the resulting mixture was heated at 60° C. for 9 h. The mixture was cooled to 25° C. and was then partitioned between ethyl acetate (10 mL) and water (5 mL). The organic layer was then concentrated to give a crude oil which was chromatographed on silica gel (30% EtOAc-70% hexanes) to give the furanone (265 mg, 90%).

Step G: Preparation of 3-phenyl-4-(4-methylsulfonyl)phenyl-2(5H)-furanone

The sulfide (1 g) and tetrabutylammonium bromide (0.034 g) were dissolved in methylene chloride(15 ml) at 25° C. Water (25 ml) and oxone (2.7 g) were added and the temperature was maintained at 25-30° C. for 24 h. The layers were separated and the organic layer was washed with water (20 ml). The organic was concentrated to dryness in vacuo to afford the desired sulphone (0.9 g).

### Example 2

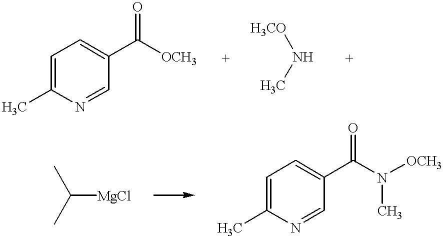

| | |
|---|---|
| Methyl 6-methylnicotinate | 21.56 g (0.143 mol) |
| N,O-Dimethylhydroxyamine | 13.9 g (0.229 mol) |
| Tetrahydrofuran | 150 mL |
| Isopropylmagnesium chloride | 110 mL (0.220 mol) |
| (2.0 M in THF) | |
| Toluene | 180 ml |

A solution of Methyl 6-methylnicotinate (21.56 g), and N,O-dimethylhydroxylamine (13.9 g) in THF (150 mL) was cooled to −10° C. Isopropylmagnesium chloride (110 mL) was added over 2.5 h. The reaction mixture was poured into aqueous acetic acid (10 vol %, 126 mL) at 5° C. Toluene (60 mL) was added to the mixture, then the layers were separated. The aqueous layer was extracted with toluene (2×60 mL) and the solvent removed. Solid impurities were removed by filtration and the filtrate was concentrated to afford the Weinreb amide as a light orange oil.

### Example 3

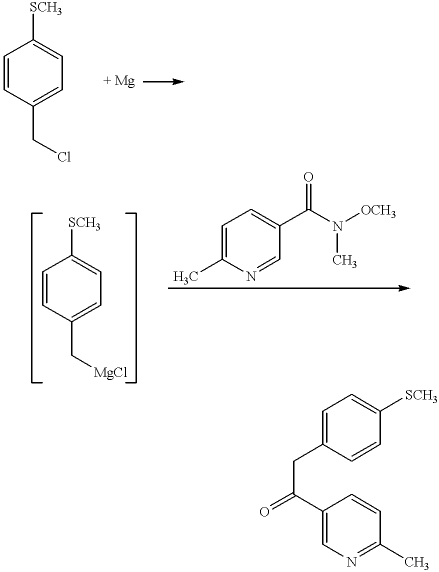

| | | |
|---|---|---|
| 4-thiomethylbenzyl chloride | 566 g | (3.28 mol) |
| Magnesium | | 191 g (7.86 mol) |
| Toluene | | 9 L |
| Tetrahydrofuran | | 0.545 L |
| Weinreb amide | | 300 g (1.66 mol) |

A mixture of magnesium (191 g, 7.86 mol) toluene (4 L), 4-thiomethylbenzyl chloride (566 g, 3.28 mol) and tetrahydrofuran (0.545L, 6.73 mol) were charged over 3-4 hours. An additional flask was charged with Weinreb amide (prepared as described in Example 2) (300 g, 1.66 mol) and toluene (1.7L) and cooled to −20° C. The Grignard solution prepared above was added over 30 minutes and the mixture was aged for 1 hour. The reaction mixture was quenched by the addition of 50% aqueous acetic acid (0.5L). Toluene (1L) and water (1L) were added and the layers were separated. The aqueous layer was extracted with toluene (2×2L). The combined organic extracts were extracted with dilute hydrochloric acid (1×2L). Ethyl acetate was added to the aqueous layer and the pH was adjusted with ammonia (0.6L). The phases were separated and the aqueous layer was extracted with ethyl acetate (2×1.25L). The combined extracts were concentrated on a rotary evaporator to provide the ketosulfide as a light yellow solid.

### Example 4

Oxidation

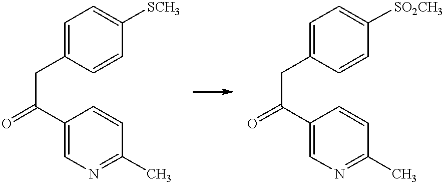

| | |
|---|---|
| Ketosulfide | 270 g (1.05 mol) |
| Methanol | 2.70 L |
| Sodium tungstate | 6.0 g (0.02 mol) |
| Water | 5.20 L |
| Sulfuric Acid (2 N) | 0.02 L |
| Hydrogen peroxide (30%) | 380 mL (3.0 mol) |

A mixture of ketosulfide (prepared as described in Example 3) (270 g, 1.05 mol), sulfuric acid (2N) (20 mL) ,and methanol (2.70L) was heated at 55° C. An aqueous solution of sodium tungstate (6.0 g, 0.02 mol) was added then hydrogen peroxide (380 mL) was added over 1 hour. Water (3L) was added and the mixture was cooled to ambient temperature then filtered. The solids were washed with water (2L) and dried under vacuum with a stream of nitrogen to give the ketosulfone as a colorless solid.

### Example 5

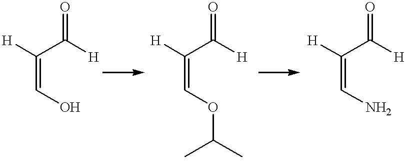

| | |
|---|---|
| Chloromalonaldehyde | 400 g (3.76 mol) |
| Aqueous ammonia (30%, 14.8 N) | 370 mL (5.48 mol) |
| Isopropyl alcohol | 6.4 L |

To a flask was charged with chloromalonaldehyde (400 g, 3.76 mol), and isopropyl alcohol (400 mL). The solution was concentrated under reduced pressure with a continuous, slow feed of isopropyl alcohol (4.0 L total). The resulting dark brown liquid was diluted with isopropyl alcohol (400 mL). The mixture was added to a cooled (5° C.) solution of 30% aqueous ammonia (370 mL) in isopropyl alcohol (2 L). The mixture was aged for 3 hours and the product was collected by filtration

### Example 6

5-Chloro-3(methylsulfonyl)phenyl-2-(3-pyridyl)-pyridine

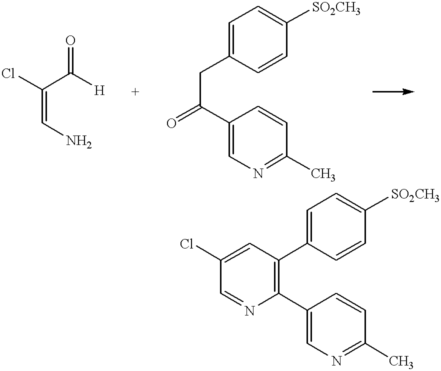

| | |
|---|---|
| 3-Amino-2-chloroacrolein | 140 g (1.31 mol) |
| Ketosulfone | 136 g (4.69 mol) |
| Methanesulfonic acid | 99.2 g (10.3 mol) |
| n-Propionic acid | 690 mL (92.5 mol) |
| Ammonium hydroxide (14.8 M) | 600 mL (88.8 mol) |
| Toluene | 1.35 L |

A mixture of n-propionic acid (400 mL), 3-amino-2-chloroacrolein (prepared as described in Example 5) (140 g, 1.31 mol), ketosulfone (prepared as described in Example 4) (136 g, 0.469 mol), toluene (1.35 L), propionic acid (690 mL, 92.5 mol), methanesulfonic acid (67 mL, 10.3 mol) was heated to reflux (114° C.) for 12 hours with the azeotropic removal of water. The reaction solution was cooled to ambient temperature and diluted with isopropyl acetate (1 L). Water (1 L) was added and the aqueous phase was neutralized with concentrated ammonium hydroxide solution (600 mL). The organic layer was washed with a 1:1 mixture of brine/water (2×1 L) and water (1 L). The combined aqueous layers were extracted with isopropyl acetate (900 mL). The combined organic layers were treated with Darco G-60 then concentrated. Recrytallization from isopropylacetate/ hexanes provided the title compound as a colorless solid mp 135° C. (DSC).

### Example 7

A number of routes are available for the preparation of chloromalondialdehyde.

Preparation from 1,1,2,3,3-Pentachloropropane

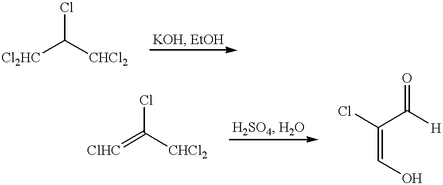

A detailed experimental is published in Houben-Weyl-Muller: Methoden der Organischen Chemie, 4th Edit., Vol 7/1, Thieme Verlag, Stuttgart, 1954, page 119. The starting material 1,1,2,3,3-pentachloropropane is commercially available from Pfaltz and Bauer.

Preparation from Mucochloric Acid

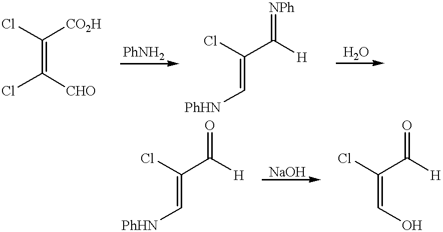

The following is a slight variation of the original procedure of
Dieckmann (Ber. Deut. Chem. Ges. 1904, 37,4638).

| | |
|---|---|
| Mucochloric acid | 50.0 g (0.30 mol) |
| Aniline | 54 mL (0.60 mol) |
| Water | 1000 mL |

To a solution of aniline in water at 85° C. in a vigorously stirred 2 L flask was added mucochloric acid in small portions over 30 min. On addition of the mucochloric acid, a yellow color develops, which quickly dissipated. The reaction mixture stayed heterogeneous and filtration of an aliquot after 30 min heating indicated completion of the reaction.

The reaction mixture was heated at 90° C. for 60 min., cooled to 50° C. and filtered. The filtercake was washed with 50 mL of 2N HCl and 100 mL of H₂O. The product was dried in a N₂ stream to give 3-anilido-2-chloro-acrolein as a gray solid. ¹³C NMR (D₆-DMSO in ppm):108, 117, 124, 129, 140. 147, 182.

| | | |
|---|---|---|
| 3-Anilido-2-chloro-acrolein | 57 g | (0.30 mol) |
| 5N NaOH solution | 120 mL | (0.6 mol) |

A solution of 3-anilido-2-chloro-acrolein in 120 mL of 5N NaOH was heated to 100° C. for 90 min. The dark black solution was extracted twice with 50 mL each of MTBE.

The first organic wash removed most of the dark color from the solution, and the second organic wash was only lightly colored.

On cooling the aqueous phase, a crystalline precipitate formed. This product was the 3-chloromalondialdehyde Na salt.

The aqueous phase was acidified by the addition of 60 mL of 37% HCl solution. The aqueous phase was extracted (MTBE/THF 50/50, 400 mL total) and the combined organic phases were dried over MgSO₄. After treatment with Darco G60 and filtration through a plug of SiO₂, the solution was evaporated to give of chloromalondialdehyde as a dark solid. Recrystallization from ca. 10 mL of MTBE gave 11.13 g of pure chloromalondialdehyde as a tan solid. ¹³C NMR (D₆-DMSO in ppm): 113, 175 (broad).

Preparation from Chloroacetylchloride

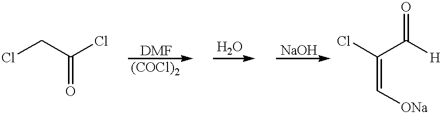

Arnold (Collect. Czech. Chem. Commun. 1961, 26, 3051) mentions the formation of 3-dimethylamino-2-chloro-acrolein by reaction of chloroacetic acid with the Vilsmeyer reagent derived from POCl₃ and DMF. A variation and extension of his procedure prepares chloromalondialdehyde as its Na salt.

Oxalylchloride (280 mL, 3.2 mol) was added at 10° C. to 1000 mL of DMF. The reaction was highly exothermic and a heavy precipitate formed. After a 2 h age, chloroacetylchloride (110 mL, 1.4 mol) was added and the reaction mixture was warmed to 75° C. for 3 hours. Analysis of an aliquot by ¹H NMR indicated complete consumption of the chloroacetylchloride and the reaction mixture was quenched by addition into 1 L of H20. To the cooled solution was added 500 mL of a 50% NaOH solution. The reaction mixture is heated to reflux for 5 hours. On cooling a precipitate formed, which was filtered and washed with water. The tan solid was dried in a N₂ stream to give a tan solid.

### Example 8

5-Chloro-3(methylsulfonyl)phenyl-2-(3-pyridyl)-pyridine;

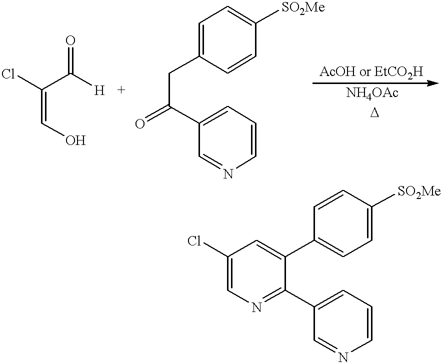

| | |
|---|---|
| 2-Chloromalondialdehyde | 4.8 g (0.045 mol) |
| Ketosulfone | 5.0 g (0.018 mol) |
| Propionic acid | 30 mL |
| Ammonium Acetate | 8.4 g (0.11 mol) |

A mixture of ketosulfone (prepared as described in Example 4) (5.0 g), 2-chloromalondialdehyde (prepared as described in Example 7) (4.8 g) and ammonium acetate were heated to 130° C. The acetic acid produced was removed by distillation and heating continued at 136° C. for 15 hours. The reaction mixture was basified with sodium carbonate, water was added and the product was extracted into dichloromethane (2×150 mL). The organic layers were carbon treated (Dowex ), dried (MgSO₄) and the solvent removed to afford the title compound as an off white solid.

### Example 9

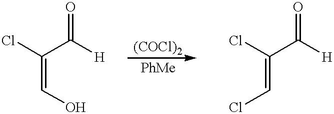

| | |
|---|---|
| 2-Choromalondialdehyde | 220 mg (2.1 mmol) |
| Oxalyl Chloride | 180 mL (2.1 mmol) |
| Toluene | 3 mL |
| N,N-Dimethylformamide | 20 mL |

N,N-dimethyl formamide was added to a slurry of 2- chloromalondialdehyde (220 mg) in toluene. Oxalyl chloride was added and the reaction mixture was stirred until complete dissolution occurred.

### Example 10

5-Chloro-3(methylsulfonyl)phenyl-2-(3-pyridyl)-pyridine;

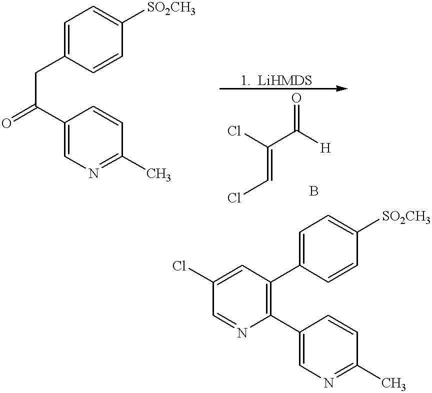

| | |
|---|---|
| Ketone B | 500 mg (1.8 mmol) |
| Lithium bis(trimethylsilyl)amide (1 M in THF) | 1.8 mL (1.8 mmol) |
| Tetrahydrofuran | 15 mL |
| 2,3-Dichloroacrolein in toluene | 2.1 mmol in 3 mL |
| | toluene |
| Ammonium acetate | 1.0 g |

Lithium bis(trimethylsilyl)amide (1.8 mL;1 M in THF) was added dropwise to ketosulfone (prepared as described in Example 4) (500 mg) in THF (15 mL) at −78° C. The reaction mixture was warmed to ambient temperature for 1 hour to form the lithium enolate of the ketosulfone before recooling to −78° C. A solution of 2,3-dichloroacrolein was added and the temperature allowed to warm to room temperature. After 1 hour ammonia gas was passed through the solution and after 30 minutes ammonium acetate (1 g) was added. The reaction mixture was warmed to 60° C. for 1 hour and poured into aqueous sodium hydroxide (2 M; 100 mL). The product was extracted with dichloromethane (2×150 mL), dried (MgSO4) and the solvent removed to afford the title compound.

### Example 11

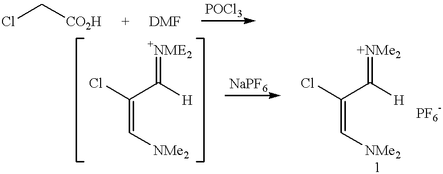

Chloroacetyl chloride (14.50 g, 0.112 mol) was added to dimethylformamide (50 mL) and the mixture was heated to 75° C. to give a clear yellow solution. Phosphorus oxychloride (18.9 g, 0.123 mol) was added at 5 mL/h. The reaction mixture is aged for 3 h then cooled to ambient temperature. The reaction mixture and 5N sodium hydroxide (70 mL) were added concurrently over 1 hr to a mixture of water (200 mL) and sodium hexafluorophosphate (21 g, 0.125 mol) at <9° C. The reaction flask was washed with dimethylformamide (2 mL) and added to the quench. The mixture was aged for 40 minutes then filtered.

The crude solid was washed with water (100 mL). The solid was recrystallized from water (224 mL) and isopropanol (56 mL) by heating to 70° C. The mixture was cooled to 4° C. then filtered. The solid was washed with water/isopropanol (100 mL, 20:1) and dried to give Intermediate 1 as a light yellow solid

### Example 12

5-Chloro-3(methylsulfonyl)phenyl-2-(3-pyridyl)-pyridine;

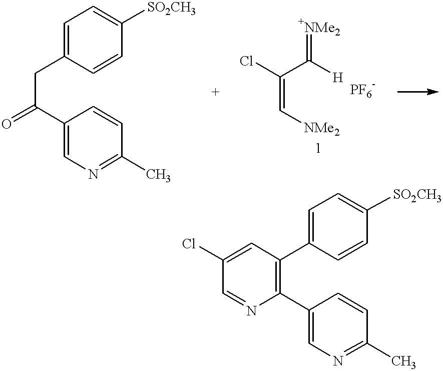

To a suspension of ketosulfone (prepared as described in Example 4) (500 mg) (1.5 kg, 5.12 mol) in THF (10L) was added potassium butoxide (617 g, 5.5 mol) in THF (5.38L, 5.38 mol) at <15° C. Intermediate 1 (prepared as described in Example 11) (1.65 kg,. 4.6 mol) was added and the reaction mixture was aged at ambient temperature. The reaction mixture was transferred to a solution of acetic acid (2.0L) in THF (5 L) and the mixture was stirred for 1 hr. Concentrated aqueous ammonium hydroxide (4 L) was added and the mixture was heated at reflux for 3 hrs. The mixture was cooled to 22° C. and the layers were separated. The organic layer was concentrated to 3 L and isopropyl acetate (5L) was added. The resulting solution was again concentrated to 3-4 L and isopropyl acetate (19L) was added. The solution was washed with saturated sodium bicarbonate (2×9.5L) and water (2×9.5L), concentrated to dryness and purified to provide the title compound as a solid.

### Example 13

Preparation of [N-Ac-(4-trans-L-Hyp)]-Ala-Ser-Chg-Gln-Ser-Leu-Dox (SEQ.ID.NO.: 22)

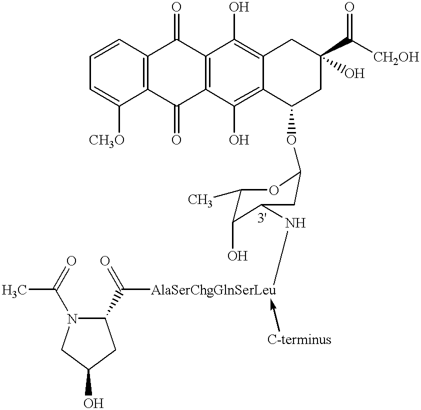

Step A: [N-Ac-(4trans-L-Hyp(Bzl))]-Ala-Ser(Bzl)Chg-Gln-Ser(Bzl)Leu-PAM Resin (13-1).

Starting with 0.5 mmol (0.67 g) Boc-Leu-PAM resin, the protected peptide was synthesized on a 430A ABI peptide synthesizer. The protocol used a 4 fold excess (2 mmol) of each of the following protected amino acids: Boc-Ser(Bzl), Boc-Gln, Boc-Chg, Boc-Ala, N-Boc-(4-trans-L-Hyp(Bzl)). Coupling was achieved using DCC and HOBT activation in methyl-2-pyrrolidinone. Acetic acid was used for the introduction of the N terminal acetyl group. Removal of the Boc group was performed using 50% TFA in methylene chloride and the TFA salt neutralized with diisopropylethylamine. At the completion of the synthesis the peptide resin was dried to yield Intermediate 13- 1.

Step B: [N-Ac-(4-trans-L-Hyp)]-Ala-Ser-Chg-Gln-Ser-Leu-OH (13-2)

The protected peptide resin (13-1), 1.2 g, was treated with HF (20 ml) for 1 hr at 0° C. in the presence of anisole (2 ml). After evaporation of the HF, the residue was washed with ether, filtered and extracted with H₂O (200 ml). The filtrate was lyophilyzed to yield Intermediate 13-2.

Step C: [N-Ac-(4-trans-L-Hyp)]-Ala-Ser-Chg-Gln-Ser-Leu-Dox

The above described intermediate (13-2), 1.157 g (1.45 mmol) was dissolved in DMSO (30 ml) and diluted with DMF (30 ml). To the solution was added doxorubicin hydrochloride, 516 mg (0.89 mmol) followed by 0.310 ml of diisopropylethylamine (1.78 mmol). The stirred solution was cooled (0° C.) and 0.276 ml of diphenylphosphoryl azide (1.28 mmol) added. After 30 minutes, an additional 0.276 ml (1.28 mmol) of DPPA was added and the pH adjusted to 7.5 (pH paper) with diisopropylethylamine (DIEA). The pH of the cooled reaction (0° C.) was maintained at 7.5 with DIEA for the next 3 hrs. and the reaction stirred at 0-4° C. overnight. After 18 hrs., the reaction (found to be complete by analytical HPLC, system A) was concentrated to an oil. Purification of the crude product was achieved by preparative HPLC, Buffer A=0.1% NH₄OAc-H₂O; B=CH₃CN. The crude product was dissolved in 400 ml of 100% A buffer, filtered and purified on a C-18 reverse phase HPLC radial compression column (Waters, Delta-Pak, 15 μM, 100Å). A step gradient of 100% A to 60% A was used at a flow rate of 75 ml/min (UV=214 nm). Homogeneous product fractions (evaluated by HPLC, system A) were pooled and freeze-dried. The product was dissolved in H₂O (300 ml), filtered and freeze-dried to provide the purified title compound.

PHYSICAL PROPERTIES
The physical/chemical properties of the product of Step C are shown
below:

| | |
|---|---|
| Molecular Formula: | C₆₂H₈₅N₉O₂₃ |
| Molecular Weight: | 1323.6 |
| High Resolution ES Mass Spec: | 1341.7 (NH₄⁺) |
| HPLC: | System A |
| Column: | Vydac 15 cm #218TP5415, C18 |
| Eluant: | Gradient 95:5 (A:B) to 5:95 (A:B) over |
| | 45 min. A = 0.1% TFA/H₂O, B = |
| | 0.1% |
| | TFA/Acetonitrile |
| Flow: | 1.5 ml/min. |
| Wavelength: | 214 nm, 254 nm |
| Retention Time: | 18.2 min. |

Amino Acid Compositional Analysis¹:

| Theory | Found |
|---|---|
| Ala (1) | 1.00 |
| Ser (2) | 1.88 |
| Chg (1) | 0.91 |
| Gln² (1) | 1.00 (as Glu) |
| Hyp (1) | 0.80 |
| Leu (1) | 1.01 |
| Peptide Content: | 0.657 μmol/mg |

Note: ¹20 hr., 100° C., 6N HCl
²Gln converted to Glu

### Example 14

Preparation of [N-Glutaryl-(4-trans-L-Hyp)]-Ala-Ser-Chg-Gln-Ser-Leu-Dox (SEQ.ID.NO.: 25) (Compound B)

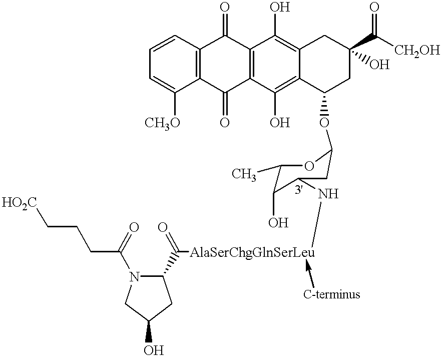

Step A: [N-Glutaryl(OFm)-(4-trans-L-Hyp)]-Ala-Ser-Chg-Gln-Ser-Leu-PAM Resin

Starting with 0.5 mmol (0.67 g) Boc-Leu-PAM resin, the protected peptide was synthesized on a 430A ABI peptide synthesizer. The protocol used a 4 fold excess (2 mmol) of each of the following protected amino acids: Fmoc-Ser(tBu), Fmoc-Gln(Trt), Fmoc-Chg, Fmoc-Ala, Boc-(4-trans-L-Hyp). Coupling was achieved using DCC and HOBT activation in methyl-2-pyrrolidinone. The intermediate mono fluorenylmethyl ester of glutaric acid [Glutaryl(OFm)] was used for the introduction of the N-terminal glutaryl group. Removal of the Fmoc group was performed using 20% piperidine. The acid sensitive protecting groups, Boc, Trt and tBu, were removed with 50% TFA in methylene chloride. Neutralization of the TFA salt was with diisopropylethylamine. At the completion of the synthesis, the peptide resin was dried to yield the title compound.

Step B: [N-Glutargl(OFm)-(4-trans-L-Hyp)]-Ala-Ser-Chg-Gln-Ser-Leu-OH

The protected peptide resin from Step A, 1.2 g, was treated with HF (20 ml) for 1 hr at 0° C. in the presence of anisole (2 ml). After evaporation of the HF, the residue was washed with ether, filtered and extracted with DMF. The DMF filtrate (75 ml) was concentrated to dryness and triturated with H₂O. The insoluble product was filtered and dried to provide the title compound.

Step C: [N-Glutaryl(OFm)-(4-trans-L-Hyp)]-Ala-Ser-Chg-Gln-Ser-Leu-Dox

The above prepared intermediate from Step B, (1.33 g, 1.27 mmol) was dissolved in DMSO (6 ml) and DMF (69 ml). To the solution was added doxorubicin hydrochloride, 599 mg (1.03 mmol) followed by 376 μl of diisopropylethylamine (2.16 mmol). The stirred solution was cooled (0° C.) and 324 μl of diphenylphosphoryl azide (1.5 mmol) added. After 30 minutes, an additional 324 μl of DPPA was added and the pH adjusted to 7.5 (pH paper) with diisopropylethyl-amine (DIEA). The pH of the cooled reaction (0° C.) was maintained at ˜7.5 with DIEA for the next 3 hrs and the reaction stirred at 0-4° C. overnight. After 18 hrs., the reaction (found to be complete by analytical HPLC, system A) was concentrated to provide the title compound as an oil.

Step D: [N-Glutaryl-(4-trans-L-Hyp)]-Ala-Ser-Chg-Gln-Ser-Leu-Dox

The above product from Step C was dissolved in DMF (54 ml), cooled (0° C.) and 14 ml of piperidine added. The solution was concentrated to dryness and purified by preparative HPLC. (A=0.1% NH₄OAc-H₂O; B=CH₃CN.) The crude product was dissolved in 100 ml of 80% A buffer, filtered and purified on a C-18 reverse phase HPLC radial compression column (Waters, Delta-Pak, 15μ, 100Å). A step gradient of 80% A to 67% A was used at a flow rate of 75 ml/min (uv=214 nm). Homogeneous product fractions (evaluated by HPLC, system A) were pooled and freeze-dried. The product was further purified using the above HPLC column. Buffer A=15% acetic acid-H₂O; B=15% acetic acid-methanol. The product was dissolved in 100 ml of 20% B/80% A buffer and purified. A step gradient of 20% B to 80% B was used at a flow rate of 75 ml/min (uv=260 nm). Homogeneous product fractions (evaluated by HPLC, system A) were pooled, concentrated and freeze-dried from H₂O to yield the purified title compound.

| | |
|---|---|
| High Resolution ES Mass Spec: | 1418.78 (Na⁺) |
| HPLC: | System A |
| Column: | Vydac 15 cm #218TP5415, C18 |
| Eluant: | Gradient 95:5 (A:B) to 5:95 (A:B) over |
| | 45 min. |
| | A = 0.1% TFA/H₂O, B = 0.1% TFA/ |
| | Acetonitrile |
| Flow: | 1.5 ml/min. |
| Wavelength: | 214 nm, 254 nm |
| Retention Time: | 18.3 min. |

Amino Acid Compositional Analysis¹:

| Theory | Found |
|---|---|
| Ala (1) | 0.99 |
| Ser (2) | 2.02 |
| Chg (1) | 1.00 |
| Gln² (1) | 1.01 (as Glu) |
| Hyp (1) | 0.99 |
| Leu (1) | 1.00 |
| Peptide Content: | 0.682 μmol/mg |

Note: ¹20 hr., 100° C., 6N HCl
²Gln converted to Glu

### Example 14A

Preparation of [N-Glutaryl-(4-trans-L-Hyp)]-Ala-Ser-Chg-Gln-Ser-Leu-Dox Sodium Salt (SEQ.ID.NO.: 25)

Preparation N-(N′-(Fm-Glutaryl)-trans-4-hydroxy-L-prolinyl-alanyl)serine

Step 1: N-Boc-trans-4-hydroxy-L-proline

A solution of trans-4-hydroxy-L-proline (3.0 kg, 22.88 M) in 1 M aqueous sodium hydroxide (25.2 L) and tert-butanol (12.0 L) was treated with a solution of di-tert-butyldicarbonate (5.09 kg) in tert-butanol (6.0 L) at 20° C. over 20 minutes. Upon complete addition, the resulting solution was stirred at 20° C. for 2 hours. The solution was extracted with hexane (2×15.0 L) and then acidified to pH 1 to 1.5 by cautious addition of a solution of potassium hydrogen sulphate (3.6 kg) in water (15.0 L). The mixture was extracted with ethyl acetate (3×15.0 L). The combined ethyl acetate extracts were washed with water (2×1.0 L) and dried by azeotropic distillation at atmospheric pressure (final KF of ethyl acetate solution <0.1%)

The ethyl acetate solution was then concentrated by atmospheric distillation to a volume of 15.0 L, diluted with hexane (8.0 L), seeded and stirred at 20° C. for 1 hour. Hexane (22.5 L) was added over 2 hours, the slurry was cooled to 0° C. for 1 hour and the solid collected by filtration. The product was washed with cold (0° C.) 2:1 hexane/ethyl acetate (15.0 L) and dried in vacuo at 45° C. to afford the title compound as a white crystalline solid.

Step 2: N-Boc-trans-4-hydroxy-L-proline Pentafluorophenyl ester

Boc-trans-4-hydroxy-L-proline (3.5 kg) (prepared as described in Step 1) and pentafluorophenol (3.06 kg) were dissolved in ethyl acetate (52 L). The solution was treated with a solution of dicyclohexylcarbodiimide (3.43 kg) in ethyl acetate (8 L) and the mixture was stirred at room temperature for 2 hours. The resulting slurry was cooled to 0° C., filtered and the solids washed with ethyl acetate (15 L). The filtrate was evaporated at atmospheric pressure to a volume of 10 L and diluted with hexane (100 L). The resulting mixture was stirred at room temperature overnight and then cooled to 0° C. for 1 hour. The solid was collected by filtration, washed with cold (° C.) 10:1 hexane/ethyl acetate (I5 L) and dried at 45° C. in vacuo to afford the title compound as a white crystalline solid.

Step 3: N-(trans-4-hydroxy-L-prolinyl-alanyl)serine hydrochloride

N-alanylserine (1.5 kg, 8.515 M) and Boc-trans-4-hydroxy-L-proline (3.72 kg) (prepared as described in step 2) were heated at 50° C. in dimethylformamide (15 L) for 3 hours. The solution was cooled to 20° C., treated with concentrated hydrochloric acid (7.5 L) and stirred at room temperature for 24 hours. The resulting slurry was diluted with isopropanol (30 L), stirred at room temperature for 30 minutes and then cooled to 0° C. for 1 hour. The solid was collected by filtration and washed with isopropanol (20 L). The solid was dried in vacuo at 40° C. to afford the title compound as a white crystalline solid.

Step 4: Fluorenylmethyl Glutarate

9-Fluorenyl methanol (2.0 kg), glutaric anhydride (2.33 kg) and sodium bicarbonate (1.71 kg) were stirred together in N-methylpyrrolidinone (8.0 L) at room temperature for 72 hours. The slurry was filtered and the solids washed with isopropyl acetate (2×10.0 L). The filtrate was washed with 1.0 M hydrochloric acid (3×10.0 L). The organic layer was extracted with 1.0 M aqueous sodium hydroxide (3×8.0 L). The combined basic extracts were covered with isopropyl acetate (20.0 L) and acidified to pH 2 with 2.0 M hydrochloric acid (12.5 L). The phases were separated and the aqueous phase was extracted with isopropyl acetate (10.0 L).

The combined organic phases were washed with water (10.0 L) and dried by azeotropic distillation at <60° C. under reduced pressure (KF <0.05%). The solution was then concentrated under reduced pressure (<60° C.) to a volume of 7.0 L. The solution was diluted with hexane (6.0 L), seeded and stirred at room temperature for 30 minutes. The resulting slurry was diluted by addition of hexane (42.0 L) over 40 minutes. The slurry was cooled to 0° C. for 1 hour and the solid collected by filtration and washed with cold (0° C.) 8:1 hexane/iPAc (20.0 L). The solid was dried in vacuo at 45° C. to afford the title compound as a pale cream solid.

Step 5: Fluorenylmethyl Glutarate Pentafluorophenyl Ester

Fluorenylmethyl glutarate (2.5 kg) (prepared as described in Step 4) and pentafluorophenol (1.63 kg) were dissolved in ethyl acetate (25 L). The solution was treated with a solution of dicyclohexylcarbodiimide (1.83 kg) in ethyl acetate (7.5 L) and the mixture was stirred at 20° C. overnight. The resulting slurry was filtered and the solids were washed through with ethyl acetate (10 L). The filtrate was evaporated at atmospheric pressure to a volume of 7.5 L and diluted with hexane (75 L). The slurry was filtered at 60-65° C. then allowed to cool to room temperature and stirred overnight. The slurry was cooled to 0° C. for 1 hour, the solid collected by filtration and washed with 10:1 hexane/ethyl acetate (15 L). The solid was dried in vacuo at 45° C. to afford the title compound as a white crystalline solid.

Step 6: N-(N′-(Fm-Glutaryl)-trans-4-hydroxy-L-prolinyl-alanyl)serine

N-(trans-4-hydroxy-L-prolinyl-alanyl)serine hydrochloride (2.3 kg) (prepared as described in Step 3) was suspended in dimethylformamide (22 L) and the slurry was treated with N-ethylmorpholine (911 ml) followed by a solution of fluorenylmethyl glutarate pentafluorophenyl ester (3.5 kg) (prepared as described in Step 5) in dimethylformamide (14 L). The mixture was heated at 50° C. for 3 hours and the resulting solution evaporated to residue under reduced pressure. The residue was partitioned between water (80 L) and tert-butyl methyl ether (34 L). The phases were separated and the aqueous layer was extracted with tert-butyl methyl ether (34 L). The aqueous solution was seeded and stirred at room temperature overnight. The solid was collected by filtration (slow) and washed with water (25 L). The damp filter cake was dissolved in isopropanol (90 L) with warming and the solution concentrated to half volume by distillation at atmospheric pressure. Additional portions of isopropanol (3×45 L) were added and the batch was concentrated to cα half volume by atmospheric distillation after addition of each portion (Final KF of liquors <0.5%). The slurry was diluted with isopropanol (23 L), stirred at 20° C. overnight, cooled to 0° C. for 1 hour and the solid collected by filtration. The cake was washed with isopropanol (20 L) and the solid dried in vacuo at 45° C. to afford the crude product as a white solid.

Step 7: Recrystallisation of N-(N′-(Fm-Glutaryl)-trans-4-hydroxy-L-prolinyl-alanyl) serine

N-(N′-(Fm-Glutaryl)-trans-4-hydroxy-L-prolinyl-alanyl)serine (3.4 kg) (prepared as described in Step 6) was dissolved in methanol (51 L) at reflux. The solution was filtered and concentrated by atmospheric distillation to a volume of 17 L (5 ml/g). The solution was diluted with ethyl acetate (102 L) allowed to cool to 20° C. and stirred overnight. The resulting slurry was cooled to 0° C. for 1 hour and the solid was collected by filtration. The cake was washed with cold (0° C.) 10:1 ethyl acetate/methanol (20 L) and dried in vacuo at 45° C. to afford the product as a white solid.

Preparation N-(cyclohexylglycyl-glutaminyl-serinyl)leucine benzyl ester hydrochloride (SEQ.ID.NO.: 51)

Step 8: N-(serinyl)leucine benzyl ester hydrochloride

Leucine benzyl ester p-tosylate (1000 g) and HOBt (412 g) were slurried in isopropyl acetate (12 L). The mixture was cooled to 0° C. in an ice-bath and a slurry of sodium bicarbonate (469.7 g) in water (1 L), N-BOC-L-serine (573.6 g) in water (2 L) and EDC.HCl (560.2 g) in water (2 L) were added. The mixture was allowed to warm to 20° C. over 30 minutes and aged at 20° C. for 2 hours (<1 A% Leu-OBn remaining). If the reaction was not complete after 2 hours, further NaHCO₃ and EDC.HCl were added. The phases were separated and the organic layer was washed sequentially with saturated sodium bicarbonate (2×3.75 L), 0.5 M sodium hydrogen sulphate (2×3.75 L) and water (2×2.5 L).

The wet, isopropyl acetate solution was concentrated under reduced pressure to 3 L and the water content checked. (KF=0.12%. It is important that this solution is dry prior to the addition of hydrogen chloride in isopropyl acetate). The solution was transferred to a 20 L round bottom flask under a nitrogen atmosphere and cooled to 0° C. To the solution was added 3.6 M HCl in isopropyl acetate (7 L, 10 mol equiv. HCl). The product began to crystallize after 5 minutes. The reaction was aged at 0° C. for 1 hr, and then allowed to warm to room temperature.

The slurry was cooled to 0-5° C., diluted with heptane (2.5 L) and aged at 0° C. for 30 minutes. The product was collected by filtration, washed with cold isopropyl acetatelheptane (4:1) (2.5 L) and dried in vacuo at 35° C., with a nitrogen sweep.

Step 9: N-(N′-(Boc)-glutaminyl-serinyl)leucine benzyl ester

N-(serinyl)leucine benzyl ester hydrochloride (350 g) (prepared as described in Step 8), HOBt (157.7 g) and N-Boc-L-glutamine (262.5 g) were slurried in DMF (2.5 L) and the mixture was cooled to 0° C. N-Ethylmorpholine (245.5 g) and EDC.HCl (214 g) were added and the mixture was aged at 0° C. for 2.5 hours. Water (14.7 L) was added over 20 minutes and the white slurry aged at 0° C. for 1 hour. The product collected by filtration and washed with water (3.2 L). The cake was dried in the fume-hood overnight. The isolated N-BOC-Gln-Ser-Leu-OBn, which contained DMF and HOBt, was combined with a second batch of identical size, and swished in water (12 L) at 20° C. for 1 hour. The product was collected by filtration, washed with water (2.5 L) and air-dried in a fume-hood over the weekend. The batch was dried in vacuo, at 42° C., with a nitrogen bleed.

Step 10: N-(glutaminyl-serinyl)leucine benzyl ester hydrochloride

N-(N′-(Boc)-glutaminyl-serinyl)leucine benzyl ester (715 g, 1.33 M) (prepared as described in Step 9) was suspended in iPAc (3.5 L) at room temperature. To the slurry was added a 3.8 M solution of HCl in iPAc (3.5 L, 13.3 M) whereupon all the solids dissolved. After a short time, the product crystallized. The mixture was stirred at room temperature for 3.75 hours when HPLC showed complete reaction. The slurry was diluted with iPAc (4.0 L), stirred for 1 hour at room temperature and the solid collected by filtration under nitrogen. The product is very hygroscopic in the presence of excess HCl and must be collected under dry nitrogen.

The cake was washed with iPAc (4.0 L), the solid dried on the filter under nitrogen for 2 hours and then dried in vacuo at 45° C.

Step 11: N-(N′-(Boc)-cyclohexylglycylglutaminyl-serinyl)leucine benzyl ester(SEQ.ID.NO.: 52)

N-(glutaminyl-serinyl)leucine benzyl ester hydrochloride (2.6 kg) (prepared as described in Step 10), N-Boc-L-cyclohexylglycine (1.414 kg) and HOBt hydrate (168 g) were dissolved in DMF (13.0 L). N-ethylmorpholine (1.266 kg, 11.0 M) and EDC hydrochloride (1.265 kg) were added and the mixture stirred at 20° C. for 3 hours. The solution was diluted with ethyl acetate (13.0 L) and water (26.0 L) added. The product precipitated and the slurry was stirred at room temperature for 1 hour. The solid was collected by filtration, washed with 1:1 ethyl acetate/water (60 L) dried on the filter under nitrogen for 24 hours and dried in vacuo at 45°. The title compound was obtained as a white solid.

Step 12: N-(cyclohexylglycyl-glutaminyl-serinyl)leucine benzyl ester hydrochloride (SEQ.ID.NO.: 51)

N-(N′-(Boc)-cyclohexylglycylglutaminyl-serinyl)leucine benzyl ester (1850 g) (prepared as described in Step 11) was slurried in isopropyl acetate (3.2 L). The slurry was cooled to 0° C. in an ice bath and 3.8 M HCl/isopropyl acetate (3.7 L, 11.4 mol equiv.) was added over 5 minutes, maintaining the temperature between 8 and 10° C. The starting material had dissolved after 15-20 minutes. The solution was seeded and the reaction aged at 8-10° C. for 2 hrs, (<1A % N-Boc-tetrapeptide-OBn remaining). The batch was filtered, under a nitrogen blanket, washed with cold (10° C.) isopropyl acetate (4×3 L) then dried on the filter under nitrogen. The solid was dried in vacuo, at 40° C.

The crude N-(cyclohexylglycyl-glutaminyl-serinyl)leucine benzyl ester hydrochloride (2.2 Kg) was slurried in methanol (22.3 L) at room temperature. The batch was stirred for 1 hour and then ethyl acetate (44.6 L) was added over 30 minutes. The batch was cooled to 0-5° C., aged for one hour, then filtered and washed with cold (0-5° C.) methanol/ethyl acetate (6 L, 1:2). The solid was dried on the filter, under nitrogen, for 45 minutes and then dried in vacuo, at 40° C., with a nitrogen sweep.

The N-(cyclohexylglycyl-glutaminyl-serinyl)leucine benzyl ester hydrochloride (1.478 Kg) was slurried in methanol (14.8 L) at room and the batch stirred for 1 hr. Ethyl acetate (29.6 L) was added over 30 minutes, the batch was cooled to 0-5° C. and aged for an hour. The solid collected by filtration, washed with cold (0-5° C.) methanol/ethyl acetate (4.5 L, 1:2), dried on the filter for 45 minutes, under nitrogen, and then dried under vacuum, at 40° C. This material was then utilized in subsequent reactions.

Preparation N-( N′-(Fm-Glutaryl)-trans-4-hydroxy-L-prolinyl-alanyl-serine-cyclohexylglycyl-glutaminyl-serinyl) leucine (Compound 12) (SEQ.ID.NO.: 53)

Step 13: N-(N′-(Fm-Glutaryl)-trans-4-hydroxy-L-prolinyl-alanyl-serine-cyclohexylglycyl-glutaminyl-serinyl) leucine benzyl ester (SEQ.ID.NO.: 54)

N-(cyclohexylglycyl-glutaminyl-serinyl)leucine benzyl ester hydrochloride (500 g) (prepared as described above), N-(N′-(Fm-Glutaryl)-trans-4-hydroxy-L-prolinyl-alanyl) serine (490 g) (prepared as described above) and HOAt (160 g) were slurried in DMF (8.2 L) and cooled to 2° C. in an ice bath. N-ethylmorpholine (135 ml) was added followed by EDC.HCl (210 g). The mixture was stirred at 0-2° C. for 2 hours and sampled. HPLC showed 0.2 A % tetrapeptide remaining. The reaction mixture was diluted with ethyl acetate (4 L) and transferred to a 30-gallon glass vessel through a 5 μ in-line filter. The flask and lines were rinsed with ethyl acetate/DMF (1:1, 500 ml) and ethyl acetate (4 L). Water (16.4 L) was added over 25 minutes (temperature 11° C. to 23° C.) and the mixture stirred slowly, at 20° C., for 30 minutes. The product was collected by filtration, washed with water (3 L), ethyl acetate (1 L) and water (2×3 L), then dried on the filter under nitrogen, and dried in vacuo at 45° C.

Alternate Step 13: Fm-Glutaryl-Hyp-Ala-Ser-Chg-Gln-Ser-Leu-O-benzyl (SEQ.ID.NO.: 54)

HCl.H-Chg-Gln-Ser-Leu-OBn (100 g), Fm-Glutaryl-Hyp-Ala-Ser-OH (98 g) and 4-hydroxypyridine-N-oxide (HOPO, 18.2 g) were slurried in DMF (1.6 L) and cooled to 2° C. in an ice bath. N-ethylmorpholine (27 ml) was added followed by EDC.HCl (42 g). The mixture was stirred at 2-5° C. for 4 hours and sampled. HPLC showed 0.6 A % tetrapeptide remaining. The reaction mixture was diluted with ethyl acetate (1.64 L), water (3.3 L) was added over 70 minutes and the mixture stirred slowly, at 20° C., for 60 minutes. The product was collected by filtration, washed with water (1.5 L), ethyl acetate (1 L) and water (3×1 L), then dried on the filter under nitrogen, and dried in vacuo at 45° C.

Step 14: N-(N′-(Fm-Glutaryl)-trans-4-hydroxy-L-prolinyl-alanyl-serine-cyclohexylglycyl-glutaminyl-serinyl) leucine (SEQ.ID.NO.: 53)

N-(N′-(Fm-Glutaryl)-trans-4-hydroxy-L-prolinyl-alanyl-serine-cyclohexylglycyl-glutaminyl-serinyl) leucine benzyl ester (1.1 Kg) (prepared as described in Step 13) was dissolved in dimethylacetamide (7.8 L) containing methanesulphonic acid (93.5 ml). 5% Pd/C (110 g, 10 wt%), slurried in DMA (1.0 L), was added and the mixture hydrogenated at atmospheric pressure for 1 hour 40 minutes. The reaction mixture was sampled: HPLC showed no starting material remaining.

The reaction mixture was filtered through a pre-wetted (DMA) pad of hyflo (500 g) to remove the catalyst. The hyflo pad washed with DMA (2.2 L) and then ethyl acetate (5.5 L). The filtrate was diluted with ethyl acetate (5.5 L) and stirred for 15 minutes. Water (44 L) was added over 40 minutes and the batch age for 1 hour. The solid collected by filtration, washed with water (1×10 L, 3×20 L), dried on the filter under a nitrogen blanket and dried in vacuo at 45° C.

Step 14A: Alternative Preparation of N-(N′-(Fm-Glutaryl)-trans-4-hydroxy-L-prolinyl-alanyl-serine-cyclohexylglycyl-glutaminyl-serinyl)leucine (SEQ.ID.NO.: 53)

Fm-Glutaryl-Hyp-Ala-Ser-Chg-Gln-Ser-Leu-OBn (prepared as described in Step 13 or Alternate Step 13) (200 g) was dissolved in dimethylacetamide (1.9 L) at 45-50° C. 5% Pd/C (20 g, 10 wt %) slurried in DMA (100 ml) was added and the slurry was cooled to −5 to −10° C. The mixture was hydrogenated at atmospheric pressure maintaining the temperature between −10 and −5° C. for 5.5 hours.

The mixture was filtered while cold through a pre-wetted pad of Hyflo.

The filtrate was diluted with ethyl acetate (2.5 L) and water (8.0 L) was added. The batch was aged for a further 1 hour and the solid was collected by filtration. The cake was washed with water and sucked down on the filter and then dried in vacuo at 45° C. with a nitrogen sweep.

Step 15: N-(N′-(Fm-Glutaryl)-trans-4-hydroxy-L-prolinyl-alanyl-serine-cyclohexylglycyl-glutaminyl-serinyl) leucine Swish Purification

Crude N-(N′-(Fm-Glutaryl)-trans-4-hydroxy-L-prolinyl-alanyl-serine-cyclohexylglycyl-glutaminyl-serinyl) leucine (2.58 kg) (prepared as described in Step 14) was sieved.

The solid (2.56 Kg) was swished in ethyl acetate for 3 hours. The solid was collected by filtration, washed with ethyl acetate (26 L), dried on the filter under nitrogen and dried in vacuo at 40° C. The product was analyzed for purity by HPLC:

Step 16: Preparation of [N-Glutaryl(OFm)-(4-trans-L-Hyp)]-Ala-Ser-Chg-Gln-Ser-Leu-Dox (Compound 13) (SEQ.ID.NO.: 53)

To a 3 necked, 12 L round bottom flask equipped with mechanical stirrer, thermocouple, and nitrogen inlet was charged DMF (5.1 L) and HOAt (43.4 g, 319 mmoles, 1.2 equivalents). The yellow solution was inerted with nitrogen and warmed to 40° C. Heptapeptide prepared as described in Step 15(357.34 g, 266 mmoles) was added portion-wise to the warm solution; after stirring for 30 minutes at 40° C., a light yellow, opaque, homogeneous mixture resulted.

The mixture was cooled to room temperature, doxorubicin was added (158.9 g, 274 mmoles, 1.03 equivalents), and the red slurry was further cooled to −5° C. One equivalent of collidine (35 ml) was added followed by 0.8 equivalents of EDC (40.8 g, 213 mmoles) followed by the remaining two equivalents of collidine (70 ml). The red slurry was aged at −5° C. to −3° C.

The reaction was monitored by HPLC. After 1 hour, conversion had reached 58 A % Compound 13 and the remaining 0.5 eq. EDC (30.6 g, 160 mmoles) was charged.

After aging for a total of 3 hours, conversion had reached 90 A % Compound 13, 2.5 A % Heptapeptide and the reaction was warmed to 0° C. Aging for another 2 hours reduced peptide level to 0.73A % and the reaction was quenched as follows.

In a 50 L, 4 necked round bottom flask equipped with a mechanical stirrer, thermocouple, and nitrogen inlet, was charged K₂HPO₄ (67.9 g), KH₂PO₄ (283 g), and water (13 L) to give a 0.19 M pH 6.3 buffer solution. The buffer solution was inerted with nitrogen, cooled to 15-18° C., and the cold reaction mixture (−1° C.) was added to the buffer via an addition funnel over 60 minutes maintaining the slurry temperature at 15-18° C. After complete addition, the red slurry was aged 15 minutes at 18° C., and filtered. The filter cake was displacement washed with water (1×6 L), followed by slurry washing with water (6×6 L), and dried in vacuo at room temperature with a nitrogen sweep. After drying for 48 hours, a red solid with a TG. of 1.4% was obtained. The solid was analyzed by HPLC.

D-leucine Compound 13 Epimer assayed to 2.7 A %; the combined loss to the mother liquors and water washes was ca. 4% (long gradient assay). No residual peptide was detectable; the residual doxorubicin level was 1.1 A % (long gradient assay).

Step 16A: Alternate Preparation of [N-Glutaryl(OFm)-(4-trans-L-Hyp)]-Ala-Ser-Chg-Gln-Ser-Leu-Dox (Compound 13) (SEQ.ID.NO.: 53)

DMF (400 mL) was charged to a 1 L RB flask and degassed by N₂ sparge while cooling to −6° C. The Heptapeptide prepared as described in Step 15,(19.97 g, 19.06 mmol) and HOAT (3.12 g, 22.9 mmol) were then charged as solids to the cold DMF. A slurry of doxorubicin-HCl (11.05 g, 19.06 mmol) in degassed DMF (50 mL) was charged by vacuum, followed by two rinses (2×25 mL) of the slurry flask. Collidine was charged followed by a portion of EDC (2.92 g, 0.8 eq.). After 1.3 h, a second charge of EDC (2.19 g, 0.6 eq) was made. After a total age of 7.4 h the clear red solution was queched by dropwise addition to a pH 6.2 phosphate buffer (1350 mL) at 16-17° C. over 1.3 h. The resulting slurry was filtered and the filter cake was then washed with water (2000 mL). The filter cake was dried under a N₂ stream to provide the title compound as a red powder.

Step 16B: Alternate Preparation of [N-Glutaryl(OFm)-(4-trans-L-Hyp)]-Ala-Ser-Chg-Gln-Ser-Leu-Dox Compound 13 (SEQ.ID.NO.: 53)

A 1 L, 4 neck round bottom flask was set up and 20.0 g of Doxorubicin-HCl (20.0 g, 34.5 mmol) was added in a glovebox. The flask was then equipped with a truebore stirrer, N₂ inlet/vacuum inlet, and a thermocouple. DMF (472 mL) and water (4.7 mL) were premixed (20° C.) and then charged to form a red slurry. The 2,4,6-collidine (12.5 g, 103 mmol) and 2-HOPO (4.6 g, 41.4 mmol) were then, respectively, charged at ambient temperature and allowed to mix for 10 minutes. The slurry was then cooled to −5° C. and the heptapeptide, prepared as described in Step 15, (40.6 g, 35.3 mmol) was charged. It was stirred for 30 minutes at that temperature. The first, 0.8 equivalent of the EDC-HC1 (5.3 g) was charged, and the solution allowed to mix for 90 minutes. The reaction was monitored by HPLC.

After 90 minutes had elapsed, the remaining 0.6 equivalents of EDC·HCl (3.96 g) was added, the cooling bath was removed and the reaction was allowed to mix overnight at room temperature (21-22 ° C.).

The prepared buffer solution (1.20 L of the following buffer: 10.9 g K₂HPO₄, 43.54 g KH₂PO₄; pH 6.05) was added to a 3-L round bottom flask equipped with a truebore stirrer, N₂ inlet and thermocouple. The reaction solution was transferred to a 1-L addition funnel while, concurrently, the temperature of the buffer was reduced to 15-18° C.

The reaction solution was added to the buffer over 1-1.5 hours, while maintaining the temperature between 15-18° C. A precipitate resulted. The precipitated material was filtered and washed with water (2.30 L).

After drying overnight at 22° C. under N₂ and house vacuum, the solid (53.0 g) was assayed and was 91.5 A %, 88.1 wt. %. The yield after correction for purity was 86%.

Step 16C: Alternate Preparation of [N-Glutaryl(OFm)-(4-trans-L-Hyp)]-Ala-Ser-Chg-Gln-Ser-Leu-Dox Compound 13 (SEQ.ID.NO.: 53)

A 3L, 4 neck round bottom flask was set up and, concurrently, 20.0 grams of Doxorubicin HCl (34.5 mmol) was added in a glovebox to a sealed beaker or Erlenmeyer flask. The 3L flask was then equipped with a truebore stirrer, N₂ inlet/vacuum inlet, and a thermocouple. DMF (472 mL) and water (4.7 mL) were premixed (20° C.) and then partially charged (approximately ½ the volume) to the 3L vessel. 2-HOPO (4.6 g, 41.4 mmol) and HOAt (0.47 g, 3.45 mmol) were then, respectively, charged and allowed to mix for 10 minutes or until dissolved. One-quarter of the wet DMF was then added to the Dox·HCl to form a slurry, and this was then added to the 3L vessel. Finally, the 2,4,6-collidine (12.5 g, 103 mmol) was added to the 3L vessel which was then cooled to −5° C. and the heptapeptide, prepared as described in Step 15, (40.6 g , 35.3 mmol) was charged. After stirring for 30 minutes the first, 0.8 equivalents of the EDC·HCl was charged (5.3 g), followed by the final quarter of solvent. The resulting slurry was stirred for about 90 minutes and then the remaining 0.6 equivalents of EDC·HCl (3.96 g) was added. The cooling bath was removed and the reaction was allowed to mix overnight at room temperature (21-22° C.).

Ethyl acetate (354 mL) was then added to the reaction solution at 20° C.

The temperature of the reaction solution was reduced to 15-18° C. The pH6 buffer solution (1.20 L of water, 10.9 g K₂HPO₄, 43.54 g KH₂PO₄) was added slowly to the reaction solution over 1 hour, while maintaining the temperature between 15 and 18° C.

The precipitated material was filtered through a 600 mL medium sintered glass funnel, washed with water. (2.3 L) and the cake was dried overnight on the filter at ambient temperature under N₂. The solid (54.5 g) was assayed at 89.7 A %. The yield, after correction for purity, was 90.0%.

Step 17: Preparation of [N-Glutaryl-(4-trans-L-Hyp)]-Ala-Ser-Chg-Gln-Ser-Leu-Dox Piperidine salt (Compound 14) (SEQ.ID.NO.: 25)

To a 3 necked, 12 L round bottom flask equipped with mechanical stirrer, thermocouple, and nitrogen inlet was charged Compound 13 (399 g, 253.5 mmoles, TG 1.4%) and DMF (3.55 L). The red solution was inerted with nitrogen, cooled to 1° C., and a solution of piperidine (40 mL, 404 mmoles, 1.6 eq.) in DMF (400 mL) was added drop-wise over 70 minutes maintaining the batch temperature at 0-2° C. The resulting purplish solution was aged under nitrogen at 0-2° C.

The reaction was monitored by HPLC. After aging 1.5 hours at 0-2° C., conversion had reached 92.4% [A % 14/(A % 14+A % 13)]. Additional piperidine was charged after 2 hours reaction time (2.5 mL piperidine in 25 mL DMF); after aging another 2 hours, conversion had reached 98.1% and the reaction was quenched as follows.

In a 22 L, 3 necked round bottom flask equipped with mechanical stirrer, thermocouple, and nitrogen inlet was charged isopropyl acetate (12.1 L), inerted with nitrogen, and cooled to 0-5° C. To the cold i-PAc was added the cold (2° C.) reaction mixture via nitrogen pressure cannulation over 40 minutes. The resulting pink slurry was aged at 0-5° C. for thirty minutes then filtered under nitrogen. The cake was displacement washed with i-PAc (2×4 L) then slurry washed with i-PAc (3×4 L). All washes were done under a nitrogen blanket. The solid was dried in vacuo at room temperature with a nitrogen sweep for 24 hours to give of an orange solid. The solid was assayed for purity using LC.

Step 18: Preparative HPLC purification of [N-Glutaryl-(4-trans-L-Hyp)]-Ala-Ser-Chg-Gln-Ser-Leu-Dox Piperidinium salt/Free Acid (Compound 15) (SEQ.ID.NO.: 25)

The crude piperidine salt was purified by preparative HPLC on C-18 silica gel, eluting with a 0.1% aqueous ammonium acetate/acetonitrile gradient (100% NH₄OAc to 55% NH₄OAc over 80 min). The rich cuts that were >97% pure were pooled to provide the purified piperidine salt.

A portion of the purified piperidine salt of Compound 15 was rechromatographed on C-18 silica gel using a 2% aqueous HOAc/acetonitrile gradient (100% aqueousHOAc to 40% aqueous HOAc over 60 min). The fractions that were >98% pure were pooled and lyophilized, providing the pure free acid 15.

Step 18A: Alternate Preparation of [N-Glutaryl-(4-trans-L-Hyp)]-Ala-Ser-Chg-Gln-Ser-Leu-Dox Compound 15 (SEQ.ID.NO.: 25)

To a solution of the heptapeptide, prepared as described in Step 15, (1.09 g, 0.99 mmol) in DMF at 0° C. was added solid quinuclidine (0.34 g, 3.00 mmol). The solution was stirred at 0° C. for 0.5 h, and at 20° C. for 2.0 h, respectively. An aliquot was taken and assayed by LC to show no heptapeptide remained, indicating that conversion to the diacid was complete.

To this solution of [N-Glutaryl-(4-trans-L-Hyp)]-Ala-Ser-Chg-Gln-Ser-Leu was added 2,4,6-collidine (0.50 mL, 3.78 mmol), HOAt (0.163 g, 3.00 mmol) and Doxorubicin·HCl (0.58 g, 1.00 mmol), resulting in a slurry. The slurry was cooled to 0° C. and solid EDC·HCl (0.20 g, 1.04 mmol) was introduced in one portion. The reaction was aged for 2.5 h at 0° C. to lead to a homogenous solution that was assayed by LC to show the formation of Compound 15 in 78% yield.

Step 18B: Alternate Preparation of [N-Glutaryl-(4-trans-L-Hyp)]-Ala-Ser-Chg-Gln-Ser-Leu-Dox Compound 15 (SEQ.ID.NO.: 25)

To a solution of the heptapeptide, prepared as described in Step 15, (1.09 g, 0.99 mmol) in DMF at 0° C. was added a solid quinuclidine (0.34 g, 3.00 mmol). The solution was stirred at 0° C. for 0.5 h, and at 20° C. for 2.0 h, respectively. An aliquot was taken and assayed by LC to show no heptapeptide remained, indicating that conversion to the diacid was complete.

To a portion of the solution of [N-Glutaryl-(4-trans-L-Hyp)]-Ala-Ser-Chg-Gln-Ser-Leu (ca. 0.497 mmol) was added 2,4,6-collidine (0.123 mL, 0.934 mmol), 2-HOPO (52 mg, 0.467 mmol) and Doxorubicin·HCl (092 mg, 0.159 mmol) at 20° C., resulting in a slurry. The slurry was cooled to 0° C. and solid EDC·HCl (100 mg, 0.524 mmol) was introduced in one portion. The reaction was aged for 2.0 h at 0° C. and then assayed by LC to show the formation of Compound 15 in 93.9% yield and no remaining doxorubicin.

Step 19: Preparation of [N-Glutaryl-(4-trans-L-Hyp)]-Ala-Ser-Chg-Gln-Ser-Leu-Dox Sodium salt (Compound 16) (SEQ.ID.NO.: 25)

The lyophalized Compound 15 free acid (2.0 g, 1.43 mmol), prepared as described in Example 5, was dissolved in 10 mL of water and a 0.100 N aqueous NaOH solution (14.3 mL, 1.43 mmol) was added over 10 min. with vigorous stirring. The pH of the solution at the end of the addition was 6.3. The water was removed by evaporation under a nitrogen stream to provide a microcrystalline solid.

Alternatively, addition of acetone to the aqueous solution of the sodium salt resulted in precipitation of the compound from solution. The salt was collected by filtration and dried under a nitrogen stream. The solid was recrystallized from 1:12 water acetone to provide a microcrytalline solid.

Step 19A: Alternative Preparation of [N-Glutaryl-(4-trans-L-Hyp)]-Ala-Ser-Chg-Gln-Ser-Leu-Dox Sodium salt (Compound 16) (SEQ.ID.NO.: 25)

The compound 4 piperidine salt (10.37 g, 71% by wt free acid), prepared as described in Example 5, was dissolved in acetone (50 mL) and sodium acetate buffer (pH 5.2 0.2 M, 50 mL), and then stirred at 21-22° C. for 1 h. Acetone was then added (150 mL) slowly over 45 mins. The solution was then seeded with Compound 5 (50 mg) and the batch aged for 1 h at 21-22° C. Acetone (100 mL) was then added slowly over 2 h. The suspension was then cooled to 5° C. over 30 mins, and aged at 2-5° C. for 1 h. The product was isolated by filtration under an atmosphere of nitrogen, and the filter cake washed with 9:1 acetone/water (70 mL) followed by acetone (35 mL). The product was dried on the filter, under an atmosphere of nitrogen, overnight to give the sodium salt as a white crystalline solid.

Step 19B: Alternative Preparation of [N-Glutaryl-(4-trans-L-Hyp)]-Ala-Ser-Chg-Gln-Ser-Leu-Dox Sodium salt (Compound 16) (SEQ.ID.NO.: 25)

Compound 13 (0.91 g) was added to a 250 mL three necked flask, and was dissolved in dry DMF (15 mL). The solution was degassed twice and then cooled to 0° C. 1.91 mL of the 1.0M piperidine in DMF was added over 60 minutes with a syringe pump. The solution was aged until disappearance of the Compound 13 was seen by HPLC (125 min).

250 μL glacial acetic acid (6.9 eq) was then added over 10 minutes in order to keep the temperature below 5° C. 740 μL of 2 M NaOAc (2.33 eq) was then added to the solution.

Acetone (132 mL) was added slowly, however after addition of the first 30 mL a precipitate was seen. After addition of 50 mL of acetone, the mixture was seeded with 20 mg of Compound 5. The solution was aged for 30 minutes, and then the remaining acetone was added over 60 minutes, while maintaining the temperature below 5° C. The solid was filtered through a 60 mL medium sintered glass funnel, and the solid was washed with 10 mL 9:1 acetone: water. It is allowed to dry with vacuum, with a nitrogen tent to provide Compound 16 as a solid.

### Example 15

Preparation of (4-trans-L-Hyp)-Ala-Ser-Chg-Gln-Ser-Leu-Dox (SEQ.ID.NO.: 24)

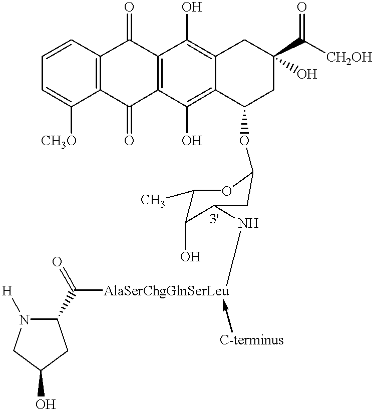

Step A: Fmoc-(4-trans-L-Hyp(Bzl))-Ala-Ser(Bzl)Chg-Gln-Ser(Bzl)Leu-PAM Resin

Starting with 0.5 mmol (0.67 g) Boc-Leu-PAM resin, the protected peptide was synthesized on a 430A ABI peptide synthesizer. The protocol used a 4 fold excess (2 mmol) of each of the following protected amino acids: Boc-Ser(Bzl), Boc-Gln, Boc-Chg, Boc-Ala, N-Boc-(4-trans-L-Hyp(Bzl)). Coupling was achieved using DCC and HOBT activation in methyl-2-pyrrolidinone. Fmoc-OSu (succinamidyl ester of Fmoc) was used for the introduction of the N-terminal protecting group. Removal of the Boc group was performed using 50% TFA in methylene chloride and the TFA salt neutralized with diisopropylethylamine. At the completion of the synthesis the peptide resin was dried to yield the title intermediate.

Step B: Fmoc-(4-trans-L-Hyp)-Ala-Ser-Chg-Gln-Ser-Leu-OH

The protected peptide resin from Step A, 1.1 g, was treated with HF (20 ml) for 1 hr at 0° C. in the presence of anisole (2 ml). After evaporation of the HF, the residue was washed with ether, filtered and extracted with H₂O (200 ml). The filtrate was lyophilyzed to yield the title intermediate.

Step C: Fmoc-(4-trans-L-Hyp)-Ala-Ser-Chg-Gln-Ser-Leu-Dox

The intermediate from Step B, 0.274 g, was dissolved in DMSO (10 ml) and diluted with DMF (10 ml). To the solution was added doxorubicin hydrochloride, 104 mg followed by 62 μL of diisopropylethylamine (DIEA). The stirred solution was cooled (0° C.) and 56 μL of diphenylphosphoryl azide added. After 30 minutes, an additional 56 μL of DPPA was added and the pH adjusted to ˜7.5 (pH paper) with DIEA. The pH of the cooled reaction (0° C.) was maintained at ˜7.5 with DIEA. After 4 hrs., the reaction (found to be complete by analytical HPLC, system A) was concentrated to an oil. HPLC conditions, system A.

Step D: (4-trans-L-Hyp)-Ala-Ser-Chg-Gln-Ser-Leu-Dox

The above product from Step C was dissolved in DMF (10 ml), cooled (0° C.) and 4 ml of piperidine added. The solution was concentrated to dryness and purified by preparative HPLC. (A=0.1% NH₄OAc-H₂O; B=CH₃CN.) The crude product was dissolved in 100 ml of 90% A buffer, filtered and purified on a C-18 reverse phase HPLC radial compression column (Waters, Delta-Pak, 15μ, 100Å). A step gradient of 90% A to 65% A was used at a flow rate of 75 ml/min (uv =214 nm). Homogeneous product fractions (evaluated by HPLC, system A) were pooled and freeze-dried.

| | |
|---|---|
| Molecular Formula: | C₆₀H₈₃N₉O₂₂ |
| Molecular Weight: | 1281.56 |
| High Resolution ES Mass | 1282.59 (MH⁺) |
| Spec: | |
| HPLC: | System A |
| Column: | Vydac 15 cm #218TP5415, C18 |
| Eluant: | Gradient 95:5 (A:B) to 5:95 (A:B) over 45 |
| | min. |
| | A = 0.1% TFA/H₂O, B = 0.1% TFA/Ace- |
| | tonitrile |
| Flow: | 1.5 ml/min. |
| Wavelength: | 214 nm, 254 nm |
| Retention Time: | 17.6 min. |

Amino Acid Compositional Analysis¹:

| Theory | Found |
|---|---|
| Ala (1) | 1.00 |
| Ser (2) | 1.94 |
| Chg (1) | 0.94 |
| Gln² (1) | 1.05 (as Glu) |
| Hyp (1) | 0.96 |
| Leu (1) | 1.03 |
| Peptide Content: | 0.690 μmol/mg |

Note: ¹20 hr., 100° C., 6N HCl
²Gln converted to Glu

### Example 16

des-Acetylvinblastine-4-O-(N-Acetyl-4-trans-L-Hyp-Ser-Ser-Chg-Gln-Ser-Ser-Pro) ester (SEQ.ID.NO.: 36)

Step A: Preparation of 4-des- Acetylvinblastine

A sample of 2.40 g (2.63 mmol) of vinblastine sulfate (Sigma V-1377) was dissolved under N₂ in 135 ml of absolute methanol and treated with 45 ml of anhydrous hydrazine, and the solution was stirred at 20-25° C. for 18 hr. The reaction was evaporated to a thick paste, which was partitioned between 300 ml of CH₂Cl₂ and 150 ml of saturated NaHCO₃. The aqueous layer was washed with 2 100-ml portions of CH₂Cl₂, and each of the 3 CH₂Cl₂ layers in turn was washed with 100 ml each of H₂O (2X) and saturated NaCl (1X). The combined organic layers were dried over anhydrous Na₂SO₄, and the solvent was removed at reduced pressure to yield the title compound as an off-white crystalline solid. This material was stored at −20° C. until use.

Step B: Preparation of 4-des- Acetylvinblastine 4-O-(Prolyl) ester

A sample of 804 mg (1.047 mmol) of 4-des- acetylvinblastine, dissolved in 3 ml of CH₂Cl₂ and 18 ml of anhydrous pyridine under nitrogen, was treated with 1.39 g of Fmoc-proline acid chloride (Fmoc-Pro-Cl, Advanced Chemtech), and the mixture was stirred for 20 hr at 25° C. When analysis by HPLC revealed the presence of unreacted starting des- acetylvinblastine, another 0.50 g of Fmoc-Pro-Cl was added, with stirring another 20 hr to complete the reaction. Water (cα. 3 ml) was added to react with the excess acid chloride, and the solution was then evaporated to dryness and partitioned between 300 ml of EtOAc and 150 ml of saturated NaHCO₃, followed by washing twice with saturated NaCl. After drying (Na₂SO₄), the solvent was removed under reduced pressure to give an orange-brown residue, to which was added 30 ml of DMF and 14 ml of piperidine, and after 5 min the solution was evaporated under reduced pressure to give a orange-yellow semi-solid residue. After drying in vacuo for about 1 hr, approx. 200 ml of H₂O and 100 ml of ether was added to this material, followed by glacial HOAc dropwise with shaking and sonication until complete dissolution had occurred and the aqueous layer had attained a stable pH of 4.5-5.0 (moistened pH range 4-6 paper). The aqueous layer was then washed with 1 100-ml portion of ether, and each ether layer was washed in turn with 50 ml of H₂O. The combined aqueous layers were subjected to preparative HPLC in 2 portions on a Waters C4 Delta-Pak column 15μM 300A (A=0.1% TFA/H₂O; B=0.1% TFA/CH₃CN), gradient elution 95→70% A/70 min. Pooled fractions yielded, upon concentration and lyophilization, the title compound.

Step C: N-Acetyl-4-trans-L-Hyp-Ser-Ser-Chg-Gln-Ser-Ser-WANG Resin

Starting with 0.5 mmole (0.61 g) of Fmoc-Ser(t-Bu)-WANG resin loaded at 0.82 mmol/g, the protected peptide was synthesized on a ABI model 430A peptide synthesizer adapted for Fmoc/t-butyl-based synthesis. The protocol used a 2-fold excess (1.0 mmol) of each of the following protected amino acids: Fmoc-Ser(t-Bu)-OH, Fmoc-Gln-OH, Fmoc-Chg-OH, Fmoc-4-trans-L-Hyp-OH; and acetic acid (double coupling). During each coupling cycle Fmoc protection was removed using 20% piperidine in N-methyl-2-pyrrolidinone (NMP), followed by washing with NMP. Coupling was achieved using DCC and HOBt activation in NMP. At the completion of the synthesis, the peptide resin was dried to yield the title compound.

Step D: N-Acetyl-4-trans-L-Hyp-Ser-Ser-Chg-Gln-Ser-Ser-OH

One 0.5-mmol run of the above peptide-resin was suspended in 25 ml of TFA, followed by addition of 0.625 ml each of H₂O and triisopropylsilane, then stirring at 25° for 2.0 hr. The cleavage mixture was filtered, the solids were washed with TFA, the solvents were removed from the filtrate under reduced pressure, and the residue was triturated with ether to give a pale yellow solid, which was isolated by filtration and drying in vacuo to afford the title compound.

HPLC conditions, system A:

Column . . . Vydac 15 cm #218TP5415, C18

Eluant . . . Gradient (95% A→50% A) over 45 min. A=0.1% TFA/H₂O, B=0.1%

TFA/acetonitrile

Flow . . . 1.5 ml/min.

High Resolution ES/FT-MS: 789.3

Step E: des- Acetylvinblastine-4-O-(N-Acetyl-4-trans-L-Hyp-Ser-Ser-Chg-Gln-Ser-Ser-Pro) ester

Samples of 522 mg (0.66 mmol) of the peptide prepared as described in step D and 555 mg (cα. 0.6 mmol) of 4-des- Acetylvinblastine 4-O-(Prolyl) ester from Step B, prepared as above, were dissolved in 17 ml of DMF under N₂. Then 163 mg (1.13 mmol) of 1-hydroxy-7-azabenzotriazole (HOAt) was added, and the pH was adjusted to 6.5-7 (moistened 5-10 range pH paper) with 2,4,6-collidine, followed by cooling to 0° C. and addition of 155 mg (0.81 mmol) of 1-(3-dimethylaminopropyl)-3-ethylcarbodiimide hydrochloride (EDC). Stirring was continued at 0-5° C. until completion of the coupling as monitored by analytical HPLC (A=0.1% TFA/H₂O; B =0.1% TFA/CH₃CN), maintaining the pH at 6.5-7 by periodic addition of 2,4,6-collidine. After 12 hr the reaction was worked up by addition of ˜4 ml of H₂O and, after stirring 1 hr, concentrated to a small volume in vacuo and dissolution in ca. 150 ml of 5% HOAc. and preparative HPLC in two portions on a Waters C₁₈ Delta-Pak column 15μM 300A (A=0.1% TFA/H₂O; B=0.1% TFA/CH₃CN, gradient elution 95→65% A /70 min). Homogeneous fractions containing the later-eluting product (evaluated by HPLC, system A, 95→65% A /30 min) from both runs were pooled and concentrated to a volume of 50 ml and passed through approx. 40 ml of AG4X4 ion exchange resin (acetate cycle), followed by freeze-drying to give the title compound as a lyophilized powder.

High Resolution ES/FT-MS: 1637.0

### Example 17

des-Acetylvinblastine-4-O-(N-Acetyl-4-trans-L-Hyp-Ser-Ser-Chg-Gln-Ser-Ser-Pro) ester acetate (SEQ.ID.NO.: 36)

A sample of 4.50 g (3.7 mmol) of 4-O-(prolyl) des- acetylvinblastine TFA salt, prepared as described in Example 16, Step B, was dissolved in 300 ml of DMF under N₂, and the solution was cooled to 0°. Then 1.72 g (10.5 mmol) of 3,4-dihydro-3-hydroxy-4-oxo-1,2,3-benzotriazine (ODHBT) was added, and the pH was adjusted to 7.0 (moistened 5-10 range pH paper) with N-methylmorpholine (NMM), followed by the addition of 4.95 g (5.23 mmol) of the N-acetyl-heptapeptide of Example 16, Step D, portionwise allowing complete dissolution between each addition. The pH was again adjusted to 7.0 with NMM, and 1.88 g (9.8 mmol) of 1-(3-dimethylaminopropyl)-3-ethylcarbodiimide hydrochloride (EDC) was added, followed by stirring of the solution at 0-5° C. until completion of the coupling as monitored by analytical HPLC (system A), maintaining the pH at ca. 7 by periodic addition of NMM. The analysis showed the major component at 26.3 min retention time preceded by a minor component (ca. 10%) at 26.1 min, identified as the D-Ser isomer of the title compound. After 20 hr the reaction was worked up by addition of 30 ml of H₂O and, after stirring 1 hr, concentrated to a small volume in vacuo and dissolution in ca. 500 ml of 20% HOAc. and preparative HPLC in 12 portions on a Waters C18 Delta-Pak column 15 mM 300A (A=0.1% TFA/H₂O; B=0.1% TFA/CH₃CN), gradient elution 85→65% A /90 min) at a flow rate of 80 ml/min.

Homogeneous fractions (evaluated by HPLC, system C) representing approx. one-fourth of the total run were pooled and concentrated to a volume of 150 ml and passed through approx. 200 ml of Bio-Rad AG4X4 ion exchange resin (acetate cycle), followed by freeze-drying of the eluant gave the acetate salt of the title compound as a lyophilized powder: retention time (system A) 26.7 min, 98.9% pure; high resolution ES/FT-MS m/e 1636.82; amino acid compositional analysis 20 hr, 100° C., 6N HCl (theory/found), Ser4/3.91 (corrected), Glu 1/0.92 (Gln converted to Glu), Chg 1/1.11, Hyp 1/1.07, Pro 1/0.99, peptide content 0.516 mmol/mg.

Further combination of homogeneous fractions and purification from side fractions, processing as above through approx. 500 ml of ion exchange resin, afforded an additional amounts of the title compound.

HPLC conditions, system A:

Column . . . Vydac 15 cm #218TP5415, C18

Flow . . . 1.5 ml/min.

Eluant . . . Gradient (95% A→50% A) over 45 min.

A=0.1 % TFA/H₂O, B=0.1% TFA/acetonitrile

Wavelenth . . . 214 nm, 280 nm

HPLC conditions, system C:

Column . . . Vydac 15 cm #218TP5415, C18

Flow . . . 1.5 ml/min.

Eluant . . . Gradient (85% A→65% A) over 30 min.

A=0.1% TFA/H₂O, B=0.1% TFA/acetonitrile

Wavelenth . . . 214 nm, 280 nm

### Example 18

Preparation of 4-des- Acetylvinblastine-23-(4′-aminomethylbicyclo-[2.2.2]octane) methylamide (BDAM-(dAc)vinblastine)

Step A: Preparation of 4-des- Acetylvinblastine-23-hydrazide

A sample of 3.99 g (4.38 mmol) of vinblastine sulfate (Sigma V-1377) was dissolved in 30.4 ml of 1:1 (v/v) absolute ethanol/anhydrous hydrazine, under N₂, and the solution was heated in an oil bath at 60-65° C. for 23 hr. Upon cooling, the solution was evaporated to a thick paste, which was partitioned between 300 ml of CH₂Cl₂ and 150 ml of saturated NaHCO₃. The aqueous layer was washed with 2 100-ml portions of CH₂Cl₂, and each of the 3 CH₂Cl₂ layers in turn was washed with 100 ml each of H₂O (2X) and saturated NaCl (1X). The combined organic layers were dried over anhydrous Na₂SO₄, and the solvent was removed in vacuo to yield, after drying 20 hr in vacuo, the title compound as a white crystalline solid. This material was dissolved in 82 ml of dry, degassed DMF for storage at −20° C. until use (conc. 36 mg/ml).

Step B: Boc-4-aminomethylbicyclo-[2.2.2]octane carboxylic acid

A sample of 8.79 g (40.0 mmol) of 4-carboxybicyclo-[2.2.2]octanemethylamine hydrochloride salt suspended in 100 ml each of THF and H₂O was treated with 20.0 ml (14.6 g=3.3 equiv.) of TEA, followed by 11.8 g (47.9 mmol) of BOC-ON reagent. All went into solution, and after stirring 24 hr the solution was concentrated in vacuo to a volume of about 50 ml and partitioned between 100 ml of ether and 300 ml of H₂O. After addition of about 2 ml of TEA the aqueous layer was washed with ether (3X), each ether in turn washed with H₂O, and the combined aqueous layer was acidified with 5% KHSO₄ to give the title compound as a white solid, isolated by filtration and drying in vacuo.

Step C: Boc-4-aminomethylbicyclo-[2.2.2]octane carboxamide

A stirred solution under N₂ of 12.0 g (42.5 mmol) of the product from step B in 100 ml of DMF was treated with 8.0 g (49.3 mmol) of carbonyldiimidazole. After 30 min the DMF was evaporated in vacuo to afford 50-60 ml of a light brown paste, which was stirred and treated with 70 ml of conc. NH₄OH rapidly added. The initial solution turned to a white paste within 30 min, after which H₂O was added up to a total volume of 400 ml to complete precipitation of product, which was triturated and isolated by filtration and washing with H₂O, and dried in vacuo to yield the title compound as a white solid.

Step D: Boc-4-aminomethylbicyclo-[2.2.2]octane nitrile

A solution of 7.52 g (26.6 mmol) of the product from step C in 50 ml of CH₂Cl₂ and 80 ml of anhydrous pyridine was treated with 11.12 g of (methoxycarbonylsulfamoyl)-triethyl-ammonium hydroxide inner salt (Burgess reagent) in 1-g portions over 5 min. After stirring for 1.5 hr, TLC (90-10-1, CHCl₃-CH₃OH-H₂O) showed complete conversion to product, and the solution was evaporated to give a paste, to which H₂O was added, up to 400 ml, with trituration and stirring to afford, after standing 20 hr at 0° C., filtration and drying in vacuo, the title compound as a white solid.

Step E: Boc-4-aminomethylbicyclo-[2.2.2]octane methylamine

A solution of 6.75 g (25.5 mmol) of the product from step D in 200 ml of CH₃OH plus 4 ml of HOAc and 2 ml of H₂O was hydrogenated over 1.63 g of PtO₂ in a Parr shaker at 55 psi for 22 hr. The catalyst was removed by filtration through Celite, and the filtrate was concentrated in vacuo to an oily residue, which was flushed/evaporated with CH₃OH (1X) and CH₂Cl₂ (2X). Product began to crystallize toward the end of the evaporation, and ether (up to 300 ml) was added to complete the precipitation. The white solid was triturated and isolated by filtration and washing with ether to give, after drying in vacuo, the title compound as the acetate salt.

400 Mhz ¹H-NMR (CDCl₃): δ (ppm, TMS) 4.5 (1 s, Boc-NH); 2.9 (2 br d, —CH2—NH-Boc); 2.45 (2 br s, —CH2—NH₂); 2.03 (3 s, CH3COOH);1.45 (9 s, Boc); 1.40 (12 s, ring CH2).

Step F: Preparation of 4-des- Acetylvinblastine-23-(4′-aminomethylbicyclo-[2.2.2]octane) methylamide (BDAM-(dAc)vinblastine)

A 30-ml aliquot of the above DMF solution of 4-des-acetylvinblastine-23-hydrazide (1.41 mmol), cooled to −15° C. under Argon, was converted to the azide in situ by acidification with 4M HCl in dioxane to pH <1.5 (moistened 0-2.5 range paper), followed by addition of 0.27 ml (1.3 equiv) of isoamyl nitrite and stirring for 1 hr at 10-15° C. The pH was brought to 7 by the addition of DIEA, and a slurry of 1.27 g (3.8 mmol) of the Boc diamine product from step E above in 20 ml of DMF was then added, and the reaction was allowed to warm slowly to 15-20° C. over 2 hr, at which point coupling was complete, as monitored by analytical HPLC (A=0.1% TFA/H₂O; B=0.1% TFA/CH₃CN). The solvent was removed in vacuo and the residue partitioned between EtOAc and 5% NaHCO₃, the organic layer washed with 5% NaCl, and the aqueous layers back-extracted with CH₂Cl₂ to assure removal of the intermediary Boc-BDAM-(dAc) vinblastine. The combined organic layers were dried over Na₂SO₄, the solvent was removed under reduced pressure, and the residue, after flush/evaporation twice from CH₂Cl₂, was dissolved in 30 ml of CH₂Cl₂ and treated with 30 ml of TFA for 30 min. The solvents were rapidly removed in vacuo, and the residue was dissolved in 300 ml of 10% HOAc for purification by preparative HPLC in 5 portions on a Waters C4 Delta-Pak column 15μM 300A (A =0.1% TFA/H₂O; B=0.1% TFA/CH₃CN), gradient elution 95→70% A /60 min, isocratic 70% /20 min. Homogeneous fractions (evaluated by HPLC, system A, 95→50% A) from the five runs were pooled and concentrated in vacuo, followed by freeze-drying to give of the title compound as the lyophilized TFA salt.

HPLC conditions, system A:

Column . . . Vydac 15 cm #218TP5415, C18

Eluant . . . Gradient (A→B) over 45 min.

A=0.1% TFA/H₂O, B=0.1% TFA/acetonitrile

Flow . . . 1.5 ml/min.

Retention time: BDAM (dAc) vinblastine 23.5 min. (95%→50% A) 97% purity

High Resolution ES/FT-MS: 905.63

Compound content by elemental analysis=0.714 μmol/mg:

N (calc)=9.28 N (found)=6.00

### Example 19

Preparation of 4-des- Acetylvinblastine-23-(N-Acetyl-Ser-Ser-Ser-Chg-Gln-Ser-Val-BDAM) amide acetate salt (SEQ.ID.NO.: 32)

Step A: N-Acetyl-Ser-Ser-Ser-Chg-Gln-Ser-Val-PAM Resin (SEQ.ID.NO.: 32)

Starting with 0.5 mmole (0.68 g) of Boc-Val-PAM resin, the protected peptide was synthesized on a ABI model 430A peptide synthesizer. The protocol used a 4-fold excess (2.0 mmol) of each of the following protected amino acids: Boc-Ser(Bzl)-OH, Boc-Gln-OH, Boc-Chg-OH; and acetic acid (2 couplings). During each coupling cycle Boc protection was removed using TFA, followed by neutralization with DIEA. Coupling was achieved using DCC and HOBt activation in N-methyl-2-pyrrolidinone. At the completion of the synthesis, the peptide resin was dried to yield the title compound.

Step B: N-Acetyl-Ser-Ser-Ser-Chg-Gln-Ser-Val-OH (SEQ.ID.NO.: 46)

Three 0.5-mmol runs of the above peptide-resin (3.5 g) were combined and treated with liquid BF (65 ml) for 1.5 hr at 0° C. in the presence of anisole (6 ml). After evaporation of the HF, the residue was washed with ether, filtered and leached with 150 ml of DMF in several portions, adding DIEA to pH ˜8, followed by removal of the DMF in vacuo to a volume of 100 ml. The concentration was determined as ca. 11.7 mg/ml (by weighing the dried resin before and after leaching. The sample purity was determined as 96% by HPLC. The solution was used directly for conjugation with BDAM-(dAc)vinblastine.

Step C: 4-Des- acetylvinblastine-23-( N-Acetyl-Ser-Ser-Ser-Chg-Gln-Ser-Val-BDAM) amide acetate salt

To 58 ml (equivalent to 0.875 mmol of peptide) of the solution from step B was added 530 mg (0.520 mmol) of BDAM-(dAc)vinblastine, prepared as described in Example 30, Step F, under N₂, cooling to 0° C., and the pH was adjusted to ˜8 (moistened 5-10 range pH paper) with DIEA. Then 0.134 ml (0.62 mmol) of DPPA was added, followed by stirring at 0-5° C. until completion of the coupling as monitored by analytical HPLC (A=0.1% TFA/H₂O; B=0.1% TFA/CH₃CN), maintaining the pH at ≧7 by periodic addition of DIEA. After 24 hr, the reaction was worked up by addition of 10 ml of H₂O, stirring 1 hr and concentration to small volume in vacuo, then dissolution in ca. 100 ml of 10% HOAc/5% CH₃CN, adjustment of the pH to 5 with NH₄HCO₃, filtration to remove insolubles, and preparative HPLC in 3 portions on a Waters C4 Delta-Pak column 15μM 300A (A=0.1% NH₄HCO₃/H₂O; B=CH₃CN), gradient elution 95→40% A /70 min. Fractions from each run containing product were pooled, acidified to pH 3 with glacial HOAc, concentrated in vacuo to a volume of 50 ml, and purified by preparative HPLC on a Waters C18 Delta-Pak column 15 μM 300A (A=0.1% TFA/H₂O; B=0.1% TFA/CH₃CN), gradient elution 95→70% A /60 min, isocratic 70% /20 min. Homogeneous fractions (evaluated by HPLC, system A, 95→50% A) from all three runs were pooled and concentrated to a volume of ˜100 ml., diluted with 5% CH₃CN, and passed through AG4X4 ion exchange resin (acetate cycle), followed by freeze-drying to give the title compound as a lyophilized powder.

HPLC conditions, system A:

Column . . . Vydac 15 cm #218TP5415, C18

Eluant . . . Gradient (A→B) over 45 min.

A=0.1% TFA/H₂O, B=0.1% TFA/acetonitrile

Flow . . . 1.5 ml/min.

Retention times: BDAM (dAc) vinblastine 23.5 min.

N-Acetyl-Ser-Ser-Ser-Chg-Gln-Ser-Val-OH 14.5 min.

4-Des- acetylvinblastine-23-( N-Acetyl-Ser-Ser-Ser-Chg-Gln-Ser-Val-BDAM) amide 29.5 min.

High Resolution ES/FF-MS: 1662.03

Amino Acid Compositional Analysis¹ (theory/found):

²Ser4/3.6 ³Glu 1/2.10 ⁴Val 1/0.7 Chg 1/0.95

Peptide content 0.504 μmol/mg

Note: ¹20 hr, 100° C., 6N HCl

²Uncorrected

³Gln converted to Glu

⁴Incomplete hydrolysis

### Example 20

Preparation of 4-des- Acetylvinblastine-23-(N-methoxy-diethylene-oxyacetyl-4-trans-L-Hyp-Ser-Ser-Chg-Gln-Ser-Val-BDAM) amide acetate salt (SEQ.ID.NO.: 33)

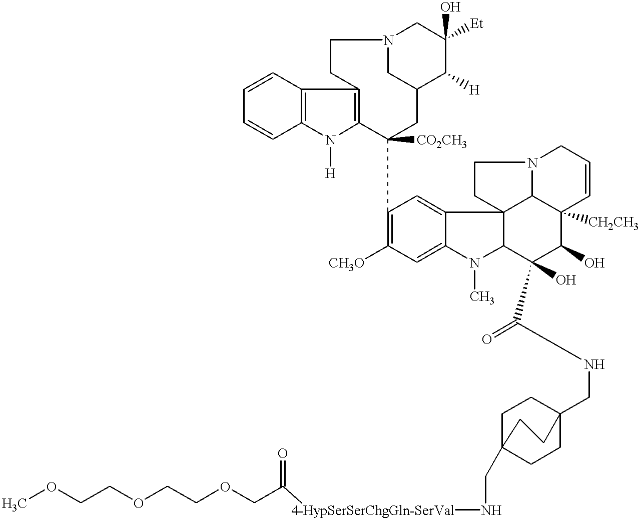

### (SEQ.ID.NO.: 33),

Step A: N-methoxydiethyleneoxyacetyl-4-trans-L-Hyp-Ser-Ser-Chg-Gln-Ser-Val-PAM Resin (SEQ.ID.NO.: 33)

Starting with 0.5 mmole (0.68 g) of Boc-Val-PAM resin, the protected peptide was synthesized on a ABI model 430A peptide synthesizer. The protocol used a 4-fold excess (2.0 mmol) of each of the following protected amino acids: Boc-Ser(Bzl)-OH, Boc-Gln-OH, Boc-Chg-OH, Boc-4-trans-Hyp(Bzl)-OH; and 2-[2-(2-methoxyethoxy)-ethoxy]acetic acid (2 couplings). During each coupling cycle Boc protection was removed using TFA, followed by neutralization with DIEA. Coupling was achieved using DCC and HOBt activation in N-methyl-2-pyrrolidinone. At the completion of the synthesis, the peptide resin was dried to yield the title compound.

Step B: N-methoxydiethyleneoxyacetyl-4-trans-L-Hyp-Ser-Ser-Chg-Gln-Ser-Val-OH (SEQ.ID.NO.: 33)

Two 0.5-mmol runs of the above peptide-resin (2.4 g) were combined and treated with liquid HF (40 ml) for 1.5 hr at 0° C. in the presence of anisole (4 ml). After evaporation of the HF, the residue was washed with ether, filtered and leached with 150 ml of H₂O in several portions, followed by preparative HPLC on a Waters C18 Delta-Pak column 15μM 100A (A=0.1% TFA/H₂O; B=0.1% TFA/CH₃CN), gradient elution 95→70% A /70 min, and pooling of homogeneous fractions and freeze drying to give the title compound as lyophilized powder. The sample purity was determined as 99% by HPLC.

Step C: 4-des- Acetylvinblastine-23-(N-methoxydiethylene-oxyacetyl-4-trans-L-Hyp-Ser-Ser-Chg-Gln-Ser-Val-BDAM) amide acetate salt

Samples of 440 mg (0.47 mmol) of the peptide from step B and 340 mg (0.33 mmol) of BDAM-(dAc)vinblastine, prepared as described in Example 30, Step F, were dissolved in 25 ml of DMF under N₂, cooling to 0° C. Then 85 mg (0.63 mmol) of 1-hydroxy-7-azabenzotriazole (HOAt) was added, and the pH was adjusted to 6.5-7 (moistened 5-10 range pH paper) with 2,4,6-collidine, followed by addition of 117 mg (0.61 mmol) of 1-(3-dimethylaminopropyl)-3-ethylcarbodiimide hydrochloride (EDC). Stirring was continued at 0-5° C. until completion of the coupling as monitored by analytical (A=0.1% TFA/H₂O; B=0.1% TFA/CH₃CN), maintaining the pH at 6.5-7 by periodic addition of 2,4,6-collidine. After 3 hr the reaction was worked up by addition of 10 ml of H₂O, stirring 1 hr and concentration to small volume in vacuo, then dissolution in ca. 70 ml of 5% HOAc. and preparative HPLC on a Waters C18 Delta-Pak column 15μM 300A (A=0.1% TFA/H₂O; B=0.1% TFA/CH₃CN), gradient elution 95→40% A /70 min). Homogeneous fractions (evaluated by HPLC, system A, 95→50% A) from all three runs were pooled and concentrated to a volume of 50 ml and passed through AG4X4 ion exchange resin (acetate cycle), followed by freeze-drying to give the title compound as a lyophilized powder.

HPLC conditions, system A:

Column . . . Vydac 15 cm #218TP5415, C18

Eluant . . . Gradient (A→B) over 45 min.

A=0. 1% TFA/H₂O, B=0.1% TFA/acetonitrile

Flow . . . 1.5 ml/min.

Retention times: BDAM (dAc) vinblastine 23.5 min.

N-methoxydiethyleneoxyacetyl-4-trans-L-Hyp-Ser-Ser-Chg-Gln-Ser-Val-OH 16.2 min.

4-des- Acetylvinblastine-23-(N-methoxydiethyleneoxyacetyl-4-trans-L-Hyp-Ser-Ser-Chg-Gln-Ser-Val-BDAM) amide 29.6 min.

High Resolution ES/FT-MS: 1805.95

Amino Acid Compositional Analysis¹ (theory/found):

²Ser3/1.7 ³Glu 1/1.01 ⁴Val 1/0.93 Chg 1/0.98 Hyp 1/1.01

Peptide content=0.497 μmol/mg

Note: ¹20 hr, 100° C., 6N HCl

²Uncorrected

³Gln converted to Glu

Incomplete hydrolysis

### Example 21

Preparation of 4-des- Acetylvinblastine-23-(N-Acetyl-4-trans-L-Hyp-Ser-Ser-Chg-Gln-Ser-HCAP) amide acetate salt (21-7) (SEQ.ID.NO. 50)

Step A: N-Acetyl-4-trans-L-Hyp-Ser-Ser-Chg-Gln-OH (20-1) (SEQ.ID.NO. 47)

Starting with 0.5 mmole (0.80 g) of Fmoc-Gln(Trt)-Wang resin, the protected peptide was synthesized on a ABI model 430A peptide synthesizer. The protocol used a 4-fold excess (2.0 mmol) of each of the following protected amino acids: Fmoc-Ser(tBu)-OH, Fmoc-Chg-OH, Fmoc-4-trans-Hyp(tBu)-OH and acetic acid (2 couplings). During each coupling cycle Fmoc protection was removed using 20% piperidine in DMF. Coupling was achieved using DCC and HOBt activation in N-methyl-2-pyrrolidinone. At the completion of the synthesis, the peptide resin was dried. 1.3 g peptide-resin was treated with 95% TFA :2.5% H₂O:2.5% Triisopropylsilane (20 ml) for 2 hr at r.t. under argon. After evaporation of the TFA, the residue was washed with ether, filtered and dried to give crude peptide which was purified by preparatory HPLC on a Delta-Pak C18 column with 0.1% trifluoroacetic acid—aqueous acetonitrile solvent systems using 100-70% A, 60 min linear gradient. Fractions containing product of at least 99% (HPLC) purity were combined to give the title compound.

FABMS: 615.3

Peptide Content: 1.03 nmole/mg.

HPLC: 99% pure @214 nm, retention time=10.16 min, (Vydac C₁₈, gradient of 95% A/B to 50% A/B over 30 min, A=0.1%TFA-H₂O, B=0.1% TFA-CH₃CN)

Step B: N-Boc-(1S,2R)-(+)-Norephedrine (21-2)

A solution of 1.51 g (10 mmol) of (1S,2R)-(+)-Norephedrine in a mixture of 1,4 dioxane (20 ml), water (10 ml) and 1N NaOH (10 ml) was stirred and cooled in an ice-water bath. Di-(t-butyl) dicarbonate (2.4 g, 11 mmol) was added in portions over approx. 20 min. The reaction was stirred in the cold for 2 hrs., then at room temp. for an additional 1 h. The solution was concentrated to remove most of the dioxane, cooled in an ice bath and covered with a layer of ethyl acetate (30 ml) and acidified to pH 2 with 1N KHSO₄. The aqueous phase was extracted 2x with EtOAc. The combined extracts were washed with water, brine and were concentrated and dried to provide the desired product as a white crystalline solid (21-2). FABMS:

Step C: N-Boc-HCAP (21-3)

A solution of 2.38 g of N-Boc-(1S,2R)-(+)-Norephedrine (20-2) in 50 ml acetic acid/10 ml H₂O was hydrogenated at 60 psi on a Parr apparatus over 500 mg of Ir black catalyst for 24 hrs. The reaction was filtered through a Celite pad, and the filtrate concentrated in vacuo to give a tan foam (21-3). FABMS: 258.2

Step D: N-Benzyloxycarbonyl-Ser-N-t-Boc-HCAP ester (21-4)

A solution of 1.95 g (6.6 mmol) of N-Z-Ser(tBu)-OH, 1.54 g (6.0 mmol) of N-Boc-HCAP (21-3), 1.26 g (6.6 mmol) of EDC, and 146 mg (1.2 mmol) of DMAP in 30 ml of anh. CH2Cl2 was treated and the resulting solution stirred at room temp. in an N₂ atmosphere for 12 h. The solvent was removed in vacuo, the residue dissolved in ethyl acetate (150 ml) and the solution extracted with 0.5 N NaHCO₃ (50 ml), water (50 ml) and brine, then dried and concentrated to provide the crude coupling product (21-4).

Step E: H-Ser(tBu)-N-t-Boc-HCAP ester (21-5)

A 2.0 g of (21-4) in a solution of 90 ml EtOH, 20 ml water, and 10 ml acetic acid was hydrogenated on a Parr apparatus at 50 psi over 200 mg of Pd(OH)₂ catalyst for 3 h. The reaction was filtered through a Celite pad, and the filtrate wasconcentrated to small volume in vacuo, then purified by preparatory HPLC on a Delta-Pak C18 column with 0.1% trifluoroacetic acid—aqueous acetonitrile solvent systems using 95-50% A, 60 min linear gradient. Fractions containing product of at least 99% (HPLC) purity were combined to give the intermediate (21-5). FABMS: 401.3

Step F: N-Acetyl-4-trans-L-Hyp-Ser-Ser-Chg-Gln-Ser-HCAP amine (21-6) (SEQ.ID.NO. 50)

A solution of 614 mg (1.0 mmol) of N-Acetyl-4-trans-L Hyp-Ser-Ser-Chg-Gln-OH (21-1), 400 mg (1.0 mmol) of H-Ser(tBu)-N-t-Boc-HCAP ester (21-5), 229 mg (1.2 mmol) of EDC, and 81 mg (0.5 mmol) of ODBHT (3,4-dihydro-3-hydroxy-4-oxo-1,2,3-benzotriazine), in 7 ml of DMF was stirred at 0° C. in an N₂ atmosphere for 10 h. The solvent was removed in vacuo, the residue was washed with ether and dried. The crude product was treated with 95% TFA :5% H₂O (20 ml) for 2 hr at r.t. under argon. After evaporation of the TFA, the residue was purified by preparatory HPLC on a Delta-Pak C18 column with 0.1% trifluoroacetic acid—aqueous acetonitrile solvent systems using 95-50% A, 60 min linear gradient. Fractions containing product of at least 99% (HPLC) purity were combined to give the intermediate compound (21-6).

FABMS: 841.8

Peptide Content: 863.39 NMole/mg.

HPLC: 99% pure @214 nm, retention time=13.7 min, (Vydac C₁₈, gradient of 95% A/B to 5% A/B over 30 min, A=0.1%TFA-H₂O, B=0.1% TFA-CH₃CN)

Step G: 4-des- Acetylvinblastine-23-(N-Ac-4-trans-L-Hyp-Ser-Ser-Chg-Gln-Ser-HCAP) amide acetate salt (21-7)

A solution of 0.461 of 4-des- acetylvinblastine-23-hydrazide (0.6 mmol) in 10 ml DMF cooled to −15° C. under Argon, was converted to the azide in situ by acidification with 4M HCl in dioxane to pH <1.5 (moistened 0-2.5 range paper), followed by addition of 0.105 ml (1.3 equiv) of isoamyl nitrite and stirring for 1 hr at 10-15° C. The pH was brought to 7 by the addition of DIEA, and 555 mg (0.66 mmol) of amine derivative (21-6) from step F was then added, and the reaction was stirred at 0° C. for 24 hrs, and purified by preparatory HPLC on a 15 μM,100A, Delta-Pak C18 column with 0.1% trifluoroacetic acid—aqueous acetonitrile solvent systems using 95-50% A, 60 min linear gradient. Homogeneous fractions were pooled and concentrated in vacuo, followed by freeze-drying to give the title compound as the TFA salt which was converted to the corresponding HOAc salt by AG 4×4 resin (100-200 mesh, free base form, BIO-RAD) (21-7).

ES⁺: 1576.7

Peptide Content: 461.81 NMole/mg.

Ser 3.04; Hyp 1.07; Chg 1.02; Glu 1.00

HPLC: 99% pure @214 nm, retention time=18.31 min, (Vydac C₁₈, gradient of 95% A/B to 5% A/B over 30 min, A=0.1% TFA-H₂O, B=0.1% TFA-CH₃CN)

### Example 22

Preparation of 4-des- Acetylvinblastine-23-(N-Acetyl-Ser-Chg-Gln-Ser-Ser-Pro-HCAP) amide acetate salt (22-7) (SEQ.ID.NO. 48)

Step A: N-Acetyl-Ser-Chg-Gln-Ser-Ser-OH (22-1) (SEQ.ID.NO. 49)

Starting with 0.5 mmole (0.80 g) of Fmoc-Ser(tBu)-Wang resin, the protected peptide was synthesized on a ABI model 430A peptide synthesizer. The protocol used a 4-fold excess (2.0 mmol) of each of the following protected amino acids: Fmoc-Ser(tBu)-OH, Fmoc-Gln-OH, Fmoc-Chg-OH, Fmoc-Ser(tBu)-OH and acetic acid (2 couplings). During each coupling cycle Fmoc protection was removed using 20% piperidine in DMF. Coupling was achieved using DCC and HOBt activation in N-methyl-2-pyrrolidinone. At the completion of the synthesis, the peptide resin was dried. 1.3 g peptide-resin was treated with 95% TFA:2.5% H₂O:2.5% Triisopropylsilane (20 ml) for 2 hr at r.t. under argon. After evaporation of the TFA, the residue was washed with ether, filtered and dried to give crude peptide which was purified by preparatory HPLC on a Delta-Pak C18 column with 0.1% trifluoroacetic acid—aqueous acetonitrile solvent systems using 100-70% A, 60 min linear gradient. Fractions containing product of at least 99% (HPLC) purity were combined to give the title compound.

FABMS: 589.5

Peptide Content: 1.01 NMole/mg.

HPLC: 99% pure @214 nm, retention time=10.7 min, (Vydac C₁₈, gradient of 95% A/B to 50% A/B over 30 min, A=0.1% TFA-H₂O, B=0.1% TFA-CH₃CN)

Step B: N-Boc-(1S,2R)-(+)-Norephedrine (22-2)

A solution of 1.51 g (10 mmol) of (1S,2R)-(+)-Norephedrine in a mixture of 1,4 dioxane (20 ml), water (10 ml) and 1N NaOH (10 ml) is stirred and cooled in an ice-water bath. Di-(t-butyl) dicarbonate (2.4 g, 11 mmol) was added in portions over approx. 20 min. The reaction was stirred in the cold for 2 hrs., then at room temp. for an additional 1 h. The solution was concentrated to remove most of the dioxane, cooled in an ice bath and covered with a layer of ethyl acetate (30 ml) and acidified to pH 2 with 1N KHSO₄. The aqueous phase was extracted 2x with EtOAc. The combined extracts were washed with water, brine and were concentrated and dried to provide the desired product as a white crystalline solid. FABMS: 252

Step C: N-Boc-HCAP (22-3)

A solution of 2.38 g of N-Boc-(1S,2R)-(+)-Norephedrine (21-2) in 50 ml acetic acid/10 ml H₂O was hydrogenated at 60 psi on a Parr apparatus over 500 mg of Ir black catalyst for 24 hrs. The reaction was filtered through a Celite pad, and the filtrate concentrated in vacuo to give a tan foam. FABMS: 258.2

Step D: N-Benzyloxycarbonyl-Pro-N-t-Boc-HCAP ester (22-4)

A solution of 1.62 g (6.6 mmol) of N-Z-Pro-OH, 1.54 g (6.0 mmol) of N-Boc-HCAP (21-3), 1.26 g (6.6 mmol) of EDC, and 146 mg (1.2 mmol) of DMAP in 30 ml of anh. CH₂Cl₂ was treated and the resulting solution stirred at room temp. in an N₂ atmosphere for 12 h. The solvent was removed in vacuo, the residue dissolved in ethyl acetate (150 ml) and the solution extracted with 0.5 N NaHCO₃ (50 ml), water (50 ml) and brine, then dried and concentrated to provide the crude coupling product.

Step E: H-Pro-N-t-Boc-HCAP ester (22-5)

A 2.0 g of (22-4) in a solution of 90 ml EtOH, 20 ml water, and 10 ml acetic acid was hydrogenated on a Parr apparatus at 50 psi over 200 mg of Pd(OH)₂ catalyst for 3 h. The reaction was filtered through a Celite pad, and the filtrate was concentrated to small volume in vacuo, then purified by preparatory HPLC on a Delta-Pak C18 column with 0.1% trifluoroacetic acid—aqueous acetonitrile solvent systems using 95-50% A, 60 min linear gradient. Fractions containing product of at least 99% (HPLC) purity were combined to give the title compound (22-5). FABMS: 356.3

Step F: N-Acetyl -Ser-Chg-Gln-Ser-Ser-Pro-HCAP amine (22-6) (SEQ.ID.NO.: 48)

A solution of 589 mg (1.0 mmol) of N-Acetyl-Ser-Chg-Gln-Ser-Ser-OH (22-1), 356 mg (1.0 mmol) of H-Pro-N-t-Boc-HCAP ester (22-5), 229 mg (1.2 mmol) of EDC, and 81 mg (0.5 mmol) of ODBHT (3,4-dihydro-3-hydroxy-4-oxo-1,2,3-benzotriazine), in 7 ml of DMF was stirred at 0° C. in an N₂ atmosphere for 10 h. The solvent was removed in vacuo, the residue was washed with ether and dried. The crude product was treated with 95% TFA :5% H₂O (20 ml) for 2 hr at r.t. under argon. After evaporation of the TFA, the residue was purified by preparatory HPLC on a Delta-Pak C18 column with 0.1% trifluoroacetic acid—aqueous acetonitrile solvent systems using 95-50% A, 60 min linear gradient. Fractions containing product of at least 99% (HPLC) purity were combined to give the title compound (22-6).

FABMS: 825.5

Peptide Content: 893.6 NMole/mg.

HPLC: 99% pure @214 nm, retention time=15.2 min, (Vydac C₁₈, gradient of 95% A/B to 5% A/B over 30 min, A=0.1% TFA-H₂O, B=0.1% TFA-CH₃CN)

Step G: 4-des- Acetylvinblastine-23-(N-Ac -Ser-Chg-Gln-Ser-Ser-Pro-HCAP) amide acetate salt (22-7)

A solution of 0.461 of 4-des- acetylvinblastine-23-hydrazide (0.6 mmol) in 10 ml DMF cooled to −15° C. under Argon, was converted to the azide in situ by acidification with 4M HCl in dioxane to pH<1.5 (moistened 0-2.5 range paper), followed by addition of 0.105 ml (1.3 equiv) of isoamyl nitrite and stirring for 1 hr at 10-15° C. The pH was brought to 7 by the addition of DIEA, and 545 mg (0.66 mmol) of amine derivative (22-6) from step F was then added, and the reaction was stirred at 0° C. for 24 hrs, and purified by preparatory HPLC on a 15 μM,100A, Delta-Pak C18 column with 0.1% trifluoroacetic acid—aqueous acetonitrile solvent systems using 95-50% A, 60 min linear gradient. Homogeneous fractions were pooled and concentrated in vacuo, followed by freeze-drying to give the title compound as the TFA salt which was converted to title compound by AG 4×4 resin (100-200 mesh, free base form, BIO-RAD) (22-7)

ES⁺: 1560.9

Peptide Content: 586.8 NMole/mg.

Ser 3.04; Chg 1.01; Glu 1.00; Pro 0.97

HPLC: 99% pure @214 nm, retention time=13.4 min, (Vydac C₁₈, gradient of 95% A/B to 5% A/B over 30 min, A=0.1% TFA-H₂O, B=0.1% TFA-CH₃CN)

Biological Assays

The ability of the compounds useful in the methods of the present invention to inhibit angiogenesis can be demonstrated using the following assays.

### COX-2 Inhibitor Assays

The compound of Formula I can be tested using the following assays to determine their COX-2 inhibiting activity.

### Inhibition of Cyclooxygenase Activity

Compounds are tested as inhibitors of cyclooxygenase activity in whole cell cyclooxygenase assays. Both of these assays measure prostaglandin E₂ synthesis in response to AA, using a radioimmunoassay. Cells used for these assays are human osteosarcoma 143 cells (which specifically express COX-2) and human U-937 cells (which specifically express COX-1). In these assays, 100% activity is defined as the difference between prostaglandin E₂ synthesis in the absence and presence of arachidonate.

Whole Cell Assays

For cyclooxygenase assays, osteosarcoma cells are cultured in 1 mL of media in 24-well multidishes (Nunclon) until confluent (1-2×10⁵ cells/well). U-937 cells are grown in spinner flasks and resuspended to a final density of 1.5×10⁶ cells/mL in 24-well multidishes (Nunclon). Following washing and resuspension of osteosarcoma and U-937 cells in 1 mL of BBSS, 1 μL of a DMSO solution of test compound or DMSO vehicle is added, and samples gently mixed. All assays are performed in triplicate. Samples are then incubated for 5 or 15 minutes at 37° C., prior to the addition of AA. AA (peroxide-free, Cayman Chemical) is prepared as a 10 mM stock solution in ethanol and further diluted 10-fold in HBSS. An aliquot of 10 μL of this diluted solution is added to the cells to give a final AA concentration of 10 μM. Control samples are incubated with ethanol vehicle instead of AA. Samples are again gently mixed and incubated for a further 10 min. at 37° C. For osteosarcoma cells, reactions are then stopped by the addition of 100 μL of 1N HCl, with mixing and by the rapid removal of the solution from cell monolayers. For U-937 cells, reactions are stopped by the addition of 100 μL of 1N HCl, with mixing. Samples are then neutralized by the addition of 100 μL of 1N NaOH and PGE₂ levels measured by radioimmunoassay.

Whole cell assays for COX-2 and COX-1 using CHO transfected cell lines

Chinese hamster ovary (CHO) cell lines which have been stably transfected with an eukaryotic expression vector pCDNAIII containing either the human COX-1 or COX-2 cDNA's are used for the assay. These cell lines are referred to as CHO [hCOX-1] and CHO [hCOX-2], respectively. For cyclooxygenase assays, CHO[hCOX-1] cells from suspension cultures and CHO[hCOX-2] cells prepared by trypsinization of adherent cultures are harvested by centrifugation (300 ×g, 10 min) and washed once in HBSS containing 15 mM HEPES, pH 7.4, and resuspended in HBSS, 15 mM HEPES, pH 7.4, at a cell concentration of 1.5×10⁶ cells/ml. Drugs to be tested are dissolved in DMSO to 66.7-fold the highest test drug concentration. Compounds are typically tested at 8 concentrations in duplicate using serial 3-fold serial dilutions in DMSO of the highest drug concentration. Cells (0.3×10⁶ cells in 200 μl) are preincubated with 3 μl of the test drug or DMSO vehicle for 15 min at 37° C. Working solutions of peroxide-free AA (5.5 μM and 110 μM AA for the CHO [hCOX-1] and CHO [COX-2] assays, respectively) are prepared by a 10-fold dilution of a concentrated AA solution in ethanol into HBSS containing 15 mM HEPES, pH 7.4. Cells are then challenged in the presence or absence of drug with the AA/HBSS solution to yield a final concentration of 0.5 μM AA in the CHO[hCOX-1] assay and a final concentration of 10 μM AA in the CHO[hCOX-2] assay. The reaction is terminated by the addition of 10 μl 1 N HCl followed by neutralization with 20 μl of 0.5 N NaOH. The samples are centrifuged at 300 ×g at 4° C. for 10 min, and an aliquot of the clarified supernatant is appropriately diluted for the determination of PGE₂ levels using an enzyme-linked immunoassay for PGE₂ (Correlate PGE₂ enzyme immunoassay kit, Assay Designs, Inc.). Cyclooxygenase activity in the absence of test compounds is determined as the difference in PGE₂ levels of cells challenged with AA versus the PGE₂ levels in cells mock-challenged with ethanol vehicle. Inhibition of PGE₂ synthesis by test compounds is calculated as a percentage of the activity in the presence of drug versus the activity in the positive control samples.

Assay of COX-1 Activity from U937 cell microsomes

U 937 cells are pelleted by centrifugation at 500 ×g for 5 min and washed once with phosphate-buffered saline and repelleted. Cells are resuspended in homogenization buffer consisting of 0.1 M Tris-HCl, pH 7.4, 10 mM EDTA, 2 μg/ml leupeptin, 2 μg/ml soybean trypsin inhibitor, 2 μg/ml aprotinin and 1 mM phenyl methyl sulfonyl fluoride. The cell suspension is sonicated 4 times for 10 sec and is centrifuged at 10,000 ×g for 10 min at 4° C. The supernatant is centrifuged at 100,000 ×g for 1 hr at 4° C. The 100,000 ×g microsomal pellet is resuspended in 0.1 M Tris-HCl, pH 7.4, 10 mM EDTA to approximately 7 mg protein/ml and stored at −80° C.

Microsomal preparations are thawed immediately prior to use, subjected to a brief sonication, and then diluted to a protein concentration of 125 μg/ml in 0.1 M Tris-HCl buffer, pH 7.4 containing 10 mM EDTA, 0.5 mM phenol, 1 mM reduced glutathione and 1 μM hematin. Assays are performed in duplicate in a final volume of 250 μl. Initially, 5 μl of DMSO vehicle or drug in DMSO are added to 20 μl of 0.1 M Tris-HCl buffer, pH 7.4 containing 10 mM EDTA in wells of a 96-deepwell polypropylene titre plate. 200 μl of the microsomal preparation are then added and pre-incubated for 15 min at room temperature before addition of 25 μl of 1 M arachidonic acid in 0.1 M Tris-HCl and 10 mM EDTA, pH 7.4. Samples are incubated for 40 min at room temperature and the reaction is stopped by the addition of 25 μl of 1 N HCl. Samples are neutralized with 25 μl of 1 N NaOH prior to quantitation of PGE₂ content by radioimmunoassay (Dupont-NEN or Amersham assay kits). Cyclooxygenase activity is defined as the difference between PGE₂ levels in samples incubated in the presence of arachidonic acid and ethanol vehicle.

Assay of the activity of purified human COX-2

The enzyme activity is measured using a chromogenic assay based on the oxidation of N,N,N′,N′-tetramethyl-p-phenylenediamine (TMPD) during the reduction of PGG₂ to PGH₂ by COX-2 (Copeland et al. (1994) Proc. Natl. Acad. Sci. 91, 11202-11206).

Recombinant human COX-2 is purified from Sf9 cells as previously described (Percival et al (1994) Arch. Biochem. Biophys. 15, 111-118). The assay mixture (180 μL) contains 100 mM sodium phosphate, pH 6.5, 2 mM genapol X-100, 1 μM hematin, 1 mg/ml gelatin, 80-100 units of purified enzyme (One unit of enzyme is defined as the amount of enzyme required to produce an O.D. change of 0.001/min at 610 nm) and 4 μL of the test compound in DMSO. The mixture is pre-incubated at room temperature (22° C.) for 15 minutes prior to initiation of the enzymatic reaction by the addition of 20 μL of a sonicated solution of 1 mM AA and 1 mM TMPD in assay buffer (without enzyme or hematin). The enzymatic activity is measured by estimation of the initial velocity of TMPD oxidation over the first 36 sec of the reaction. A non-specific rate of oxidation is observed in the absence of enzyme (0.007-0.010 O.D./min) and is subtracted before the calculation of the % inhibition. IC₅₀ values are derived from 4-parameter least squares non-linear regression analysis of the log-dose vs % inhibition plot.

### Human Whole Blood Assay

Rationale

Human whole blood provides a protein and cell-rich milieu appropriate for the study of biochemical efficacy of anti-inflammatory compounds such as selective COX-2 inhibitors. Studies have shown that normal human blood does not contain the COX-2 enzyme. This is consistent with the observation that COX-2 inhibitors have no effect on PGE₂ production in normal blood. These inhibitors are active only after incubation of human whole blood with LPS, which induces COX-2. This assay can be used to evaluate the inhibitory effect of selective COX-2 inhibitors on PGE₂ production. As well, platelets in whole blood contain a large amount of the COX-1 enzyme. Immediately following blood clotting, platelets are activated through a thrombin-mediated mechanism. This reaction results in the production of thromboxane B₂ (TxB₂) via activation of COX-1. Thus, the effect of test compounds on TxB₂ levels following blood clotting can be examined and used as an index for COX-1 activity. Therefore, the degree of selectivity by the test compound can be determined by measuring the levels of PGE₂ after LPS induction (COX-2) and TxB₂ following blood clotting (COX-1) in the same assay.

### Method

Step A: COX-2 (LPS-induced PGE₂ production)

Fresh blood is collected in heparinized tubes by venipuncture from both male and female volunteers. The subjects have no apparent inflammatory conditions and have not taken any NSAIDs for at least 7 days prior to blood collection. Plasma is immediately obtained from a 2 mL blood aliquot to use as blank (basal levels of PGE₂). The remaining blood is incubated with LPS (100 μg/ml final concentration, Sigma Chem, #L-2630 from E. coli; diluted in 0.1% BSA (Phosphate buffered saline) for 5 minutes at room temperature. Five hundred μL aliquots of blood are incubated with either 2 μL of vehicle (DMSO) or 2 μL of a test compound at final concentrations varying from 10 nM to 30 μM for 24 hours at 37° C. At the end of the incubation, the blood is centrifuged at 12,000 ×g for 5 minutes to obtain plasma. A 100 μL aliquot of plasma is mixed with 400 μL of methanol for protein precipitation. The supernatant is obtained and is assayed for PGE₂ using a radioimmunoassay kit (Amersham, RPA#530) after conversion of PGE₂ to its methyl oximate derivative according to the manufacturer's procedure.

Step B: COX-1 (Clotting-induced TxB₂ production)

Fresh blood is collected into vacutainers containing no anticoagulants. Aliquots of 500 μL are immediately transferred to siliconized microcentrifuge tubes preloaded with 2 μL of either DMSO or a test compound at final concentrations varying from 10 nM to 30 μM. The tubes are vortexed and incubated at 37° C. for 1 hour to allow blood to clot. At the end of incubation, serum is obtained by centrifugation (12,000 ×g for 5 min.). A 100 μL aliquot of serum is mixed with 400 μL of methanol for protein precipitation. The supernatant is obtained and is assayed for TxB₂ using an enzyme immunoassay kit (Cayman, #519031) according to the manufacturer's instruction.

### Rat Paw Edema Assay

Protocol

Male Sprague-Dawley rats (150-200 g) are fasted overnight and are given, po, either vehicle (1% methocel or 5% Tween 80) or a test compound. One hr later, a line is drawn using a permanent marker at the level above the ankle in one hind paw to define the area of the paw to be monitored. The paw volume (V₀) is measured using a plethysmometer (Ugo-Basile, Italy) based on the principle of water displacement. The animals are then injected subplantarly with 50 μl of 1% carrageenan solution in saline (FMC Corp, Maine) into the paw using an insulin syringe with a 25-gauge needle (i.e. 500 μg carrageenan per paw). Three hr later, the paw volume (V₃) is measured and the increases in paw volume (V₃-V₀) are calculated. The animals are sacrificed by CO₂ asphyxiation and the absence or presence of stomach lesions scored. Data is compared with the vehicle-control values and percent inhibition calculated. All treatment groups are coded to eliminate observer bias.

### NSAID-Induced Gastropathy in Rats

Rationale

The major side effect of conventional NSAIDs is their ability to produce gastric lesions in man. This action is believed to be caused by inhibition of COX-1 in the gastrointestinal tract. Rats are particularly sensitive to the actions of NSAIDs. In fact, rat models have been used commonly in the past to evaluate the gastrointestinal side effects of current conventional NSAIDs. In the present assay, NSAID-induced gastrointestinal damage is observed by measuring fecal ⁵¹Cr excretion after systemic injection of ⁵¹Cr-labeled red blood cells. Fecal ⁵¹Cr excretion is a well-established and sensitive technique to detect gastrointestinal integrity in animals and man.

Methods

Male Sprague Dawley rats (150 - 200 g) are administered orally a test compound either once (acute dosing) or b.i.d. for 5 days (chronic dosing). Immediately after the administration of the last dose, the rats are injected via a tail vein with 0.5 mL of ⁵¹Cr-labeled red blood cells from a donor rat. The animals are placed individually in metabolism cages with food and water ad lib. Feces are collected for a 48 h period and ⁵¹Cr fecal excretion is calculated as a percent of total injected dose. ⁵¹Cr-labeled red blood cells are prepared using the following procedures. Ten mL of blood is collected in heparinized tubes via the vena cava from a donor rat. Plasma is removed by centrifugation and replenished with equal volume of HBSS. The red blood cells are incubated with 400μ Ci of sodium ⁵¹chromate for 30 min. at 37° C. At the end of the incubation, the red blood cells are washed twice with 20 mL HBSS to remove free sodium ⁵¹chromate. The red blood cells are finally reconstituted in 10 mL HBSS and 0.5 mL of the solution (about 20μ Ci) is injected per rat.

### Protein-losing Gastropathy in Squirrel Monkeys

Rationale

Protein-losing gastropathy (manifested as appearance of circulating cells and plasma proteins in the GI tract) is a significant and dose-limiting adverse response to standard non-steroidal anti-inflammatory drugs (NSAIDs). This can be quantitatively assessed by intravenous administration of ⁵¹CrCl₃ solution. This isotopic ion can avidly bind to cell and serum globins and cell endoplasmic reticulum. Measurement of radioactivity appearing in feces collected for 24 h after administration of the isotope thus provides a sensitive and quantitative index of protein-losing gastropathy.

Methods

Groups of male squirrel monkeys (0.8 to 1.4 kg) are treated by gavage with either 1% methocell or 5% Tween 80 in H₂O vehicles, (3 mL/kg b.i.d.) or test compounds at doses from 1-100 mg/kg b.i.d. for 5 days. Intravenous ⁵¹Cr (5 μCi/kg in 1 ml/kg phosphate buffer saline (PBS)) is administered 1 h after the last drug/vehicle dose, and feces collected for 24 h in a metabolism cage and assessed for excreted ⁵¹Cr by gamma-counting. Venous blood is sampled 1 h and 8 h after the last drug dose, and plasma concentrations of drug measured by RP-HPLC.

LPS-Induced Pyrexia in Conscious Rats

Male Sprague-Dawley rats (150 - 200 g) were fasted for 16-18 h before use. At approximately 9:30 a.m., the animals were placed temporarily in plexiglass restrainers and their baseline rectal temperature was recorded using a flexible temperature probe (YSI series 400) connected to a digital thermometer (Model 08502, Cole Parmer). The same probe and thermometer were used for all animals to reduce experimental error. The animals were returned to their cages after the temperature measurements. At time zero, the rats were injected intraperitoneally with either saline or LPS (2 mg/kg, Sigma Chem) and the rectal temperature was remeasured at 5, 6 and 7 h following LPS injection. After the measurement at 5 h, when the increase in temperature had reached a plateau, the LPS-injected rats were given either the vehicle (1% methocel) or a test compound orally to determine whether the compound could reverse the pyrexia. Percent reversal of the pyrexia was calculated using the rectal temperature obtained at 7 h in the control (vehicle-treated) group as the reference (zero reversal) point. Complete reversal of pyrexia to the pre-LPS baseline value is taken as 100%.

LPS-Induced Pyrexia in Conscious Squirrel Monkeys

Temperature probes were surgically implanted under the abdominal skin in a group of squirrel monkeys (Saimiri sciureus) (1.0-1.7 kg). This allows for the monitoring of body temperature in conscious, unrestrained monkeys by a telemetric sensing system (Data Sciences International, Minnesota). The animals were fasted and were placed in individual cages for acclimatization 13-14 h before use. Electronic receivers were installed on the side of the cages which pick up signals from the implanted temperature probes. At approximately 9:00 a.m. on the day of the experiment, the monkeys were restrained temporarily in training chairs and were given a bolus I.V. injection of LPS, (6 mg/kg, dissolved in sterile saline). The animals were returned to their cages and body temperature was recorded continuously every 5 min. Two h after injection of LPS, when the body temperature had increased by 1.5-2° C., the monkeys were dosed orally with either vehicle (1% methocel) or a test compound (3 mg/kg). One hundred minutes later, the difference between the body temperature and the baseline value was determined. Percent inhibition was calculated taking the value in the control group as 0% inhibition.

Acute Inflammatory Hyperalgesia Induced by Carrageenan in Rats

Experiments were performed using male Sprague Dawley rats (90-110 g). Hyperalgesia to mechanical compression of the hind paw was induced by intraplantar injection of carrageenan (4.5 mg into one hind paw) 3 h previously. Control animals received an equivalent volume of saline (0.15 ml intraplantar). A test compound (0.3-30 mg/kg, suspended in 0.5% methocel in distilled water) or vehicle (0.5% methocel) was administered orally (2 ml/kg) 2 h after carrageenan. The vocalisation response to compression of the hind paw was measured 1 h later using a Ugo Basile algesiometer.

Statistical analysis for carrageenan-induced hyperalgesia was performed using one-way ANOVA (BMDP Statistical Software Inc.). Hyperalgesia was determined by subtracting the vocalisation threshold in saline injected rats from that obtained in animals injected with carrageenan. Hyperalgesia scores for drug-treated rats were expressed as a percentage of this response. ID₅₀ values (the dose producing 50% of the maximum observed response) were then calculated by nonlinear least squares regression analysis of mean data using GraFit (Erithacus Software).

Adjuvant-Induced Arthritis in Rats

Seventy, 6.5-7.5 week old, female Lewis rats (body weight 146-170 g) were weighed, ear marked, and assigned to groups (a negative control group in which arthritis was not induced, a vehicle control group, a positive control group administered indomethacin at a total daily dose of 1 mg/kg and four groups administered with a test compound at total daily doses of 0.10-3.0 mg/kg) such that the body weights were equivalent within each group. Six groups of 10 rats each were injected into a hind paw with 0.5 mg of Mycobacterium butyricum in 0.1 ml of light mineral oil (adjuvant), and a negative control group of 10 rats was not injected with adjuvant. Body weights, contralateral paw volumes (determined by mercury displacement plethysmography) and lateral radiographs (obtained under Ketamine and Xylazine anesthesia) were determined before (day -1) and 21 days following adjuvant injection, and primary paw volumes were determined before (day -1) and on days 4 and 21 following adjuvant injection. The rats were anesthetized with an intramuscular injection of 0.03 - 0.1 ml of a combination of Ketamine (87 mg/kg) and Xylazine (13 mg/kg) for radiographs and injection of adjuvant. The radiographs were made of both hind paws on day 0 and day 21 using the Faxitron (45 kVp, 30 seconds) and Kodak X-OMAT TL film, and were developed in an automatic processor. Radiographs were evaluated for changes in the soft and hard tissues by an investigator who was blinded to experimental treatment. The following radiographic changes were graded numerically according to severity: increased soft issue volume (0-4), narrowing or widening of joint spaces (0-5) subchondral erosion (0-3), periosteal reaction (0-4), osteolysis (0-4) subluxation (0-3), and degenerative joint changes (0-3). Specific criteria were used to establish the numerical grade of severity for each radiographic change. The maximum possible score per foot was 26. A test compound at total daily doses of 0.1, 0.3, 1, and 3 mg/kg/day, Indomethacin at a total daily dose of 1 mg/kg/day, or vehicle (0.5% methocel in sterile water) were administered per os b.i.d. beginning post injection of adjuvant and continuing for 21 days. The compounds were prepared weekly, refrigerated in the dark until used, and vortex mixed immediately prior to administration.

Two-factor (‘treatment’ and ‘time’) analysis of variance with repeated measures on ‘time’ were applied to the % changes for body weight and foot volumes and to the rank-transformed radiographic total scores. A post hoc Dunnett's test was conducted to compare the effect of treatments to vehicle. A one-way analysis of variance was applied to the thymic and spleen weights followed by the Dunnett's test to compare the effect of treatments to vehicle. Dose-response curves for % inhibition in foot volumes on days 4, 14 and 21 were fitted by a 4-parameter logistic function using a nonlinear least squares' regression. ID₅₀ was defined as the dose corresponding to a 50% reduction from the vehicle and was derived by interpolation from the fitted 4-parameter equation.

### Pharmacokinetics in Rats

Per Os Pharmacokinetics in Rats

Male Sprague Dawley rats (325-375 g) are fasted overnight prior to each PO blood level study.

The rats are placed in the restrainer one at a time and the box firmly secured. The zero blood sample is obtained by nicking a small (1 mm or less) piece off the tip of the tail. The tail is then stroked with a firm but gentle motion from the top to the bottom to milk out the blood. Approximately 1 mL of blood is collected into a heparinized vacutainer tube.

Compounds are prepared as required, in a standard dosing volume of 10 mL/kg, and administered orally by passing a 16 gauge, 3″ gavaging needle into the stomach.

Subsequent bleeds are taken in the same manner as the zero bleed except that there is no need to nick the tail again. The tail is cleaned with a piece of gauze and milked/stroked as described above into the appropriately labelled tubes.

Immediately after sampling, blood is centrifuged, separated, put into clearly marked vials and stored in a freezer until analysed.

Typical time points for determination of rat blood levels after PO dosing are:

0, 15 min, 30 min, 1 h, 2 h, 4 h, 6 h

After the 4 hr time point bleed, food is provided to the rats ad libitum. Water is provided at all times during the study.

Vehicles:

The following vehicles may be used in PO rat blood level determinations:

PEG 200/300/400: restricted to 2 mL/kg

Methocel 0.5% - 1.0%: 10 mL/kg

Tween 80: 10 mL/kg

Compounds for PO blood levels can be in suspension form. For better dissolution, the solution can be placed in a sonicator for approximately 5 minutes.

For analysis, aliquots are diluted with an equal volume of acetonitrile and centrifuged to remove protein precipitate. The supernatant is injected directly onto a C-18 HPLC column with UV detection. Quantitation is done relative to a clean blood sample spiked with a known quantity of drug. Bioavailability (F) is assessed by comparing area under the curve (AUC) i.v. versus p.o.

$$F = \frac{AUCpo}{AUCiv} \times \frac{DOSEiv}{DOSEpo} \times 100 \%$$

Clearance rates are calculated from the following relation:

$$CL = \frac{DOSEiv \text{(mg/kg)}}{AUCiv}$$

The units of CL are mL/h·kg (milliliters per hour kilogram)

Intravenous Pharmacokinetics in Rats

The animals are housed, fed and cared for according to the Guidelines of the Canadian Council on Animal Care.

Male Sprague Dawley (325-375 g) rats are placed in plastic shoe box cages with a suspended floor, cage top, water bottle and food.

The compound is prepared as required, in a standard dosing volume of 1 mL/kg.

Rats are bled for the zero blood sample and dosed under CO₂ sedation. The rats, one at a time, are placed in a primed CO₂ chamber and taken out as soon as they have lost their righting reflex. The rat is then placed on a restraining board, a nose cone with CO₂ delivery is placed over the muzzle and the rat restrained to the board with elastics. With the use of forceps and scissors, the jugular vein is exposed and the zero sample taken, followed by a measured dose of compound which is injected into the jugular vein. Light digital pressure is applied to the injection site, and the nose cone is removed. The time is noted. This constitutes the zero time point.

The 5 min bleed is taken by nicking a piece (I1-2 mm) off the tip of the tail. The tail is then stroked with a firm but gentle motion from the top of the tail to the bottom to milk the blood out of the tail. Approximately 1 mL of blood is collected into a heparinized collection vial. Subsequent bleeds are taken in the same fashion, except that there is no need to nick the tail again. The tail is cleaned with a piece of gauze and bled, as described above, into the appropriate labelled tubes.

Typical time points for determination of rat blood levels after I.V. dosing are either:

0, 5 min, 15 min, 30 min, 1 h, 2 h, 6 h

or

0, 5 min, 30 min, 1 h, 2 h, 4 h, 6 h.

Vehicles:

The following vehicles may be used in IV rat blood level determinations:

| | |
|---|---|
| Dextrose: | 1 mL/kg |
| Moleculosol 25%: | 1 mL/kg |
| DMSO (dimethylsulfoxide): | Restricted to a dose volume of 0.1 mL per |
| | animal |
| PEG 200: | Not more than 60% mixed with 40% |
| | sterile water-1 mL/kg |

With Dextrose, either sodium bicarbonate or sodium carbonate can be added if the solution is cloudy.

For analysis, aliquots are diluted with an equal volume of acetonitrile and centrifuged to remove protein precipitate. The supernatant is injected directly onto a C-18 HPLC column with UV detection. Quantitation is done relative to a clean blood sample spiked with a known quantity of drug. Bioavailability (F) is assessed by comparing area under the curve (AUC) i.v. versus p.o.

$$F = \frac{AUCpo}{AUCiv} \times \frac{DOSEiv}{DOSEpo} \times 100 \%$$

Clearance rates are calculated from the following relation:

$$CL = \frac{DOSEiv \text{(mg/kg)}}{AUCiv}$$

The units of CL are mL/h·kg (milliliters per hour kilogram)

### PSA Conjgate Assays

Assessment of the Recognition of Oligopeptide-Cytotoxic Drug Conjugates by Free PSA

The PSA conjugates, prepared as described above and in particular in Examples 13-22, are individually dissolved in PSA digestion buffer (50 mM tris(hydroxymethyl)-aminomethane pH7.4, 140 mM NaCl) and the solution added to PSA at a molar ration of 100 to 1. Alternatively, the PSA digestion buffer utilized is 50 mM tris(hydroxymethyl)-aminomethane pH7.4, 140 mM NaCl. The reaction is quenched after various reaction times by the addition of trifluoroacetic acid (TFA) to a final 1% (volume/volume). Alternatively the reaction is quenched with 10 mM ZnCl₂. The quenched reaction is analyzed by HPLC on a reversed-phase C18 column using an aqueous 0.1% TFA/acetonitrile gradient. The amount of time (in minutes) required for 50% cleavage of the noted oligopeptide-cytotoxic agent conjugates with enzymatically active free PSA were then calculated.

In vitro Assay of Cytotoxicity of Peptidyl Derivatives of Doxorubicin:

The cytotoxicities of a cleaveable oligopeptide-doxorubicin conjugates, prepared as described above and in particular in Examples 13-15, against a line of cells which is known to be killed by unmodified doxorubicin are assessed with an Alamar Blue assay. Specifically, cell cultures of LNCap prostate tumor cells (which express enzymatically active PSA) or DuPRO cells in 96 well plates are diluted with medium (Dulbecco's Minimum Essential Medium-α [MEM-α]) containing various concentrations of a given conjugate (final plate well volume of 200 μl). The cells are incubated for 3 days at 37° C., 20 μl of Alamar Blue is added to the assay well. The cells are further incubated and the assay plates are read on a EL-310 ELISA reader at the dual wavelengths of 570 and 600 nm at 4 and 7 hours after addition of Alamar Blue. Relative percentage viability at the various concentration of conjugate tested is then calculated versus control (no conjugate) cultures.

In vitro Assay of Cytotoxicity of Peptidyl Derivatives of Vinca Drugs

The cytotoxicities of a cleaveable oligopeptide-vinca drug conjugates, prepared as described above and in particular in Examples 16-22, against a line of cells which is known to be killed by unmodified vinca drug was assessed with an Alamar Blue assay. Specifically, cell cultures of LNCap prostate tumor cells, Colo320DM cells (designated C320) or T47D cells in 96 well plates are diluted with medium containing various concentrations of a given conjugate (final plate well volume of 200 μl). The Colo320DM cells, which do not express free PSA, are used as a control cell line to determine non-mechanism based toxicity. The cells are incubated for 3 days at 37° C., 20 μl of Alamar Blue is added to the assay well. The cells are further incubated and the assay plates are read on a EL-310 ELISA reader at the dual wavelengths of 570 and 600 nm at 4 and 7 hours after addition of Alamar Blue. Relative percentage viability at the various concentration of conjugate tested is then calculated versus control (no conjugate) cultures and an EC₅₀ was determined.

In vivo Efficacy of Peptidyl -Cytotoxic Agent Conjugates

LNCaP.FGC or DuPRO-1 cells are trypsinized, resuspended in the growth medium and centifuged for 6 mins. at 200 xg. The cells are resuspended in serum-free α-MEM and counted. The appropriate volume of this solution containing the desired number of cells is then transferred to a conical centrifuge tube, centrifuged as before and resuspended in the appropriate volume of a cold 1:1 mixture of α-MEM-Matrigel. The suspension is kept on ice until the animals are inoculated.

Harlan Sprague Dawley male nude mice (10-12 weeks old) are restrained without anesthesia and are inoculated with 0.5 mL of cell suspension on the left flank by subcutaneous injection using a 22G needle. Mice are either given approximately 5×10⁵ DuPRO cells or 1.5×10⁷ LNCaP.FGC cells.

Following inoculation with the tumor cells the mice are treated under one of two protocols:

Protocol A:

One day after cell inoculation the animals are dosed with a 0.1-0.5 mL volume of test conjugate, unconjugated cytotoxic agent or vehicle control (sterile water). Dosages of the conjugate and unconjugated cytotoxic agent are initially the maximum non-lethal amount, but may be subsequently titrated lower. Identical doses are administered at 24 hour intervals for 5 days. After 10 days, blood samples are removed from the mice and the serum level of PSA is determined. Similar serum PSA levels are determined at 5-10 day intervals. At the end of 5.5 weeks the mice are sacrificed and weights of any tumors present are measured and serum PSA again determined. The animals' weights are determined at the beginning and end of the assay.

Protocol B:

Ten days after cell inoculation, blood samples are removed from the animals and serum levels of PSA are determined. Animals are then grouped according to their PSA serum levels. At 14-15 days after cell inoculation, the animals are dosed with a 0.1-0.5 mL volume of test conjugate, unconjugated cytotoxic agent or vehicle control (sterile water). Dosages of the conjugate and unconjugated cytotoxic agent are initially the maximum non-lethal amount, but may be subsequently titrated lower. Identical doses are administered at 24 hour intervals for 5 days. Serum PSA levels are determined at 5-10 day intervals. At the end of 5.5 weeks the mice are sacrificed, weights of any tumors present are measured and serum PSA again determined. The animals' weights are determined at the beginning and end of the assay.

In vivo Efficacy of Administration of a Combination of a PSA Conjugate and an NSAID

Male nude mice (4 groups of 15) are injected subcutaneously with 1.5×10⁷ LNCaP.FGC cells (available from the American Type Culture Collection, ATCC No. CRL-1740; see also J. S. Horoszewicz et al. Cancer Res., 43:1809-1818 (1983)) in 80% Matrigel.

Five days after the tumor cell implantation, a test NSAID is incorporated into the rodent chow food that is given to the mice ad libitum for the remainder of the test period. The concentration of the NSAID in the food is adjusted to provide a therapeutically minimal or subminimal plasma concentration of the NSAID. For example, if the compound of Example 1 is being tested in combination with a PSA conjugate, the concentration of the compound in the food is adjusted so that a continuous plasma concentration of between 0.3-1.0 μM is maintained.

Administration of the NSAID is as follows:

Group A: Food containing test NSAID compound

Group B: Food without NSAID

Group C: Food containing test NSAID compound

Group D: Food without NSAID

Beginning at the same time as administration of the NSAID, a solution of test PSA conjugate is administered to Groups A and B. Vehicle is administered to Groups C and D. The PSA conjugate is administered IV as a therapeutically minimal dose. For example, when the PSA conjugate described in Example 14 is tested, a 0.20 mL of a solution of test PSA conjugate, (3-5 mpk, 34.1 mL D5W +80 μL 7.5% sodium bicarbonate) is administered to Groups A and B. Vehicle (0.20 mL) is administered to Groups C and D.

Three days after the initial dosing of the NSAID and the PSA conjugate, three mice from each group are bled from the tail vein to assess serum levels of the test NSAID.

After the initial dose of PSA conjugate, the animals are administered PSA conjugate solution either as four additional doses (one/day) of the test PSA conjugate solution or vehicle are administered to the respective Groups over four consecutive days, or once a week for four consecutive weeks At the end of 5-6 weeks after the inoculation with the LNCaP cells the mice are bled from the tail vein and the plasma PSA level is measured using a Tandem®-E PSA ImmunoEnzyMetri Assay kit (Hybritech). The plasma concentration of the NSAID is also determined at this time. The mice are then sacrificed, weighed, tumors excised and weighed.

In vitro determination of proteolytic cleavage of conjugates by endogenous non-PSA proteases

Step A: Preparation of proteolytic tissue extracts

All procedures are carried out at 4° C. Appropriate animals are sacrificed and the relevant tissues are isolated and stored in liquid nitrogen. The frozen tissue is pulverized using a mortar and pestle and the pulverized tissue is transfered to a Potter-Elvejeh homogenizer and 2 volumes of Buffer A (50 mM Tris containing 1.15% KCl, pH 7.5) are added. The tissue is then disrupted with 20 strokes using first a lose fitting and then a tight fitting pestle. The homogenate is centrifuged at 10,000 x g in a swinging bucket rotor (HB4-5), the pellet is discarded and the re-supernatant centrifuged at 100,000 x g (Ti 70). The supernatant (cytosol) is saved.

The pellet is resuspended in Buffer B (10 mM EDTA containing 1.15% KCl, pH 7.5) using the same volume used in step as used above with Buffer A. The suspension is homogenized in a dounce homogenizer and the solution centrifuged at 100,000x g. The supernatant is discarded and the pellet resuspended in Buffer C(10 mM potassium phosphate buffer containing0.25 M sucrose, pH 7.4), using ½ the volume used above, and homogenized with a dounce homogenizer.

Protein content of the two solutions (cytosol and membrane) is determined using the Bradford assay. Assay aliquots are then removed and frozen in liquid N₂. The aliquots are stored at −70° C.

Step B: Proteolytic cleavage assay

For each time point, 20 microgram of PSA conjugate and 150 micrograms of tissue protein, prepared as described in Step A and as determined by Bradford in reaction buffer are placed in solution of final volume of 200 microliters in buffer (50 mM TRIS, 140 mM NaCl, pH 7.2). Assay reactions are run for 0, 30, 60, 120, and 180 minutes and are then quenched with 9 microliters of 0.1 M ZnCl₂ and immediately placed in boiling water for 90 seconds. Reaction products are analyzed by HPLC using a VYDAC C18 15 cm column in water/acetonitrile (5% to 50% acetonitrile over 30 minutes).

What is claimed is:

1. A method treating cancer in a mammal in need thereof which comprises administering to said mammal amounts of at least one NSAID compound and at least one PSA conjugate.

2. The method according to claim 1 wherein an amount of an NSAID compound and an amount of an PSA conjugate are administered consecutively.

3. The method according to claim 1 wherein an amount of an NSAID compound and an amount of an PSA conjugate are administered simultaneously.

4. The method according to claim 1 wherein the cancer is a cancer related to cells that express enzymatically active PSA.

5. The method according to claim 1 wherein the cancer is prostate cancer.

6. The method according to claim 1 wherein the PSA conjugate is selected from:
a) a compound represented by the formula IV: 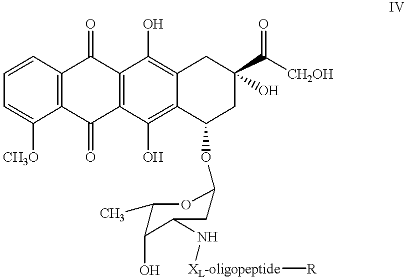
wherein:
oligopeptide is an oligopeptide which is selectively recognized by the free prostate specific antigen (PSA) and is capable of being proteolytically cleaved by the enzymatic activity of the free prostate specific antigen;
X_{L} is absent or is an amino acid selected from:
a) phenylalanine,
b) leucine,
c) valine,
d) isoleucine,
e) (2-naphthyl)alanine,
f) cyclohexylalanine,
g) diphenylalanine,
h) norvaline, and
j) norleucine;
R is hydrogen or —(C═O)R¹; and
R¹ is C₁-C₆-alkyl or aryl,
or the pharmaceutically acceptable salt thereof;
b) a compound represented by the formula V: 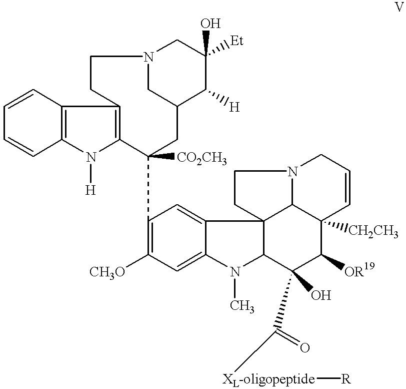
wherein:
oligopeptide is an oligopeptide which is selectively recognized by the free prostate specific antigen (PSA) and is capable of being proteolytically cleaved by the enzymatic activity of the free prostate specific antigen;
X_{L} is absent or is an amino acid selected from:
a) phenylalanine,
b) leucine,
c) valine,
d) isoleucine,
e) (2-naphthyl)alanine,
f) cyclohexylalanine,
g) diphenylalanine,
h) norvaline, and
j) norleucine; or
X_{L} is —NH—(CH₂)ₙ—NH—
R is hydrogen or —(C═O)R¹;
R¹ is C₁-C₆-alkyl or aryl;
R¹⁹ is hydrogen or acetyl; and
n is 1, 2, 3, 4 or 5,
or the pharmaceutically acceptable salt thereof;
c) a compound represented by the formula VI: 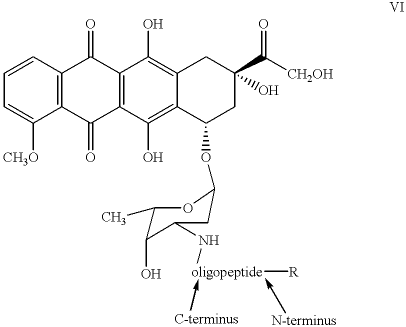
wherein:
oligopeptide is an oligopeptide which is selectively recognized by the free prostate specific antigen (PSA) and is capable of being proteolytically cleaved by the enzymatic activity of the free prostate specific antigen, wherein the oligopeptide comprises a cyclic amino acid of the formula: 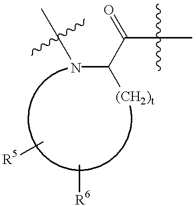
and wherein the C-terminus carbonyl is covalently bound to the amine of doxorubicin;
R is selected from
a) hydrogen,
b) —(C═O)R^{1a}, 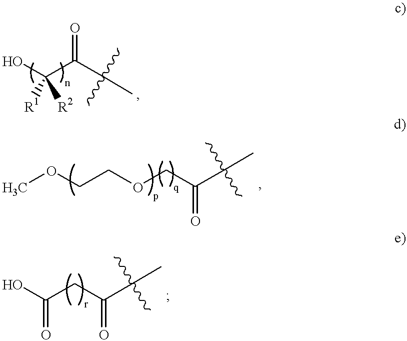
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated aryl, polyhydroxylated aryl or aryl;
R⁵ is selected from HO— and C₁-C₆ alkoxy;
R⁶ is selected from hydrogen, halogen, C₁-C₆ alkyl, HO— and C₁-C₆ alkoxy; and
n is 1, 2, 3 or 4;
p is zero or an integer between 1 and 100;
q is 0 or 1, provided that if p is zero, q is 1;
r is an integer between 1 and 10; and
t is 3 or 4;
or a pharmaceutically acceptable salt thereof;
d) a compound represented by the formula VII: 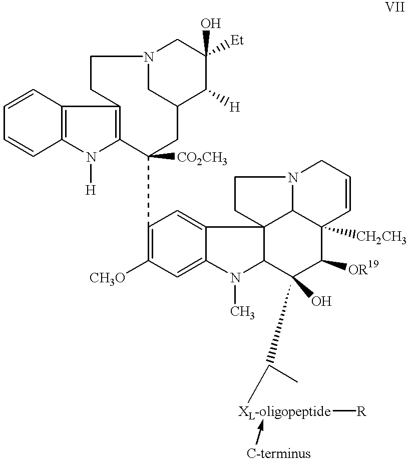
wherein:
oligopeptide is an oligopeptide which is selectively recognized by the free prostate specific antigen (PSA) and is capable of being proteolytically cleaved by the enzymatic activity of the free prostate specific antigen, and the oligopeptide comprises a cyclic arnino acid of the formula: 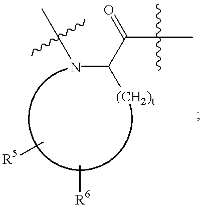
X_{L} is —NH—(CH₂)ᵤ—NH—
R is selected from
a) hydrogen,
b) —(C═O)R^{1a}, 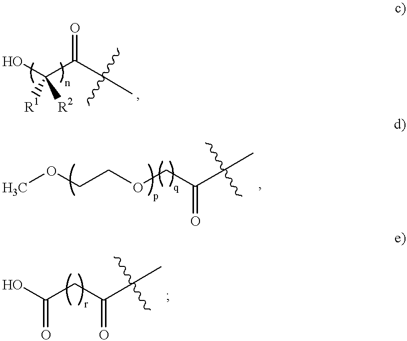
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated aryl, polyhydroxylated aryl or aryl,
R¹⁹ is hydrogen, (C₁-C₃ alkyl)-CO, or chlorosubstituted (C₁-C₃ alkyl)-CO;
n is 1, 2, 3 or 4;
p is zero or an integer between 1 and 100;
q is 0 or 1, provided that if p is zero, q is 1;
r is 1, 2 or 3;
t is 3 or 4;
u is 1, 2, 3, 4 or 5,
or the pharmaceutically acceptable salt thereof;
e) a compound represented by the formula VIII: 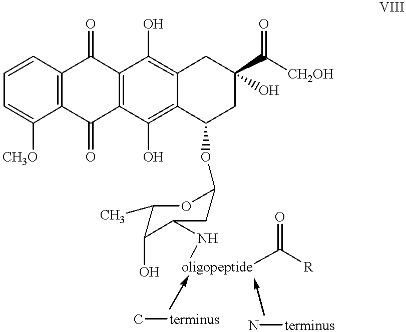
wherein:
oligopeptide is an oligopeptide which is selectively recognized by the free prostate specific antigen (PSA) and is capable of being proteolytically cleaved by the enzymatic activity of the free prostate specific antigen, and wherein the C-terminus carbonyl is covalently bound to the amine of doxorubicin and the N-terminus amine is covalently bound to the carbonyl of the blocking group;
R is selected from 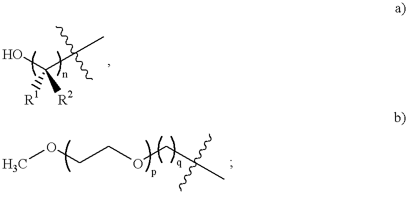
R¹ and R² are independently selected from: hydrogen, OH, C₁-C₆ alkyl, C₁-C₆ alkoxy, C₁-C₆ aralkyl and aryl;
n is 2, 3 or 4;
p is zero or an integer between 1 and 100;
q is 0 or 1,provided that if p is zero, q is 1;
or the pharmaceutically acceptable salt thereof;
f) a compound represented by the formula IX: 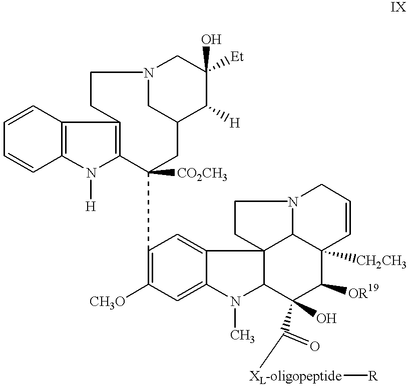
wherein:
oligopeptide is an oligopeptide which is selectively recognized by the free prostate specific antigen (PSA) and is capable of being proteolytically cleaved by the enzymatic activity of the free prostate specific antigen;
X_{L} is —NH—(CH₂)ᵣ—NH—
R is selected from 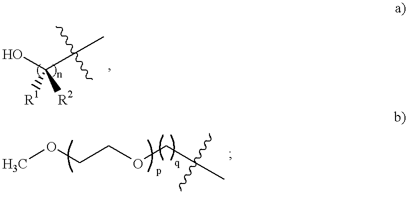
R¹ and R² are independently selected from: hydrogen, OH, C₁-C₆ alkyl, C₁-C₆ alkoxy, C₁-C₆ aralkyl and aryl;
R¹⁹ is hydrogen, (C₁-C₃ alkyl)-CO, or chlorosubstituted (C₁-C₃ alkyl)-CO;
n is 1, 2, 3 or 4;
p is zero or an integer between 1 and 100;
q is 0 or 1, provided that if p is zero, q is 1;
r is 1, 2, 3, 4 or 5,
or the pharmaceutically acceptable salt thereof;
g) a compound represented by the formula X: 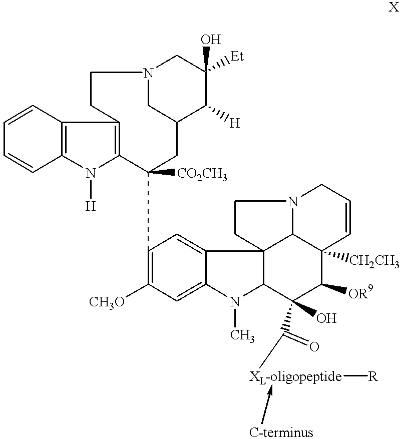
wherein:
oligopeptide is an oligopeptide which is selectively recognized by the free prostate specific antigen (PSA) and is capable of being proteolytically cleaved by the enzymatic activity of the free prostate specific antigen,
X_{L} is —NH—(CH₂)ᵤ—W—(CH₂)ᵤ—NH—
R is selected from
a) hydrogen,
b) —(C═O)R^{1a}, 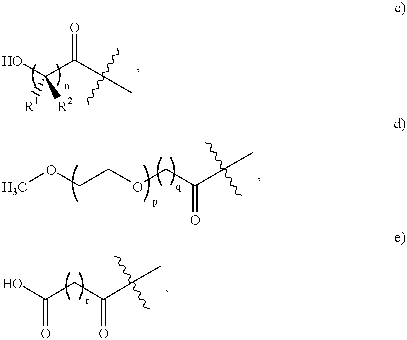
f) ethoxysquarate, and
g) cotininyl;
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;
R⁹ is hydrogen, (C₁-C₃ alkyl)-CO, or chlorosubstituted (C₁-C₃ alkyl)-CO;
W is selected from cyclopentyl, cyclohexyl, cycloheptyl or bicyclo[2.2.2]octanyl;
n is 1, 2, 3 or 4;
p is zero or an integer between 1 and 100;
q is 0 or 1, provided that if p is zero, q is 1;
r is 1, 2 or 3;
t is 3 or 4;
u is 0, 1, 2 or 3,
or the pharmaceutically acceptable salt thereof; and
h) a compound represented by the formula XI: 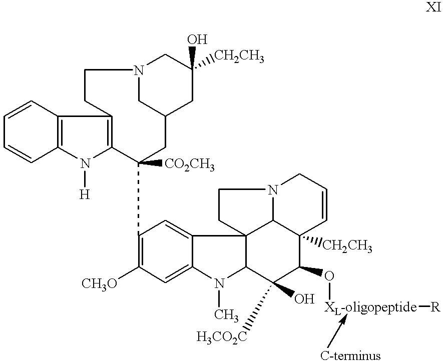
wherein:
oligopeptide is an oligopeptide which is selectively recognized by the free prostate specific antigen (PSA) and is capable of being proteolytically cleaved by the enzymatic activity of the free prostate specific antigen,
X_{L} is selected from: a bond, —C(O)—(CH₂)ᵤ—W—(CH₂)ᵤ—O— and —C(O)—(CH₂)ᵤ—W—(CH₂)ᵤ—NH—;
R is selected from
a) hydrogen,
b) —(C═O)R^{1a}, 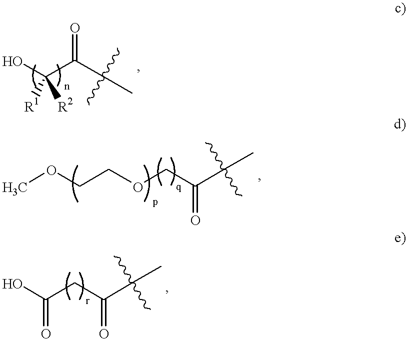
f) ethoxysquarate, and
g) cotininyl;
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;
R⁹ is hydrogen, (C₁-C₃ alkyl)-CO, or chlorosubstituted (C₁-C₃ alkyl)-CO;
W is selected from a branched or straight chain C₁-C₆-alkyl, cyclopentyl, cyclohexyl, cycloheptyl or bicyclo[2.2.2]octanyl;
n is 1, 2, 3 or 4;
p is zero or an integer between 1 and 100;
q is 0 or 1, provided that if p is zero, q is 1;
r is 1, 2 or 3;
t is 3 or 4;
u is 0, 1, 2 or 3;
or the pharmaceutically acceptable salt or optical isomer thereof.

7. The method according to claim 6 wherein the PSA conjugate is selected from: 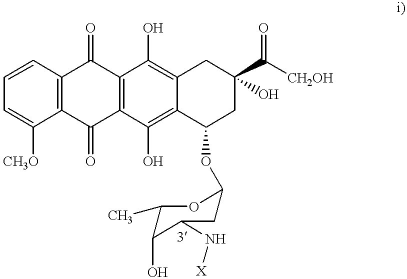
wherein X is:
AsnLysIleSerTyrGlnSer—(SEQ.ID.NO.: 1),
AsnLysIleSerTyrGlnSerSer—(SEQ.ID.NO.: 2),
AsnLysIleSerTyrGlnSerSerSer—(SEQ.ID.NO.:3 ),
AsnLysIleSerTyrGlnSerSerSerThr—(SEQ.ID.NO.:4),
AsnLysIleSerTyrGlnSerSerSerThrGlu—(SEQ.ID.NO.: 5),
AlaAsnLysIleSerTyrGlnSerSerSerThrGlu—(SEQ.ID.NO.: 6),
Ac—AlaAsnLysIleSerTyrGlnSerSerSerThr—(SEQ.ID.NO.: 7),
Ac—AlaAsnLysIleSerTyrGlnSerSerSerThrLeu—(SEQ.ID.NO.: 8),
Ac—AlaAsnLysAlaSerTyrGlnSerAlaSerThrLeu—(SEQ.ID.NO.: 9),
Ac—AlaAsnLysAlaSerTyrGlnSerAlaSerLeu—(SEQ.ID.NO.: 10),
Ac—AlaAsnLysAlaSerTyrGlnSerSerSerLeu—(SEQ.ID.NO.: 11),
Ac—AlaAsnLysAlaSerTyrGlnSerSerLeu—(SEQ.ID.NO.: 12),
Ac—SerTyrGlnSerSerSerLeu—(SEQ.ID.NO.: 13),
Ac—hArgTyrGlnSerSerSerLeu—(SEQ.ID.NO.: 14).
Ac—LysTyrGlnSerSerSerLeu—(SEQ.ID.NO.: 15),
Ac—LysTyrGlnSerSerNle—(SEQ.ID.NO.: 16), 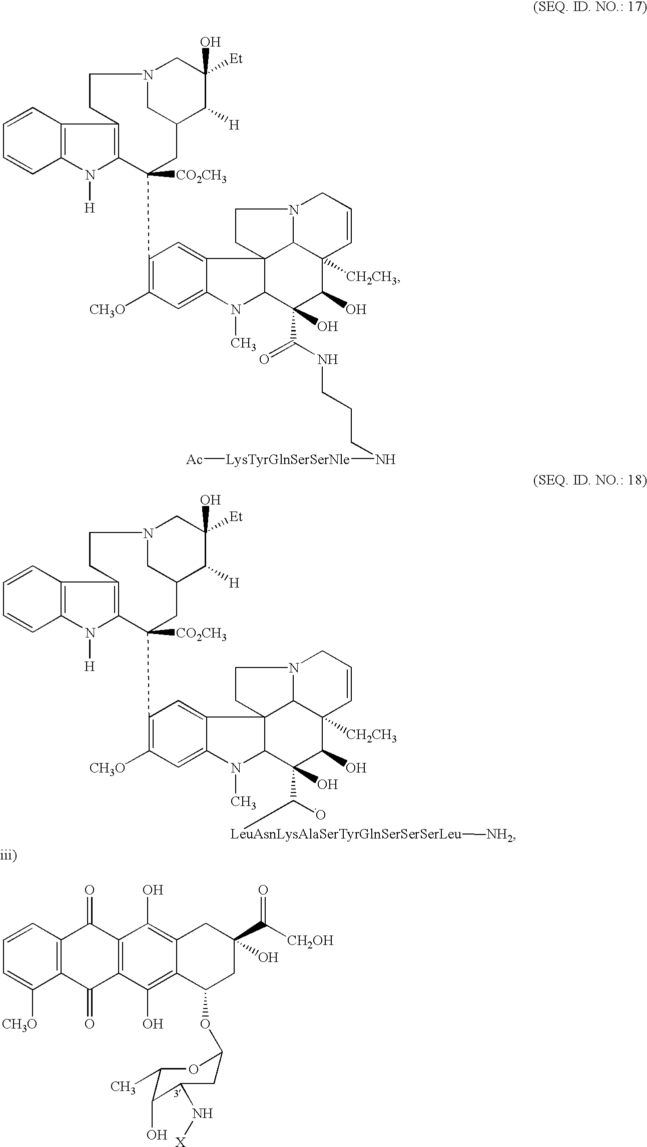
wherein X is: 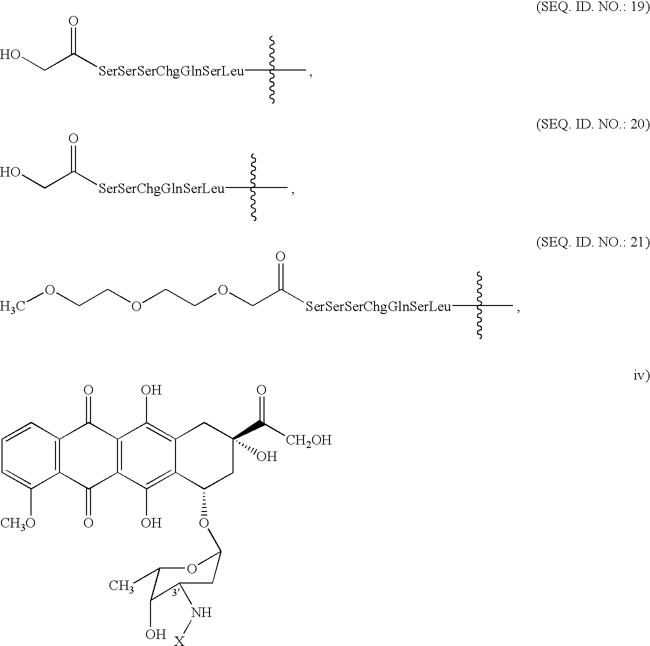
wherein X is: 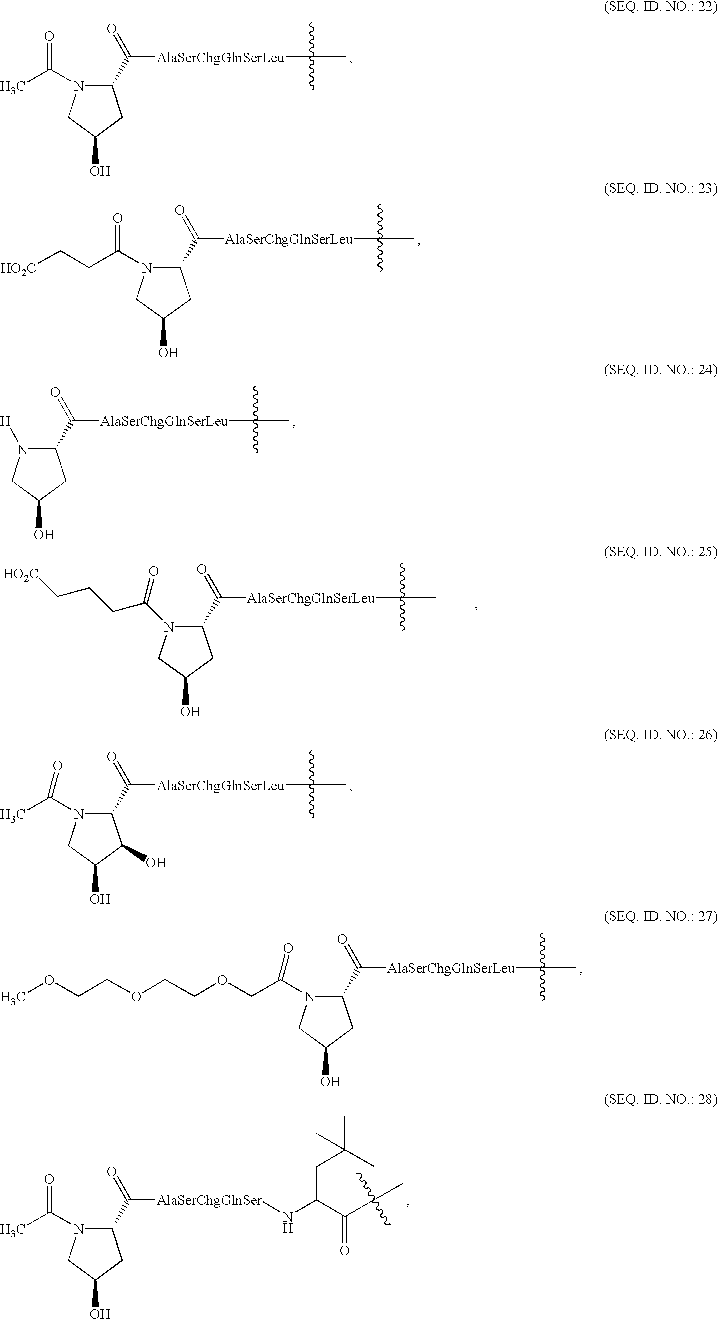
wherein X is 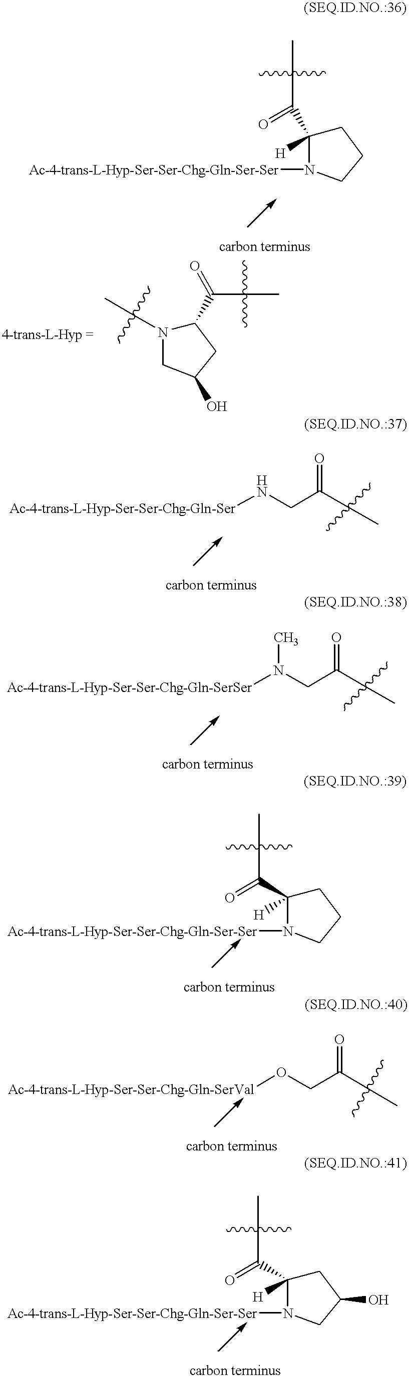
or a pharmaceutically acceptable salt or optical isomer thereof.

8. The method according to claim 7 wherein the PSA conjugate is: 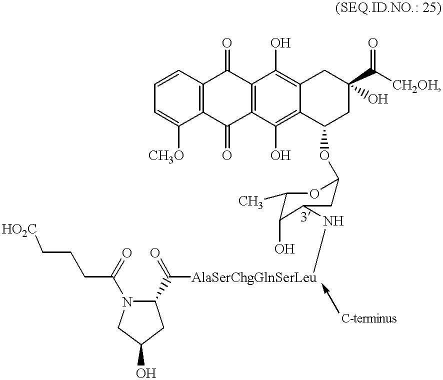
or a pharmaceutically acceptable salt thereof.

9. The method according to claim 1 wherein the NSAID compound is selected from aspirin, ibuprofen, INDOMETHACIN, SULINDAC, DOLOBID, DICLOFENAC, NAPROXEN, PIROXICAN, ETODOLAC, KETOPROFEN, FLURBIPROFEN, MELOXICAM, FLOSULIDE ,NABUMETONE and a COX-2 inhibiting agent.

10. The method according to claim 1 wherein the NSAID compound is a COX-2 inhibiting agent.

11. The method according to claim 10 wherein the NSAID compound is a COX-2 selective inhibiting agent.

12. The method according to claim 11 wherein the COX-2 selective inhibiting agent is selected from:
a) a compound of the formula I 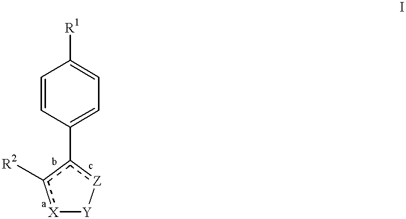
or a pharmaceutically acceptable salt thereof wherein: X-Y-Z-is selected from the group consisting of:
(a) —CH₂CH₂CH₂—,
(b) —C(O)CH₂CH₂—,
(c) —CH₂CH₂C(O)—,
(d) —CR⁵(R^{5′})-O—C(O)—,
(e) —C(O)—O—CR⁵(R^{5′})-,
(f) —CH₂—NR³-CH₂—,
(g) —CR⁵(R^{5′})-NR³-C(O)—,
(h) —CR⁴=CR^{4′}-S—,
(i) —S—CR⁴=CR^{4′}-,
(j) —S—N═CH—,
(k) —CH═N—S—,
(l) —N═CR⁴-O—,
(m) —O—CR⁴=N—
(n) —N═CR⁴-NH—,
(o) —N═CR⁴-S—, and
(p) —S—CR⁴=N—,
(q) —C(O)—NR³-CR⁵(R^{5′})-,
(r) —NR³-CH═CH— provided R¹ is other than —S(O)₂Me,
(s) —CH═CH—NR³- provided R¹ is other than —S(O)₂Me,when side b is a double bond, and sides a an c are single bonds; and X-Y-Z-is selected from the group consisting of:
(a) ═CH—O—CH═, and
(b) ═CH—NR³-CH═,
(c) ═N—S—CH═,
(d) ═CH—S—N═,
(e) ═N—O—CH═,
(f) ═CH—O—N═,
(g) ═N—S—N═,
(h) ═N—O—N═, when sides a and c are double bonds and side b is a single bond;R¹ is selected from the group consisting of
(a) S(O)₂CH₃,
(b) S(O)₂NH₂,
(c) S(O)₂NHC(O)CF₃,
(d) S(O)(NH)CH₃,
(e) S(O)(NH)NH₂,
(f) S(O)(NH)NHC(O)CF₃,
R² is selected from the group consisting of
(a) C₁₋₆alkyl,
(b) C₃, C₄, C₅, C₆, and C₇, cycloalkyl,
(c) mono-, di- or tri-substituted phenyl wherein the substituent is selected from the group consisting of
(1) hydrogen,
(2) halo,
(3) C₁₋₆alkoxy,
(4) C₁₋₆alkylthio,
(5) CN,
(6) CF₃,
(7) C₁₋₆alkyl,
(8) N₃,
(9) —CO₂H,
(10) —CO₂—C₁₋₄alkyl,
(11) —C(R⁵)(R⁶)-OH,
(12) —C(R⁵)(R⁶)-O—C₁₋₄alkyl, and
(13) —C₁₋₆alkyl-CO₂-R⁵;
(d) mono-, di- or tri-substituted heteroaryl wherein the heteroaryl is a monocyclic aromatic ring of 5 atoms, said ring having one hetero atom which is S, O, or N, and optionally 1, 2, or 3 additional N atoms; or the heteroaryl is a monocyclic ring of 6 atoms, said ring having one hetero atom which is N, and optionally 1, 2 or 3 additional N atoms; said substituents are selected from the group consisting of
(1) hydrogen,
(2) halo, including fluoro, chloro, bromo and iodo,
(3) C₁₋₆alkyl,
(4) C₁₋₆alkoxy,
(5) C₁₋₆alkylthio,
(6) CN,
(7) CF₃,
(8) N₃,
(9) —C(R⁵)(R⁶)-OH,
(10) —C(R⁵)(R⁶)-O—C₁₋₄alkyl;
R³ is selected from the group consisting of
(a) hydrogen,
(b) CF₃,
(c) CN,
(d) C₁₋₆alkyl,
(e) hydroxyC₁₋₆alkyl, and
(f) —C(O)—C₁₋₆alkyl,
(g) optionally substituted
(1) —C₁₋₅ alkyl-Q,
(2) —C₁₋₃alkyl-O—C₁₋₃alkyl-Q,
(3) —C₁₋₃alkyl-S—C₁₋₃alkyl-Q,
(4) —C₁₋₅ alkyl-O—Q, or
(5) —C₁₋₅ alkyl-S—Q,
wherein the substituent resides on the alkyl and the substituent is C₁₋₃alkyl;
(h) —Q
R⁴ and R^{4′} are each independently selected from the group consisting of
(a) hydrogen,
(b) CF₃,
(c) CN,
(d) C₁₋₆alkyl,
(e) —Q,
(f) —O—Q;
(g) —S—Q, and
(h) optionally substituted
(1) —C₁₋₅ alkyl-Q,
(2) —O—C₁₋₅ alkyl-Q,
(3) —S—C₁₋₅ alkyl-Q,
(4) —C₁₋₃alkyl-O—C₁₋₃alkyl-Q,
(5) —C₁₋₃alkyl-S—C₁₋₃alkyl-Q,
(6) —C₁₋₅ alkyl-O—Q,
(7) —C₁₋₅ alkyl-S—Q,
wherein the substituent resides on the alkyl and the substituent is C₁₋₃alkyl, and
R⁵, R^{5′} and R⁶, R⁷ and R⁸ are each independently selected from the group consisting of
(a) hydrogen,
(b) C₁₋₆alkyl,
or R⁵ and R⁶ or R⁷ and R⁸ together with the carbon to which they are attached form a monocyclic saturated carbon ring of 3, 4, 5, 6 or 7 atoms;
Q is CO₂H, CO₂-C₁₋₄alkyl, tetrazolyl-5-yl, C(R⁷)(R⁸)(OH), or C(R⁷)(R⁸)(O-C₁₋₄alkyl);
provided that when X-Y-Z is —S—CR⁴=CR^{4′}, then R⁴ and R^{4′} are other than CF₃;
b) a compound of the formula II 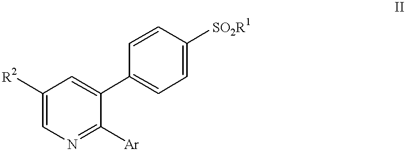
wherein:
R¹ is selected from the group consisting of
(a) CH₃,
(b) NH₂,
(c) NHC(O)CF₃,
(d) NHCH₃;
Ar is a mono-, di-, or trisubstituted phenyl or pyridinyl (or the N-oxide thereof), wherein the substituents are chosen from the group consisting of
(a) hydrogen,
(b) halo,
(c) C₁₋₆alkoxy,
(d) C₁₋₆alkylthio,
(e) CN,
(f) C₁₋₆alkyl,
(g) C₁₋₆fluoroalkyl,
(h) N₃,
(i) —CO₂R³,
(j) hydroxy,
(k) —C(R⁴)(R⁵)-OH,
(l) —C₁₋₆alkyl-CO₂-R⁶,
(m) C₁₋₆fluoroalkoxy;
R² is chosen from the group consisting of
(a) halo,
(b) C₁₋₆alkoxy,
(c) C₁₋₆alkylthio,
(d) CN,
(e) C₁₋₆alkyl,
(f) C₁₋₆fluoroalkyl,
(g) N₃,
(h) —CO₂R⁷,
(i) hydroxy,
(j) —C(R⁸)(R⁹)-OH,
(k) —C₁₋₆alkyl-CO₂-R¹⁰,
(l) C₁₋₆fluoroalkoxy,
(m) NO₂,
(n) NR¹¹R¹²,and
(o) NHCOR¹³,
R³, R⁴, R⁵, R⁶, R⁷, R⁸, R⁹, R¹⁰, R¹¹, R¹², R¹³, are each independantly chosen from the group consisting of
(a) hydrogen, and
(b) C₁₋₆alkyl,
or R⁴ and R⁵, R⁸ and R⁹ or R¹¹ and R¹² together with the atom to which they are attached form a saturated monocyclic ring of 3, 4, 5, 6 or 7 atoms;
c) a compound of the formula III: 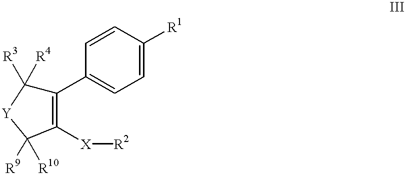
or a pharmaceutically acceptable salt thereofwherein:
X is selected from the group consisting of
(a) CH₂,
(b) CHOH,
(c) CO,
(d) O,
(e) S, and
(f) N(R¹⁵),
with the proviso that when R³ and R⁴ are other than
(1) both hydrogen,
(2) both C₁₋₁₀ alkyl, or
(3) joined together with the carbon to which they are attached form a saturated monocyclic carbon ring of 3, 4, 5, 6 or 7 atoms, then
X is selected from CO, O, S or N(R¹⁵);
Y is selected from the group consisting of
(a) C(R¹¹)(R¹²),
(b) CO,
(c) O, and
(d) S;
R¹ is selected from the group consisting of
(a) SO₂CH₃,
(b) SO₂NR¹⁶R¹⁷,
(c) SO₂NHC(O)CF₃,
(d) S(O)(NH)NH₂,
(e) S(O)(NH)NHC(O)CF₃,
(f) P(O)(CH₃)NH₂, and
(g) P(O)(CH₃)₂,
R² is selected from the group consisting of
(a) C₁₋₁₀alkyl,
(b) mono-, di- or tri-substituted phenyl or naphthyl wherein the substituents are selected from the group consisting of
(1) hydrogen,
(2) halo,
(3) C₁₋₁₀alkoxy,
(4) C₁₋₁₀alkylthio,
(5) CN,
(6) C₁₋₆ fluoroalkyl
(7) C₁₋₁₀ alkyl,
(8) N₃,
(9) —CO₂H,
(10) —CO₂—C₁₋₁₀alkyl,
(11) —C(R⁵)(R⁶)-OH,
(12) —C(R⁵)(R⁶)-O—C₁₋₄alkyl, and
(13) —C₁₋₆alkyl-CO₂-R⁵,
(14) benzyloxy,
(15) —O—(C₁₋₆alkyl)-CO₂R⁵, and
(16) —O—(C₁₋₆alkyl)-NR⁵R⁶,
(c) mono- , di- or tri-substituted heteroaryl wherein the heteroaryl is a monocyclic aromatic ring of 5 atoms, said ring having one hetero atom which is S, O, or N, and optionally 1, 2, or 3 additional N atoms; or the heteroaryl is a monocyclic ring of 6 atoms, said ring having one hetero atom which is N, and optionally 1, 2, or 3 additional N atoms, wherein the substituents are selected from the group consisting of
(1) hydrogen,
(2) halo,
(3) C₁₋₁₀alkyl,
(4) C₁₋₁₀alkoxy,
(5) C₁₋₁₀alkylthio,
(6) CN,
(7) CF₃,
(8) N₃,
(9) —C(R⁵)(R⁶)-OH,
(10) —C(R⁵)(R⁶)-O—C₁₋₁₀alkyl, and
(11) C₁₋₆fluoroalkyl;
(d) a mono- or di- substituted benzoheterocycle in which the heterocycle is a 5, 6, or 7-membered ring which may contain 1 or 2 heteroatoms chosen independently from O, S, or N and which may contain a carbonyl group or a sulfonyl group; wherein the substituents are selected from the group consisting of
(1) hydrogen,
(2) halo,
(3) C₁₋₁₀alkyl,
(4) C₁₋₁₀alkoxy,
(5) C₁₋₁₀alkylthio,
(6) CN,
(7) CF₃,
(8) N₃,
(9) —C(R⁵)(R⁶)-OH,
(10) —C(R⁵)(R⁶)-O—C₁₋₁₀alkyl, and
(11) C₁₋₆fluoroalkyl;
(e) a heterocycloalkyl group of 5, 6 or 7 members which contains 1 or 2 heteroatoms chosen from O, S, or N and optionally contains a carbonyl group or a sulfonyl group.
(f) a mono- or di- substituted benzocarbocycle in which the carbocycle is a 5, 6, or 7-membered ring which optionally contains a carbonyl group, wherein the substituents are selected from the group consisting of
(1) hydrogen,
(2) halo,
(3) C₁₋₁₀alkyl,
(4) C₁₋₁₀alkoxy,
(5) C₁₋₁₀alkylthio,
(6) CN,
(7) CF₃,
(8) N₃,
(9) —C(R⁵)(R⁶)-OH,
(10) —C(R⁵)(R⁶)-O—C₁₋₁₀alkyl, and
(11) C₁₋₆fluoroalkyl;
(g) a mono- or di-substituted bicyclic heteroaryl of 8, 9, or 10 members, containing 2 to 5 heteroatoms chosen independently from O, S or N, and in which each ring contains at least one heteroatom, wherein the substituents are selected from the group consisting of
(1) hydrogen,
(2) halo,
(3) C₁₋₁₀alkyl,
(4) C₁₋₁₀alkoxy,
(5) C₁₋₁₀alkylthio,
(6) CN,
(7) CF₃,
(8) N₃,
(9) —C(R⁵)(R⁶)-OH,
(10) —C(R⁵)(R⁶)-O—C₁₋₁₀alkyl, and
(11) C₁₋₆fluoroalkyl;
R³ is hydrogen, C₁₋₁₀ alkyl, CH₂OR⁷, CN, CH₂CN, C₁₋₆fluoroalkyl, F, CON(R⁷)₂, mono- or di-substituted phenyl, mono or di-substituted benzyl, mono- or di-substituted heteroaryl, mono or di-substituted heteroarylmethyl, wherein the substituents are selected from the group consisting of
(1) hydrogen,
(2) halo,
(3) C₁₋₆alkyl,
(4) C₁₋₆alkoxy,
(5) C₁₋₆alkylthio,
(6) CN,
(7) CF₃,
(8) N₃,
(9) —C(R⁵)(R⁶)-OH,
(10) —C(R⁵)(R⁶)-O—C₁₋₄alkyl, and
(11) C₁₋₆fluoroalkyl;
R⁴is
(a) hydrogen
(b) C₁₋₁₀alkyl,
(c) C₁₋₁₀alkoxy,
(d) C₁₋₁₀alkylthio,
(e) —OH,
(f) —OCOR⁷,
(g) —SH,
(h) —SCOR⁷,
(i) —OCO₂R⁸,
(j) ——SCO₂R⁸,
(k) OCON(R⁷)₂,
(l) SCON(R⁷)₂, and
(m) C₁₋₆fluoroalkyl;
or R³ andR⁴ together with the carbon to which they are attached form a saturated monocyclic carbon ring of 3, 4, 5, 6 or 7 atoms; R⁵ and R⁶ are each independently selected from the group consisting of
(a) hydrogen, and
(b) C₁₋₁₀alkyl, or R⁵ and R⁶ together with the atom to which they are attached form a saturated monocyclic ring of 3, 4, 5, 6 or 7 atoms; each R⁷ is independently selected from the group consisting of
(a) hydrogen,
(b) C₁₋₆alkyl,
(c) phenyl or monosubstituted phenyl wherein the substituents may be halo, C₁₋₆alkyl, C₁₋₆alkoxy, C₁₋₆alkylthio, CN, or CF₃, and
(d) benzyl or monosubstituted benzyl wherein the substituents may be halo, C₁₋₆alkyl, C₁₋₆alkoxy, C₁₋₆alkylthio, CN, or CF₃, or two R₇ groups taken together with the nitrogen to which they are attached form a saturated monocyclic ring of 5, 6 or 7 atoms, optionally containing an additional O, S or NR⁵;each R⁸ is independently selected from the group consisting of
(a) C₁₋₆alkyl,
(b) phenyl or monosubstituted phenyl wherein the substituents may be halo, C₁₋₆alkyl, C₁₋₆alkoxy, C₁₋₆alkylthio, CN, or CF₃, and
(c) benzyl or monosubstituted benzyl wherein the substituents may be halo, C₁₋₆alkyl, C₁₋₆alkoxy, C₁₋₆alkylthio, CN, or CF₃;
R⁹ and R¹⁰ are independently selected from the group consisting of:
(a) hydrogen, and
(b) C₁₋₇alkyl, or
R⁹ and R¹⁰ together with the carbon atom to which they are attached form a carbonyl or thiocarbonyl group; R¹¹ and R¹² are independently
(a) hydrogen,
(b) mono- or di-substituted phenyl or mono- or di-substituted benzyl or mono- or di-substituted heteroaryl or mono- or di-substituted heteroarylmethyl, wherein the substituents are selected from the group consisting of
(1) hydrogen,
(2) fluoro, chloro, bromo and iodo,
(3) C₁₋₆alkyl,
(4) C₁₋₆alkoxy,
(5) C₁₋₆alkylthio,
(6) CN,
(7) CF₃,
(8) N₃,
(9) —C(R¹³)(R¹⁴)-OH,
(10) —C(R¹³)(RI⁴)-O—C₁₋₄alkyl, and
(11) C₁₋₆fluoroalkyl, or
(c) C₁₋₇alkyl, CH₂OR⁷, CN, CH₂CN, C₁₋₆fluoroalkyl, CON(R⁷)₂, F, or OR⁷; or
R¹¹ and R¹² together with the carbon to which they are attached form a saturated monocyclic carbon ring of 3, 4, 5, 6 or 7 atoms; R¹³ and R¹⁴ are independently selected from the group consisting of:
(a) hydrogen,
(b) C₁₋₇alkyl, or
R¹³ and R¹⁴ together with the carbon to which they are attached form a carbonyl, —C(═S)—, or a saturated monocyclic carbon ring of 3, 4, 5, 6, or 7 atoms. R¹⁵ is selected from the group consisting of:
(a) hydrogen,
(b) C₁₋₁₀alkyl,
(c) mono-, di- or tri-substituted phenyl or naphthyl wherein the substituents are selected from the group consisting of
(1) hydrogen,
(2) halo,
(3) C₁₋₁₀alkoxy,
(4) C₁₋₁₀alkylthio,
(5) CN,
(6) C₁₋₆ fluoroalkyl
(7) C₁₋₁₀alkyl,
(8) N₃,
(9) —CO₂H,
(10) —CO₂—C₁₋₁₀alkyl,
(11) —C(R⁵)(R⁶)-OH,
(12) —C(R⁵)(R⁶)-O—C₁₋₄alkyl, and
(13) —C₁₋₆alkyl-CO₂-R⁵;
(14) benzyloxy,
(15) —O—(C₁₋₆alkyl)-CO₂R⁵, and
(16) —O—(C₁₋₆alkyl)-NR⁵R⁶,
(d) mono-, di- or tri-substituted heteroaryl wherein the heteroaryl is a monocyclic aromatic ring of 5 atoms, said ring having one hetero atom which is S, O, or N, and optionally 1, 2, or 3 additional N atoms; or the heteroaryl is a monocyclic ring of 6 atoms, said ring having one hetero atom which is N, and optionally 1, 2, or 3 additional N atoms, wherein the substituents are selected from the group consisting of
(1) hydrogen,
(2) halo,
(3) C₁₋₁₀alkyl,
(4) C₁₋₁₀alkoxy,
(5) C₁₋₁₀alkylthio,
(6) CN,
(7) CF₃,
(8) N₃,
(9) —C(R⁵)(R⁶)-OH,
(10) —C(R⁵)(R⁶)-O—C₁₋₁₀alkyl, and
(11) C₁₋₆fluoroalkyl;
(e) a mono- or di- substituted benzoheterocycle in which the heterocycle is a 5, 6, or 7-membered ring which may contain 1 or 2 heteroatoms chosen independently from O, S, or N and which may contain a carbonyl group or a sulfonyl group; wherein the substituents are selected from the group consisting of
(1) hydrogen,
(2) halo,
(3) C₁₋₁₀alkyl,
(4) C₁₋₁₀alkoxy,
(5) C₁₋₁₀alkylthio,
(6) CN,
(7) CF₃,
(8) N₃,
(9) —C(R⁵)(R⁶)-OH,
(10) —C(R⁵)(R⁶)-O—C₁₋₁₀alkyl, and
(11) C₁₋₆fluoroalkyl;
(f) a heterocycloalkyl group of 5, 6 or 7 members which contains 1 or 2 heteroatoms chosen from O, S, or N and optionally contains a carbonyl group or a sulfonyl group.
(g) a mono- or di- substituted benzocarbocycle in which the carbocycle is a 5, 6, or 7-membered ring which optionally contains a carbonyl group, wherein the substituents are selected from the group consisting of
(1) halo,
(3) C₁₋₁₀alkyl,
(4) C₁₋₁₀alkoxy,
(5) C₁₋₁₀alkylthio,
(6) CN,
(7) CF₃,
(8) N₃,
(9) —C(R⁵)(R⁶)-OH,
(10) —C(R⁵)(R⁶)-O—C₁₋₄alkyl, and
(11) C₁₋₆fluoroalkyl;
R¹⁶ and R¹⁷ are independently selected from the group consisting of
(a) hydrogen
(b) C₁₋₁₀alkyl,
(c) C₁₋₁₀alkanoic acid,
(d) C₁₋₁₀alkyl amine,
(e) phenyl or monosubstituted phenyl wherein the substituents are halo, C₁₋₁₀alkyl, C₁₋₁₀alkoxy, C₁₋₁₀alkylthio, C₁₋₁₀alkanoic acid, C₁₋₁₀alkylamine, CN, CO₂H or CF₃, and
(f) benzyl or monosubstituted benzyl wherein the substituents are halo, C₁₋₁₀alkyl, C₁₋₁₀alkoxy, C₁₋₁₀alkylthio, C₁₋₁₀alkanoic acid, C₁₋₁₀alkylamine, CN, COOH or CF₃, orR16 and R17 together with the nitrogen to which they are attached form a saturated monocyclic ring of 5, 6 or 7 atoms, optionally containing an additional O, S or NR⁵.

13. The method according to claim 11 wherein the COX-2 selective inhibiting agent is selected from:
3-(3-Fluorophenyl)-4-(4-(methylsulfonyl)phenyl)-2-(5H)-furanone,
3-(3,4-Difluorophenyl)-4-(4-(methylsulfonyl)phenyl)-2-(5H)-furanone,
3-(3,4-Dichlorophenyl)-4-(4-(methylsulfonyl)phenyl)-2-(5H)-furanone,
3-phenyl-4-(4-(methylsulfonyl)phenyl)-2-(5H)-furanone and
5,5-Dimethyl-3-(3-fluorophenyl)-4-(methylsulfonyl)phenyl)-2-(5H)-furanone
3-(4-Methylsulfonyl)phenyl-2-phenyl-5-trifluoromethylpyridine;
2-(3-Chlorophenyl)-3-(4-methylsulfonyl)phenyl-5-trifluoromethyl-pyridine;
2-(4-Chlorophenyl)-3-(4-methylsulfonyl)phenyl-5-trifluoromethyl-pyridine;
2-(4-Fluorophenyl)-3-(4-methylsulfonyl)phenyl-5-trifluoromethyl-pyridine;
3-(4-Methylsulfonyl)phenyl-2-(3-pyridinyl)-5-trifluoromethylpyridine;
5-Methyl-3-(4-methylsulfonyl)phenyl-2-phenylpyridine;
2-(4-Chlorophenyl)-5-methyl-3-(4-methylsulfonyl) phenylpyridine;
5-Methyl-3-(4-methylsulfonyl)phenyl-2-(3-pyridinyl) pyridine;
5-Chloro-2-(4-chlorophenyl)-3-(4-methylsulfonyl) phenylpyridine;
5-Chloro-3-(4-methylsulfonyl)phenyl-2-(2-pyridinyl) pyridine;
5-Chloro-3-(4-methylsulfonyl)phenyl-2-(3-pyridinyl) pyridine;
5-Chloro-3-(4-methylsulfonyl)phenyl-2-(4-pyridinyl) pyridine;
5-Chloro-3-(4-methylsulfonyl)phenyl-2-(2-methyl-5-pyridinyl)pyridine;
2-(4-Chlorophenyl)-3-(4-methylsulfonyl)phenylpyridinyl-5-carboxylic acid methyl ester;
2-(4-Chlorophenyl)-3-(4-methylsulfonyl)phenylpyridinyl-5-carboxylic acid;
5-Cyano-2-(4-chlorophenyl)-3-(4-methylsulfonyl) phenylpyridine;
5-Chloro-3-(4-methylsulfonyl)phenyl-2-(3-pyridyl)pyridine hydromethanesulfonate;
5-Chloro-3-(4-methylsulfonyl)phenyl-2-(3-pyridyl)pyridine hydrochloride;
5-Chloro-3-(4-methylsulfonyl)phenyl-2-(2-methyl-5-pyridinyl)pyridine Hydrochloride;
5-Chloro-3-(4-methylsulfonyl)phenyl-2-(2-ethyl-5-pyridinyl)pylidine; and
5-Chloro-3-(4-methylsulfonyl)phenyl-2-(2-ethyl-5-pyridinyl)pyridine hydromethanesulfonate.
3-(3,4-Difluorophenoxy)-5,5-dimethyl-4-(4-(methylsulfonyl)phenyl)-5H-furan-2-one,
3-(3-Fluorophenoxy)-5,5-dimethyl-4-(4-(methylsulfonyl)phenyl)- 5H-furan-2-one,
3-(3,5-Difluorophenoxy)-5,5-dimethyl-4-(methylsulfonyl) phenyl)- 5H-furan-2-one,
3-Phenoxy-5,5-dimethyl-4-(4-(methylsulfonyl)phenyl)-5H-furan-2-one,(5) 3-(2,4-Difluorophenoxy)-5,5-dimethyl-4-(4-(methylsulfonyl) phenyl)-5H-furan-2-one,
3-(4-Chlorophenoxy)-5,5-dimethyl-4-(4-(methylsulfonyl) phenyl)- 5H-furan-2-one,
3-(3,4-Dichlorophenoxy)-5,5-dimethyl-4-(methylsulfonyl) phenyl)-5H-furan-2-one,
3-(4-Fluorophenoxy)-5,5-dimethyl-4-(4-(methylsulfonyl) phenyl)-5H-furan-2-one,
3-(4-Fluorophenyti)-5,5-dimethyl-4-(4-(methylsulfonyl)phenyl)-5H-furan-2-one,
3-(3,5-Difluorophenylthio)-5,5-dimethyl-4-(4-(methylsulfonyl)phenyl)-5H-furan-2-one,
3-Phenylthio-5,5-dimethyl-4-(4-(methylsulfonyl)phenyl)-5H-furan-2-one,
3-(N-Phenylamnino)-5,5-dimethyl-4-(4-(methylsulfonyl) phenyl)-5H-furan-2-one,
3-Cyclohexyloxy-5,5-dimethyl-4-(4-(methylsulfonyl)phenyl)-5H-furan-2-one,
3-Phenylthio-4-(4-(methylsulfonyl)phenyl)-5H-furan-2-one,
3-Benzyl-5,5-dimethyy-4-(4-(methylsulfonyl)phenyl)-5H-furan-2-one,
3-(3,4-Difluorophenylhydroxymethyl)-5,5-dimethyl-4-(4-(methylsulfonyl)phenyl)-5H-furan-2-one,
3-(3,4-Difluorobenzoyl)-5,5-dimethyl-4-(4-(methylsulfonyl)phenyl)-5H-furan-2-one,
3-Benzoyl-5,5-dimethyl-4-(4-(methylsulfonyl)phenyl)-5H-furan-2-one,
4-(4-(Methylsulfonyl)phenyl)-3-phenoxy-1-oxaspiro[4.4]non-3-en-2-one,
4-(4-(Methylsulfonyl)phenyl)-3-phenylthio-1-oxaspiro[4.4]non-3-en-2-one,
4-(2-Oxo-3-phenylthio-1-oxa-spiro[4,4]non-3-en-4-yl) benzenesulfonamide,
3-(4-Fluorobenzyl)-5,5-dimethyl-4-(4-(methylsulfonyl) phenyl)-5H-furan-2-one,
3-(3,4-Difluorophenoxy)-5-methoxy-5-methyl-4-(4-(methylsulfonyl)phenyl)-5H-furan-2-one,
3-(5-Chloro-2-pyridyloxy)-5,5-dimethyl-4-(4-(methylsulfonyl)phenyl)-5H-furan-2-one,
3-(2-pyridyloxy)-5,5-dimethyl-4-(4-(methylsulfonyl)phenyl)-5H-furan-2-one,
3-(6-Methyl-2-pyridyloxy)-5,5-dimethyl-4-(4-(methylsulfonyl) phenyl)-5H-furan-2-one,
3-(3-Isoquinolinoxy)-5,5-dimethyl-4-(4-(methylsulfonyl)phenyl)-5H-furan-2-one,
3-(4-(Methylsulfonyl)phenyl)-2-phenoxycyclopent-2-enone, and
3-(4-(Methylsulfonyl)phenyl)-2-(3,4-difluorophenoxy) cyclopent-2-enone.
(a) 5,5-Dimethyl-4-(4-methylsulfonylphenyl)-3-(5-bromopyridin-2-yloxy)-5H-furan-2-one, and
(b) 5,5-Dimethyl-4-(4-methylsulfonylphenyl)-3-(2-propoxy)-5H-furan-2-one, or
2-(3,4-difluorophenoxy)-3-(4-methylsulfonylphenyl)-cyclopent-2-enone,
3-(5-Benzothiophenyloxy)-5,5-dimethyl-4-(4-(methylsulfonyl) phenyl)-5H-furan-2-one,
5,5-dimethyl-4-(4-methylsulfonyl-phenyl)-3-(pyridyl-4-oxy)-5H-furan-2-one,
5,5-dimethyl-4-(4-methylsulfonyl-phenyl)-3-(pyridyl-3-oxy)-5H-furan-2-one,
3-(2-Methyl-5-pyridyloxy)-5,5-dimethyl-4-(4-(methylsulfonyl) phenyl)-5H-furan-2-one,
3(2-Fluoro-4-trifluoromethyl)phenoxy-4-(4-methylsulfonyl)phenyl)-5,5-dimethyl-5H-furan-2-one,
3-(5-Chloro-2-pyridylthio)-5,5-dimethyl-4-(4-methylsulfonyl)phenyl-5H-furan-2-one,
2-(3,5-Difluorophenoxy)-3-(4-methylsulfonylphenyl)-cyclopent-2-enone,
3-(2-Pyrimidinoxy)-5,5-dimethyl-4-(4-methylsulfonyl)phenyl-5H-furan-2-one,
3-(3-Methyl-2-pyridyloxy)-5,5-dimethyl-4-(4-methylsulfonyl)phenyl-5H-furan-2-one,
3-(3-Chloro-5-pyridyloxy)-5,5-dimethyl-4-(4-(methylsulfonyl) phenyl)-5H-furan-2-one,
3-(3-(1,2,5-Thiadiazolyl)oxy)-4-(4-(methylsulfonyl)phenyl)-5,5-dimethyl-5H-furan-2-one,
3-(5-Isoquinolinoxy)-5,5-dimethyl-4-(4-methylsulfonyl)phenyl-5H-furan-2-one,
3-(6-Amino-2-pyridyloxy)-5,5-dimethyl-4-(4-(methylsulfonyl) phenyl)-5H-furan-2-one,
3-(3-Chloro-4-fluoro)phenoxy-4-(methylsulfonyl)phenyl)-5 ,5-dimethyl-5H-furan-2-one,
3-(6-Quinolinoxy)-5,5-dimethyl-4-(4-(methylsulfonyl)phenyl)-5H-furan-2-one,
3-(5-Nitro-2-pyridyloxy)-5,5-dimethyl-4-(4-methylsulfonyl)phenyl-5H-furan-2-one,
3-(2-Thiazolylthio)-5,5-dimethyl-4-(4-(methylsulfonyl)phenyl)-5H-furan-2-one,
3-(3-Chloro-5-pyridyloxy)-5,5-dimethyl-4-(4-(methylsulfonyl) phenyl)-5H-furan-2-one,
5,5-Dimethyl-4-(4-methylsulfonylphenyl)-3-(2-propoxy)-5H-furan-2-one,
3-(3-Trifluoromethyl)phenoxy-4-(4-methylsulfonyl)phenyl)-5,5-dimethyl-5H-furan-2-one,
5,5-Dimethyl-(4-(4-methylsulfonyl)phenyl)-3-(piperidine- 1 -carbonyl)-5-H-furan-2-one,
5,5-Dimethyl-3-(2-Butoxy)-4-(4-methylsulfonylphenyl)-5H-furan-2-one,
5,5-Dimethyl-4-(4-methylsulfonylphenyl)-3-(3-pentoxy)-5H-furan-2-one,
2-(5-Chloro-2-pyridyloxy)-3-(4-methylsulfonyl)phenylcyclopent-2-enone,
3-(4-Methyl-2-pyridyloxy)-5,5-dimethyl-4-(4-methylsulfonyl)phenyl-5H-furan-2-one,
(5R)-3-(3,4-Difluorophenoxy)-5-ethyl-5-methyl-4-(4-methylsulfonyl)phenyl-5H-furan-2-one,
(5R)-3-(4-Chlorophenoxy)-5-ethyl-5-methyl-4-(4-methylsulfonyl)phenyl-5H-furan-2-one,
3-(2-Methyl-3-pyridyloxy)-5,5-dimethyl-4-(4-methylsulfonyl)phenyl-5H-furan-2-one,
(30) 3-(4-Methyl-5-nitro-2-pyridyloxy)-5 ,5-dimethyl-4-(4-methylsulfonyl)phenyl-5H-furan-2-one,
3-(5-Chloro-4-methyl-2-pyridyloxy)-5,5-dimethyl-4-(4-methylsulfonyl)phenyl-5H-furan-2-one,
3-(5-Fluoro-4-methyl-2-pyridyloxy)-5,5-dimethyl-4-(4-methylsulfonyl)phenyl-5H-furan-2-one,
3-(3 -Chloro-2-pyridyloxy)-5,5-dimethyl-4-(4-methylsulfonyl)phenyl-5H-furan-2-one,
3-(4-Fluorophenoxy)-5-methyl-4-(4-methylsulfonyl)phenyl-5-propyl-5H-furan-2-one,
3- (N,N-Diethylamino)-5,5-dimethyl-4-(4-(methylsulfonyl) phenyl)-5H-furan-2-one,
5,5-dimethyl-4-(4-methylsulfonyl-phenyl)-3-(3,5-dichloro-2-pyridyloxy)-5H-furan-2-one,
(5R)-3-(4-Bromophenoxy)-5-ethyl-5-methyl4-(4-methylsulfonyl)phenyl-5H-furan-2-one,
(5R)-3-(4-Methoxyphenoxy)-5-ethyl-5-methyl-4-(4-methylsulfonyl)phenyl-5H-furan-2-one,
(5R)-3-(5-Chloro-2-pyridyloxy)-5-methyl-4-(4-methylsulfonyl)phenyl-5-(2,2,2-trifluoroethyl)-5H-furan-2-one,
3-(5-Chloro-2-pyridyloxy)-5-methyl-4-(4-methylsulfonyl)phenyl-5-propyl-5H-furan-2-one,
3-(1-Cyclopropyl-ethoxy)-5,5-dimethyl-4-(4-methyl sulfonyl)phenyl)-5H-furan-2-one,
5-Methyl-4-(4-(methylsulfonyl)phenyl)-3-(2-(propoxy)-5-(2-trifluoroethyl)-5H-furan-2-one,
5(R)-5-ethyl-5-methyl-4-(4-(methylsulfonyl)phenyl)-3-(2-propoxy)-5H-furan-2-one,
5,5-dimethyl-3-(2,2-dimethylpropyloxy)-4-(4-(methylsulfonyl)phenyl)-5H-furan-2-one,
5(R) 3-(1-cyclopropyl-ethoxy)-5-ethyl-5-methyl-4-(4-(methyl sulfonyl)phenyl-5H-furan-2-one,
5(S) 5-Ethyl-5-methyl-4-(4-(methylsulfonyl)phenyl-3-(2-propoxy)-5H-furan-2-one,
3-(1-cyclopropylethoxy)-5,5-dimethyl-4-(4-(methylsulfonyl)phenyl)-5H-furan-2-one,
3-(1-cyclopropylethoxy)-5,5-dimethyl-4-(4-(methylsulfonyl)phenyl)-5H-furan-2-one,
5,5-dimethyl-3-(isobutoxy)-4-(4-(methylsulfonyl)phenyl)-5H-furan-2-one,
3-(4-Bromophenoxy)-5,5-dimethyl-4-(4-methylsulfonyl)phenyl-5h-furan-2-one,
3-(2-Quinolinoxy)-5,5-dimethyl-4-(4-(methylsulfonyl)phenyl)-5H-furan-2-one,
3-(2-Chloro-5-pyridyloxy)-5,5-dimethyl-4-(4-(methylsulfonyl)phenyl)-5H-furan-2-one,
3-(6-benzothiazolyloxy)-5,5-dimethyl-4-(4-(methylsulfonyl) phenyl)-5H-furan-2-one,
3-(6-Chloro-2-pyridyloxy)-5,5-dimethyl-4-(4-(methylsulfonyl) phenyl)-5H-furan-2-one,
3-(4-Quinazolyloxy)-5,5-dimethyl-4-(4-(methylsulfonyl)phenyl)-5H-furan-2-one,
(5R)-3-(5-Fluoro-2-pyridyloxy)-5-ethyl-5 -methyl-4-(4-methylsulfonyl)phenyl-5H-furan-2-one,
(5R)-3-(4-Fluorophenoxy)-5-ethyl-5-methyl-4-(4-methyl sulfonyl)phenyl-5H-furan-2-one,
(5R)-3-(5-Fluoro-2-pyridyloxy)-5-methyl-4-(4-methylsulfonyl)phenyl-5-(2,2,2-trifluoroethyl)-5H-furan-2-one,
3-(1-Isoquinolinyloxy)-5,5-dimethyl-4-(methylsulfonyl)phenyl-5H-furan-2-one,
(5R)-3-(4-fluorophenoxy)-5-methyl-4-(4-methylsulfonyl)phenyl-5-(2,2,2-trifluoroethyl)-5H-furan-2-one,
3-(3-Fluoro-2-pyridyloxy)-5,5-dimethyl-4-(4-methylsulfonyl) phenyl-5H-furan-2-one,
(5R)-3-(3,4-difluorophenoxy)-5-methyl-4-(4-methylsulfonyl) phenyl-5-(2,2,2-trifluoroethyl)-5H-furan-2-one,
(5R)-3-(5-chloro-2-pyridyloxy)-5-ethyl-5-methyl-4-(4-methylsulfonyl)phenyl-5H-furan-2-one,
3-(3,4-difluorophenoxy)-5-methyl-5-trifluoromethyl-4-(4-methylsulfonyl)phenyl-5H-furan-2-one,
3-(3,4-Difluorophenoxy)-5-methyl-4-(4-(methylsulfonyl)phenyl)-5-propyl-5H-furan-2-one,
3-Cyclobutyloxy-5,5-dimethyl-4-(4-methylsulfonylphenyl-5H-furan-2-one,
3-(1-Indanyloxy)-5,5-dimethyl-4-(4-(methylsulfonyl)phenyl)-5H-furan-2-one,
3-(2-Indanyloxy)-5,5-dimethyl-4-(4-methylsulfonyl)phenyl)-5H-furan-2-one,
3-Cyclopentyloxy-5,5-dimethyl-4-(4-methylsulfonylphenyl)5H-furan-2-one,
3-(3,3-Dimethylcyclopentyloxy)-5,5-dimethyl-4-(4-methylsulfonyl-phenyl)-5H-furan-2-one,
3-Isopropoxy-5-methyl-4-(4-methylsulfonylphenyl)-5-propyl-5H-furan-2-one,
3-(2-Methoxy-5-pyridyloxy)-5,5-dimethyl-4-(4-methylsulfonyl)phenyl-5H-furan-2-one,
3-(5-Methyl-2-pyridyloxy)-5,5-dimethyl-4-(4-methylsulfonyl)phenyl-5H-furan-2-one,
(5RS)-3-(3,4-Difluorophenoxy)-5-methyl-4-(4-methylsulfonyl)phenyl-5-(2,2,2-trifluoroethyl)-5H-furan-2-one,
3 -(3-Chloro-4-methoxyphenoxy)-5,5-dimethyl -4-(4-methylsulfonyl)phenyl-5H-furan-2-one,
(5R)-3-(3-Chloro-4-methoxyphenoxy)-5-ethyl-5-methyl-4-(4-methylsulfonyl)phenyl-5H-furan-2-one,
(5R)-3-(4-Chlorophenoxy)-5-trifluoroethyl-5-methyl-4-(4-methylsulfonyl)phenyl-5H-furan-2-one,
(5R)-3-(4-Bromophenoxy)-5-trifluoroethyl-5-methyl-4-(4-methylsulfonyl)phenyl-5H-furan-2-one,
5-Cyclopropylmethyl-3-(3,4-difluorophenoxy)-5-methyl-(4-methylsulfonyl)phenyl-5H-furan-2-one,
(5R) -3-(3-Fluorophenoxy)-5-ethyl-5-methyl-4-(4-methylsulfonyl)phenyl-5H-furan-2-one,
(5R)-3-(4-Chloro-3-fluorophenoxy)-5-ethyl-5 -methyl-4-(4-methylsulfonyl)phenyl-5H-furan-2-one,
(5R)-3-Phenoxy-5-ethyl-5-methyl-4-(4-methylsulfonyl)phenyl-5H-furan-2-one,
(5R)-3-(4-Chloro-3-methylphenoxy)-5-ethyl-5-methyl-4-(4-methylsulfonyl)phenyl-5H-furan-2-one,
3-(4-Chloro-3-methylphenoxy)-5-5-dimethyl-4-(4-methylsulfonyl)phenyl-5H-furan-2-one,
(5R)-3-(5-bromo-2-pyridyloxy)-4-(4-methylsulfonylphenyl)-5-methyl-5-(2,2,2-trifluoroethyl)-5H-furan-2-one,
(5R)-3-(5-bromo-2-pyridyloxy)-4-(4-methylsulfonylphenyl)-5-ethyl-5-methyl-5H-furan-2-one,
3-(5-chloro-6-methyl-2-pyridyloxy)-5,5-dimethyl-4-(4-methylsulfonyl)phenyl-5H-furan-2-one,
3-(5-cyclopropyl-2-pyridyloxy)-5,5-dimethyl-4-(4-methylsulfonyl)phenyl-5H-furan-2-one,
3-(1-cyclopropylethoxy)-4-(4-methylsulfonyl)phenyl-5H-furan-2-one, and
3-(cyclopropylmethoxy)-4-(4-methylsulfonyl)phenyl-5H-furan-2-one.
or a pharmaceutically acceptable salt or optical isomer thereof.

14. The method according to claim 11 wherein the COX-2 selective inhibiting agent is selected from: 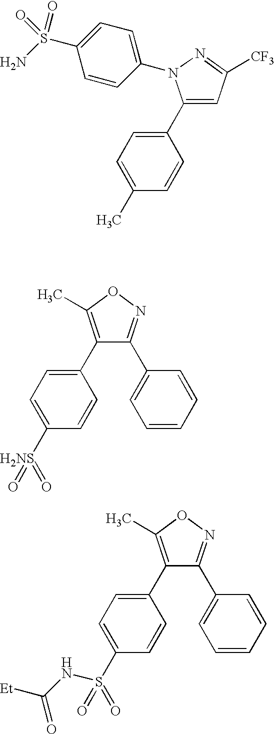
or a pharmaceutically acceptable salt thereof.

15. The method according to claim 12 wherein the COX-2 selective inhibiting agent is selected from:
3-phenyl-4-(4-(methylsulfonyl)phenyl)-2-(5H)-furanone 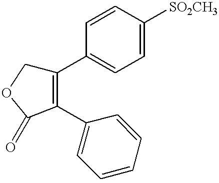
5-Chloro-3-(4-methylsulfonyl)phenyl-2-(2-methyl-5-pyridinyl)pyridine; 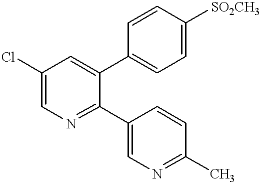
or a pharmaceutically acceptable salt thereof.

16. A pharmaceutical composition for achieving a therapeutic effect in a mammal in need thereof which comprises amounts of at least one NSAID compound and at least one PSA conjugate.

17. The pharmaceutical composition according to claim 16 comprising an amount of an NSAID and an amount of a PSA conjugate.

18. The pharmaceutical composition according to claim 16 wherein the therapeutic effect is selected from inhibition of cancerous tumor growth and the regression of cancerous tumors.

19. The pharmaceutical composition according to claim 18 wherein the cancerous tumor is a cancer related to cells that express enzymatically active PSA.

20. The pharmaceutical composition according to claim 19 wherein the cancer is prostate cancer.

21. A method of preparing a pharmaceutical composition for treatment of cancer in a mammal in need thereof which comprises mixing amounts of at least one NSAID compound and at least one PSA conjugate.

22. The method of preparing a pharmaceutical composition according to claim 21 comprising mixing an amount of an NSAID compound and an amount of an PSA conjugate.

23. A method of treating cancer in a mammal in need thereof which comprises administering to said mammal amounts of at least one NSAID compound and at least one PSA conjugate and applying to the mammal radiation therapy.

24. The method according to claim 23 wherein an amount of an NSAID compound and an amount of a PSA conjugate are administered simultaneously.

25. The method according to claim 23 wherein an amount of an NSAID compound and an amount of a PSA conjugate are administered consecutively.

26. A method for treating prostatic disease in a mammal in need thereof which comprises administering to said mammal amounts of at least one NSAID and at least one PSA conjugate.

27. The method according to claim 26 wherein the prostatic disease is selected from benign prostatic hyperplasia, prostatic intraepithelial meoplasia and prostate cancer.


SEQUENCE LISTING (54 sequences)

SEQ ID NO: 1 - protein, 7 aa, Artificial Sequence
  completely synthetic amino acid sequence
NKISYQS

SEQ ID NO: 2 - protein, 8 aa, Artificial Sequence
  completely synthetic amino acid sequence
NKISYQSS

SEQ ID NO: 3 - protein, 9 aa, Artificial Sequence
  completely synthetic amino acid sequence
NKISYQSSS

SEQ ID NO: 4 - protein, 10 aa, Artificial Sequence
  completely synthetic amino acid sequence
NKISYQSSST

SEQ ID NO: 5 - protein, 11 aa, Artificial Sequence
  completely synthetic amino acid sequence
NKISYQSSSTE

SEQ ID NO: 6 - protein, 12 aa, Artificial Sequence
  completely synthetic amino acid sequence
ANKISYQSSSTE

SEQ ID NO: 7 - protein, 11 aa, Artificial Sequence
  completely synthetic amino acid sequence
ANKISYQSSST

SEQ ID NO: 8 - protein, 12 aa, Artificial Sequence
  completely synthetic amino acid sequence
ANKISYQSSSTL

SEQ ID NO: 9 - protein, 12 aa, Artificial Sequence
  completely synthetic amino acid sequence
ANKASYQSASTL

SEQ ID NO: 10 - protein, 11 aa, Artificial Sequence
  completely synthetic amino acid sequence
ANKASYQSASL

SEQ ID NO: 11 - protein, 11 aa, Artificial Sequence
  completely synthetic amino acid sequence
ANKASYQSSSL

SEQ ID NO: 12 - protein, 10 aa, Artificial Sequence
  completely synthetic amino acid sequence
ANKASYQSSL

SEQ ID NO: 13 - protein, 7 aa, Artificial Sequence
  completely synthetic amino acid sequence
SYQSSSL

SEQ ID NO: 14 - protein, 7 aa, Artificial Sequence
  completely synthetic amino acid sequence
RYQSSSL

SEQ ID NO: 15 - protein, 7 aa, Artificial Sequence
  completely synthetic amino acid sequence
KYQSSSL

SEQ ID NO: 16 - protein, 6 aa, Artificial Sequence
  completely synthetic amino acid sequence
KYQSSL

SEQ ID NO: 17 - protein, 7 aa, Artificial Sequence
  completely synthetic amino acid sequence
KYQSSSL

SEQ ID NO: 18 - protein, 11 aa, Artificial Sequence
  completely synthetic amino acid sequence
LNKASYQSSSL

SEQ ID NO: 19 - protein, 7 aa, Artificial Sequence
  completely synthetic amino acid sequence
XSSXQSL

SEQ ID NO: 20 - protein, 6 aa, Artificial Sequence
  completely synthetic amino acid sequence
XSXQSL

SEQ ID NO: 21 - protein, 7 aa, Artificial Sequence
  completely synthetic amino acid sequence
XSSXQSL

SEQ ID NO: 22 - protein, 7 aa, Artificial Sequence
  completely synthetic amino acid sequence
XASXQSL

SEQ ID NO: 23 - protein, 7 aa, Artificial Sequence
  completely synthetic amino acid sequence
XASXQSL

SEQ ID NO: 24 - protein, 7 aa, Artificial Sequence
  completely synthetic amino acid sequence
PASXQSL

SEQ ID NO: 25 - protein, 7 aa, Artificial Sequence
  completely synthetic amino acid sequence
XASXQSL

SEQ ID NO: 26 - protein, 7 aa, Artificial Sequence
  completely synthetic amino acid sequence
XASXQSL

SEQ ID NO: 27 - protein, 7 aa, Artificial Sequence
  completely synthetic amino acid sequence
XASXQSL

SEQ ID NO: 28 - protein, 7 aa, Artificial Sequence
  completely synthetic amino acid sequence
XASXQSX

SEQ ID NO: 29 - protein, 7 aa, Artificial Sequence
  completely synthetic amino acid sequence
XASXQSL

SEQ ID NO: 30 - protein, 7 aa, Artificial Sequence
  completely synthetic amino acid sequence
XASXQSV

SEQ ID NO: 31 - protein, 7 aa, Artificial Sequence
  completely synthetic amino acid sequence
PASXQSL

SEQ ID NO: 32 - protein, 7 aa, Artificial Sequence
  completely synthetic amino acid sequence
SSSXQSV

SEQ ID NO: 33 - protein, 7 aa, Artificial Sequence
  completely synthetic amino acid sequence
XSSXQSV

SEQ ID NO: 34 - protein, 7 aa, Artificial Sequence
  completely synthetic amino acid sequence
SSSXQSL

SEQ ID NO: 35 - protein, 7 aa, Artificial Sequence
  completely synthetic amino acid sequence
XSSXQSL

SEQ ID NO: 36 - protein, 8 aa, Artificial Sequence
  completely synthetic amino acid sequence
XSSXQSSP

SEQ ID NO: 37 - protein, 7 aa, Artificial Sequence
  completely synthetic amino acid sequence
XSSXQSG

SEQ ID NO: 38 - protein, 8 aa, Artificial Sequence
  completely synthetic amino acid sequence
XSSXQSSG

SEQ ID NO: 39 - protein, 8 aa, Artificial Sequence
  completely synthetic amino acid sequence
XSSXQSSP

SEQ ID NO: 40 - protein, 7 aa, Artificial Sequence
  completely synthetic amino acid sequence
XSSXQSV

SEQ ID NO: 41 - protein, 8 aa, Artificial Sequence
  completely synthetic amino acid sequence
XSSXQSSP

SEQ ID NO: 42 - protein, 7 aa, Artificial Sequence
  completely synthetic amino acid sequence
XSSXQSP

SEQ ID NO: 43 - protein, 7 aa, Artificial Sequence
  completely synthetic amino acid sequence
XSSXQSP

SEQ ID NO: 44 - protein, 8 aa, Artificial Sequence
  completely synthetic amino acid sequence
XSSXQSSP

SEQ ID NO: 45 - protein, 7 aa, Artificial Sequence
  completely synthetic amino acid sequence
XSSXQSV

SEQ ID NO: 46 - protein, 7 aa, Artificial Sequence
  completely synthetic amino acid sequence
XSSXQSL

SEQ ID NO: 47 - protein, 5 aa, Artificial Sequence
  completely synthetic amino acid sequence
XSSXQ

SEQ ID NO: 48 - protein, 6 aa, Artificial Sequence
  completely synthetic amino acid sequence
XXQSSX

SEQ ID NO: 49 - protein, 5 aa, Artificial Sequence
  completely synthetic amino acid sequence
XXQSS

SEQ ID NO: 50 - protein, 6 aa, Artificial Sequence
  completely synthetic amino acid sequence
XSSXQX

SEQ ID NO: 51 - protein, 4 aa, Artificial Sequence
  completely synthetic amino acid sequence
XQSX

SEQ ID NO: 52 - protein, 4 aa, Artificial Sequence
  completely synthetic amino acid sequence
XQSX

SEQ ID NO: 53 - protein, 7 aa, Artificial Sequence
  completely synthetic amino acid sequence
XASXQSL

SEQ ID NO: 54 - protein, 7 aa, Artificial Sequence
  completely synthetic amino acid sequence
XASXQSX